(19) United States
(12) Patent Application Publication
(10) Pub. No.: US 2023/0159509 A1
(43) Pub. Date: May 25, 2023

(54) PYRIDINESULFONAMIDE DERIVATIVES AS TRAP1 MODULATORS AND USES THEREOF

(71) Applicant: Amathus Therapeutics, Inc., North Cambridge, MA (US)

(72) Inventors: Edward Holson, Newton Highlands, MA (US); Charles Blum, Lexington, MA (US)

(73) Assignee: Amathus Therapeutics, Inc., North Cambridge, MA (US)

(21) Appl. No.: 17/912,842

(22) PCT Filed: Mar. 19, 2021
(86) PCT No.: PCT/US21/23174

§ 371 Date: Sep. 19, 2022

Related U.S. Application Data
(60) Provisional application No. 62/992,750, filed on Mar. 20, 2020.

(51) Int. Cl. C07D 413/12; C07D 401/12; C07D 417/12; C07D 213/82; C07D 401/14; C07D 417/14; C07D 471/04; C07D 491/052; C07D 405/12; C07D 495/04; C07D 409/12; C07D 413/14; C07D 495/18; C07D 487/04; C07D 405/14; C07D 513/04; C07D 498/04

(52) U.S. Cl. CPC C07D 413/12; C07D 401/12; C07D 417/12; C07D 213/82; C07D 401/14; C07D 417/14; C07D 471/04; C07D 491/052; C07D 405/12; C07D 495/04; C07D 409/12; C07D 413/14; C07D 495/18; C07D 487/04; C07D 405/14; C07D 513/04; C07D 498/04; C07B 2200/05

(57) ABSTRACT

The present disclosure provides compounds of Formula (I): and pharmaceutically acceptable salts, solvates, hydrates, polymorphs, co-crystals, tautomers, stereoisomers, isotopically labeled compounds, and prodrugs thereof. The provided compounds may be tumor necrosis factor (“TNF”) receptor associated protein 1 (“TRAP1”) modulators (e.g., TRAP1 activators). The provided compounds may also rescue the activity in PTEN-induced kinase 1 (“PINK1”) loss of function contexts. The provided compounds may also improve mitochondrial health, function, quality, quantity, and/or activity, and/or reduce the production of reactive oxygen species. The provided compounds may also refold or solubilize aggregated or misfolded proteins such as a-synuclein. The present disclosure also provides pharmaceutical compositions comprising the provided compounds; kits comprising the provided compounds or pharmaceutical compositions; and methods of using the provided compounds and pharmaceutical compositions (e.g., for treating a disease in a subject in need thereof).

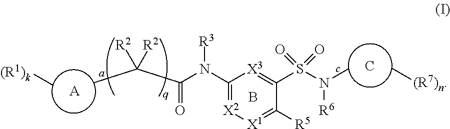

### RELATED APPLICATIONS

This application claims priority to U.S. Provisional Patent Application No. 62/992,750, filed Mar. 20, 2020, which is incorporated herein by reference.

### BACKGROUND

Tumor necrosis factor (“TNF”) receptor associated protein 1 (“TRAP1”) has been reported to be a chaperone responsible for maintaining the mitochondrial quality and energy metabolism found in the mitochondrial matrix (Altieri et al., Biochim Biophys Acta 2012, 1823: 767-73; Rasola et al., Trends Cell Biol., 2014, 24, 455-463). TRAP1 may have a regulatory role in stress sensing in mitochondria allowing cellular adaption to the environment (Fitzgerald et al., Brain 2017: 140; 2444-2459). It has been reported that TRAP1 may be associated with a range of diseases, such as Parkinson's disease (Pridgeon et al., PLoS Biol., 2007, 5, e172; van Ham et al., PLoS Genetics, 2008, Volume 4, Issue 3, e1000027; Costa et al., Cell Death and Disease (2013) 4, e467; Fitzgerald et al., Brain, 2017, 140, 2444-2459; Rai et al., Frontiers in Aging Neuroscience, 2018, Volume 10, Article 221); congenital abnormalities of the kidney and urinary tract (“CAKUT”) and VACTERL association (Saisawat et al., Kidney International (2014) 85, 1310-1317); pain, fatigue and gastrointestinal dysmotility (Boles et al., Mitochondrion 23 (2015) 64-70); severe autoinflammatory disease (Standing et al., Life Science Alliance 3 (2019) e201900376 [p1-12]). There is a need for developing improved therapy of diseases associated with TRAP1.

### SUMMARY OF THE DISCLOSURE

In one aspect, the present disclosure provides a compound of Formula (I):

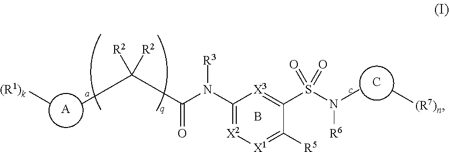

or a pharmaceutically acceptable salt, solvate, hydrate, polymorph, co-crystal, tautomer, stereoisomer, isotopically labeled compound, or prodrug thereof. The provided compounds may be tumor necrosis factor (“TNF”) receptor associated protein 1 (“TRAP1”) modulators (e.g., TRAP1 activators). In certain embodiments, the provided compounds increase the activity (e.g., ATPase activity) of TRAP1. In certain embodiments, the provided compounds increase the expression of TRAP1. TRAP1 has been reported to be a chaperone responsible for maintaining mitochondrial homeostasis and energy metabolism. As a chaperone, TRAP1 may be able to support the folding of proteins (e.g., in an ATP-dependent manner), stabilize proteins, and/or prevent aggregation of proteins (e.g., aggregation of proteins into nonfunctional structures). The provided compounds may also be able to support the folding of proteins (e.g., in an ATP-dependent manner), stabilize proteins, and/or prevent aggregation of proteins (e.g., aggregation of proteins into nonfunctional structures). The provided compounds may also be able to refold and/or solubilize aggregated or misfolded proteins (e.g., α-synuclein). The provided compounds may also be able to reduce the production of reactive oxygen species. The provided compounds may also increase the health, quality, function (e.g., mitochondrial respiration), quantity, and/or activity of mitochondria, and/or rescue dysfunction in mitochondria. The provided compounds may also be able to rescue the activity in PTEN-induced kinase 1 (“PINK1”) loss of function contexts.

Therefore, the provided compounds may be useful in treating or preventing diseases in a subject in need thereof. The disease may be associated with: decreased activity of TRAP1, decreased expression of TRAP1, protein misfolding, protein aggregation, increased production of reactive oxygen species, decreased health of mitochondria, decreased quality of mitochondria, reduced function of mitochondria, decreased quantity of mitochondria, and/or decreased activity of mitochondria. The subject may be a human.

In another aspect, the present disclosure provides pharmaceutical compositions comprising a provided compound and optionally a pharmaceutically acceptable excipient.

In another aspect, the present disclosure provides kits comprising a provided compound or pharmaceutical composition, and instructions for using the provided compound or pharmaceutical composition.

In another aspect, the present disclosure provides methods of increasing the expression and/or activity of TRAP1 in a subject in need thereof, the methods comprising administering to the subject in need thereof an effective amount of a provided compound or pharmaceutical composition.

In another aspect, the present disclosure provides methods of increasing the health, quality, function, quantity, and/or activity of mitochondria in a subject in need thereof, the methods comprising administering to the subject in need thereof an effective amount of a provided compound or pharmaceutical composition.

In another aspect, the present disclosure provides methods of increasing the expression and/or activity of TRAP1 in a cell, tissue, or biological sample, the methods comprising contacting the cell, tissue, or biological sample with an effective amount of a provided compound or pharmaceutical composition.

In another aspect, the present disclosure provides methods of increasing the health, quality, function, quantity, and/or activity of mitochondria in a cell, tissue, or biological sample, the methods comprising contacting the cell, tissue, or biological sample with an effective amount of a provided compound or pharmaceutical composition.

In another aspect, the present disclosure provides methods of treating a disease in a subject in need thereof, the methods comprising administering to the subject in need thereof an effective amount of a provided compound or pharmaceutical composition.

In another aspect, the present disclosure provides methods of preventing a disease in a subject in need thereof, the methods comprising administering to the subject in need thereof an effective amount of a provided compound or pharmaceutical composition.

The details of one or more embodiments of the present disclosure are set forth herein. Other features, objects, and advantages of the present disclosure will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{h} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, a formula depicted herein includes compounds that do not include isotopically enriched atoms and also compounds that include isotopically enriched atoms. Compounds that include isotopically enriched atoms may be useful as, for example, analytical tools, and/or probes in biological assays.

The term “aliphatic” includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups (e.g., halo, such as fluorine). As will be appreciated by one of ordinary skill in the art, “aliphatic” is intended herein to include alkyl, alkenyl, alkynyl, cycloalkyl, cycloalkenyl, and cycloalkynyl moieties.

When a range of values (“range”) is listed, it is intended to encompass each value and sub-range within the range. A range is inclusive of the values at the two ends of the range unless otherwise provided. For example, “an integer between 1 and 4” refers to 1, 2, 3, and 4. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₂ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). The attachment point of alkyl may be a single bond (e.g., as in —CH₃), double bond (e.g., as in ═CH₂), or triple bond (e.g., as in ≡CH). The moieties ═CH₂ and ≡CH are also alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms, one or more (e.g., two, three, or four, as valency permits) carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified

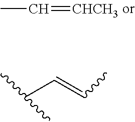

may be in the (E)- or (Z)-configuration.

“Alkynyl” refers to a radical of a straight-chain or branched hydrocarbon group having from 2 to 20 carbon atoms, one or more (e.g., two, three, or four, as valency permits) carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 13 ring carbon atoms (“C₃₋₁₃ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged, or spiro ring system such as a bicyclic system (“bicyclic carbocyclyl”). Carbocyclyl can be saturated, and saturated carbocyclyl is referred to as “cycloalkyl.” In some embodiments, carbocyclyl is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. Carbocyclyl can be partially unsaturated. Carbocyclyl may include zero, one, or more (e.g., two, three, or four, as valency permits) C═C double bonds in all the rings of the carbocyclic ring system that are not aromatic or heteroaromatic. Carbocyclyl including one or more (e.g., two or three, as valency permits) C═C double bonds in the carbocyclic ring is referred to as “cycloalkenyl.” Carbocyclyl including one or more (e.g., two or three, as valency permits) C≡C triple bonds in the carbocyclic ring is referred to as “cycloalkynyl.” “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl is substituted or unsubstituted, 3- to 7-membered, and monocyclic. In certain embodiments, the carbocyclyl is substituted or unsubstituted, 5- to 13-membered, and bicyclic.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. In certain embodiments, the carbocyclyl includes oxo substituted thereon.

“Heterocyclyl” or “heterocyclic” refers to a radical of a 3- to 13-membered non-aromatic ring system having ring carbon atoms and 1 to 4 ring heteroatoms, wherein each heteroatom is independently selected from nitrogen, oxygen, and sulfur (“3-13 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”). A heterocyclyl group can be saturated or can be partially unsaturated. Heterocyclyl may include zero, one, or more (e.g., two, three, or four, as valency permits) double bonds in all the rings of the heterocyclic ring system that are not aromatic or heteroaromatic. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. In certain embodiments, the heterocyclyl is substituted or unsubstituted, 3- to 7-membered, and monocyclic. In certain embodiments, the heterocyclyl is substituted or unsubstituted, 5- to 13-membered, and bicyclic. In certain embodiments, the heterocyclyl includes oxo substituted thereon.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include azirdinyl, oxiranyl, or thiiranyl. Exemplary 4-membered heterocyclyl groups containing one heteroatom include azetidinyl, oxetanyl and thietanyl. Exemplary 5-membered heterocyclyl groups containing one heteroatom include tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include dioxolanyl, oxasulfuranyl, disulfuranyl, and oxazolidin-2-one. Exemplary 5-membered heterocyclyl groups containing three heteroatoms include triazolinyl, oxadiazolinyl, and thiadiazolinyl. Exemplary 6-membered heterocyclyl groups containing one heteroatom include piperidinyl, tetrahydropyranyl, dihydropyridinyl, and thianyl. Exemplary 6-membered heterocyclyl groups containing two heteroatoms include piperazinyl, morpholinyl, dithianyl, and dioxanyl. Exemplary 6-membered heterocyclyl groups containing two heteroatoms include triazinanyl. Exemplary 7-membered heterocyclyl groups containing one heteroatom include azepanyl, oxepanyl and thiepanyl. Exemplary 8-membered heterocyclyl groups containing one heteroatom include azocanyl, oxecanyl, and thiocanyl. Exemplary 5-membered heterocyclyl groups fused to a C₆ aryl ring (also referred to herein as a 5,6-bicyclic heterocyclic ring) include indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In certain embodiments, the heteroaryl group is 5-6 membered, monocyclic. In certain embodiments, the heteroaryl group is 8-14 membered, bicyclic.

Exemplary 5-membered heteroaryl groups containing one heteroatom include pyrrolyl, furanyl and thiophenyl. Exemplary 5-membered heteroaryl groups containing two heteroatoms include imidazolyl, pyrazolyl, oxazolyl, isoxazolyl, thiazolyl, and isothiazolyl. Exemplary 5-membered heteroaryl groups containing three heteroatoms include triazolyl, oxadiazolyl, and thiadiazolyl. Exemplary 5-membered heteroaryl groups containing four heteroatoms include tetrazolyl. Exemplary 6-membered heteroaryl groups containing one heteroatom include pyridinyl. Exemplary 6-membered heteroaryl groups containing two heteroatoms include pyridazinyl, pyrimidinyl, and pyrazinyl. Exemplary 6-membered heteroaryl groups containing three or four heteroatoms include triazinyl and tetrazinyl, respectively. Exemplary 7-membered heteroaryl groups containing one heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R⁹⁹ substituents can be joined to form ═O or ═S; wherein X⁻ is a counterion.

In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, the carbon atom substituents are independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻,B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and Rad are as defined above.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

In certain embodiments, the substituent present on a nitrogen atom is a nitrogen protecting group (also referred to as an amino protecting group). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, a nitrogen protecting group is Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the oxygen atom substituents are independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl, TBDPS, TBDMS, TIPS, TES, TMS, MOM, THP, t-Bu, Bn, allyl, acetyl, pivaloyl, or benzoyl.

In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group. In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the sulfur atom substituents are independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). Sulfur protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

The “molecular weight” of —R, wherein —R is any monovalent moiety, is calculated by subtracting the atomic weight of a hydrogen atom from the molecular weight of the molecule R—H. The “molecular weight” of -L-, wherein -L- is any divalent moiety, is calculated by subtracting the combined atomic weight of two hydrogen atoms from the molecular weight of the molecule H-L-H.

In certain embodiments, the molecular weight of a substituent is lower than 200, lower than 150, lower than 100, lower than 50, or lower than 25 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, and/or iodine atoms. In certain embodiments, a substituent consists of carbon, hydrogen, and/or fluorine atoms. In certain embodiments, a substituent does not comprise one or more, two or more, or three or more hydrogen bond donors. In certain embodiments, a substituent does not comprise one or more, two or more, or three or more hydrogen bond acceptors.

These and other exemplary substituents are described in more detail in the Detailed Description, Examples, Figures, and Claims. The present disclosure is not intended to be limited in any manner by the above exemplary listing of substituents.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The provided compounds may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.xH₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R.0.5H₂O)), and polyhydrates (x is a number greater than 1, e.g., dihydrates (R.2H₂O) and hexahydrates (R.6H₂O)).

The term “tautomers” refer to compounds that are interchangeable forms of a particular compound structure, and that vary in the displacement of hydrogen atoms and electrons. Thus, two structures may be in equilibrium through the movement of 7 electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refer to compounds, including derivatives of the provided compounds, which have cleavable groups and become by solvolysis or under physiological conditions the provided compounds which are pharmaceutically active in vivo. Such examples include, but are not limited to, ester derivatives and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. C₁ to C₈ alkyl, C₂-C₈ alkenyl, C₂-C₈ alkynyl, aryl, C₇-C₁₂ substituted aryl, and C₇-C₁₂ arylalkyl esters of the provided compounds may be preferred.

“TRAP1,” or “tumor necrosis factor (‘TNF’) receptor associated protein 1,” is also known as HSP75 and is a protein encoded by the TRAP1 gene. In humans, the Ensembl of the TRAP1 gene is ENSG00000126602. See, e.g., Song et al., The Journal Of Biological Chemistry, 1995, Vol. 270, No. 8, pp. 3574-3581; Felts et al., The Journal Of Biological Chemistry, 2000, Vol. 275, No. 5, pp. 3305-3312.

“PINK1,” or “PTEN-induced kinase 1,” is a mitochondrial serine/threonine-protein kinase encoded by the PINK1 gene. In humans, the Ensembl of the PINK1 gene is ENSG00000158828. See, e.g., Unoki et al., Oncogene, 2001, 20(33):4457-65; Valente et al., Ann. Neurol., 2004, 56(3):336-41.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the subject is a mammal. The subject may be a male or female and at any stage of development. A non-human animal may be a transgenic animal.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “administer,” “administering,” or “administration,” refers to implanting, absorbing, ingesting, injecting, inhaling, or otherwise introducing a compound, or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

The terms “condition,” “disease,” and “disorder” are used interchangeably.

An “effective amount” of a provided compound refers to an amount sufficient to elicit the desired biological response, i.e., treating the condition. As will be appreciated by those of ordinary skill in this art, the effective amount of a provided compound may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of a compound may reduce the tumor burden or stop the growth or spread of a tumor.

A “therapeutically effective amount” of a provided compound is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a provided compound is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher's disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. The cancer may be a solid tumor. The cancer may be a hematological malignancy. Exemplary cancers include acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, dermatitis (e.g., stasis dermatitis, allergic contact dermatitis, atopic dermatitis, irritant contact dermatitis, neurodermatitis perioral dermatitis, seborrheic dermatitis), hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, necrotizing enterocolitis, inflammatory rosacea. An ocular inflammatory disease includes post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including Alzheimer's disease, Parkinson's disease, amyotrophic lateral sclerosis, tauopathies (including frontotemporal dementia), and Huntington's disease. Examples of neurological diseases include headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawl symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “psychiatric disease” refers to a disease of the mind and includes diseases and disorders listed in the Diagnostic and Statistical Manual of Mental Disorders-Fourth Edition (DSM-IV), published by the American Psychiatric Association, Washington D. C. (1994). Psychiatric diseases include anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “metabolic disease” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include the endocrine (hormonal) control system (e.g., the insulin pathway, the enteroendocrine hormones including GLP-1, PYY or the like), the neural control system (e.g., GLP-1 in the brain), or the like. Examples of metabolic disorders include diabetes (e.g., Type I diabetes, Type II diabetes, gestational diabetes), hyperglycemia, hyperinsulinemia, insulin resistance, and obesity.

### BRIEF DESCRIPTION OF THE DRAWINGS

The accompanying drawings, which constitute a part of this specification, illustrate several embodiments of the invention and together with the description, serve to explain the principles of the invention. Unless otherwise provided, “uM” refers to “μM”, and “Cmp” refers to “Compound”.

FIG. 1 shows survival of rat TH dopaminergic neurons injured with MPP⁺ (4 μM, 4 h), with and without application of compound 1178.

FIG. 2 shows ROS production in primary rat dopaminergic neurons after an injury with MPP⁺ (4 μM, 4 h), and reduction of reactive oxygen species (ROS) in presence of compound 1178.

FIG. 3 shows ROS production in rat primary mesencephalic culture after an injury with MPP⁺ (4 μM, 4 h), and reduction ROS in the presence of compound 1178.

FIG. 4 shows neurite network of primary rat TH+ dopaminergic neurons injured with MPP⁺ (4 μM, 24 h), and protection of the neurite network in presence of compound 1178.

FIG. 5 shows neurite network of primary rat TH+ dopaminergic neurons injured with MPP⁺ (4 μM, 48 h) and protection of neurite network with compound 1178.

FIG. 6 shows aSyn aggregation in primary rat TH+ neurons injured with MPP+(4 μM, 48 h) and response from compound 1178.

FIG. 7 shows survival of primary rat TH+ dopaminergic neurons injured with 6-OHDA (20 μM, 4 h), with and without application of compound 1178.

FIG. 8 shows ROS production in primary rat dopaminergic neurons after an injury with 6-OHDA (20 μM, 4 h), and reduction in ROS in presence of compound 1178.

FIG. 9 shows a reduction in α-synuclein aggregation in human embryonic stem cells overexpressing α-synuclein with an AAV vector with treatment with compound 1178.

FIG. 10 shows the activation of TRAP1 (Eₘₐₓ and EC₅₀) in the ADP-Glo biochemical assay by compound 1178.

FIG. 11 shows the effect of compound 1178 in Hsp90-β ADP-Glo biochemical assay.

FIG. 12 shows the effect of compound 1178 in Grp94 ADP-Glo biochemical assay.

FIG. 13 shows titration of fluorescent nanoBRET tracer molecule (probe-385) with nanoLuc TRAP1 in SHSY5Y cells and displacement of the tracer with compound 1178 (20 PM).

FIG. 14 shows a concentration response curve of compound 1178 displacing the nanoBRET tracer molecule (probe-385, 1 μM) from nanoLuc TRAP1 in live SHSY5Y cells.

FIG. 15 shows brain and plasma concentration time curves following a 30 mg/kg IP dose of compound 2116 in rats.

FIG. 16 shows effect of compound 1206 in preventing cyst formation in a Pkd 1 KO mouse cell derived cyst forming assay.

FIGS. 17A to 17C show effects of compound 1206 in a mouse model of polycystic kidney disease (Tam=tamoxifen). FIG. 17A shows the 2 KW/BW results (KW=kidney weight; BW=body weight). FIG. 17B shows the BUN (blood urea nitrogen) results. FIG. 17C shows the cystic index results.

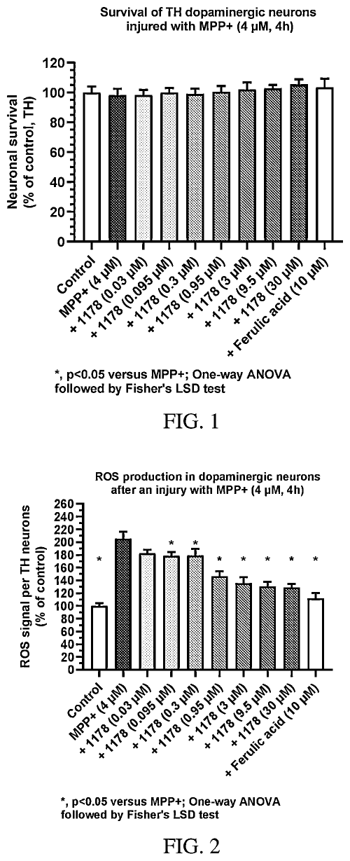

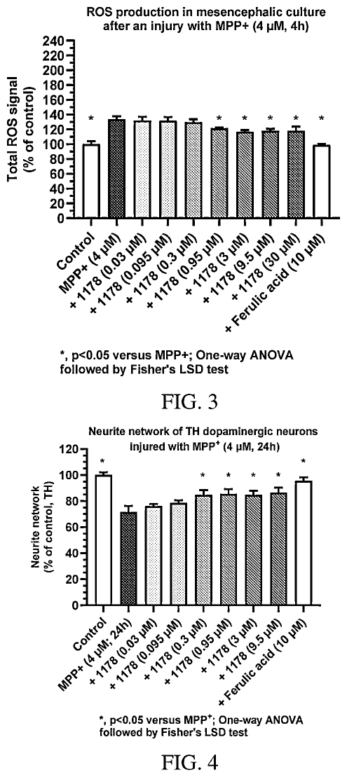

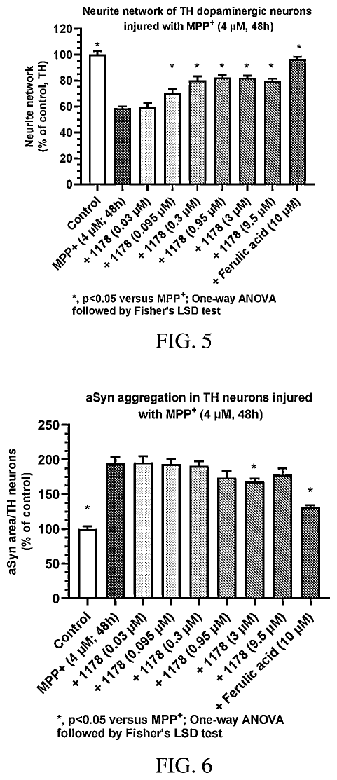

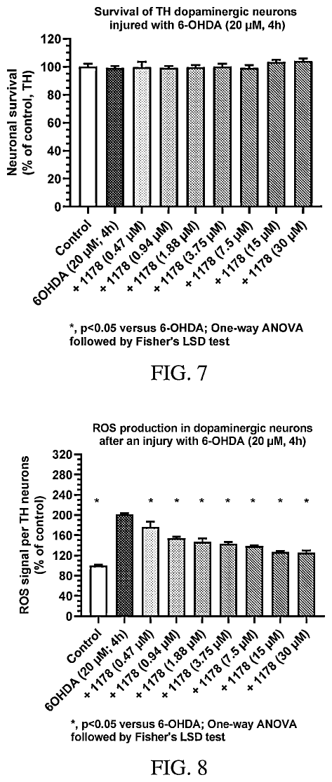

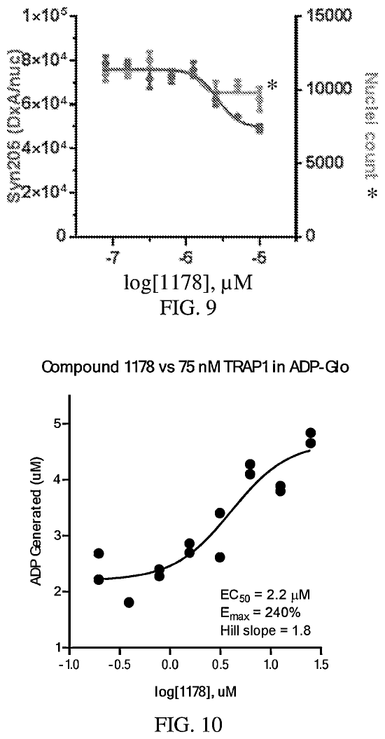

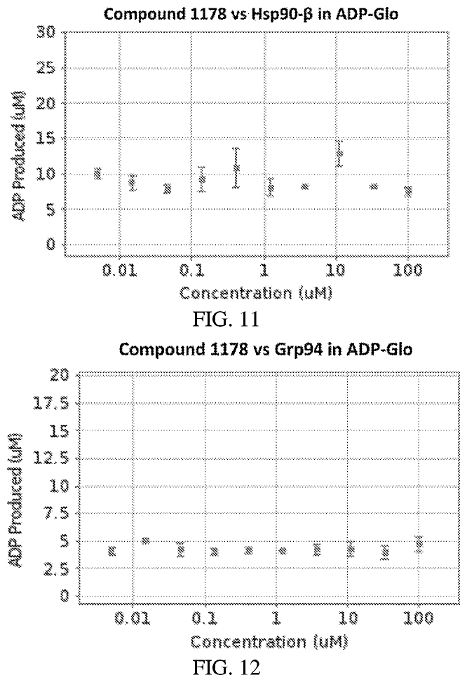

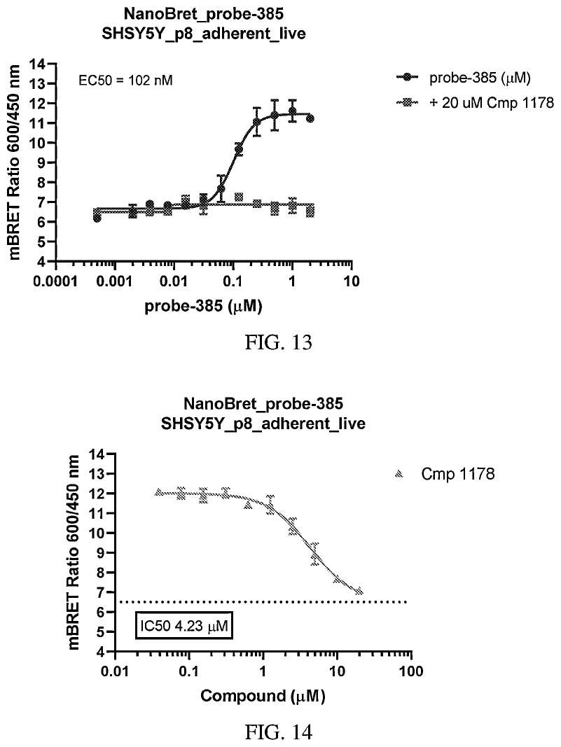

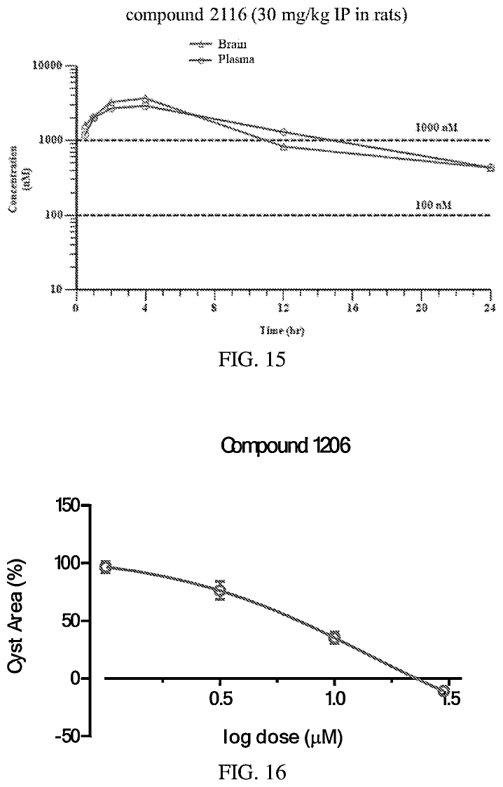

### DETAILED DESCRIPTION OF CERTAIN EMBODIMENTS OF THE DISCLOSURE

In one aspect, the present disclosure provides compounds of Formula (I), and pharmaceutically acceptable salts, solvates, hydrates, polymorphs, co-crystals, tautomers, stereoisomers, isotopically labeled compounds, and prodrugs thereof.

In other aspects, the present disclosure provides pharmaceutical compositions comprising a provided compound; kits comprising a provided pharmaceutical composition or compound; and methods of using the provided compounds, pharmaceutical compositions, and kits.

### Compounds

In one aspect, the present disclosure provides a compound of Formula (I):

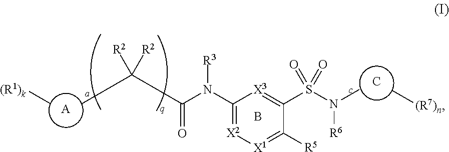

or a pharmaceutically acceptable salt, solvate, hydrate, polymorph, co-crystal, tautomer, stereoisomer, isotopically labeled compound, or prodrug thereof, wherein:

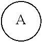

is aryl or heteroaryl;

each [symbol] is independently a single or double bond, as valency permits;

when attached to a carbon atom, each R¹ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)₂R^{a}, —NR^{a}S(═O)₂OR^{a}, —NR^{a}S(═O)₂N(R^{a})₂, —OS(═O)₂R^{a}, —OS(═O)₂OR^{a}, —OS(═O)₂N(R^{a})₂, —S(═O)₂R^{a}, —S(═O)₂OR^{a}, —S(═O)₂N(R^{a})₂, —P(═O)(R^{a})₂, or ═O, as valency permits;

when attached to a nitrogen atom, each R¹ is independently substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —S(═O)₂R^{a}, —S(═O)₂OR^{a}, —S(═O)₂N(R^{a})₂, —P(═O)(R^{a})₂, a nitrogen protecting group, or ═O, as valency permits;

or one R¹ and R³ are joined with their intervening atoms to form substituted or unsubstituted heterocyclyl;

each R^{a} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on a nitrogen atom are joined with the nitrogen atom to form substituted or unsubstituted heterocyclyl or substituted or unsubstituted heteroaryl;

k is 0 or an integer between 1 and 13, inclusive, as valency permits;

each R² is independently hydrogen, halogen, or substituted or unsubstituted alkyl;

q is 0 or 1;

R³ is hydrogen, substituted or unsubstituted alkyl, or a nitrogen protecting group;

one of X¹, X², and X³ is N or N(O);

each of the other two of X¹, X², and X³ is CR⁴;

each R⁴ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)₂R^{a}, —NR^{a}S(═O)₂OR^{a}, —NR^{a}S(═O)₂N(R^{a})₂, —OS(═O)₂R^{a}, —OS(═O)₂OR^{a}, —OS(═O)₂N(R^{a})₂, —S(═O)₂R^{a}, —S(═O)₂OR^{a}, or —S(═O)₂N(R^{a})₂;

R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)₂R^{a}, —NR^{a}S(═O)₂OR^{a}, —NR^{a}S(═O)₂N(R^{a})₂, —OS(═O)₂R^{a}, —OS(═O)₂OR^{a}, —OS(═O)₂N(R^{a})₂, —S(═O)₂R^{a}, —S(═O)₂OR^{a}, or —S(═O)₂N(R^{a})₂;

R⁶ is hydrogen, substituted or unsubstituted alkyl, or a nitrogen protecting group;

or R⁶ and one R⁷ are joined with their intervening atoms to form substituted or unsubstituted heterocyclyl;

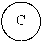

is aryl or heteroaryl;

when attached to a carbon atom, each R⁷ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)₂R^{a}, —NR^{a}S(═O)₂OR^{a}, —NR^{a}S(═O)₂N(R^{a})₂, —OS(═O)₂R^{a}, —OS(═O)₂OR^{a}, —OS(═O)₂N(R^{a})₂, —S(═O)₂R^{a}, —S(═O)₂OR^{a}, —S(═O)₂N(R^{a})₂, —P(═O)(R^{a})₂, or ═O, as valency permits;

when attached to a nitrogen atom, each R⁷ is independently substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —S(═O)₂R^{a}, —S(═O)₂OR^{a}, —S(═O)₂N(R^{a})₂, —P(═O)(R^{a})₂, a nitrogen protecting group, or ═O, as valency permits; and

n is 0 or an integer between 1 and 13, inclusive, as valency permits;

provided that the compound is not of the formula:

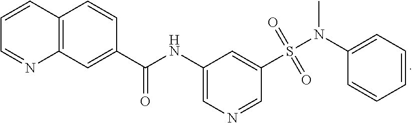 [SMILES: CN(c1ccccc1)S(=O)(=O)c1cncc(NC(=O)c2ccc3cccnc3c2)c1]

When Formula (I) includes two or more instances of a moiety, unless otherwise provided, any two instances of the moiety may be the same or different from each other.

In certain embodiments,

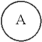

(“Ring A”) is aryl. In certain embodiments, Ring A is phenyl. In certain embodiments,

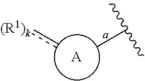

is unsubstituted phenyl. In certain embodiments,

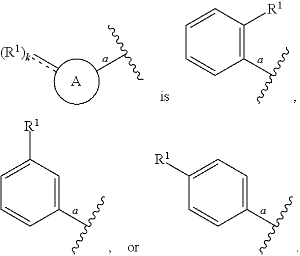

In certain embodiments,

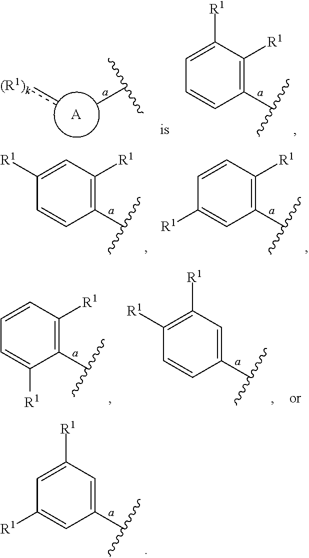

In certain embodiments,

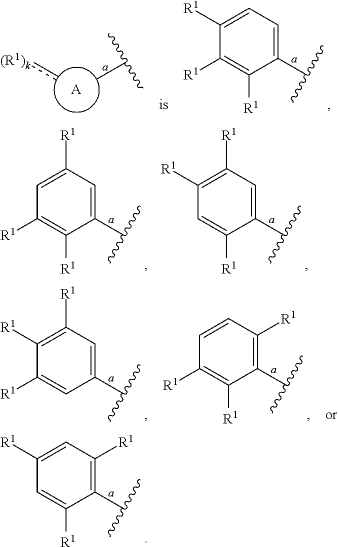

In certain embodiments, Ring A is heteroaryl. In certain embodiments, Ring A is 5- or 6-membered monocyclic heteroaryl. In certain embodiments, Ring A is furanyl, thienyl, pyrrolyl, imidazolyl, oxazolyl, isoxazolyl, thiazolyl, isothiazolyl, or

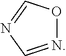

In certain embodiments,

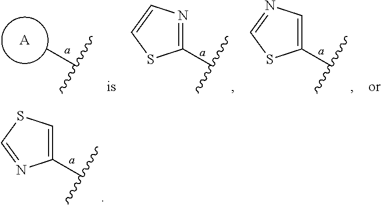

In certain embodiments,

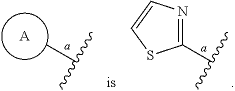

In certain embodiments,

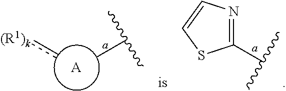

In certain embodiments,

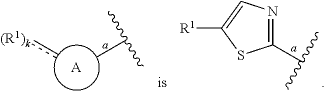

In certain embodiments,

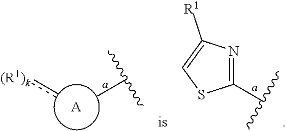

In certain embodiments

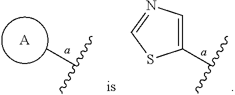

In certain embodiments,

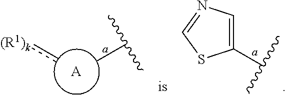

In certain embodiments,

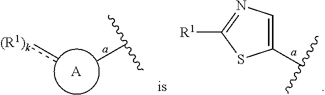

In certain embodiments,

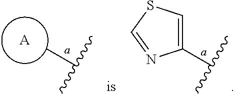

In certain embodiments,

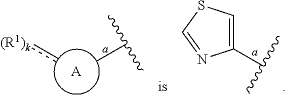

In certain embodiments,

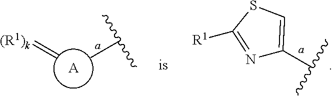

In certain embodiments,

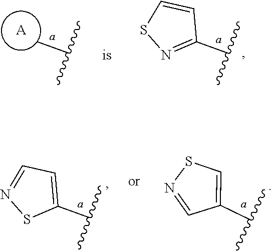

In certain embodiments,

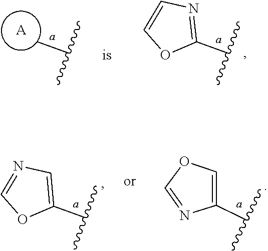

In certain embodiments,

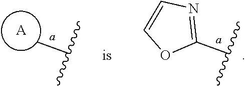

In certain embodiments,

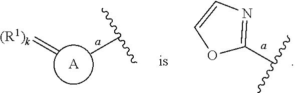

In certain embodiments,

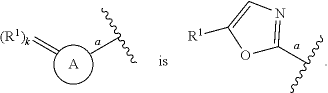

In certain embodiments,

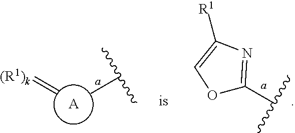

In certain embodiments,

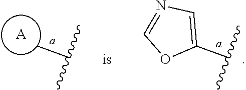

In certain embodiments,

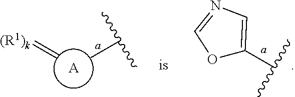

In certain embodiments,

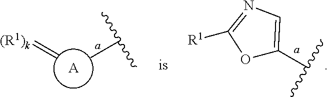

In certain embodiments,

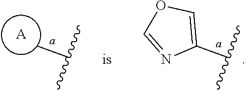

In certain embodiments,

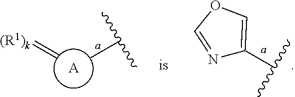

In certain embodiments,

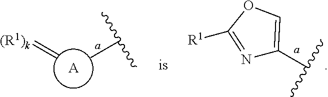

In certain embodiments,

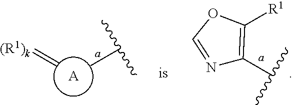

In certain embodiments,

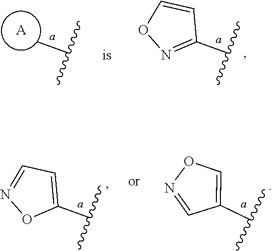

In certain embodiments,

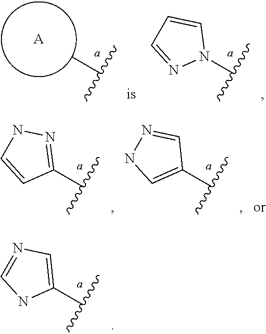

In certain embodiments,

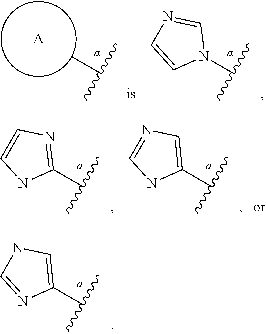

In certain embodiments,

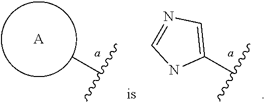

In certain embodiments,

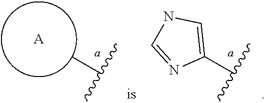

In certain embodiments,

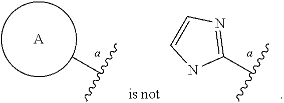

In certain embodiments,

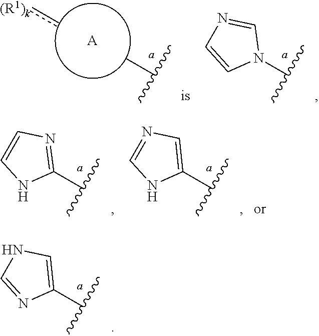

In certain embodiments,

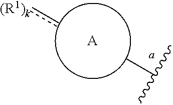

is not

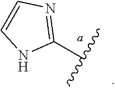 [SMILES: *c1ncc[nH]1]

In certain embodiments,

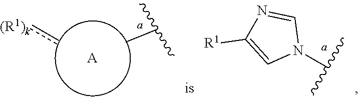

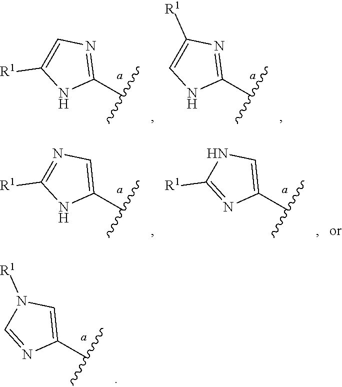

In certain embodiments,

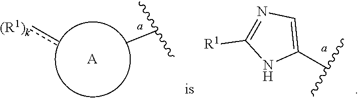

In certain embodiments,

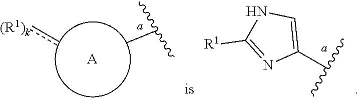

In certain embodiments,

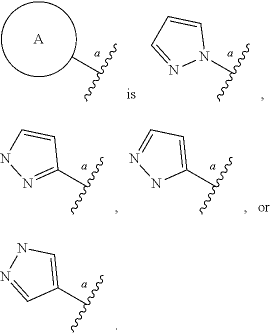

In certain embodiments,

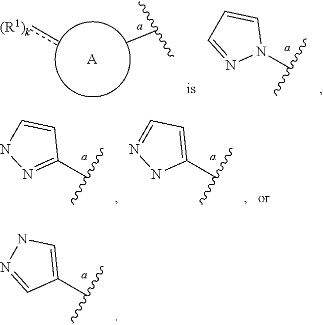

In certain embodiments

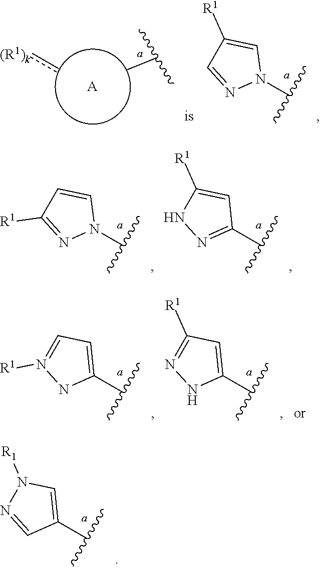

In certain embodiments,

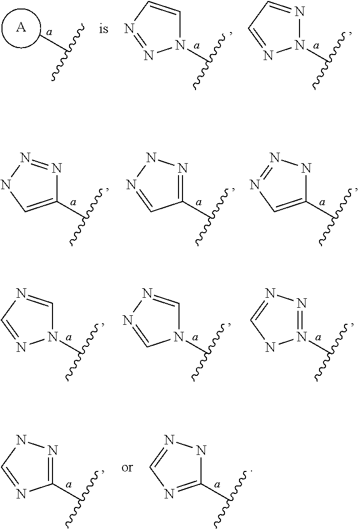

In certain embodiments,

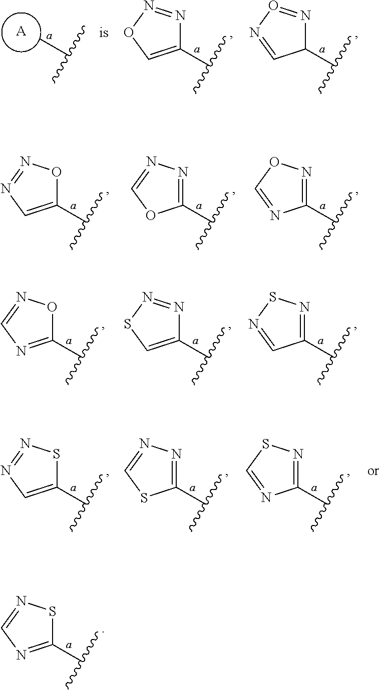

In certain embodiments,

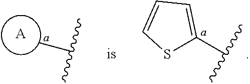

In certain embodiments,

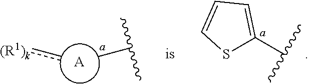

In certain embodiments,

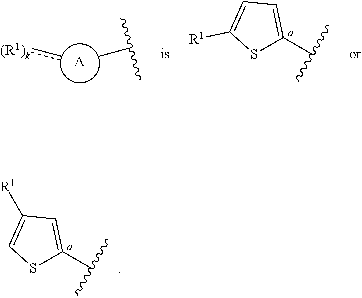

In certain embodiments,

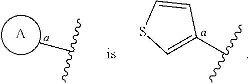

In certain embodiments,

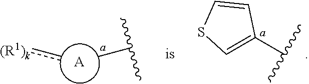

In certain embodiments,

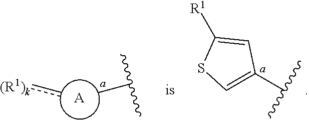

In certain embodiments,

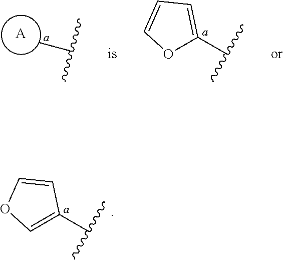

In certain embodiments,

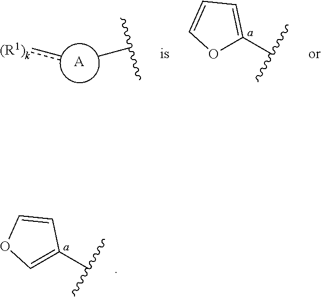

In certain embodiments,

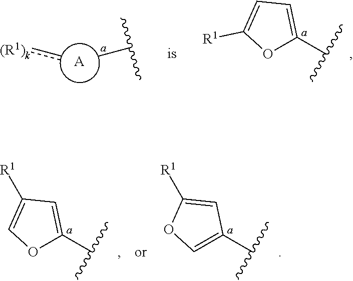

In certain embodiments,

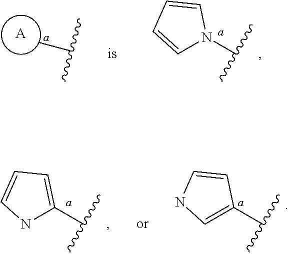

In certain embodiments,

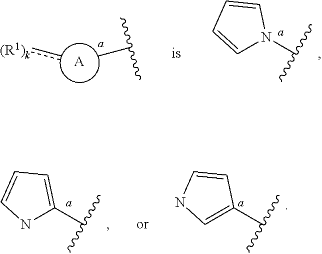

In certain embodiments,

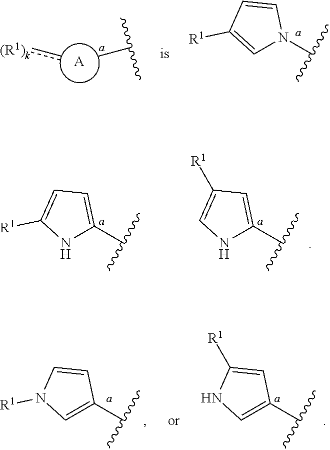

In certain embodiments,

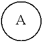

is pyridinyl, pyrazinyl, pyrimidinyl, or pyridazinyl. In certain embodiments,

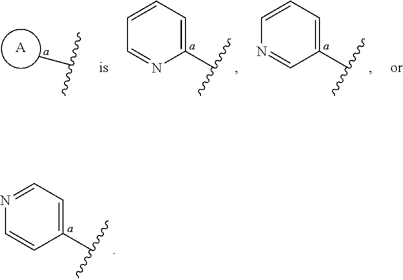

In certain embodiments,

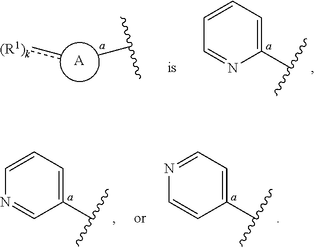

In certain embodiments,

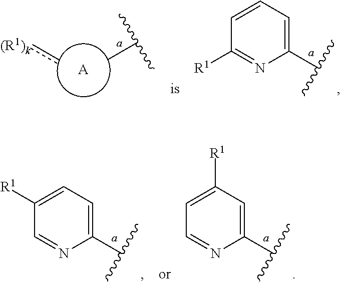

In certain embodiments,

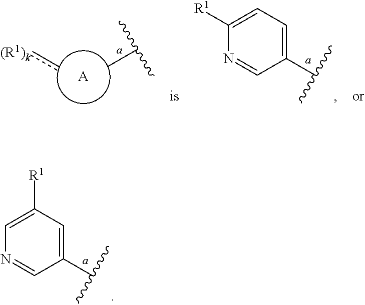

In certain embodiments,

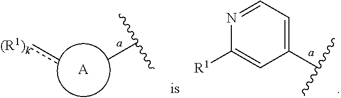

In certain embodiments,

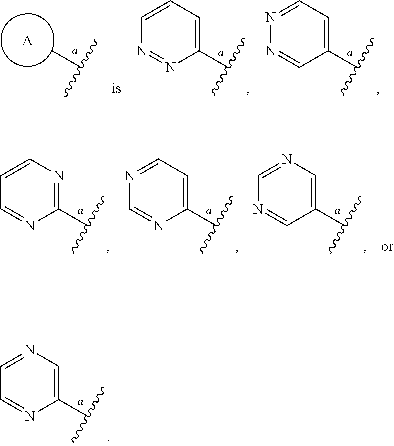

In certain embodiments, Ring A is phenyl fused with monocyclic or bicyclic, carbocyclyl, heterocyclyl, aryl, or heteroaryl, wherein bond a is attached to the phenyl; or 5- or 6-membered monocyclic heteroaryl fused with monocyclic or bicyclic, carbocyclyl, heterocyclyl, aryl, or heteroaryl, wherein bond a is attached to the 5- or 6-membered monocyclic heteroaryl. In certain embodiments, Ring A is phenyl fused with monocyclic or bicyclic, carbocyclyl, heterocyclyl, aryl, or heteroaryl, wherein bond a is attached to the phenyl. In certain embodiments, Ring A is naphthyl. In certain embodiments, Ring A is phenyl fused with naphthyl, wherein bond a is attached to the phenyl. In certain embodiments, Ring A is phenyl fused with 5- to 6-membered, monocyclic heteroaryl, wherein bond a is attached to the phenyl. In certain embodiments, Ring A is phenyl fused with 8-14 membered, bicyclic heteroaryl, wherein bond a is attached to the phenyl. In certain embodiments, Ring A is phenyl fused with 4- to 7-membered, monocyclic carbocyclyl, wherein bond a is attached to the phenyl. In certain embodiments, Ring A is phenyl fused with 6- to 13-membered, bicyclic carbocyclyl, wherein bond a is attached to the phenyl. In certain embodiments, Ring A is phenyl fused with 4- to 7-membered, monocyclic heterocyclyl, wherein bond a is attached to the phenyl. In certain embodiments, Ring A is phenyl fused with 6- to 13-membered, bicyclic heterocyclyl, wherein bond a is attached to the phenyl.

In certain embodiments, Ring A is 5- or 6-membered monocyclic heteroaryl fused with monocyclic or bicyclic, carbocyclyl, heterocyclyl, aryl, or heteroaryl, wherein bond a is attached to the 5- or 6-membered monocyclic heteroaryl. In certain embodiments, Ring A is 5- or 6-membered monocyclic heteroaryl fused with phenyl, wherein bond a is attached to the 5- or 6-membered monocyclic heteroaryl. In certain embodiments, Ring A is 5- or 6-membered monocyclic heteroaryl fused with naphthyl, wherein bond a is attached to the 5- or 6-membered monocyclic heteroaryl. In certain embodiments, Ring A is 5- or 6-membered monocyclic heteroaryl fused with another 5- to 6-membered, monocyclic heteroaryl. In certain embodiments, Ring A is 5- or 6-membered monocyclic heteroaryl fused with 8-14 membered, bicyclic heteroaryl, wherein bond a is attached to the 5- or 6-membered monocyclic heteroaryl. In certain embodiments, Ring A is 5- or 6-membered monocyclic heteroaryl fused with 4- to 7-membered, monocyclic carbocyclyl, wherein bond a is attached to the 5- or 6-membered monocyclic heteroaryl. In certain embodiments, Ring A is 5- or 6-membered monocyclic heteroaryl fused with 6- to 13-membered, bicyclic carbocyclyl, wherein bond a is attached to the 5- or 6-membered monocyclic heteroaryl. In certain embodiments, Ring A is 5- or 6-membered monocyclic heteroaryl fused with 4- to 7-membered, monocyclic heterocyclyl, wherein bond a is attached to the 5- or 6-membered monocyclic heteroaryl. In certain embodiments, Ring A is 5- or 6-membered monocyclic heteroaryl fused with 6- to 13-membered, bicyclic heterocyclyl, wherein bond a is attached to the 5- or 6-membered monocyclic heteroaryl.

In certain embodiments,

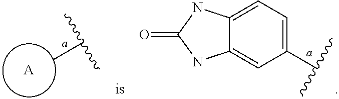

In certain embodiments,

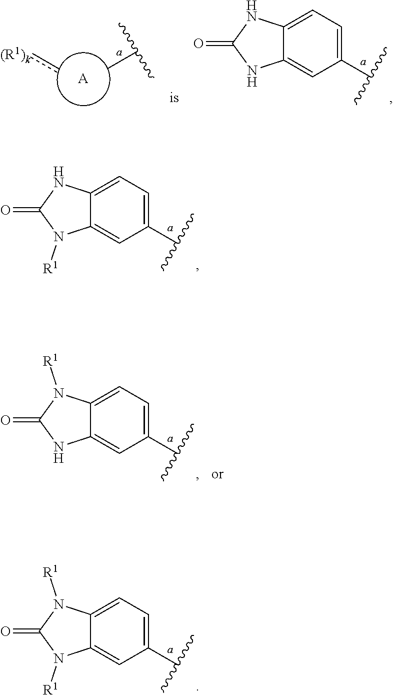

In certain embodiments,

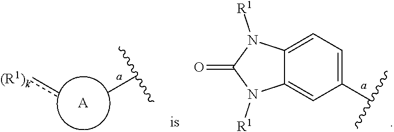

In certain embodiments,

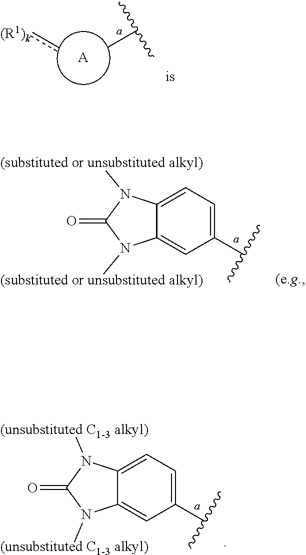

In certain embodiments,

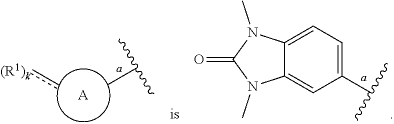

In certain embodiments,

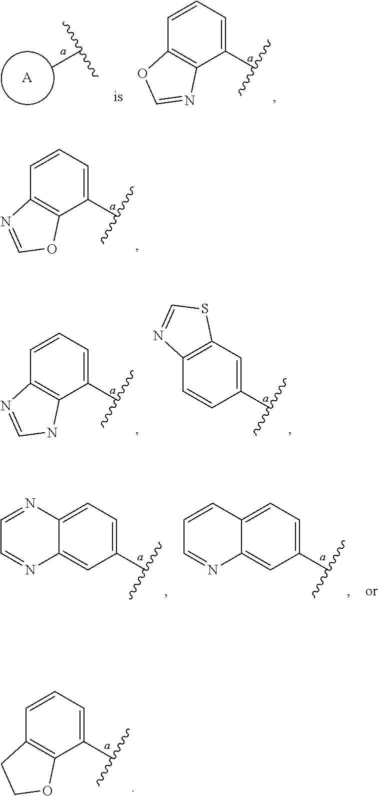

In certain embodiments,

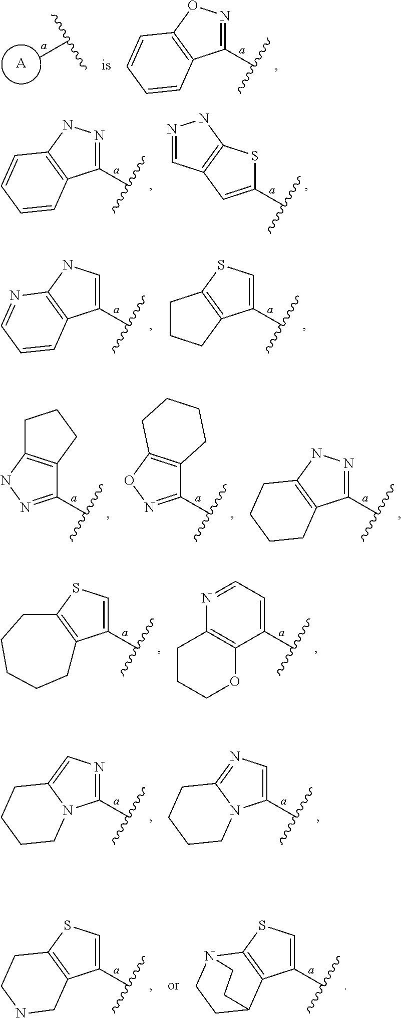

In certain embodiments,

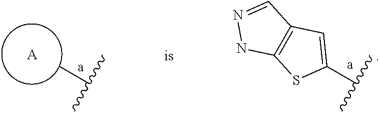

In certain embodiments,

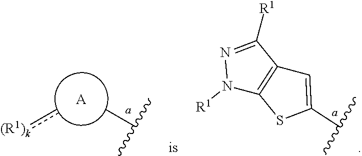

In certain embodiments,

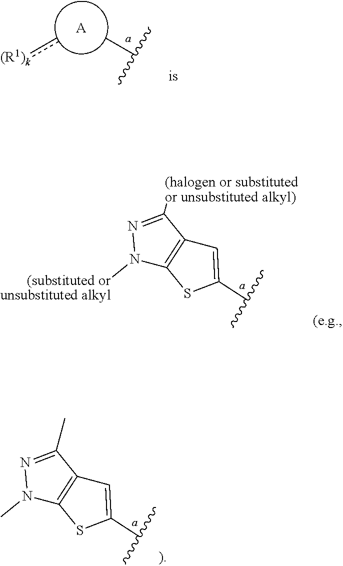

In certain embodiments,

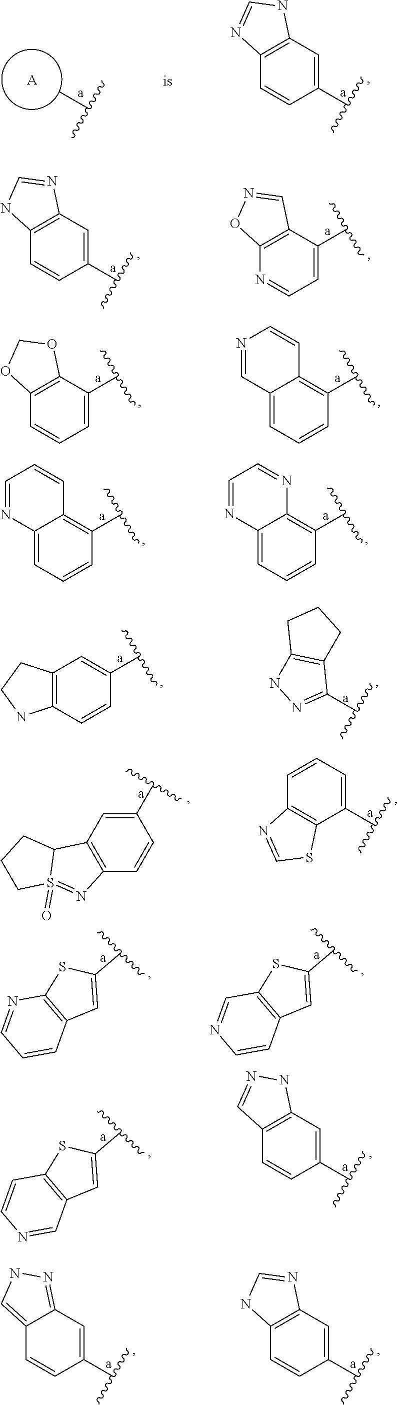

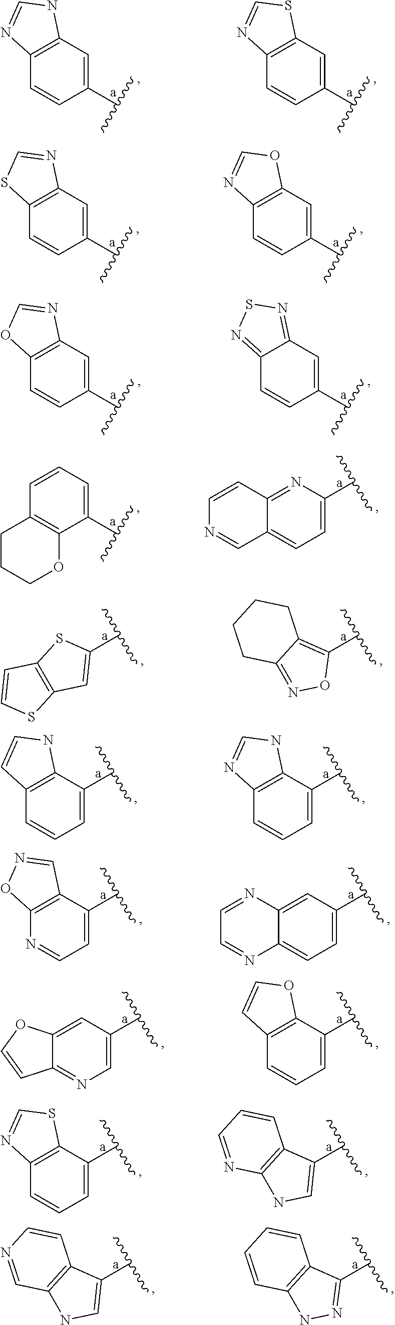

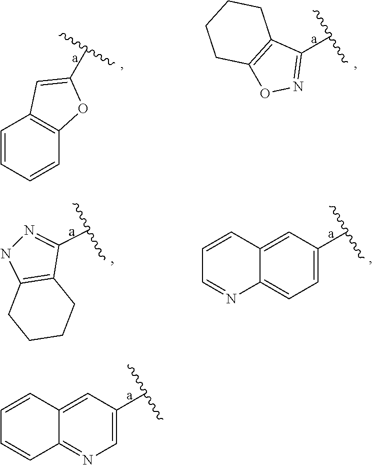 [SMILES: *c1cc2ccccc2o1 ; *c1noc2c1CCCC2 ; *c1n[nH]c2c1CCCC2 ; *c1ccc2ncccc2c1 ; *c1cnc2ccccc2c1]

In certain embodiments,

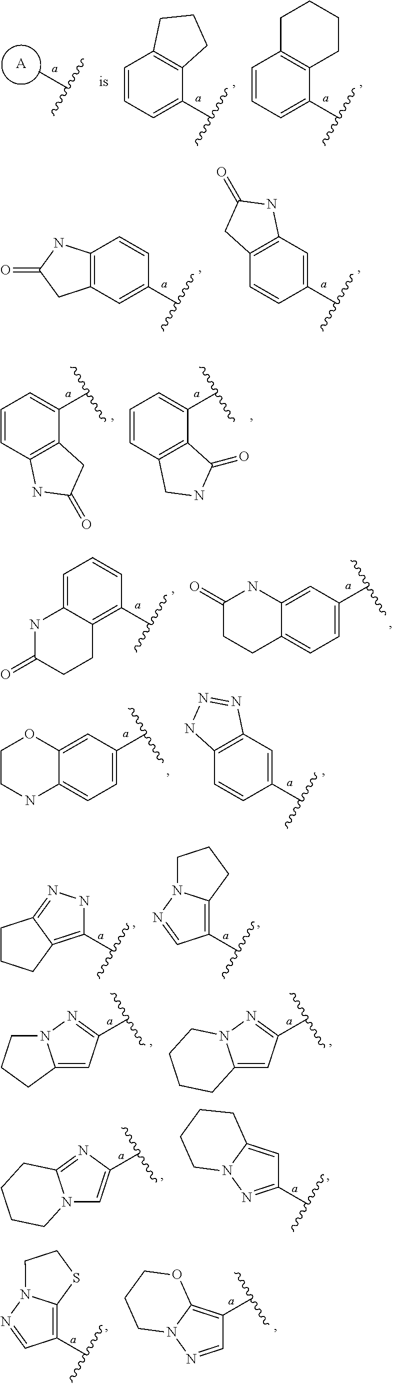

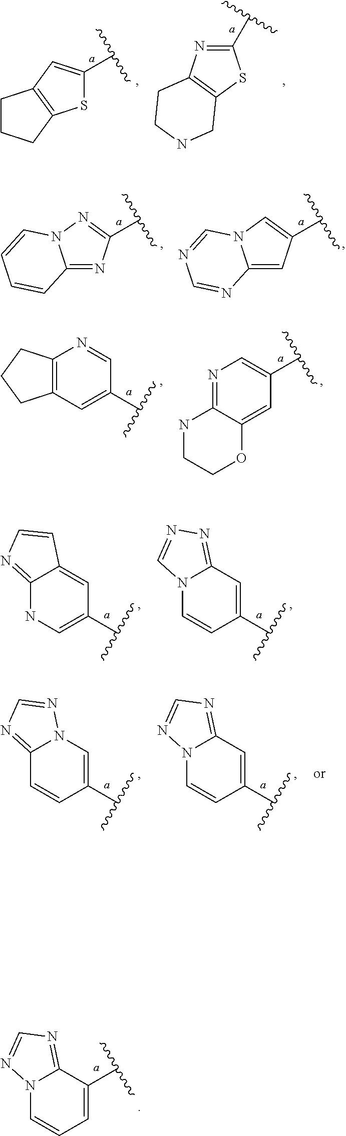

In certain embodiments,

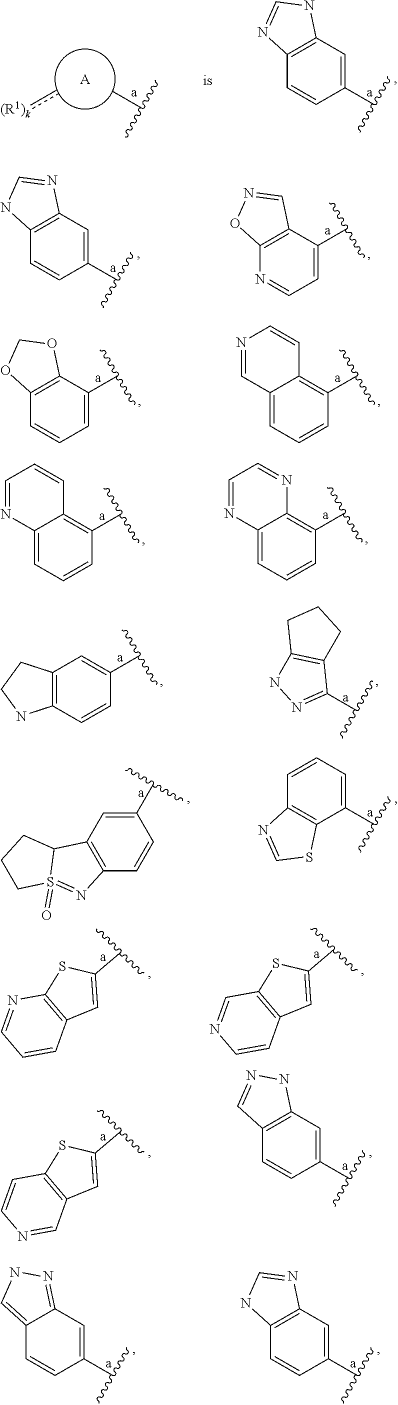

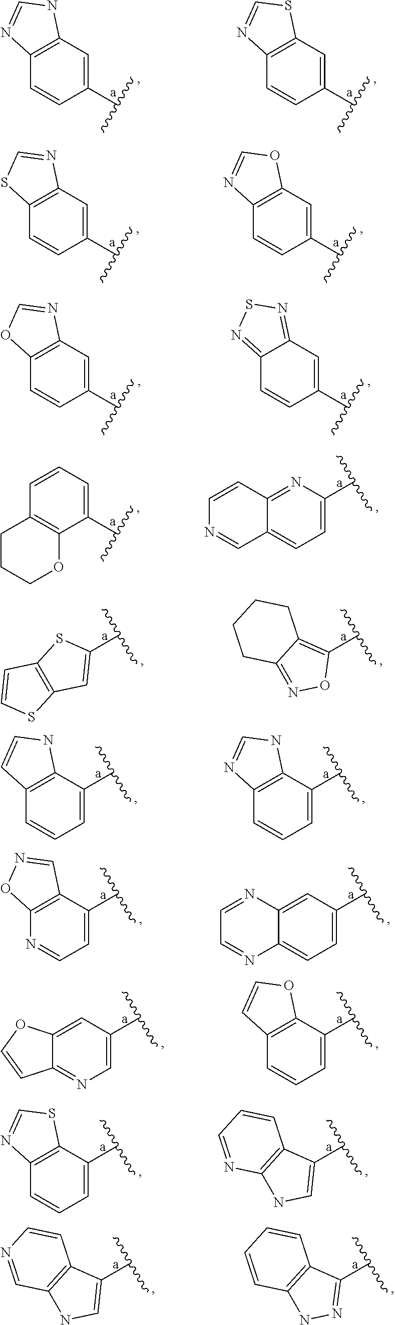

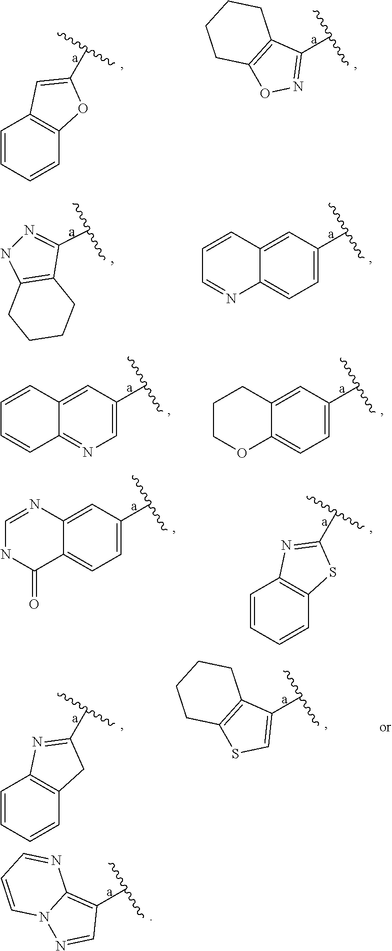

In certain embodiments, the molecular weight of each R¹ is less than 200 g/mol. In certain embodiments, the molecular weight of each R¹ is less than 150 g/mol. In certain embodiments, the molecular weight of each R¹ is less than 100 g/mol.

This paragraph applies when R¹ is attached to a carbon atom. In certain embodiments, at least one instance of R¹ is halogen (e.g., F, Cl, or Br). In certain embodiments, at least one instance of R¹ is unsubstituted alkyl (e.g., unsubstituted C₁₋₆ alkyl). In certain embodiments, at least one instance of R¹ is Me. In certain embodiments, at least one instance of R¹ is Et, Pr, or Bu. In certain embodiments, at least one instance of R¹ is substituted alkyl (e.g., alkyl substituted with one or more instances of halogen (e.g., F)). In certain embodiments, at least one instance of R¹ is substituted C₁₋₆ alkyl. In certain embodiments, at least one instance of R¹ is substituted methyl (e.g., fluorinated methyl or Bn). In certain embodiments, at least one instance of R¹ is substituted ethyl, substituted propyl, or substituted butyl. In certain embodiments, at least one instance of R¹ is substituted or unsubstituted alkenyl. In certain embodiments, at least one instance of R¹ is substituted or unsubstituted, C₂₋₆ alkenyl (e.g., substituted or unsubstituted vinyl or substituted or unsubstituted allyl). In certain embodiments, at least one instance of R¹ is substituted or unsubstituted alkynyl. In certain embodiments, at least one instance of R¹ is substituted or unsubstituted, C₂₋₆ alkynyl (e.g., substituted or unsubstituted ethynyl). In certain embodiments, at least one instance of R¹ is substituted or unsubstituted carbocyclyl (e.g., substituted or unsubstituted, monocyclic, 3- to 7-membered carbocyclyl). In certain embodiments, at least one instance of R¹ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R¹ is substituted or unsubstituted heterocyclyl (e.g., substituted or unsubstituted, 3- to 7-membered, monocyclic heterocyclyl). In certain embodiments, at least one instance of R¹ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R¹ is substituted or unsubstituted aryl. In certain embodiments, at least one instance of R¹ is substituted or unsubstituted phenyl. In certain embodiments, at least one instance of R¹ is unsubstituted phenyl. In certain embodiments, at least one instance of R¹ is substituted or unsubstituted naphthyl. In certain embodiments, at least one instance of R¹ is substituted or unsubstituted heteroaryl. In certain embodiments, at least one instance of R¹ is substituted or unsubstituted, 5- to 6-membered, monocyclic heteroaryl. In certain embodiments, at least one instance of R¹ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R¹ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R¹ is substituted or unsubstituted, 9- to 10-membered, bicyclic heteroaryl. In certain embodiments, at least one instance of R¹ is —OR^{a} (e.g., —OH, —O(substituted or unsubstituted, C₁₋₆ alkyl) (e.g., —OMe, —OCF₃, -OEt, —OPr, -OBu, or -OBn), or —O(substituted or unsubstituted phenyl) (e.g., —OPh)). In certain embodiments, at least one instance of R¹ is —O(substituted or unsubstituted alkyl). In certain embodiments, at least one instance of R¹ is —O(alkyl substituted with at least one —OH). In certain embodiments, at least one instance of R¹ is —OCH₂CH(OH)CH₂OH. In certain embodiments, at least one instance of R¹ is —O(unsubstituted alkyl). In certain embodiments, at least one instance of R¹ is —OMe. In certain embodiments, at least one instance of R¹ is —SR^{a}(e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, at least one instance of R¹ is —N(R^{a})₂ (e.g., —NH₂, —NH (substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, at least one instance of R¹ is —CN or —SCN. In certain embodiments, at least one instance of R¹ is —NO₂. In certain embodiments, at least one instance of R¹ is —C(═NR^{a})R^{a}, —C(═NR^{a})OR^{a}, or —C(═NR^{a})N(R^{a})₂. In certain embodiments, at least one instance of R¹ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R¹ is —C(═O)OR^{a} (e.g., —C(═O)OH, —C(═O)O (substituted or unsubstituted alkyl) (e.g., —C(═O)OMe), or —C(═O)O (substituted or unsubstituted phenyl)). In certain embodiments, at least one instance of R¹ is —C(═O)N(R^{a})₂ (e.g., —C(═O)NH₂, —C(═O)NH (substituted or unsubstituted alkyl) (e.g., —C(═O)NHMe), —C(═O)NH (substituted or unsubstituted phenyl), —C(═O)N (substituted or unsubstituted alkyl)-(substituted or unsubstituted alkyl), or —C(═O)N (substituted or unsubstituted phenyl)-(substituted or unsubstituted alkyl)). In certain embodiments, at least one instance of R¹ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, at least one instance of R¹ is —NR^{a}C(═O)OR^{a}. In certain embodiments, at least one instance of R¹ is —NR^{a}C(═O)N(R^{a})₂ (e.g., —NHC(═O)NH₂, —NHC(═O)NH (substituted or unsubstituted, C₁₋₆ alkyl) (e.g., —NHC(═O)NHMe)). In certain embodiments, at least one instance of R¹ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O (substituted or unsubstituted alkyl) or —OC(═O)O (substituted or unsubstituted phenyl)), or —OC(═O)N(R^{a})₂ (e.g., —OC(═O)NH₂, —OC(═O)NH (substituted or unsubstituted alkyl), —OC(═O)NH (substituted or unsubstituted phenyl), —OC(═O)N (substituted or unsubstituted alkyl)-(substituted or unsubstituted alkyl), or —OC(═O)N (substituted or unsubstituted phenyl)-(substituted or unsubstituted alkyl)). In certain embodiments, at least one instance of R¹ is —NR^{a}S(═O)₂R^{a} (e.g., —NHS(═O)₂R^{a}, —NHS(═O)₂ (substituted or unsubstituted alkyl or substituted or unsubstituted phenyl)). In certain embodiments, at least one instance of R¹ is —NR^{a}S(═O)₂OR^{a} (e.g., —NHS(═O)₂OR^{a}, —NHS(═O)₂OH, —NHS(═O)₂O (substituted or unsubstituted alkyl or substituted or unsubstituted phenyl)). In certain embodiments, at least one instance of R¹ is —NR^{a}S(═O)₂N(R^{a})₂ (e.g., —NHS(═O)₂N(R^{a})₂, —NHS(═O)₂NH₂, —NHS(═O)₂NH (substituted or unsubstituted alkyl or substituted or unsubstituted phenyl), —NHS(═O)₂N (substituted or unsubstituted alkyl)₂, —NHS(═O)₂N (substituted or unsubstituted alkyl)(substituted or unsubstituted phenyl)). In certain embodiments, at least one instance of R¹ is —OS(═O)₂R^{a} (e.g., —OS(═O)₂ (substituted or unsubstituted alkyl or substituted or unsubstituted phenyl)). In certain embodiments, at least one instance of R¹ is —OS(═O)₂OR^{a} (e.g., —OS(═O)₂OH, —OS(═O)₂O (substituted or unsubstituted alkyl or substituted or unsubstituted phenyl)). In certain embodiments, at least one instance of R¹ is —OS(═O)₂N(R^{a})₂ (e.g., —OS(═O)₂NH₂, —OS(═O)₂NH (substituted or unsubstituted alkyl or substituted or unsubstituted phenyl), —OS(═O)₂N (substituted or unsubstituted alkyl)₂, —OS(═O)₂N (substituted or unsubstituted alkyl)(substituted or unsubstituted phenyl)). In certain embodiments, at least one instance of R¹ is —S(═O)₂R^{a} (e.g., —S(═O)₂ (substituted or unsubstituted alkyl or substituted or unsubstituted phenyl)). In certain embodiments, at least one instance of R¹ is —S(═O)₂OR^{a} (e.g., —S(═O)₂OH, —S(═O)₂O (substituted or unsubstituted alkyl or substituted or unsubstituted phenyl)). In certain embodiments, at least one instance of R¹ is —S(═O)₂N(R^{a})₂ (e.g., —S(═O)₂NH₂, —S(═O)₂NH (substituted or unsubstituted alkyl or substituted or unsubstituted phenyl), —S(═O)₂N (substituted or unsubstituted alkyl)₂, —S(═O)₂N (substituted or unsubstituted alkyl)(substituted or unsubstituted phenyl)). In certain embodiments, at least one instance of R¹ is —P(═O)(R^{a})₂ (e.g., —P(═O)(substituted or unsubstituted phenyl)₂). In certain embodiments, at least one instance of R¹ is ═O. In certain embodiments, at least one instance of R¹ is halogen, substituted or unsubstituted alkyl, —OR^{a}, —C(═O)R^{a}, —C(═O)OR^{a}, substituted or unsubstituted, 3- to 7-membered, monocyclic heterocyclyl, substituted or unsubstituted phenyl, or substituted or unsubstituted, 5- to 6-membered, monocyclic heteroaryl. In certain embodiments, each R¹ is independently halogen, substituted or unsubstituted alkyl, —OR^{a}, —C(═O)R^{a}, —C(═O)OR^{a}, substituted or unsubstituted, 3- to 7-membered, monocyclic heterocyclyl, substituted or unsubstituted phenyl, or substituted or unsubstituted, 5- to 6-membered, monocyclic heteroaryl. In certain embodiments, at least one R¹ is halogen, substituted or unsubstituted alkyl (e.g., unsubstituted C₁₋₆ alkyl), or substituted or unsubstituted phenyl. In certain embodiments, each R¹ is independently halogen, substituted or unsubstituted alkyl (e.g., unsubstituted C₁₋₆ alkyl), or substituted or unsubstituted phenyl.

This paragraph applies when R¹ is attached to a nitrogen atom. In certain embodiments, at least one instance of R¹ is unsubstituted alkyl (e.g., unsubstituted C₁₋₆ alkyl). In certain embodiments, at least one instance of R¹ is Me. In certain embodiments, at least one instance of R¹ is Et, Pr, or Bu. In certain embodiments, at least one instance of R¹ is substituted alkyl (e.g., alkyl substituted with one or more instances of halogen (e.g., F)). In certain embodiments, at least one instance of R¹ is substituted C₁₋₆ alkyl. In certain embodiments, at least one instance of R¹ is substituted methyl (e.g., fluorinated methyl or Bn). In certain embodiments, at least one instance of R¹ is substituted ethyl, substituted propyl, or substituted butyl. In certain embodiments, at least one instance of R¹ is substituted or unsubstituted alkenyl. In certain embodiments, at least one instance of R¹ is substituted or unsubstituted, C₂₋₆ alkenyl (e.g., substituted or unsubstituted vinyl or substituted or unsubstituted allyl). In certain embodiments, at least one instance of R¹ is substituted or unsubstituted alkynyl. In certain embodiments, at least one instance of R¹ is substituted or unsubstituted, C₂₋₆ alkynyl (e.g., substituted or unsubstituted ethynyl). In certain embodiments, at least one instance of R¹ is substituted or unsubstituted carbocyclyl (e.g., substituted or unsubstituted, monocyclic, 3- to 7-membered carbocyclyl). In certain embodiments, at least one instance of R¹ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R¹ is substituted or unsubstituted heterocyclyl (e.g., substituted or unsubstituted, 3- to 7-membered, monocyclic heterocyclyl). In certain embodiments, at least one instance of R¹ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R¹ is substituted or unsubstituted aryl. In certain embodiments, at least one instance of R¹ is substituted or unsubstituted phenyl. In certain embodiments, at least one instance of R¹ is substituted or unsubstituted naphthyl. In certain embodiments, at least one instance of R¹ is substituted or unsubstituted heteroaryl. In certain embodiments, at least one instance of R¹ is substituted or unsubstituted, 5- to 6-membered, monocyclic heteroaryl. In certain embodiments, at least one instance of R¹ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R¹ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R¹ is substituted or unsubstituted, 9- to 10-membered, bicyclic heteroaryl. In certain embodiments, at least one instance of R¹ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R¹ is —C(═O)OR^{a} (e.g., —C(═O)OH, —C(═O)O (substituted or unsubstituted alkyl) (e.g., —C(═O)OMe), or —C(═O)O (substituted or unsubstituted phenyl)). In certain embodiments, at least one instance of R¹ is —C(═O)N(R^{a})₂ (e.g., —C(═O)NH₂, —C(═O)NH (substituted or unsubstituted alkyl) (e.g., —C(═O)NHMe), —C(═O)NH (substituted or unsubstituted phenyl), —C(═O)N (substituted or unsubstituted alkyl)-(substituted or unsubstituted alkyl), or —C(═O)N (substituted or unsubstituted phenyl)-(substituted or unsubstituted alkyl)). In certain embodiments, at least one instance of R¹ is —S(═O)₂R^{a} (e.g., —S(═O)₂ (substituted or unsubstituted alkyl or substituted or unsubstituted phenyl)). In certain embodiments, at least one instance of R¹ is —S(═O)₂OR^{a} (e.g., —S(═O)₂OH, —S(═O)₂O (substituted or unsubstituted alkyl or substituted or unsubstituted phenyl)). In certain embodiments, at least one instance of R¹ is —S(═O)₂N(R^{a})₂ (e.g., —S(═O)₂NH₂, —S(═O)₂NH (substituted or unsubstituted alkyl or substituted or unsubstituted phenyl), —S(═O)₂N (substituted or unsubstituted alkyl)₂, —S(═O)₂N (substituted or unsubstituted alkyl)(substituted or unsubstituted phenyl)). In certain embodiments, at least one instance of R¹ is —P(═O)(R^{a})₂ (e.g., —P(═O)(substituted or unsubstituted phenyl)₂). In certain embodiments, at least one instance of R¹ is a nitrogen protecting group (e.g., Bn, Boc, Cbz, Fmoc, trifluoroacetyl, triphenylmethyl, acetyl, or Ts). In certain embodiments, at least one instance of R¹ is ═O.

In certain embodiments, one R¹ and R³ are joined with their intervening atoms to form substituted or unsubstituted heterocyclyl (e.g., substituted or unsubstituted, 3- to 7-membered, monocyclic heterocyclyl).

In certain embodiments, when R¹ of [symbol] R¹ is a monovalent substituent, [symbol] is a single bond. In certain embodiments, when R¹ of [symbol] R¹ is a divalent substituent (e.g., ═O), [symbol] is a double bond. In certain embodiments, when R⁷ of [symbol] R⁷ is a monovalent substituent, [symbol] is a single bond. In certain embodiments, when R⁷ of [symbol] R⁷ is a divalent substituent (e.g., ═O), [symbol] is a double bond.

In certain embodiments, at least one instance of R^{a} is hydrogen. In certain embodiments, each instance of R^{a} is hydrogen. In certain embodiments, at least one instance of R^{a} is not hydrogen. In certain embodiments, no instance of R^{a} is hydrogen. In certain embodiments, at least one instance of R^{a} is substituted alkyl (e.g., alkyl substituted with one or more instances of halogen (e.g., F)). In certain embodiments, at least one instance of R^{a} is unsubstituted alkyl. In certain embodiments, at least one instance of R^{a} is unsubstituted C₁₋₆ alkyl. In certain embodiments, at least one instance of R^{a} is Me. In certain embodiments, at least one instance of R^{a} is Et, Pr, or Bu. In certain embodiments, at least one instance of R^{a} is substituted C₁₋₆ alkyl. In certain embodiments, at least one instance of R^{a} is substituted methyl (e.g., fluorinated methyl or Bn). In certain embodiments, at least one instance of R^{a} is substituted ethyl, substituted propyl, or substituted butyl. In certain embodiments, at least one instance of R^{a} is substituted or unsubstituted alkenyl. In certain embodiments, at least one instance of R^{a} is substituted or unsubstituted, C₂₋₆ alkenyl (e.g., substituted or unsubstituted vinyl or substituted or unsubstituted allyl). In certain embodiments, at least one instance of R^{a} is substituted or unsubstituted alkynyl. In certain embodiments, at least one instance of R^{a} is substituted or unsubstituted, C₂₋₆ alkynyl (e.g., substituted or unsubstituted ethynyl). In certain embodiments, at least one instance of R^{a} is substituted or unsubstituted carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R^{a} is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R^{a} is substituted or unsubstituted heterocyclyl (e.g., substituted or unsubstituted, 3- to 7-membered, monocyclic heterocyclyl). In certain embodiments, at least one instance of R^{a} is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R^{a} is substituted or unsubstituted aryl. In certain embodiments, at least one instance of R^{a} is substituted or unsubstituted phenyl. In certain embodiments, at least one instance of R^{a} is substituted or unsubstituted naphthyl. In certain embodiments, at least one instance of R^{a} is substituted or unsubstituted heteroaryl. In certain embodiments, at least one instance of R^{a} is substituted or unsubstituted, 5- to 6-membered, monocyclic heteroaryl. In certain embodiments, at least one instance of R^{a} is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R^{a} is substituted or unsubstituted pyridinyl, substituted or unsubstituted pyrazinyl, substituted or unsubstituted pyrimidinyl, or substituted or unsubstituted pyridazinyl. In certain embodiments, at least one instance of R^{a} is substituted or unsubstituted, 9- to 10-membered, bicyclic heteroaryl. In certain embodiments, at least one instance of R^{a} is a nitrogen protecting group (e.g., Bn, Boc, Cbz, Fmoc, trifluoroacetyl, triphenylmethyl, acetyl, or Ts) when attached to a nitrogen atom. In certain embodiments, at least one instance of R^{a} is an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom. In certain embodiments, two instances of R^{a} are joined to form substituted or unsubstituted heterocyclyl (e.g., substituted or unsubstituted, 3- to 7-membered, monocyclic heterocyclyl). In certain embodiments, two instances of R^{a} are joined to form substituted or unsubstituted heteroaryl (e.g., substituted or unsubstituted, 5- to 6-membered, monocyclic heteroaryl).

In certain embodiments, k is 0. In certain embodiments, k is 1. In certain embodiments, k is 2. In certain embodiments, k is 3. In certain embodiments, k is 4. In certain embodiments, k is 5. In certain embodiments, k is 0, 1, or 2. In certain embodiments, k is such an integer between 1 and 13, inclusive, that Ring A is fully substituted.

In certain embodiments, at least one instance of R² is hydrogen. In certain embodiments, each instance of R² is hydrogen. In certain embodiments, at least one instance of R² is halogen. In certain embodiments, at least one instance of R² is F. In certain embodiments, each R² is F. In certain embodiments, at least one instance of R² is C₁. In certain embodiments, at least one instance of R² is Br. In certain embodiments, at least one instance of R² is substituted alkyl (e.g., alkyl substituted with one or more instances of halogen (e.g., F)). In certain embodiments, at least one instance of R² is unsubstituted alkyl. In certain embodiments, at least one instance of R² is unsubstituted C₁₋₆ alkyl. In certain embodiments, at least one instance of R² is Me. In certain embodiments, each R² is Me. In certain embodiments, at least one instance of R² is Et, Pr, or Bu. In certain embodiments, at least one instance of R² is substituted C₁₋₆ alkyl. In certain embodiments, at least one instance of R² is substituted methyl (e.g., fluorinated methyl or Bn). In certain embodiments, at least one instance of R² is substituted ethyl, substituted propyl, or substituted butyl. In certain embodiments, one R² is hydrogen, and the other R² is unsubstituted C₁₋₆ alkyl (e.g., Me). In certain embodiments, one R² is hydrogen, and the other R² is halogen (e.g., F).

In certain embodiments, q is 0. In certain embodiments, q is 1.

In certain embodiments, R³ is hydrogen. In certain embodiments, R³ is substituted or unsubstituted alkyl (e.g., substituted or unsubstituted, C₁₋₆ alkyl). In certain embodiments, R³ is Me. In certain embodiments, R³ is Et, Pr, Bu, substituted methyl (e.g., fluorinated methyl or Bn), substituted ethyl, substituted propyl, or substituted butyl. In certain embodiments, R³ is a nitrogen protecting group (e.g., Bn, Boc, Cbz, Fmoc, trifluoroacetyl, triphenylmethyl, acetyl, or Ts).

In certain embodiments, X¹ is N. In certain embodiments, X² is N. In certain embodiments, X³ is N. In certain embodiments, X¹ is N(O). In certain embodiments, X² is N(O). In certain embodiments, X³ is N(O).

In certain embodiments, at least one instance of R⁴ is hydrogen. In certain embodiments, each instance of R⁴ is hydrogen. In certain embodiments, at least one instance of R⁴ is not hydrogen. In certain embodiments, no instance of R⁴ is hydrogen. In certain embodiments, at least one instance of R⁴ is halogen. In certain embodiments, at least one instance of R⁴ is F. In certain embodiments, at least one instance of R⁴ is C₁. In certain embodiments, at least one instance of R⁴ is Br. In certain embodiments, at least one instance of R⁴ is unsubstituted alkyl (e.g., unsubstituted C₁₋₆ alkyl). In certain embodiments, at least one instance of R⁴ is Me. In certain embodiments, at least one instance of R⁴ is Et, Pr, or Bu. In certain embodiments, at least one instance of R⁴ is substituted alkyl (e.g., alkyl substituted with one or more instances of halogen (e.g., F)). In certain embodiments, at least one instance of R⁴ is substituted C₁₋₆ alkyl. In certain embodiments, at least one instance of R⁴ is substituted methyl (e.g., fluorinated methyl or Bn). In certain embodiments, at least one instance of R⁴ is substituted ethyl, substituted propyl, or substituted butyl. In certain embodiments, at least one instance of R⁴ is substituted or unsubstituted alkenyl. In certain embodiments, at least one instance of R⁴ is substituted or unsubstituted, C₂₋₆ alkenyl (e.g., substituted or unsubstituted vinyl or substituted or unsubstituted allyl). In certain embodiments, at least one instance of R⁴ is substituted or unsubstituted alkynyl. In certain embodiments, at least one instance of R⁴ is substituted or unsubstituted, C₂₋₆ alkynyl (e.g., substituted or unsubstituted ethynyl). In certain embodiments, at least one instance of R⁴ is substituted or unsubstituted carbocyclyl (e.g., substituted or unsubstituted, monocyclic, 3- to 7-membered carbocyclyl). In certain embodiments, at least one instance of R⁴ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R⁴ is substituted or unsubstituted heterocyclyl (e.g., substituted or unsubstituted, 3- to 7-membered, monocyclic heterocyclyl). In certain embodiments, at least one instance of R⁴ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R⁴ is substituted or unsubstituted aryl. In certain embodiments, at least one instance of R⁴ is substituted or unsubstituted phenyl. In certain embodiments, at least one instance of R⁴ is unsubstituted phenyl. In certain embodiments, at least one instance of R⁴ is substituted or unsubstituted naphthyl. In certain embodiments, at least one instance of R⁴ is substituted or unsubstituted heteroaryl. In certain embodiments, at least one instance of R⁴ is substituted or unsubstituted, 5- to 6-membered, monocyclic heteroaryl. In certain embodiments, at least one instance of R⁴ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R⁴ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R⁴ is substituted or unsubstituted, 9- to 10-membered, bicyclic heteroaryl. In certain embodiments, at least one instance of R⁴ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, at least one instance of R⁴ is —O(substituted or unsubstituted alkyl). In certain embodiments, at least one instance of R⁴ is —O(alkyl substituted with at least one —OH). In certain embodiments, at least one instance of R⁴ is —OCH₂CH(OH)CH₂OH. In certain embodiments, at least one instance of R⁴ is —O(unsubstituted alkyl). In certain embodiments, at least one instance of R⁴ is —OMe. In certain embodiments, at least one instance of R⁴ is —SR^{a}(e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, at least one instance of R⁴ is —N(R^{a})₂ (e.g., —NH₂, —NH (substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, at least one instance of R⁴ is —CN or —SCN. In certain embodiments, at least one instance of R⁴ is —NO₂. In certain embodiments, at least one instance of R⁴ is —C(═NR^{a})R^{a}, —C(═NR^{a})OR^{a}, or —C(═NR^{a})N(R^{a})₂. In certain embodiments, at least one instance of R⁴ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R⁴ is —C(═O)OR^{a} (e.g., —C(═O)OH, —C(═O)O (substituted or unsubstituted alkyl) (e.g., —C(═O)OMe), or —C(═O)O (substituted or unsubstituted phenyl)). In certain embodiments, at least one instance of R⁴ is —C(═O)N(R^{a})₂ (e.g., —C(═O)NH₂, —C(═O)NH (substituted or unsubstituted alkyl) (e.g., —C(═O)NHMe), —C(═O)NH (substituted or unsubstituted phenyl), —C(═O)N (substituted or unsubstituted alkyl)-(substituted or unsubstituted alkyl), or —C(═O)N (substituted or unsubstituted phenyl)-(substituted or unsubstituted alkyl)). In certain embodiments, at least one instance of R⁴ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, at least one instance of R⁴ is —NR^{a}C(═O)OR^{a}. In certain embodiments, at least one instance of R⁴ is —NR^{a}C(═O)N(R^{a})₂ (e.g., —NHC(═O)NH₂, —NHC(═O)NH (substituted or unsubstituted, C₁₋₆ alkyl) (e.g., —NHC(═O)NHMe)). In certain embodiments, at least one instance of R⁴ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O (substituted or unsubstituted alkyl) or —OC(═O)O (substituted or unsubstituted phenyl)), or —OC(═O)N(R^{a})₂ (e.g., —OC(═O)NH₂, —OC(═O)NH (substituted or unsubstituted alkyl), —OC(═O)NH (substituted or unsubstituted phenyl), —OC(═O)N (substituted or unsubstituted alkyl)-(substituted or unsubstituted alkyl), or —OC(═O)N (substituted or unsubstituted phenyl)-(substituted or unsubstituted alkyl)). In certain embodiments, at least one instance of R⁴ is —NR^{a}S(═O)₂R^{a} (e.g., —NHS(═O)₂R^{a}, —NHS(═O)₂ (substituted or unsubstituted alkyl or substituted or unsubstituted phenyl)). In certain embodiments, at least one instance of R⁴ is —NR^{a}S(═O)₂OR^{a} (e.g., —NHS(═O)₂OR^{a}, —NHS(═O)₂OH, —NHS(═O)₂O (substituted or unsubstituted alkyl or substituted or unsubstituted phenyl)). In certain embodiments, at least one instance of R⁴ is —NR^{a}S(═O)₂N(R^{a})₂ (e.g., —NHS(═O)₂N(R^{a})₂, —NHS(═O)₂NH₂, —NHS(═O)₂NH (substituted or unsubstituted alkyl or substituted or unsubstituted phenyl), —NHS(═O)₂N (substituted or unsubstituted alkyl)₂, —NHS(═O)₂N (substituted or unsubstituted alkyl)(substituted or unsubstituted phenyl)). In certain embodiments, at least one instance of R⁴ is —OS(═O)₂R^{a} (e.g., —OS(═O)₂ (substituted or unsubstituted alkyl or substituted or unsubstituted phenyl)). In certain embodiments, at least one instance of R⁴ is —OS(═O)₂OR^{a} (e.g., —OS(═O)₂OH, —OS(═O)₂O (substituted or unsubstituted alkyl or substituted or unsubstituted phenyl)). In certain embodiments, at least one instance of R⁴ is —OS(═O)₂N(R^{a})₂ (e.g., —OS(═O)₂NH₂, —OS(═O)₂NH (substituted or unsubstituted alkyl or substituted or unsubstituted phenyl), —OS(═O)₂N (substituted or unsubstituted alkyl)₂, —OS(═O)₂N (substituted or unsubstituted alkyl)(substituted or unsubstituted phenyl)). In certain embodiments, at least one instance of R⁴ is —S(═O)₂R^{a} (e.g., —S(═O)₂ (substituted or unsubstituted alkyl or substituted or unsubstituted phenyl)). In certain embodiments, at least one instance of R⁴ is —S(═O)₂OR^{a} (e.g., —S(═O)₂OH, —S(═O)₂O (substituted or unsubstituted alkyl or substituted or unsubstituted phenyl)). In certain embodiments, at least one instance of R⁴ is —S(═O)₂N(R^{a})₂ (e.g., —S(═O)₂NH₂, —S(═O)₂NH (substituted or unsubstituted alkyl or substituted or unsubstituted phenyl), —S(═O)₂N (substituted or unsubstituted alkyl)₂, —S(═O)₂N (substituted or unsubstituted alkyl)(substituted or unsubstituted phenyl)).

In certain embodiments, R⁵ is hydrogen. In certain embodiments, R⁵ is not hydrogen. In certain embodiments, R⁵ is halogen. In certain embodiments, R⁵ is F. In certain embodiments, R⁵ is C₁. In certain embodiments, R⁵ is Br. In certain embodiments, R⁵ is unsubstituted alkyl (e.g., unsubstituted C₁₋₆ alkyl). In certain embodiments, R⁵ is Me. In certain embodiments, R⁵ is Et. In certain embodiments, R⁵ is Pr, or Bu. In certain embodiments, R⁵ is substituted alkyl (e.g., alkyl substituted with one or more instances of halogen (e.g., F)). In certain embodiments, R⁵ is substituted C₁₋₆ alkyl. In certain embodiments, R⁵ is substituted methyl (e.g., fluorinated methyl or Bn). In certain embodiments, R⁵ is substituted ethyl, substituted propyl, or substituted butyl. In certain embodiments, R⁵ is substituted or unsubstituted alkenyl. In certain embodiments, R⁵ is substituted or unsubstituted, C₂₋₆ alkenyl (e.g., substituted or unsubstituted vinyl or substituted or unsubstituted allyl). In certain embodiments, R⁵ is substituted or unsubstituted alkynyl. In certain embodiments, R⁵ is substituted or unsubstituted, C₂₋₆ alkynyl (e.g., substituted or unsubstituted ethynyl). In certain embodiments, R⁵ is substituted or unsubstituted carbocyclyl (e.g., substituted or unsubstituted, monocyclic, 3- to 7-membered carbocyclyl). In certain embodiments, R⁵ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, R⁵ is substituted or unsubstituted heterocyclyl (e.g., substituted or unsubstituted, 3- to 7-membered, monocyclic heterocyclyl). In certain embodiments, R⁵ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, R⁵ is substituted or unsubstituted aryl. In certain embodiments, R⁵ is substituted or unsubstituted phenyl. In certain embodiments, R⁵ is unsubstituted phenyl. In certain embodiments, R⁵ is substituted or unsubstituted naphthyl. In certain embodiments, R⁵ is substituted or unsubstituted heteroaryl. In certain embodiments, R⁵ is substituted or unsubstituted, 5- to 6-membered, monocyclic heteroaryl. In certain embodiments, R⁵ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, R⁵ is substituted or unsubstituted pyridinyl, substituted or unsubstituted pyrazinyl, substituted or unsubstituted pyrimidinyl, or substituted or unsubstituted pyridazinyl. In certain embodiments, R⁵ is substituted or unsubstituted, 9- to 10-membered, bicyclic heteroaryl. In certain embodiments, R⁵ is —OR^{a}. In certain embodiments, R⁵ is —OH. In certain embodiments, R⁵ is —O(substituted or unsubstituted alkyl). In certain embodiments, R⁵ is —O(substituted alkyl). In certain embodiments, R⁵ is —O(alkyl substituted at least with —P(R^{a})₃X (e.g., —P(substituted or unsubstituted phenyl)₃X), wherein X is a counterion). In certain embodiments, R⁵ is —O-(unsubstituted C₂₋₁₂ alkylene)—P(substituted or unsubstituted phenyl)₃X (e.g., —O-(unsubstituted C₂₋₁₂ alkylene)—P(unsubstituted phenyl)₃X). In certain embodiments, R⁵ is —O(substituted or unsubstituted, C₁₋₆ alkyl). In certain embodiments, R⁵ is —O(unsubstituted C₁₋₆ alkyl). In certain embodiments, R⁵ is —OMe, —OCF₃, —OEt, —OPr, —OBu, or —OBn). In certain embodiments, R⁵ is —O(substituted or unsubstituted phenyl) (e.g., —OPh). In certain embodiments, R⁵ is —OMe. In certain embodiments, R⁵ is —OEt. In certain embodiments, R⁵ is —SR^{a} (e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, R⁵ is —N(R^{a})₂ (e.g., —NH₂, —NH (substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, R⁵ is —CN or —SCN. In certain embodiments, R⁵ is —NO₂. In certain embodiments, R^{S} is —C(═NR^{a})R^{a}, —C(═NR^{a})OR^{a}, or —C(═NR^{a})N(R^{a})₂. In certain embodiments, R⁵ is —C(═O)R^{a} (e.g., —C(═O)(substituted or unsubstituted alkyl) (e.g., —C(═O)Me) or —C(═O)(substituted or unsubstituted phenyl)). In certain embodiments, R⁵ is —C(═O)OR^{a} (e.g., —C(═O)OH, —C(═O)O (substituted or unsubstituted alkyl) (e.g., —C(═O)OMe), or —C(═O)O (substituted or unsubstituted phenyl)). In certain embodiments, R⁵ is —C(═O)N(R^{a})₂ (e.g., —C(═O)NH₂, —C(═O)NH (substituted or unsubstituted alkyl) (e.g., —C(═O)NHMe), —C(═O)NH (substituted or unsubstituted phenyl), —C(═O)N (substituted or unsubstituted alkyl)-(substituted or unsubstituted alkyl), or —C(═O)N (substituted or unsubstituted phenyl)-(substituted or unsubstituted alkyl)). In certain embodiments, R⁵ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, R⁵ is —NR^{a}C(═O)OR^{a}. In certain embodiments, R⁵ is —NR^{a}C(═O)N(R^{a})₂ (e.g., —NHC(═O)NH₂, —NHC(═O)NH (substituted or unsubstituted, C₁₋₆ alkyl) (e.g., —NHC(═O)NHMe)). In certain embodiments, R⁵ is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O (substituted or unsubstituted alkyl) or —OC(═O)O (substituted or unsubstituted phenyl)), or —OC(═O)N(R^{a})₂ (e.g., —OC(═O)NH₂, —OC(═O)NH (substituted or unsubstituted alkyl), —OC(═O)NH (substituted or unsubstituted phenyl), —OC(═O)N (substituted or unsubstituted alkyl)-(substituted or unsubstituted alkyl), or —OC(═O)N (substituted or unsubstituted phenyl)-(substituted or unsubstituted alkyl)). In certain embodiments, R⁵ is —NR^{a}S(═O)₂R^{a} (e.g., —NHS(═O)₂R^{a}, —NHS(═O)₂ (substituted or unsubstituted alkyl or substituted or unsubstituted phenyl)). In certain embodiments, R⁵ is —NR^{a}S(═O)₂OR^{a} (e.g., —NHS(═O)₂OR^{a}, —NHS(═O)₂OH, —NHS(═O)₂O (substituted or unsubstituted alkyl or substituted or unsubstituted phenyl)). In certain embodiments, R⁵ is —NR^{a}S(═O)₂N(R^{a})₂ (e.g., —NHS(═O)₂N(R^{a})₂, —NHS(═O)₂NH₂, —NHS(═O)₂NH (substituted or unsubstituted alkyl or substituted or unsubstituted phenyl), —NHS(═O)₂N (substituted or unsubstituted alkyl)₂, —NHS(═O)₂N (substituted or unsubstituted alkyl)(substituted or unsubstituted phenyl)). In certain embodiments, R⁵ is —OS(═O)₂R^{a} (e.g., —OS(═O)₂ (substituted or unsubstituted alkyl or substituted or unsubstituted phenyl)). In certain embodiments, R⁵ is —OS(═O)₂OR^{a} (e.g., —OS(═O)₂OH, —OS(═O)₂O (substituted or unsubstituted alkyl or substituted or unsubstituted phenyl)). In certain embodiments, R⁵ is —OS(═O)₂N(R^{a})₂ (e.g., —OS(═O)₂NH₂, —OS(═O)₂NH (substituted or unsubstituted alkyl or substituted or unsubstituted phenyl), —OS(═O)₂N (substituted or unsubstituted alkyl)₂, —OS(═O)₂N (substituted or unsubstituted alkyl)(substituted or unsubstituted phenyl)). In certain embodiments, R⁵ is —S(═O)₂R^{a} (e.g., —S(═O)₂ (substituted or unsubstituted alkyl or substituted or unsubstituted phenyl)). In certain embodiments, R⁵ is —S(═O)₂OR^{a} (e.g., —S(═O)₂OH, —S(═O)₂O (substituted or unsubstituted alkyl or substituted or unsubstituted phenyl)). In certain embodiments, R⁵ is —S(═O)₂N(R^{a})₂ (e.g., —S(═O)₂NH₂, —S(═O)₂NH (substituted or unsubstituted alkyl or substituted or unsubstituted phenyl), —S(═O)₂N (substituted or unsubstituted alkyl)₂, —S(═O)₂N (substituted or unsubstituted alkyl)(substituted or unsubstituted phenyl)). In certain embodiments, R⁵ is —OR^{a} or substituted or unsubstituted alkyl.

In certain embodiments, R⁶ is hydrogen. In certain embodiments, R⁶ is substituted or unsubstituted alkyl (e.g., substituted or unsubstituted, C₁₋₆ alkyl). In certain embodiments, R⁶ is Me. In certain embodiments, R⁶ is Et, Pr, Bu, substituted methyl (e.g., fluorinated methyl or Bn), substituted ethyl, substituted propyl, or substituted butyl. In certain embodiments, R⁶ is substituted alkyl. In certain embodiments, R⁶ is alkyl substituted at least with —P(R^{a})₃X (e.g., —P(substituted or unsubstituted phenyl)₃X), wherein X is a counterion). In certain embodiments, R⁶ is -(unsubstituted C₂₋₁₂ alkylene)—P(substituted or unsubstituted phenyl)₃X (e.g., -(unsubstituted C₂₋₁₂ alkylene)—P(unsubstituted phenyl)₃X). In certain embodiments, R⁶ is a nitrogen protecting group (e.g., Bn, Boc, Cbz, Fmoc, trifluoroacetyl, triphenylmethyl, acetyl, or Ts).

In certain embodiments, R⁶ and one R⁷ are joined with their intervening atoms to form substituted or unsubstituted heterocyclyl (e.g., substituted or unsubstituted, 3- to 7-membered, monocyclic heterocyclyl).

In certain embodiments,

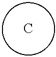

(“Ring C”) is aryl. In certain embodiments, Ring C is phenyl. In certain embodiments,

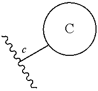

is unsubstituted phenyl. In certain embodiments,

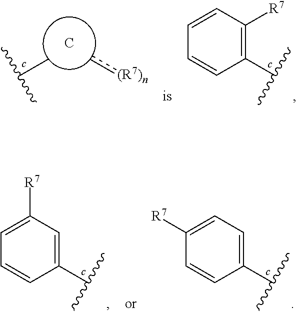

In certain embodiments,

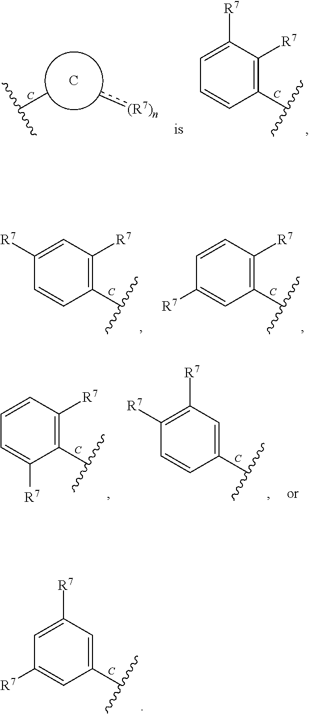

In certain embodiments,

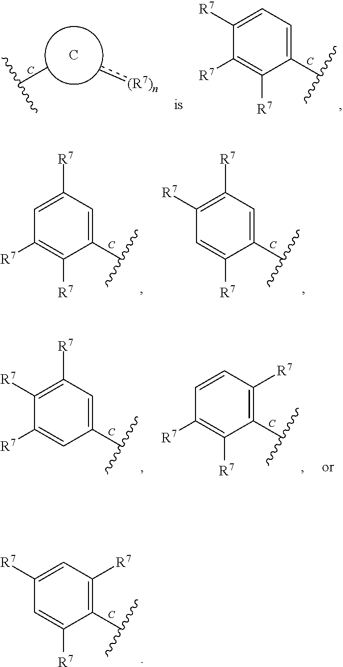

In certain embodiments,

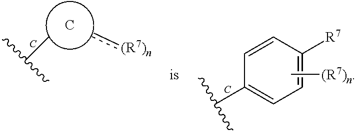

In certain embodiments,

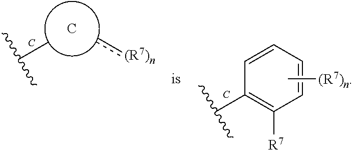

In certain embodiments,

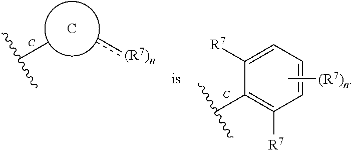

In certain embodiments,

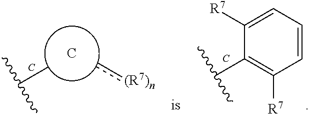

In certain embodiments,

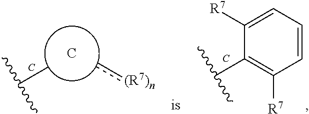

wherein each R⁷ is independently halogen or substituted or unsubstituted alkyl (e.g., unsubstituted C₁₋₆ alkyl). In certain embodiments,

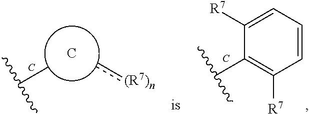

wherein each R⁷ is independently substituted or unsubstituted alkyl (e.g., unsubstituted C₁₋₆ alkyl). In certain embodiments,

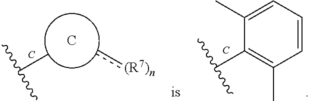

In certain embodiments,

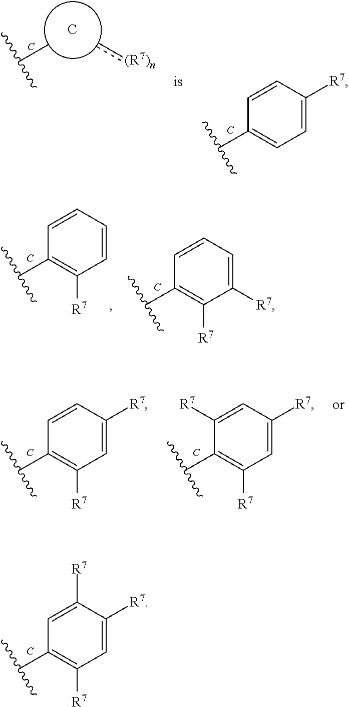

In certain embodiments,

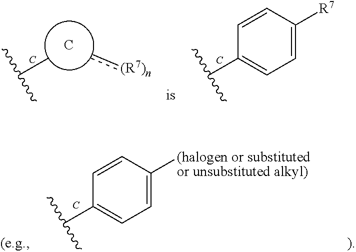

In certain embodiments,

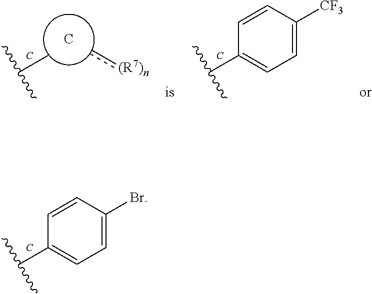

In certain embodiments,

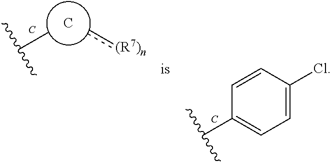

In certain embodiments,

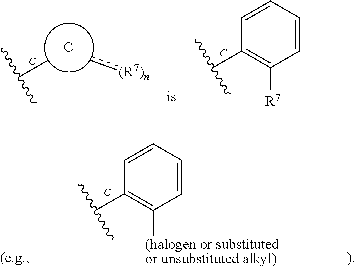

In certain embodiments,

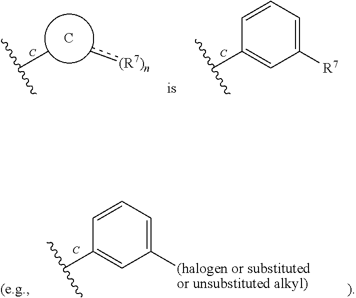

In certain embodiments, Ring C is heteroaryl. In certain embodiments, Ring C is 5- or 6-membered monocyclic heteroaryl. In certain embodiments, Ring C is pyrimidinyl, thienyl, pyrazolyl, tetrazolyl, isoxazolyl, or

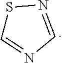

In certain embodiments, Ring C is pyridinyl, pyrazinyl, pyrimidinyl, or pyridazinyl. In certain embodiments, Ring C is pyridinyl. In certain embodiments,

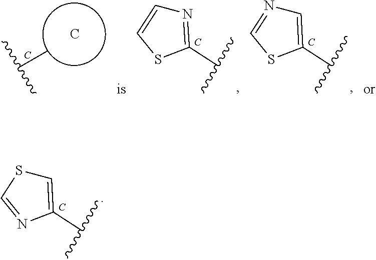

In certain embodiments,

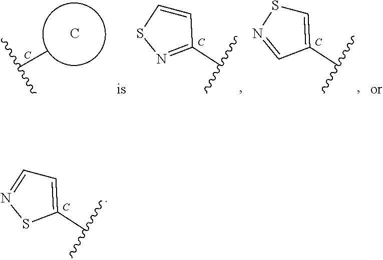

In certain embodiments,

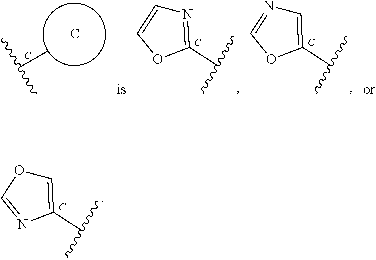

In certain embodiments,

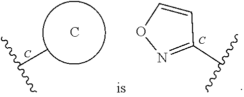

In certain embodiments,

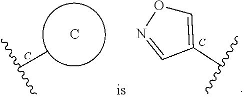

In certain embodiments,

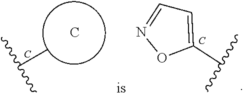

In certain embodiments,

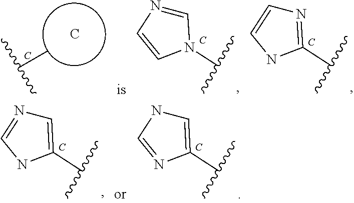

In certain embodiments,

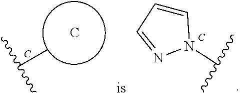

In certain embodiments,

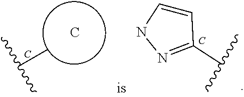

In certain embodiments,

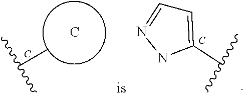

In certain embodiments,

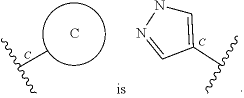

In certain embodiments,

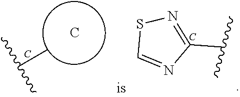

In certain embodiments,

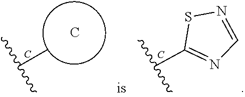

In certain embodiments,

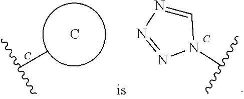

In certain embodiments,

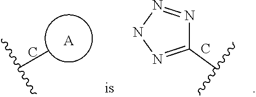

In certain embodiments,

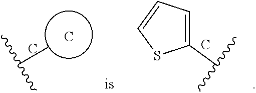

In certain embodiments,

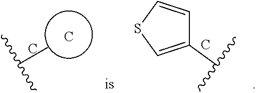

In certain embodiments,

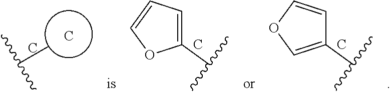

In certain embodiments,

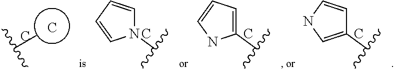

In certain embodiments,

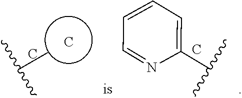

In certain embodiments,

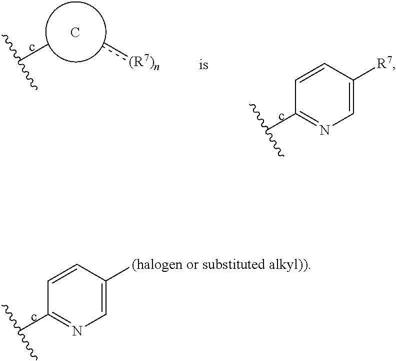

In certain embodiments,

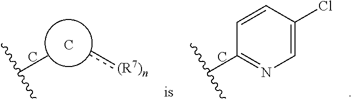

In certain embodiments,

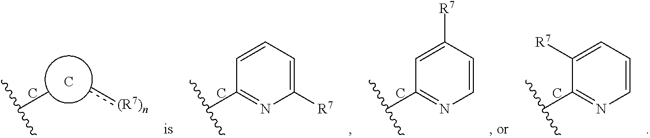

In certain embodiments,

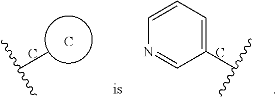

In certain embodiments,

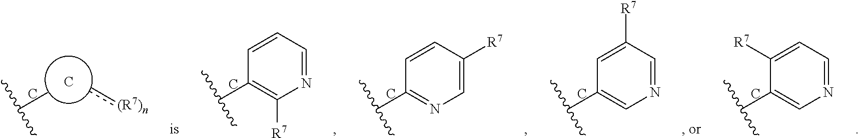

In certain embodiments,

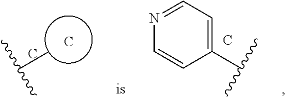

In certain embodiments,

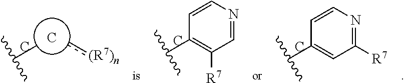

In certain embodiments,

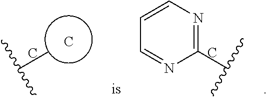

In certain embodiments,

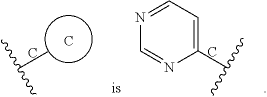

In certain embodiments,

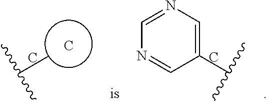

In certain embodiments, Ring C is phenyl fused with monocyclic or bicyclic, carbocyclyl, heterocyclyl, aryl, or heteroaryl, wherein bond c is attached to the phenyl; or 5- or 6-membered monocyclic heteroaryl fused with monocyclic or bicyclic, carbocyclyl, heterocyclyl, aryl, or heteroaryl, wherein bond c is attached to the 5- or 6-membered monocyclic heteroaryl.

In certain embodiments, Ring C is phenyl fused with monocyclic or bicyclic, carbocyclyl, heterocyclyl, aryl, or heteroaryl, wherein bond c is attached to the phenyl. In certain embodiments, Ring C is naphthyl. In certain embodiments, Ring C is phenyl fused with naphthyl, wherein bond c is attached to the phenyl. In certain embodiments, Ring C is phenyl fused with 5- to 6-membered, monocyclic heteroaryl, wherein bond c is attached to the phenyl. In certain embodiments, Ring C is phenyl fused with 8-14 membered, bicyclic heteroaryl, wherein bond c is attached to the phenyl. In certain embodiments, Ring C is phenyl fused with 4- to 7-membered, monocyclic carbocyclyl, wherein bond c is attached to the phenyl. In certain embodiments, Ring C is phenyl fused with 6- to 13-membered, bicyclic carbocyclyl, wherein bond c is attached to the phenyl. In certain embodiments, Ring C is phenyl fused with 4- to 7-membered, monocyclic heterocyclyl, wherein bond c is attached to the phenyl. In certain embodiments, Ring C is phenyl fused with 6- to 13-membered, bicyclic heterocyclyl, wherein bond c is attached to the phenyl.

In certain embodiments, Ring C is 5- or 6-membered monocyclic heteroaryl fused with monocyclic or bicyclic, carbocyclyl, heterocyclyl, aryl, or heteroaryl, wherein bond c is attached to the 5- or 6-membered monocyclic heteroaryl. In certain embodiments, Ring C is 5- or 6-membered monocyclic heteroaryl fused with phenyl, wherein bond c is attached to the 5- or 6-membered monocyclic heteroaryl. In certain embodiments, Ring C is 5- or 6-membered monocyclic heteroaryl fused with naphthyl, wherein bond c is attached to the 5- or 6-membered monocyclic heteroaryl. In certain embodiments, Ring C is 5- or 6-membered monocyclic heteroaryl fused with another 5- to 6-membered, monocyclic heteroaryl. In certain embodiments, Ring C is 5- or 6-membered monocyclic heteroaryl fused with 8-14 membered, bicyclic heteroaryl, wherein bond c is attached to the 5- or 6-membered monocyclic heteroaryl. In certain embodiments, Ring C is 5- or 6-membered monocyclic heteroaryl fused with 4- to 7-membered, monocyclic carbocyclyl, wherein bond c is attached to the 5- or 6-membered monocyclic heteroaryl. In certain embodiments, Ring C is 5- or 6-membered monocyclic heteroaryl fused with 6- to 13-membered, bicyclic carbocyclyl, wherein bond c is attached to the 5- or 6-membered monocyclic heteroaryl. In certain embodiments, Ring C is 5- or 6-membered monocyclic heteroaryl fused with 4- to 7-membered, monocyclic heterocyclyl, wherein bond c is attached to the 5- or 6-membered monocyclic heteroaryl. In certain embodiments, Ring C is 5- or 6-membered monocyclic heteroaryl fused with 6- to 13-membered, bicyclic heterocyclyl, wherein bond c is attached to the 5- or 6-membered monocyclic heteroaryl.

In certain embodiments,

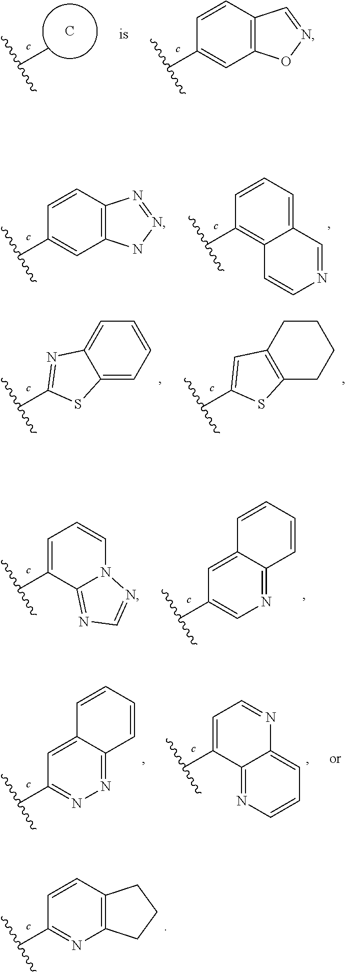

In certain embodiments, the molecular weight of each R⁷ is less than 200 g/mol. In certain embodiments, the molecular weight of each R⁷ is less than 150 g/mol. In certain embodiments, the molecular weight of each R⁷ is less than 100 g/mol.

This paragraph applies when R⁷ is attached to a carbon atom. In certain embodiments, at least one instance of R⁷ is halogen. In certain embodiments, at least one instance of R⁷ is F. In certain embodiments, at least one instance of R⁷ is C₁. In certain embodiments, at least one instance of R⁷ is Br. In certain embodiments, at least one instance of R⁷ is unsubstituted alkyl (e.g., unsubstituted C₁₋₆ alkyl). In certain embodiments, at least one instance of R⁷ is Me. In certain embodiments, at least one instance of R⁷ is Et, Pr, or Bu. In certain embodiments, at least one instance of R⁷ is substituted alkyl (e.g., alkyl substituted with one or more instances of halogen (e.g., F)). In certain embodiments, at least one instance of R⁷ is —CF₃. In certain embodiments, at least one instance of R⁷ is substituted C₁₋₆ alkyl. In certain embodiments, at least one instance of R⁷ is substituted methyl (e.g., fluorinated methyl or Bn). In certain embodiments, at least one instance of R⁷ is substituted ethyl, substituted propyl, or substituted butyl. In certain embodiments, at least one instance of R⁷ is substituted or unsubstituted alkenyl. In certain embodiments, at least one instance of R⁷ is substituted or unsubstituted, C₂₋₆ alkenyl (e.g., substituted or unsubstituted vinyl or substituted or unsubstituted allyl). In certain embodiments, at least one instance of R⁷ is substituted or unsubstituted alkynyl. In certain embodiments, at least one instance of R⁷ is substituted or unsubstituted, C₂₋₆ alkynyl (e.g., substituted or unsubstituted ethynyl). In certain embodiments, at least one instance of R⁷ is substituted or unsubstituted carbocyclyl (e.g., substituted or unsubstituted, monocyclic, 3- to 7-membered carbocyclyl). In certain embodiments, at least one instance of R⁷ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R⁷ is substituted or unsubstituted heterocyclyl (e.g., substituted or unsubstituted, 3- to 7-membered, monocyclic heterocyclyl). In certain embodiments, at least one instance of R⁷ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R⁷ is substituted or unsubstituted aryl. In certain embodiments, at least one instance of R⁷ is substituted or unsubstituted phenyl. In certain embodiments, at least one instance of R⁷ is unsubstituted phenyl. In certain embodiments, at least one instance of R⁷ is substituted or unsubstituted naphthyl. In certain embodiments, at least one instance of R⁷ is substituted or unsubstituted heteroaryl. In certain embodiments, at least one instance of R⁷ is substituted or unsubstituted, 5- to 6-membered, monocyclic heteroaryl. In certain embodiments, at least one instance of R⁷ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R⁷ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R⁷ is substituted or unsubstituted, 9- to 10-membered, bicyclic heteroaryl. In certain embodiments, at least one instance of R⁷ is —OR^{a} (e.g., —OH, —O(substituted or unsubstituted, C₁₋₆ alkyl) (e.g., —OMe, —OCF₃, —OEt, —OPr, —OBu, or —OBn), or —O(substituted or unsubstituted phenyl) (e.g., —OPh)). In certain embodiments, at least one instance of R⁷ is —O(substituted or unsubstituted alkyl). In certain embodiments, at least one instance of R⁷ is —O(alkyl substituted with at least one —OH). In certain embodiments, at least one instance of R⁷ is —OCH₂CH(OH)CH₂OH. In certain embodiments, at least one instance of R⁷ is —O(unsubstituted alkyl). In certain embodiments, at least one instance of R⁷ is —OMe. In certain embodiments, at least one instance of R⁷ is —SR^{a}(e.g., —SH, —S(substituted or unsubstituted, C₁₋₆ alkyl) (e.g., —SMe, —SCF₃, —SEt, —SPr, —SBu, or —SBn), or —S(substituted or unsubstituted phenyl) (e.g., —SPh)). In certain embodiments, at least one instance of R⁷ is —N(R^{a})₂ (e.g., —NH₂, —NH (substituted or unsubstituted, C₁₋₆ alkyl) (e.g., —NHMe), or —N(substituted or unsubstituted, C₁₋₆ alkyl)-(substituted or unsubstituted, C₁₋₆ alkyl) (e.g., —NMe₂)). In certain embodiments, at least one instance of R⁷ is —CN or —SCN. In certain embodiments, at least one instance of R⁷ is —NO₂. In certain embodiments, at least one instance of R⁷ is —C(═NR^{a})R^{a}, —C(═NR^{a})OR^{a}, or —C(═NR^{a})N(R^{a})₂. In certain embodiments, at least one instance of R⁷ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R⁷ is —C(═O)OR^{a} (e.g., —C(═O)OH, —C(═O)O (substituted or unsubstituted alkyl) (e.g., —C(═O)OMe), or —C(═O)O (substituted or unsubstituted phenyl)). In certain embodiments, at least one instance of R⁷ is —C(═O)N(R^{a})₂ (e.g., —C(═O)NH₂, —C(═O)NH (substituted or unsubstituted alkyl) (e.g., —C(═O)NHMe), —C(═O)NH (substituted or unsubstituted phenyl), —C(═O)N (substituted or unsubstituted alkyl)-(substituted or unsubstituted alkyl), or —C(═O)N (substituted or unsubstituted phenyl)-(substituted or unsubstituted alkyl)). In certain embodiments, at least one instance of R⁷ is —NR^{a}C(═O)R^{a} (e.g., —NHC(═O)(substituted or unsubstituted, C₁₋₆ alkyl) (e.g., —NHC(═O)Me) or —NHC(═O)(substituted or unsubstituted phenyl)). In certain embodiments, at least one instance of R⁷ is —NR^{a}C(═O)OR^{a}. In certain embodiments, at least one instance of R⁷ is —NR^{a}C(═O)N(R^{a})₂ (e.g., —NHC(═O)NH₂, —NHC(═O)NH (substituted or unsubstituted, C₁₋₆ alkyl) (e.g., —NHC(═O)NHMe)). In certain embodiments, at least one instance of R⁷ is —OC(═O)R^{a} (e.g., —OC(═O)(substituted or unsubstituted alkyl) or —OC(═O)(substituted or unsubstituted phenyl)), —OC(═O)OR^{a}(e.g., —OC(═O)O (substituted or unsubstituted alkyl) or —OC(═O)O (substituted or unsubstituted phenyl)), or —OC(═O)N(R^{a})₂ (e.g., —OC(═O)NH₂, —OC(═O)NH (substituted or unsubstituted alkyl), —OC(═O)NH (substituted or unsubstituted phenyl), —OC(═O)N (substituted or unsubstituted alkyl)-(substituted or unsubstituted alkyl), or —OC(═O)N (substituted or unsubstituted phenyl)-(substituted or unsubstituted alkyl)). In certain embodiments, at least one instance of R⁷ is —NR^{a}S(═O)₂R^{a} (e.g., —NHS(═O)₂R^{a}, —NHS(═O)₂ (substituted or unsubstituted alkyl or substituted or unsubstituted phenyl)). In certain embodiments, at least one instance of R⁷ is —NR^{a}S(═O)₂OR^{a} (e.g., —NHS(═O)₂OR^{a}, —NHS(═O)₂OH, —NHS(═O)₂O (substituted or unsubstituted alkyl or substituted or unsubstituted phenyl)). In certain embodiments, at least one instance of R⁷ is —NR^{a}S(═O)₂N(R^{a})₂ (e.g., —NHS(═O)₂N(R^{a})₂, —NHS(═O)₂NH₂, —NHS(═O)₂NH (substituted or unsubstituted alkyl or substituted or unsubstituted phenyl), —NHS(═O)₂N (substituted or unsubstituted alkyl)₂, —NHS(═O)₂N (substituted or unsubstituted alkyl)(substituted or unsubstituted phenyl)). In certain embodiments, at least one instance of R⁷ is —OS(═O)₂R^{a} (e.g., —OS(═O)₂ (substituted or unsubstituted alkyl or substituted or unsubstituted phenyl)). In certain embodiments, at least one instance of R⁷ is —OS(═O)₂OR^{a} (e.g., —OS(═O)₂OH, —OS(═O)₂O (substituted or unsubstituted alkyl or substituted or unsubstituted phenyl)). In certain embodiments, at least one instance of R⁷ is —OS(═O)₂N(R^{a})₂ (e.g., —OS(═O)₂NH₂, —OS(═O)₂NH (substituted or unsubstituted alkyl or substituted or unsubstituted phenyl), —OS(═O)₂N (substituted or unsubstituted alkyl)₂, —OS(═O)₂N (substituted or unsubstituted alkyl)(substituted or unsubstituted phenyl)). In certain embodiments, at least one instance of R⁷ is —S(═O)₂R^{a} (e.g., —S(═O)₂ (substituted or unsubstituted alkyl or substituted or unsubstituted phenyl)). In certain embodiments, at least one instance of R⁷ is —S(═O)₂OR^{a} (e.g., —S(═O)₂OH, —S(═O)₂O (substituted or unsubstituted alkyl or substituted or unsubstituted phenyl)). In certain embodiments, at least one instance of R⁷ is —S(═O)₂N(R^{a})₂ (e.g., —S(═O)₂NH₂, —S(═O)₂NH (substituted or unsubstituted alkyl or substituted or unsubstituted phenyl), —S(═O)₂N (substituted or unsubstituted alkyl)₂, —S(═O)₂N (substituted or unsubstituted alkyl)(substituted or unsubstituted phenyl)). In certain embodiments, at least one instance of R⁷ is —P(═O)(R^{a})₂ (e.g., —P(═O)(substituted or unsubstituted phenyl)₂). In certain embodiments, at least one instance of R⁷ is ═O. In certain embodiments, at least one R⁷ is halogen, substituted or unsubstituted alkyl, substituted or unsubstituted, 3- to 7-membered, monocyclic carbocyclyl, substituted or unsubstituted phenyl, —OR^{a}, —CN, or —N(R^{a})₂. In certain embodiments, each R⁷ is independently halogen, substituted or unsubstituted alkyl, substituted or unsubstituted, 3- to 7-membered, monocyclic carbocyclyl, substituted or unsubstituted phenyl, —OR^{a}, —CN, or —N(R^{a})₂. In certain embodiments, at least one R⁷ is chloro, fluoro, —CH₃, —CF₃, unsubstituted benzyl, unsubstituted C₂₋₆ alkyl, —OCH₃, —O(unsubstituted C₂₋₆ alkyl), —OCH₂CH₂OCH₃, —CN, —NHCH₃, or —N(CH₃)₂. In certain embodiments, at least one R⁷ is halogen, substituted or unsubstituted alkyl, —OR^{a}, or —CN. In certain embodiments, at least one R⁷ is halogen, unsubstituted C₁₋₆ alkyl, —O(unsubstituted C₁₋₆ alkyl), or —CN. In certain embodiments, each R⁷ is independently halogen, substituted or unsubstituted alkyl, —OR^{a}, or —CN. In certain embodiments, each R⁷ is independently halogen, unsubstituted C₁₋₆ alkyl, —O(unsubstituted C₁₋₆ alkyl), or —CN. In certain embodiments, at least one R⁷ is halogen or substituted or unsubstituted alkyl (e.g., unsubstituted C₁₋₆ alkyl). In certain embodiments, each R⁷ is independently halogen or substituted or unsubstituted alkyl. In certain embodiments, each R⁷ is independently halogen or unsubstituted C₁₋₆ alkyl. In certain embodiments, each R⁷ is independently C₁ or Me.

This paragraph applies when R⁷ is attached to a nitrogen atom. In certain embodiments, at least one instance of R⁷ is unsubstituted alkyl (e.g., unsubstituted C₁₋₆ alkyl). In certain embodiments, at least one instance of R⁷ is Me. In certain embodiments, at least one instance of R⁷ is Et, Pr, or Bu. In certain embodiments, at least one instance of R⁷ is substituted alkyl (e.g., alkyl substituted with one or more instances of halogen (e.g., F)). In certain embodiments, at least one instance of R⁷ is substituted C₁₋₆ alkyl. In certain embodiments, at least one instance of R⁷ is substituted methyl (e.g., fluorinated methyl or Bn). In certain embodiments, at least one instance of R⁷ is substituted ethyl, substituted propyl, or substituted butyl. In certain embodiments, at least one instance of R⁷ is substituted or unsubstituted alkenyl. In certain embodiments, at least one instance of R⁷ is substituted or unsubstituted, C₂₋₆ alkenyl (e.g., substituted or unsubstituted vinyl or substituted or unsubstituted allyl). In certain embodiments, at least one instance of R⁷ is substituted or unsubstituted alkynyl. In certain embodiments, at least one instance of R⁷ is substituted or unsubstituted, C₂₋₆ alkynyl (e.g., substituted or unsubstituted ethynyl). In certain embodiments, at least one instance of R⁷ is substituted or unsubstituted carbocyclyl (e.g., substituted or unsubstituted, monocyclic, 3- to 7-membered carbocyclyl). In certain embodiments, at least one instance of R⁷ is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R⁷ is substituted or unsubstituted heterocyclyl (e.g., substituted or unsubstituted, 3- to 7-membered, monocyclic heterocyclyl). In certain embodiments, at least one instance of R⁷ is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R⁷ is substituted or unsubstituted aryl. In certain embodiments, at least one instance of R⁷ is substituted or unsubstituted phenyl. In certain embodiments, at least one instance of R⁷ is substituted or unsubstituted naphthyl. In certain embodiments, at least one instance of R⁷ is substituted or unsubstituted heteroaryl. In certain embodiments, at least one instance of R⁷ is substituted or unsubstituted, 5- to 6-membered, monocyclic heteroaryl. In certain embodiments, at least one instance of R⁷ is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R⁷ is substituted or unsubstituted pyridinyl, substituted or unsubstituted pyrazinyl, substituted or unsubstituted pyrimidinyl, or substituted or unsubstituted pyridazinyl. In certain embodiments, at least one instance of R⁷ is substituted or unsubstituted, 9- to 10-membered, bicyclic heteroaryl. In certain embodiments, at least one instance of R⁷ is —C(═O)R^{a} (e.g., —C(═O)(substituted or unsubstituted alkyl) (e.g., —C(═O)Me) or —C(═O)(substituted or unsubstituted phenyl)). In certain embodiments, at least one instance of R⁷ is —C(═O)OR^{a} (e.g., —C(═O)OH, —C(═O)O (substituted or unsubstituted alkyl) (e.g., —C(═O)OMe), or —C(═O)O (substituted or unsubstituted phenyl)). In certain embodiments, at least one instance of R⁷ is —C(═O)N(R^{a})₂ (e.g., —C(═O)NH₂, —C(═O)NH (substituted or unsubstituted alkyl) (e.g., —C(═O)NHMe), —C(═O)NH (substituted or unsubstituted phenyl), —C(═O)N (substituted or unsubstituted alkyl)-(substituted or unsubstituted alkyl), or —C(═O)N (substituted or unsubstituted phenyl)-(substituted or unsubstituted alkyl)). In certain embodiments, at least one instance of R⁷ is —S(═O)₂R^{a} (e.g., —S(═O)₂ (substituted or unsubstituted alkyl or substituted or unsubstituted phenyl)). In certain embodiments, at least one instance of R⁷ is —S(═O)₂OR^{a} (e.g., —S(═O)₂OH, —S(═O)₂O (substituted or unsubstituted alkyl or substituted or unsubstituted phenyl)). In certain embodiments, at least one instance of R⁷ is —S(═O)₂N(R^{a})₂ (e.g., —S(═O)₂NH₂, —S(═O)₂NH (substituted or unsubstituted alkyl or substituted or unsubstituted phenyl), —S(═O)₂N (substituted or unsubstituted alkyl)₂, —S(═O)₂N (substituted or unsubstituted alkyl)(substituted or unsubstituted phenyl)). In certain embodiments, at least one instance of R⁷ is —P(═O)(R^{a})₂ (e.g., —P(═O)(substituted or unsubstituted phenyl)₂). In certain embodiments, at least one instance of R⁷ is a nitrogen protecting group (e.g., Bn, Boc, Cbz, Fmoc, trifluoroacetyl, triphenylmethyl, acetyl, or Ts). In certain embodiments, at least one instance of R⁷ is ═O.

In certain embodiments, R¹ and R³ are not joined with their intervening atoms to form substituted or unsubstituted heterocyclyl. In certain embodiments, R⁶ and R⁷ are not joined with their intervening atoms to form substituted or unsubstituted heterocyclyl. In certain embodiments, R¹ and R³ are not joined with their intervening atoms to form substituted or unsubstituted heterocyclyl; and R⁶ and R⁷ are not joined with their intervening atoms to form substituted or unsubstituted heterocyclyl.

In certain embodiments, n is 0. In certain embodiments, n is 1. In certain embodiments, n is 2. In certain embodiments, n is 3. In certain embodiments, n is 4. In certain embodiments, n is 5. In certain embodiments, n is 0, 1, or 2. In certain embodiments, n is 1 or 2. In certain embodiments, n is such an integer between 1 and 13, inclusive, that Ring C is fully substituted.

In certain embodiments, the compound is of the formula:

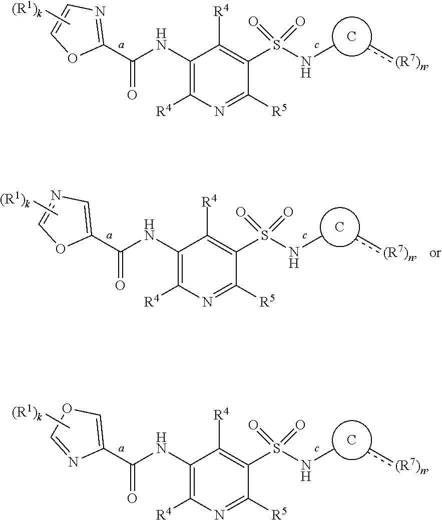

or a pharmaceutically acceptable salt, solvate, hydrate, polymorph, co-crystal, tautomer, stereoisomer, isotopically labeled compound, or prodrug thereof.

In certain embodiments, the compound is of the formula:

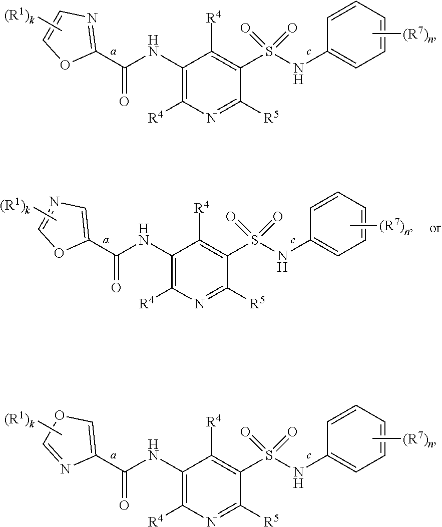

or a pharmaceutically acceptable salt, solvate, hydrate, polymorph, co-crystal, tautomer, stereoisomer, isotopically labeled compound, or prodrug thereof.

In certain embodiments, the compound is of the formula:

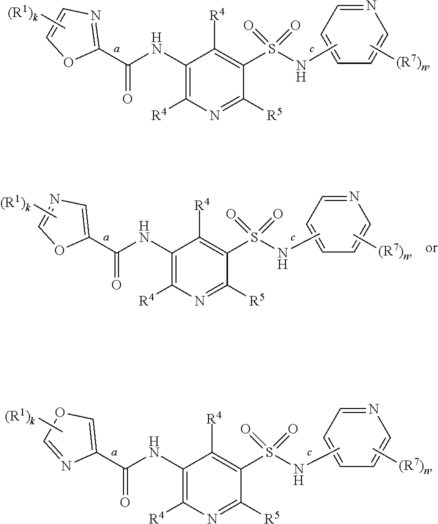

or a pharmaceutically acceptable salt, solvate, hydrate, polymorph, co-crystal, tautomer, stereoisomer, isotopically labeled compound, or prodrug thereof.

In certain embodiments, the compound is of the formula:

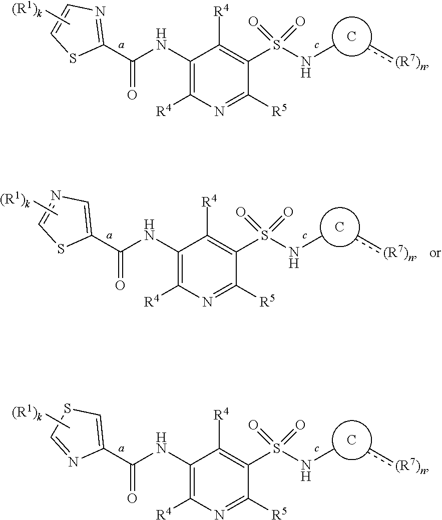

or a pharmaceutically acceptable salt, solvate, hydrate, polymorph, co-crystal, tautomer, stereoisomer, isotopically labeled compound, or prodrug thereof.

In certain embodiments, the compound is of the formula:

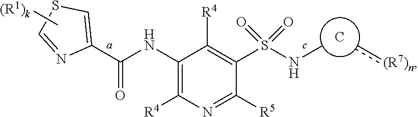

or a pharmaceutically acceptable salt, solvate, hydrate, polymorph, co-crystal, tautomer, stereoisomer, isotopically labeled compound, or prodrug thereof.

In certain embodiments, the compound is of the formula:

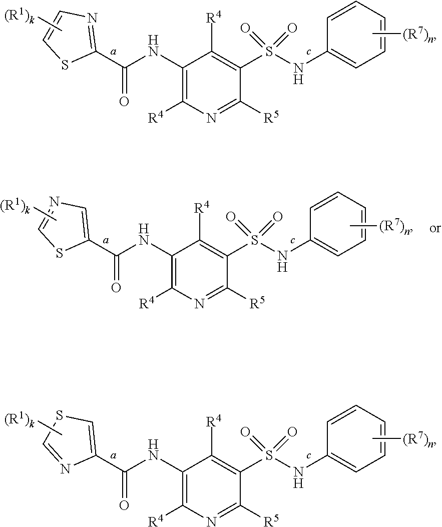

or a pharmaceutically acceptable salt, solvate, hydrate, polymorph, co-crystal, tautomer, stereoisomer, isotopically labeled compound, or prodrug thereof.

In certain embodiments, the compound is of the formula:

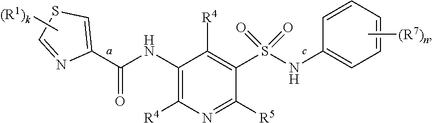

or a pharmaceutically acceptable salt, solvate, hydrate, polymorph, co-crystal, tautomer, stereoisomer, isotopically labeled compound, or prodrug thereof.

In certain embodiments, the compound is of the formula:

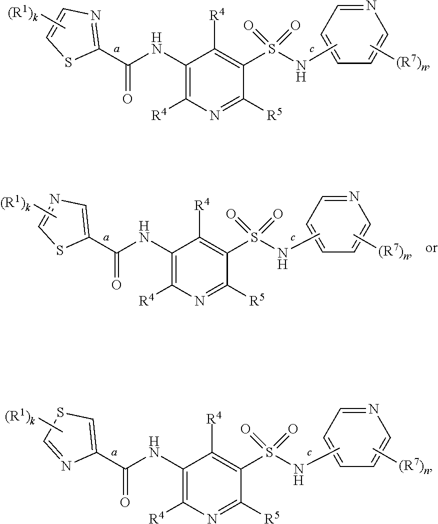

or a pharmaceutically acceptable salt, solvate, hydrate, polymorph, co-crystal, tautomer, stereoisomer, isotopically labeled compound, or prodrug thereof.

In certain embodiments, the compound is of the formula:

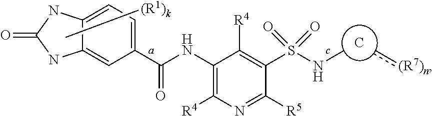

or a pharmaceutically acceptable salt, solvate, hydrate, polymorph, co-crystal, tautomer, stereoisomer, isotopically labeled compound, or prodrug thereof.

In certain embodiments, the compound is of the formula:

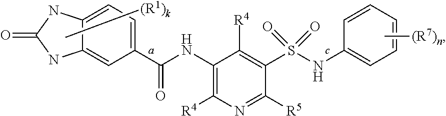

or a pharmaceutically acceptable salt, solvate, hydrate, polymorph, co-crystal, tautomer, stereoisomer, isotopically labeled compound, or prodrug thereof.

In certain embodiments, the compound is of the formula:

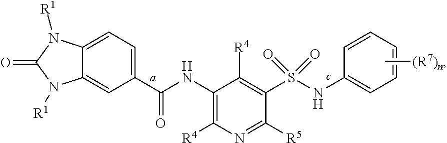

or a pharmaceutically acceptable salt, solvate, hydrate, polymorph, co-crystal, tautomer, stereoisomer, isotopically labeled compound, or prodrug thereof.

In certain embodiments, the compound is of the formula:

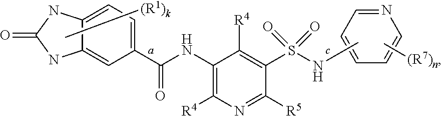

or a pharmaceutically acceptable salt, solvate, hydrate, polymorph, co-crystal, tautomer, stereoisomer, isotopically labeled compound, or prodrug thereof.

In certain embodiments,

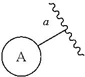

is not

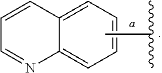

In certain embodiments,

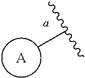

is not

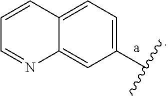 [SMILES: *c1ccc2cccnc2c1]

In certain embodiments, the compound is of the formula:

| No. | Formula |
|---|---|
| 535 | 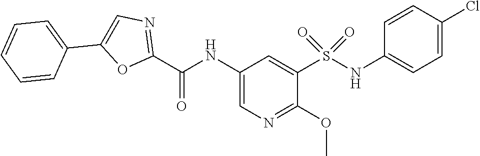 [SMILES: COc1ncc(NC(=O)c2ncc(-c3ccccc3)o2)cc1S(=O)(=O)Nc1ccc(Cl)cc1] |
| 592 | 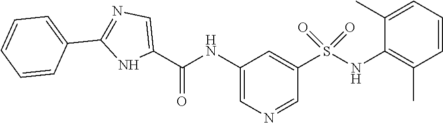 [SMILES: Cc1cccc(C)c1NS(=O)(=O)c1cncc(NC(=O)c2cnc(-c3ccccc3)[nH]2)c1] |
| 594 | 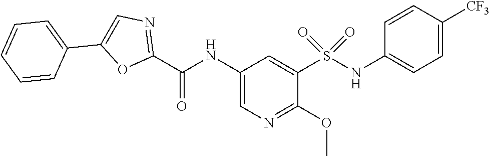 [SMILES: COc1ncc(NC(=O)c2ncc(-c3ccccc3)o2)cc1S(=O)(=O)Nc1ccc(C(F)(F)F)cc1] |
| 604 | 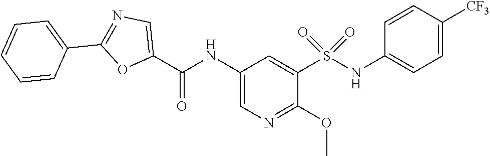 [SMILES: COc1ncc(NC(=O)c2cnc(-c3ccccc3)o2)cc1S(=O)(=O)Nc1ccc(C(F)(F)F)cc1] |
| 612 | 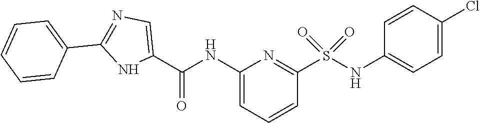 [SMILES: O=C(Nc1cccc(S(=O)(=O)Nc2ccc(Cl)cc2)n1)c1cnc(-c2ccccc2)[nH]1] |
| 670 | 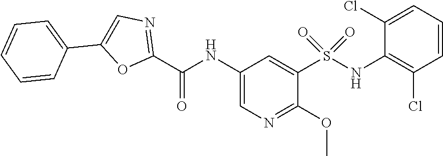 [SMILES: COc1ncc(NC(=O)c2ncc(-c3ccccc3)o2)cc1S(=O)(=O)Nc1c(Cl)cccc1Cl] |
| 1178 | 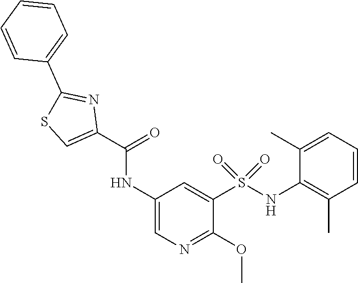 [SMILES: COc1ncc(NC(=O)c2csc(-c3ccccc3)n2)cc1S(=O)(=O)Nc1c(C)cccc1C] |
| 1193 | 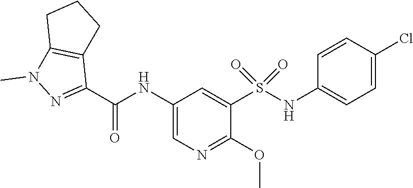 [SMILES: COc1ncc(NC(=O)c2nn(C)c3c2CCC3)cc1S(=O)(=O)Nc1ccc(Cl)cc1] |
| 1194 | 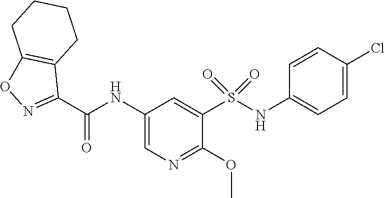 [SMILES: COc1ncc(NC(=O)c2noc3c2CCCC3)cc1S(=O)(=O)Nc1ccc(Cl)cc1] |
| 1195 | 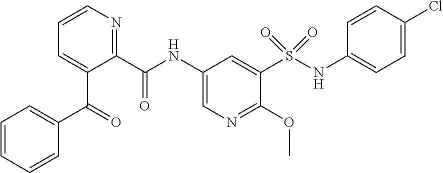 [SMILES: COc1ncc(NC(=O)c2ncccc2C(=O)c2ccccc2)cc1S(=O)(=O)Nc1ccc(Cl)cc1] |
| 1197 | 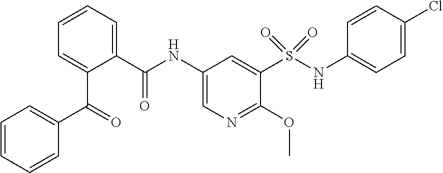 [SMILES: COc1ncc(NC(=O)c2ccccc2C(=O)c2ccccc2)cc1S(=O)(=O)Nc1ccc(Cl)cc1] |
| 1198 | 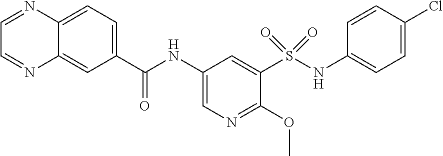 [SMILES: COc1ncc(NC(=O)c2ccc3nccnc3c2)cc1S(=O)(=O)Nc1ccc(Cl)cc1] |
| 1199 | 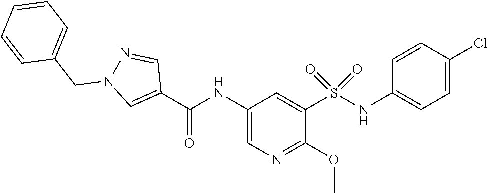 [SMILES: COc1ncc(NC(=O)c2cnn(Cc3ccccc3)c2)cc1S(=O)(=O)Nc1ccc(Cl)cc1] |
| 1200 | 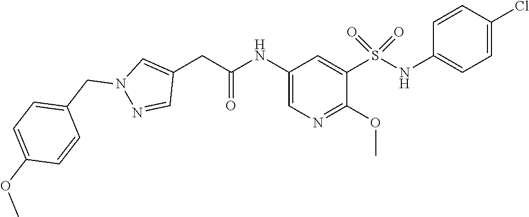 [SMILES: COc1ccc(Cn2cc(CC(=O)Nc3cnc(OC)c(S(=O)(=O)Nc4ccc(Cl)cc4)c3)cn2)cc1] |
| 1201 | 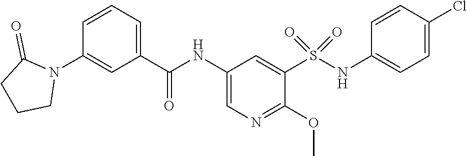 [SMILES: COc1ncc(NC(=O)c2cccc(N3CCCC3=O)c2)cc1S(=O)(=O)Nc1ccc(Cl)cc1] |
| 1202 | 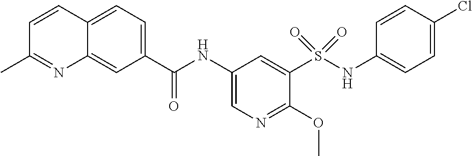 [SMILES: COc1ncc(NC(=O)c2ccc3ccc(C)nc3c2)cc1S(=O)(=O)Nc1ccc(Cl)cc1] |
| 1203 | 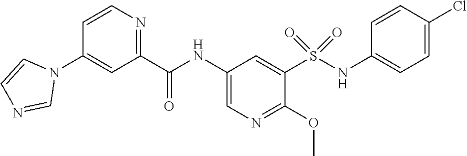 [SMILES: COc1ncc(NC(=O)c2cc(-n3ccnc3)ccn2)cc1S(=O)(=O)Nc1ccc(Cl)cc1] |
| 1204 | 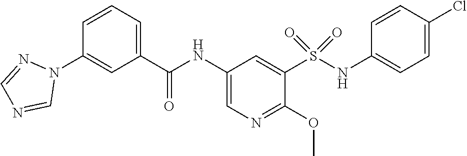 [SMILES: COc1ncc(NC(=O)c2cccc(-n3cncn3)c2)cc1S(=O)(=O)Nc1ccc(Cl)cc1] |
| 1206 | 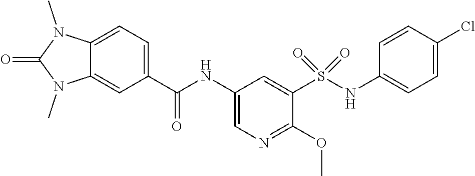 [SMILES: COc1ncc(NC(=O)c2ccc3c(c2)n(C)c(=O)n3C)cc1S(=O)(=O)Nc1ccc(Cl)cc1] |
| 1213 | 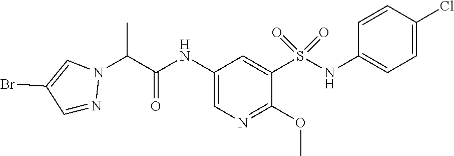 [SMILES: COc1ncc(NC(=O)C(C)n2cc(Br)cn2)cc1S(=O)(=O)Nc1ccc(Cl)cc1] |
| 1294 | 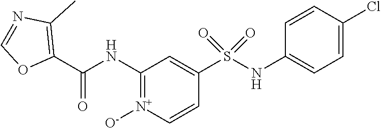 [SMILES: Cc1ncoc1C(=O)Nc1cc(S(=O)(=O)Nc2ccc(Cl)cc2)cc[n+]1[O-]] |
| 1303 | 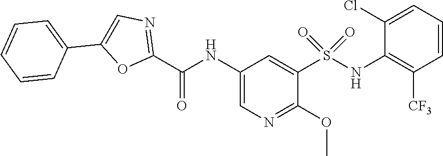 [SMILES: COc1ncc(NC(=O)c2ncc(-c3ccccc3)o2)cc1S(=O)(=O)Nc1c(Cl)cccc1C(F)(F)F] |
| 1312 | 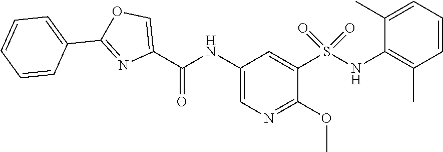 [SMILES: COc1ncc(NC(=O)c2coc(-c3ccccc3)n2)cc1S(=O)(=O)Nc1c(C)cccc1C] |
| 1321 | 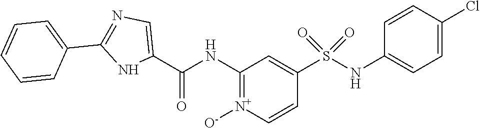 [SMILES: O=C(Nc1cc(S(=O)(=O)Nc2ccc(Cl)cc2)cc[n+]1[O-])c1cnc(-c2ccccc2)[nH]1] |
| 1350 | 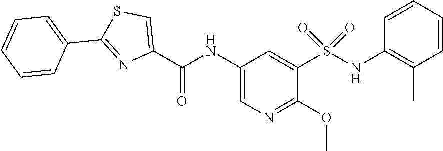 [SMILES: COc1ncc(NC(=O)c2csc(-c3ccccc3)n2)cc1S(=O)(=O)Nc1ccccc1C] |
| 1351 | 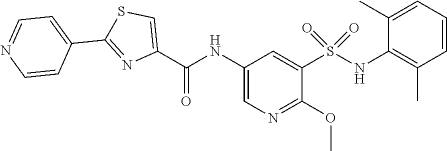 [SMILES: COc1ncc(NC(=O)c2csc(-c3ccncc3)n2)cc1S(=O)(=O)Nc1c(C)cccc1C] |
| 1358 | 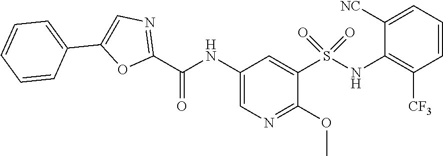 [SMILES: COc1ncc(NC(=O)c2ncc(-c3ccccc3)o2)cc1S(=O)(=O)Nc1c(C#N)cccc1C(F)(F)F] |
| 1359 | 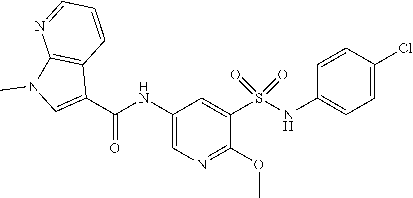 [SMILES: COc1ncc(NC(=O)c2cn(C)c3ncccc23)cc1S(=O)(=O)Nc1ccc(Cl)cc1] |
| 1361 | 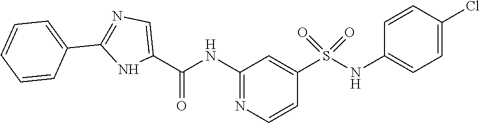 [SMILES: O=C(Nc1cc(S(=O)(=O)Nc2ccc(Cl)cc2)ccn1)c1cnc(-c2ccccc2)[nH]1] |
| 1364 | 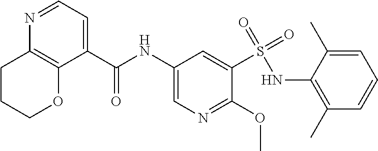 [SMILES: COc1ncc(NC(=O)c2ccnc3c2OCCC3)cc1S(=O)(=O)Nc1c(C)cccc1C] |
| 1365 | 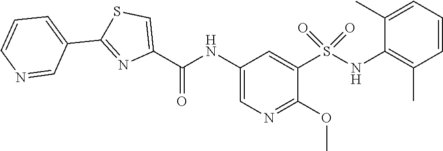 [SMILES: COc1ncc(NC(=O)c2csc(-c3cccnc3)n2)cc1S(=O)(=O)Nc1c(C)cccc1C] |
| 1366 | 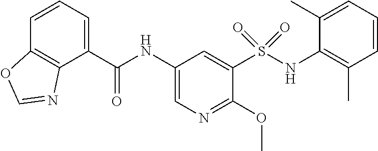 [SMILES: COc1ncc(NC(=O)c2cccc3ocnc23)cc1S(=O)(=O)Nc1c(C)cccc1C] |
| 1367 | 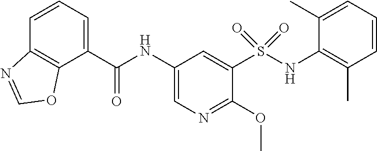 [SMILES: COc1ncc(NC(=O)c2cccc3ncoc23)cc1S(=O)(=O)Nc1c(C)cccc1C] |
| 1377 | 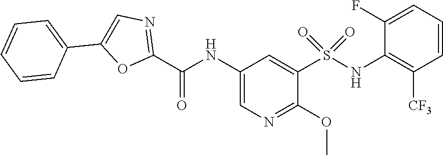 [SMILES: COc1ncc(NC(=O)c2ncc(-c3ccccc3)o2)cc1S(=O)(=O)Nc1c(F)cccc1C(F)(F)F] |
| 1379 | 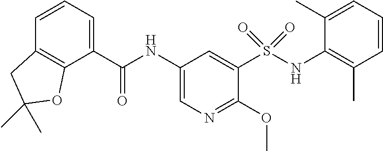 [SMILES: COc1ncc(NC(=O)c2cccc3c2OC(C)(C)C3)cc1S(=O)(=O)Nc1c(C)cccc1C] |
| 1380 | 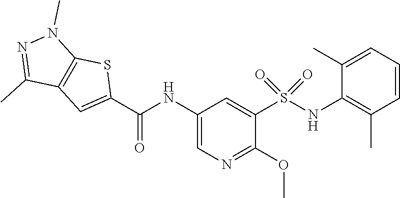 [SMILES: COc1ncc(NC(=O)c2cc3c(C)nn(C)c3s2)cc1S(=O)(=O)Nc1c(C)cccc1C] |
| 1382 | 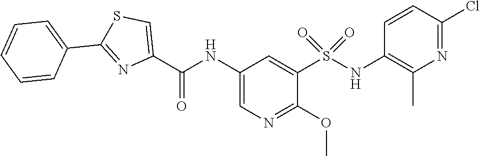 [SMILES: COc1ncc(NC(=O)c2csc(-c3ccccc3)n2)cc1S(=O)(=O)Nc1ccc(Cl)nc1C] |
| 1383 | 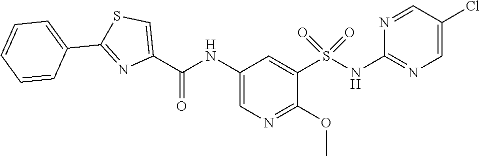 [SMILES: COc1ncc(NC(=O)c2csc(-c3ccccc3)n2)cc1S(=O)(=O)Nc1ncc(Cl)cn1] |
| 1385 | 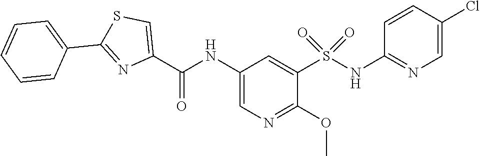 [SMILES: COc1ncc(NC(=O)c2csc(-c3ccccc3)n2)cc1S(=O)(=O)Nc1ccc(Cl)cn1] |
| 1386 | 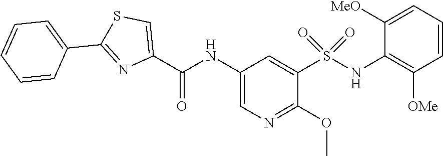 [SMILES: COc1cccc(OC)c1NS(=O)(=O)c1cc(NC(=O)c2csc(-c3ccccc3)n2)cnc1OC] |
| 1388 | 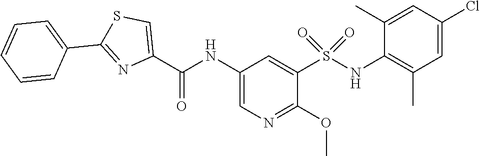 [SMILES: COc1ncc(NC(=O)c2csc(-c3ccccc3)n2)cc1S(=O)(=O)Nc1c(C)cc(Cl)cc1C] |
| 1389 | 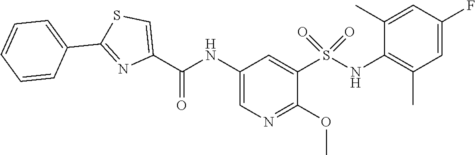 [SMILES: COc1ncc(NC(=O)c2csc(-c3ccccc3)n2)cc1S(=O)(=O)Nc1c(C)cc(F)cc1C] |
| 1390 | 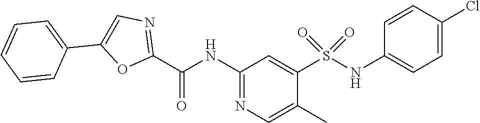 [SMILES: Cc1cnc(NC(=O)c2ncc(-c3ccccc3)o2)cc1S(=O)(=O)Nc1ccc(Cl)cc1] |
| 1393 | 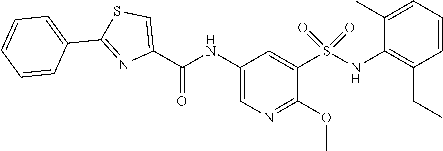 [SMILES: CCc1cccc(C)c1NS(=O)(=O)c1cc(NC(=O)c2csc(-c3ccccc3)n2)cnc1OC] |
| 1394 | 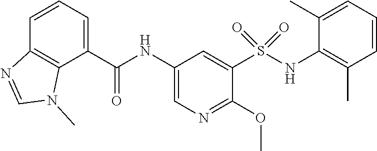 [SMILES: COc1ncc(NC(=O)c2cccc3ncn(C)c23)cc1S(=O)(=O)Nc1c(C)cccc1C] |
| 1395 | 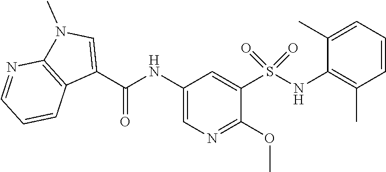 [SMILES: COc1ncc(NC(=O)c2cn(C)c3ncccc23)cc1S(=O)(=O)Nc1c(C)cccc1C] |
| 1396 | 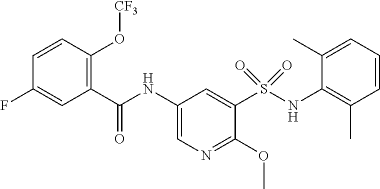 [SMILES: COc1ncc(NC(=O)c2cc(F)ccc2OC(F)(F)F)cc1S(=O)(=O)Nc1c(C)cccc1C] |
| 1398 | 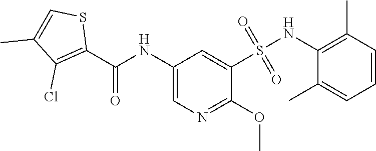 [SMILES: COc1ncc(NC(=O)c2scc(C)c2Cl)cc1S(=O)(=O)Nc1c(C)cccc1C] |
| 1400 | 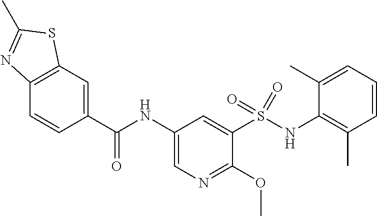 [SMILES: COc1ncc(NC(=O)c2ccc3nc(C)sc3c2)cc1S(=O)(=O)Nc1c(C)cccc1C] |
| 1401 | 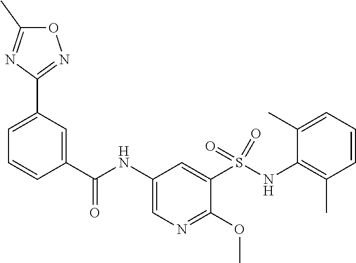 [SMILES: COc1ncc(NC(=O)c2cccc(-c3noc(C)n3)c2)cc1S(=O)(=O)Nc1c(C)cccc1C] |
| 1402 | 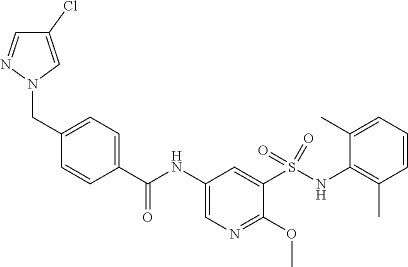 [SMILES: COc1ncc(NC(=O)c2ccc(Cn3cc(Cl)cn3)cc2)cc1S(=O)(=O)Nc1c(C)cccc1C] |
| 1403 | 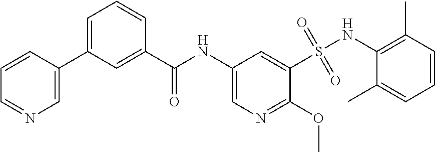 [SMILES: COc1ncc(NC(=O)c2cccc(-c3cccnc3)c2)cc1S(=O)(=O)Nc1c(C)cccc1C] |
| 1404 | 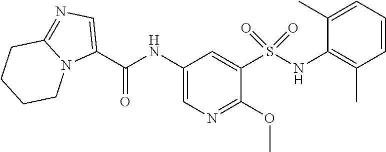 [SMILES: COc1ncc(NC(=O)c2cnc3n2CCCC3)cc1S(=O)(=O)Nc1c(C)cccc1C] |
| 1406 | 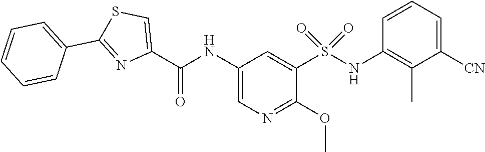 [SMILES: COc1ncc(NC(=O)c2csc(-c3ccccc3)n2)cc1S(=O)(=O)Nc1cccc(C#N)c1C] |
| 1413 | 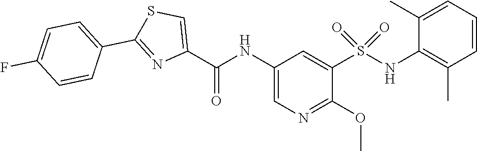 [SMILES: COc1ncc(NC(=O)c2csc(-c3ccc(F)cc3)n2)cc1S(=O)(=O)Nc1c(C)cccc1C] |
| 1417 | 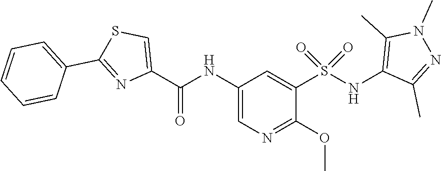 [SMILES: COc1ncc(NC(=O)c2csc(-c3ccccc3)n2)cc1S(=O)(=O)Nc1c(C)nn(C)c1C] |
| 1418 | 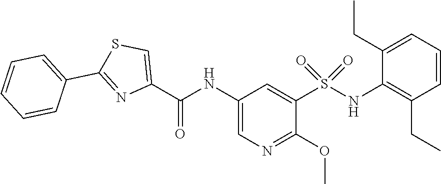 [SMILES: CCc1cccc(CC)c1NS(=O)(=O)c1cc(NC(=O)c2csc(-c3ccccc3)n2)cnc1OC] |
| 1420 | 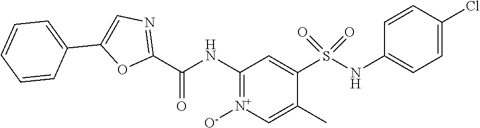 [SMILES: Cc1c[n+]([O-])c(NC(=O)c2ncc(-c3ccccc3)o2)cc1S(=O)(=O)Nc1ccc(Cl)cc1] |
| 1421 | 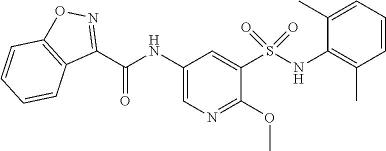 [SMILES: COc1ncc(NC(=O)c2noc3ccccc23)cc1S(=O)(=O)Nc1c(C)cccc1C] |
| 1422 | 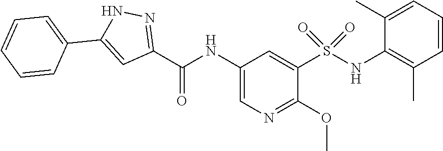 [SMILES: COc1ncc(NC(=O)c2cc(-c3ccccc3)[nH]n2)cc1S(=O)(=O)Nc1c(C)cccc1C] |
| 1423 | 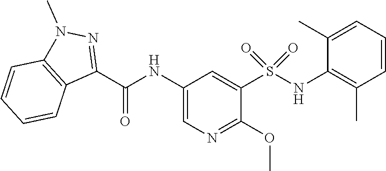 [SMILES: COc1ncc(NC(=O)c2nn(C)c3ccccc23)cc1S(=O)(=O)Nc1c(C)cccc1C] |
| 1424 | 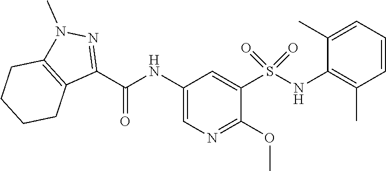 [SMILES: COc1ncc(NC(=O)c2nn(C)c3c2CCCC3)cc1S(=O)(=O)Nc1c(C)cccc1C] |
| 1425 | 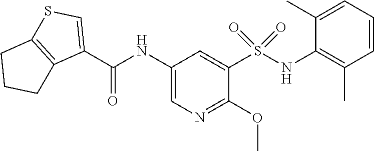 [SMILES: COc1ncc(NC(=O)c2csc3c2CCC3)cc1S(=O)(=O)Nc1c(C)cccc1C] |
| 1481 | 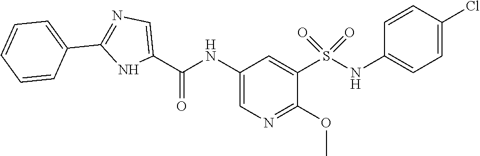 [SMILES: COc1ncc(NC(=O)c2cnc(-c3ccccc3)[nH]2)cc1S(=O)(=O)Nc1ccc(Cl)cc1] |
| 1483 | 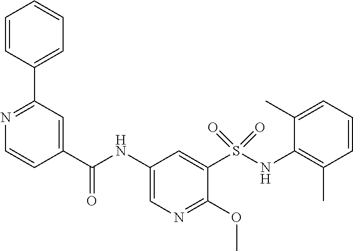 [SMILES: COc1ncc(NC(=O)c2ccnc(-c3ccccc3)c2)cc1S(=O)(=O)Nc1c(C)cccc1C] |
| 1484 | 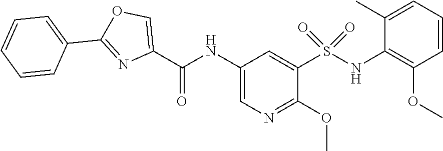 [SMILES: COc1cccc(C)c1NS(=O)(=O)c1cc(NC(=O)c2coc(-c3ccccc3)n2)cnc1OC] |
| 1485 | 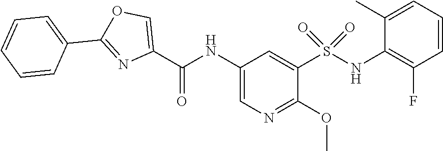 [SMILES: COc1ncc(NC(=O)c2coc(-c3ccccc3)n2)cc1S(=O)(=O)Nc1c(C)cccc1F] |
| 1486 | 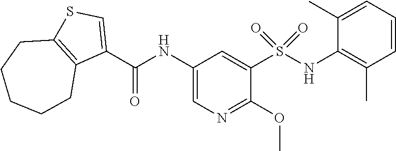 [SMILES: COc1ncc(NC(=O)c2csc3c2CCCCC3)cc1S(=O)(=O)Nc1c(C)cccc1C] |
| 1503 | 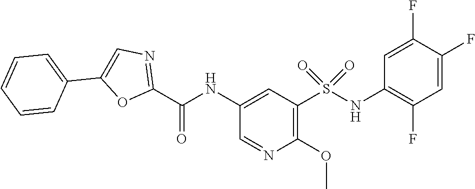 [SMILES: COc1ncc(NC(=O)c2ncc(-c3ccccc3)o2)cc1S(=O)(=O)Nc1cc(F)c(F)cc1F] |
| 1511 | 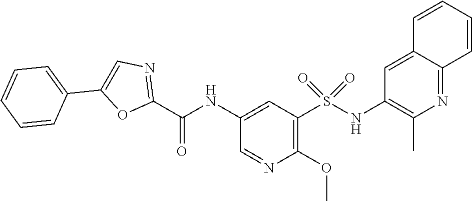 [SMILES: COc1ncc(NC(=O)c2ncc(-c3ccccc3)o2)cc1S(=O)(=O)Nc1cc2ccccc2nc1C] |
| 1513 | 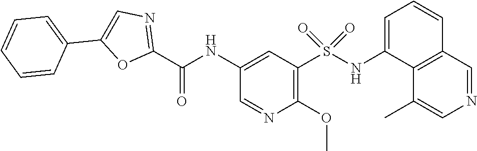 [SMILES: COc1ncc(NC(=O)c2ncc(-c3ccccc3)o2)cc1S(=O)(=O)Nc1cccc2cncc(C)c12] |
| 1514 | 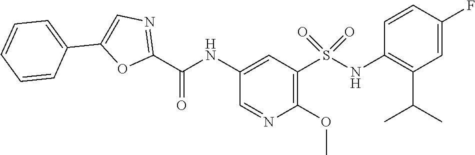 [SMILES: COc1ncc(NC(=O)c2ncc(-c3ccccc3)o2)cc1S(=O)(=O)Nc1ccc(F)cc1C(C)C] |
| 1519 | 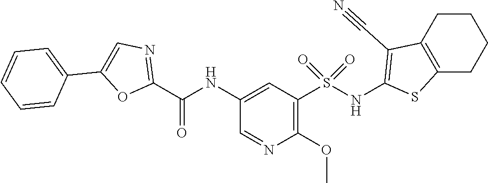 [SMILES: COc1ncc(NC(=O)c2ncc(-c3ccccc3)o2)cc1S(=O)(=O)Nc1sc2c(c1C#N)CCCC2] |
| 1525 | 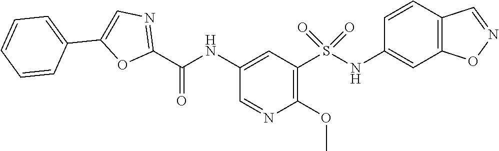 [SMILES: COc1ncc(NC(=O)c2ncc(-c3ccccc3)o2)cc1S(=O)(=O)Nc1ccc2cnoc2c1] |
| 1538 | 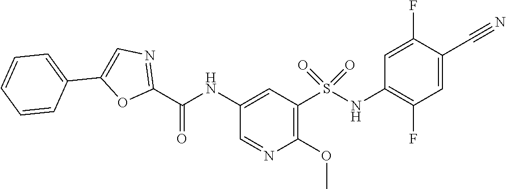 [SMILES: COc1ncc(NC(=O)c2ncc(-c3ccccc3)o2)cc1S(=O)(=O)Nc1cc(F)c(C#N)cc1F] |
| 1552 | 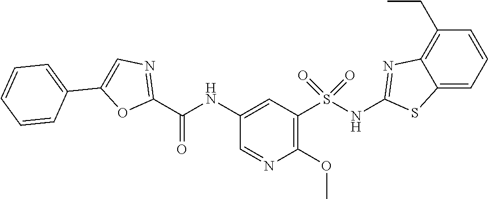 [SMILES: CCc1cccc2sc(NS(=O)(=O)c3cc(NC(=O)c4ncc(-c5ccccc5)o4)cnc3OC)nc12] |
| 1567 | 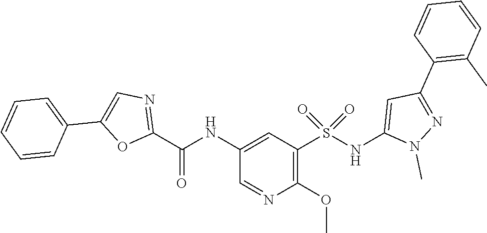 [SMILES: COc1ncc(NC(=O)c2ncc(-c3ccccc3)o2)cc1S(=O)(=O)Nc1cc(-c2ccccc2C)nn1C] |
| 1568 | 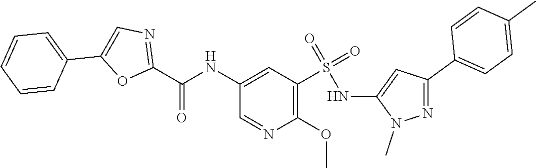 [SMILES: COc1ncc(NC(=O)c2ncc(-c3ccccc3)o2)cc1S(=O)(=O)Nc1cc(-c2ccc(C)cc2)nn1C] |
| 1571 | 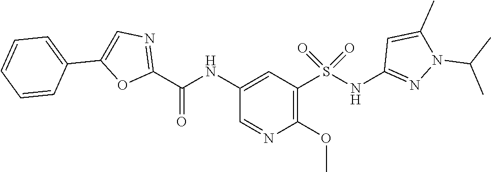 [SMILES: COc1ncc(NC(=O)c2ncc(-c3ccccc3)o2)cc1S(=O)(=O)Nc1cc(C)n(C(C)C)n1] |
| 1624 | 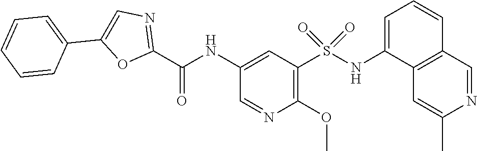 [SMILES: COc1ncc(NC(=O)c2ncc(-c3ccccc3)o2)cc1S(=O)(=O)Nc1cccc2cnc(C)cc12] |
| 1640 | 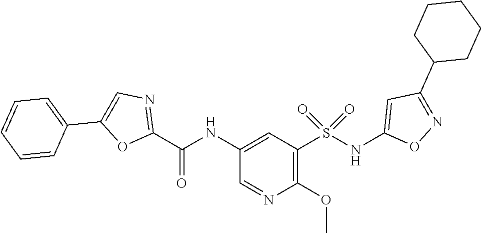 [SMILES: COc1ncc(NC(=O)c2ncc(-c3ccccc3)o2)cc1S(=O)(=O)Nc1cc(C2CCCCC2)no1] |
| 1648 | 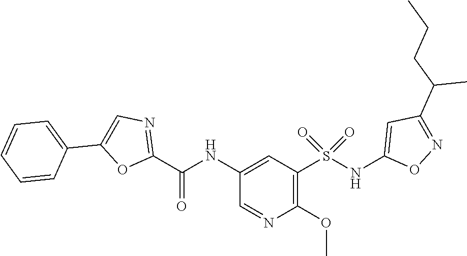 [SMILES: CCCC(C)c1cc(NS(=O)(=O)c2cc(NC(=O)c3ncc(-c4ccccc4)o3)cnc2OC)on1] |
| 1661 | 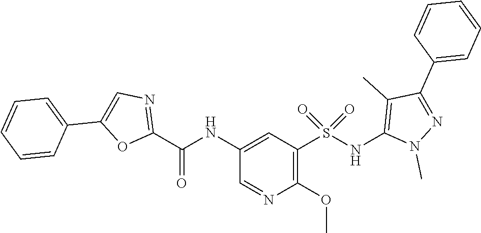 [SMILES: COc1ncc(NC(=O)c2ncc(-c3ccccc3)o2)cc1S(=O)(=O)Nc1c(C)c(-c2ccccc2)nn1C] |
| 1706 | 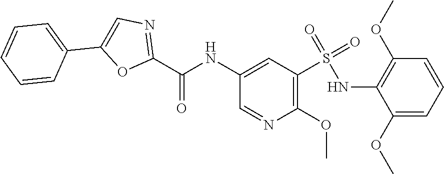 [SMILES: COc1cccc(OC)c1NS(=O)(=O)c1cc(NC(=O)c2ncc(-c3ccccc3)o2)cnc1OC] |
| 1729 | 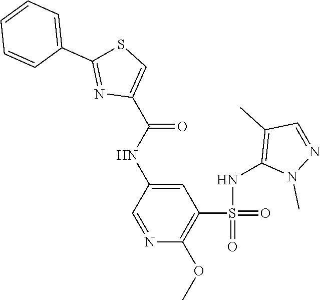 [SMILES: COc1ncc(NC(=O)c2csc(-c3ccccc3)n2)cc1S(=O)(=O)Nc1c(C)cnn1C] |
| 1746 | 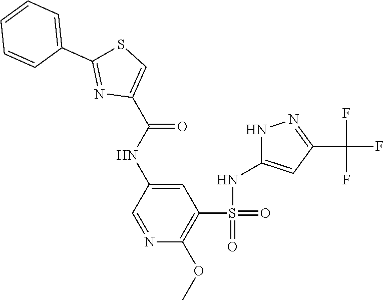 [SMILES: COc1ncc(NC(=O)c2csc(-c3ccccc3)n2)cc1S(=O)(=O)Nc1cc(C(F)(F)F)n[nH]1] |
| 1754 | 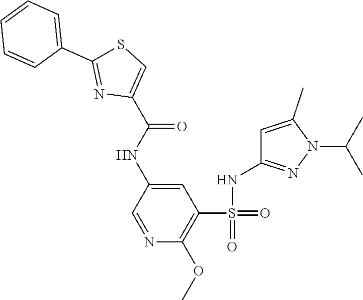 [SMILES: COc1ncc(NC(=O)c2csc(-c3ccccc3)n2)cc1S(=O)(=O)Nc1cc(C)n(C(C)C)n1] |
| 1760 | 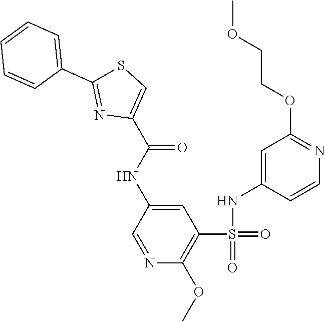 [SMILES: COCCOc1cc(NS(=O)(=O)c2cc(NC(=O)c3csc(-c4ccccc4)n3)cnc2OC)ccn1] |
| 1768 | 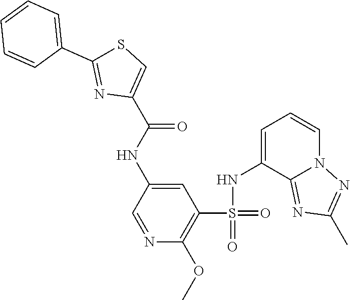 [SMILES: COc1ncc(NC(=O)c2csc(-c3ccccc3)n2)cc1S(=O)(=O)Nc1cccn2nc(C)nc12] |
| 1773 | 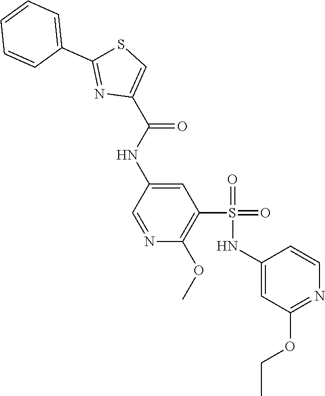 [SMILES: CCOc1cc(NS(=O)(=O)c2cc(NC(=O)c3csc(-c4ccccc4)n3)cnc2OC)ccn1] |
| 1779 | 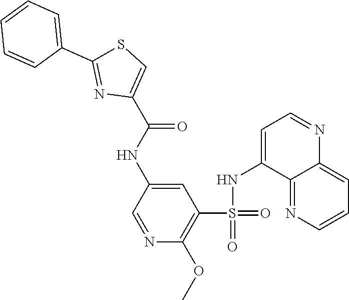 [SMILES: COc1ncc(NC(=O)c2csc(-c3ccccc3)n2)cc1S(=O)(=O)Nc1ccnc2cccnc12] |
| 1780 | 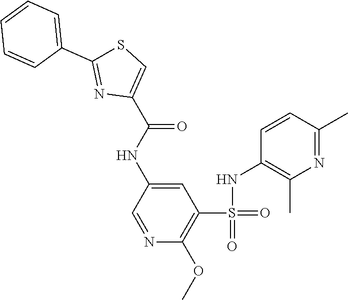 [SMILES: COc1ncc(NC(=O)c2csc(-c3ccccc3)n2)cc1S(=O)(=O)Nc1ccc(C)nc1C] |
| 1784 | 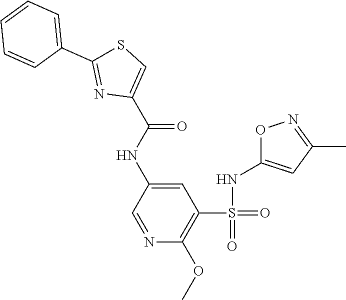 [SMILES: COc1ncc(NC(=O)c2csc(-c3ccccc3)n2)cc1S(=O)(=O)Nc1cc(C)no1] |
| 1785 | 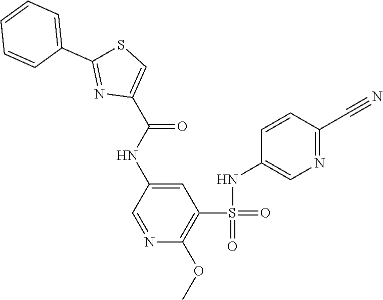 [SMILES: COc1ncc(NC(=O)c2csc(-c3ccccc3)n2)cc1S(=O)(=O)Nc1ccc(C#N)nc1] |
| 1792 | 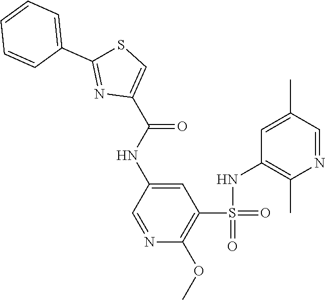 [SMILES: COc1ncc(NC(=O)c2csc(-c3ccccc3)n2)cc1S(=O)(=O)Nc1cc(C)cnc1C] |
| 1808 | 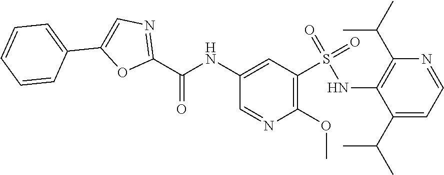 [SMILES: COc1ncc(NC(=O)c2ncc(-c3ccccc3)o2)cc1S(=O)(=O)Nc1c(C(C)C)ccnc1C(C)C] |
| 1824 | 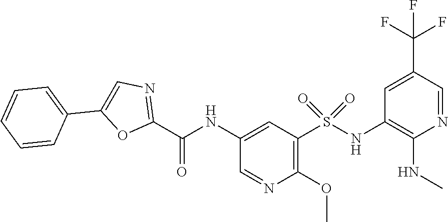 [SMILES: CNc1ncc(C(F)(F)F)cc1NS(=O)(=O)c1cc(NC(=O)c2ncc(-c3ccccc3)o2)cnc1OC] |
| 1839 | 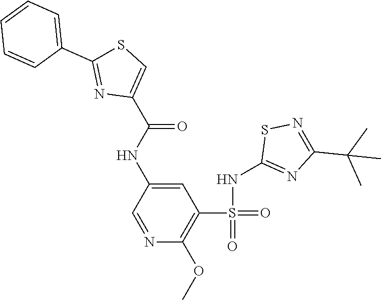 [SMILES: COc1ncc(NC(=O)c2csc(-c3ccccc3)n2)cc1S(=O)(=O)Nc1nc(C(C)(C)C)ns1] |
| 1878 | 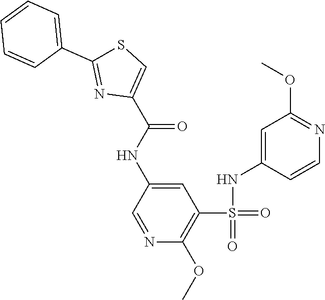 [SMILES: COc1cc(NS(=O)(=O)c2cc(NC(=O)c3csc(-c4ccccc4)n3)cnc2OC)ccn1] |
| 1895 | 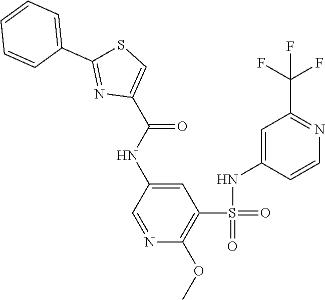 [SMILES: COc1ncc(NC(=O)c2csc(-c3ccccc3)n2)cc1S(=O)(=O)Nc1ccnc(C(F)(F)F)c1] |
| 1898 | 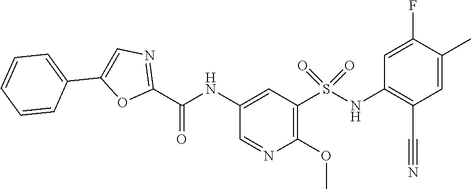 [SMILES: COc1ncc(NC(=O)c2ncc(-c3ccccc3)o2)cc1S(=O)(=O)Nc1cc(F)c(C)cc1C#N] |
| 1908 | 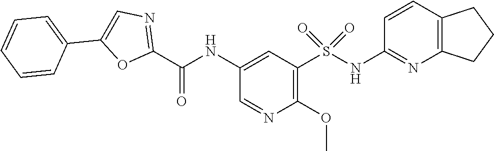 [SMILES: COc1ncc(NC(=O)c2ncc(-c3ccccc3)o2)cc1S(=O)(=O)Nc1ccc2c(n1)CCC2] |
| 1911 | 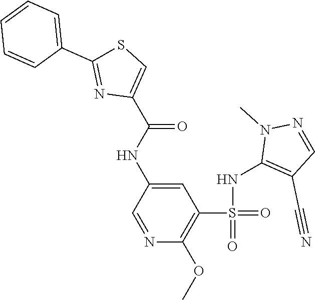 [SMILES: COc1ncc(NC(=O)c2csc(-c3ccccc3)n2)cc1S(=O)(=O)Nc1c(C#N)cnn1C] |
| 1912 | 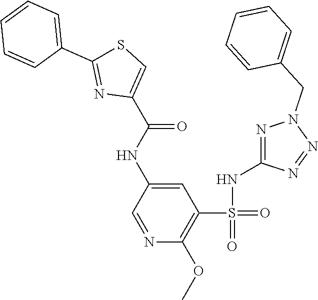 [SMILES: COc1ncc(NC(=O)c2csc(-c3ccccc3)n2)cc1S(=O)(=O)Nc1nnn(Cc2ccccc2)n1] |
| 1926 | 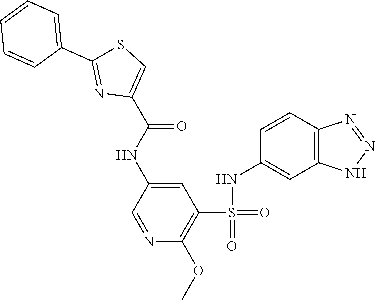 [SMILES: COc1ncc(NC(=O)c2csc(-c3ccccc3)n2)cc1S(=O)(=O)Nc1ccc2nn[nH]c2c1] |
| 1935 | 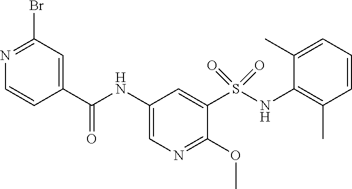 [SMILES: COc1ncc(NC(=O)c2ccnc(Br)c2)cc1S(=O)(=O)Nc1c(C)cccc1C] |
| 1937 | 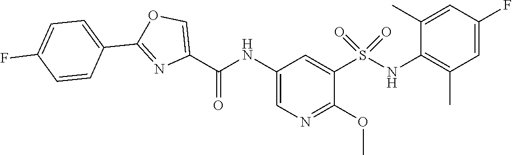 [SMILES: COc1ncc(NC(=O)c2coc(-c3ccc(F)cc3)n2)cc1S(=O)(=O)Nc1c(C)cc(F)cc1C] |
| 1940 | 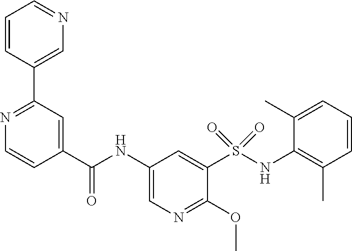 [SMILES: COc1ncc(NC(=O)c2ccnc(-c3cccnc3)c2)cc1S(=O)(=O)Nc1c(C)cccc1C] |
| 1941 | 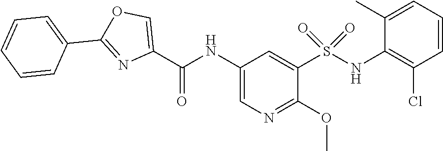 [SMILES: COc1ncc(NC(=O)c2coc(-c3ccccc3)n2)cc1S(=O)(=O)Nc1c(C)cccc1Cl] |
| 1942 | 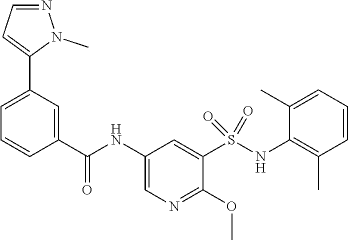 [SMILES: COc1ncc(NC(=O)c2cccc(-c3ccnn3C)c2)cc1S(=O)(=O)Nc1c(C)cccc1C] |
| 1943 | 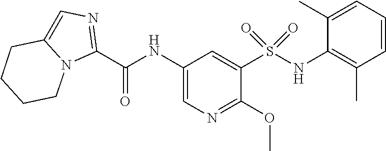 [SMILES: COc1ncc(NC(=O)c2ncc3n2CCCC3)cc1S(=O)(=O)Nc1c(C)cccc1C] |
| 1944 | 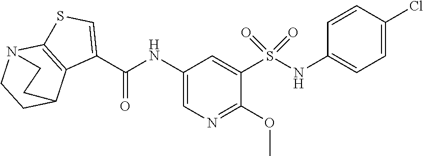 [SMILES: COc1ncc(NC(=O)c2csc3c2C2CCN3CC2)cc1S(=O)(=O)Nc1ccc(Cl)cc1] |
| 1946 | 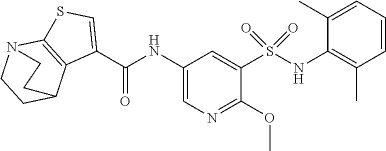 [SMILES: COc1ncc(NC(=O)c2csc3c2C2CCN3CC2)cc1S(=O)(=O)Nc1c(C)cccc1C] |
| 1949 | 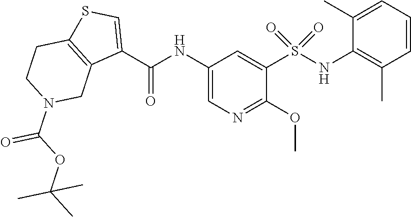 [SMILES: COc1ncc(NC(=O)c2csc3c2CN(C(=O)OC(C)(C)C)CC3)cc1S(=O)(=O)Nc1c(C)cccc1C] |
| 1950 | 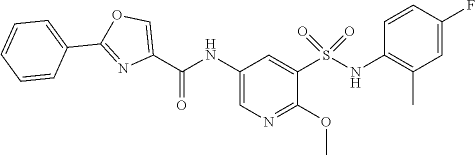 [SMILES: COc1ncc(NC(=O)c2coc(-c3ccccc3)n2)cc1S(=O)(=O)Nc1ccc(F)cc1C] |
| 1953 | 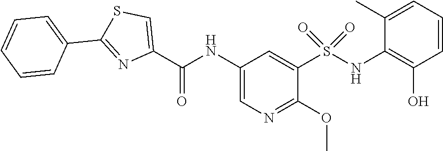 [SMILES: COc1ncc(NC(=O)c2csc(-c3ccccc3)n2)cc1S(=O)(=O)Nc1c(C)cccc1O] |
| 1954 | 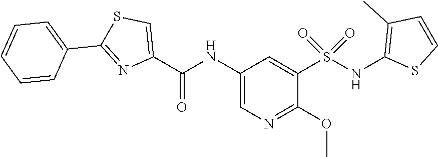 [SMILES: COc1ncc(NC(=O)c2csc(-c3ccccc3)n2)cc1S(=O)(=O)Nc1sccc1C] |
| 1956 | 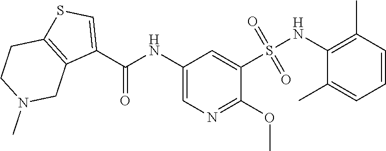 [SMILES: COc1ncc(NC(=O)c2csc3c2CN(C)CC3)cc1S(=O)(=O)Nc1c(C)cccc1C] |
| 1959 | 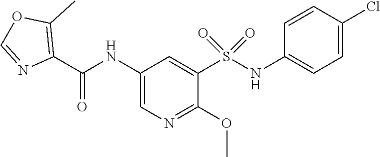 [SMILES: COc1ncc(NC(=O)c2ncoc2C)cc1S(=O)(=O)Nc1ccc(Cl)cc1] |
| 1962 | 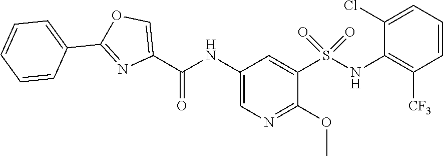 [SMILES: COc1ncc(NC(=O)c2coc(-c3ccccc3)n2)cc1S(=O)(=O)Nc1c(Cl)cccc1C(F)(F)F] |
| 1964 | 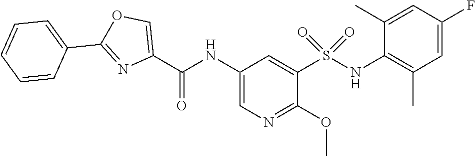 [SMILES: COc1ncc(NC(=O)c2coc(-c3ccccc3)n2)cc1S(=O)(=O)Nc1c(C)cc(F)cc1C] |
| 1965 | 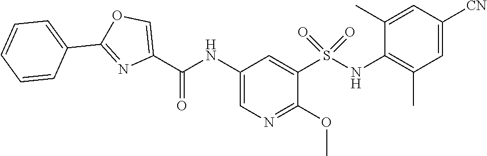 [SMILES: COc1ncc(NC(=O)c2coc(-c3ccccc3)n2)cc1S(=O)(=O)Nc1c(C)cc(C#N)cc1C] |
| 1966 | 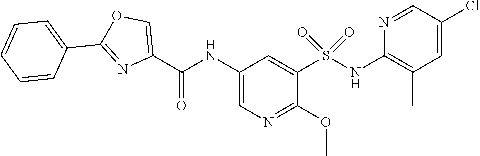 [SMILES: COc1ncc(NC(=O)c2coc(-c3ccccc3)n2)cc1S(=O)(=O)Nc1ncc(Cl)cc1C] |
| 1970 | 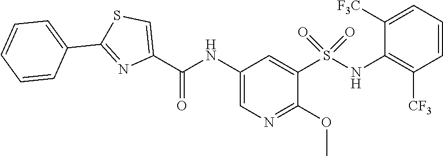 [SMILES: COc1ncc(NC(=O)c2csc(-c3ccccc3)n2)cc1S(=O)(=O)Nc1c(C(F)(F)F)cccc1C(F)(F)F] |
| 1972 | 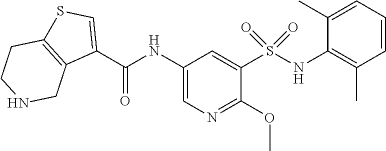 [SMILES: COc1ncc(NC(=O)c2csc3c2CNCC3)cc1S(=O)(=O)Nc1c(C)cccc1C] |
| 1977 | 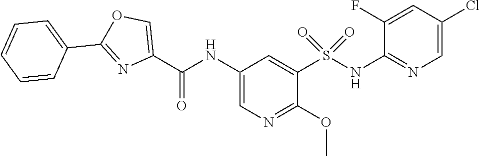 [SMILES: COc1ncc(NC(=O)c2coc(-c3ccccc3)n2)cc1S(=O)(=O)Nc1ncc(Cl)cc1F] |
| 1985 | 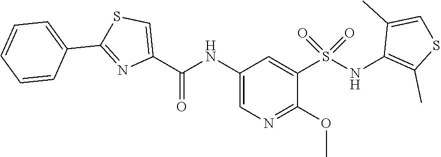 [SMILES: COc1ncc(NC(=O)c2csc(-c3ccccc3)n2)cc1S(=O)(=O)Nc1c(C)csc1C] |
| 1986 | 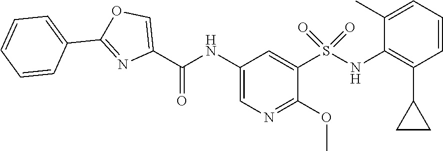 [SMILES: COc1ncc(NC(=O)c2coc(-c3ccccc3)n2)cc1S(=O)(=O)Nc1c(C)cccc1C1CC1] |
| 1990 | 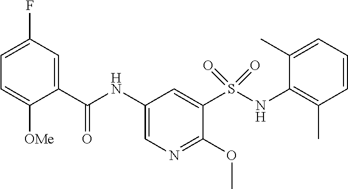 [SMILES: COc1ccc(F)cc1C(=O)Nc1cnc(OC)c(S(=O)(=O)Nc2c(C)cccc2C)c1] |
| 1991 | 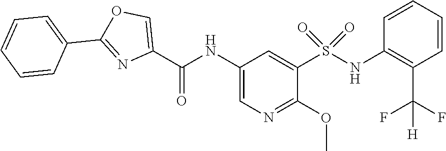 [SMILES: COc1ncc(NC(=O)c2coc(-c3ccccc3)n2)cc1S(=O)(=O)Nc1ccccc1C(F)F] |
| 2116 | 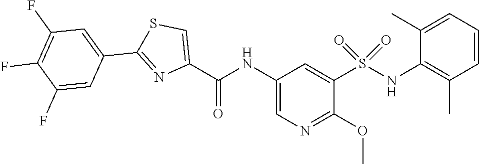 [SMILES: COc1ncc(NC(=O)c2csc(-c3cc(F)c(F)c(F)c3)n2)cc1S(=O)(=O)Nc1c(C)cccc1C] |

or a pharmaceutically acceptable salt, solvate, hydrate, polymorph, co-crystal, tautomer, stereoisomer, isotopically labeled compound, or prodrug thereof (e.g., a pharmaceutically acceptable salt thereof).

In certain embodiments, the compound is of the formula:

| No. | Formula |
|---|---|
| 2000 | 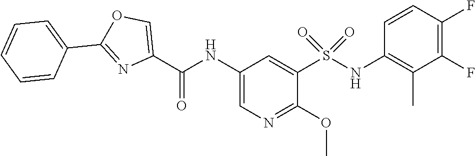 [SMILES: COc1ncc(NC(=O)c2coc(-c3ccccc3)n2)cc1S(=O)(=O)Nc1ccc(F)c(F)c1C] |
| 2002 | 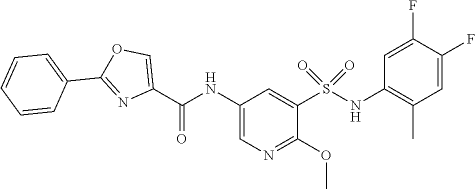 [SMILES: COc1ncc(NC(=O)c2coc(-c3ccccc3)n2)cc1S(=O)(=O)Nc1cc(F)c(F)cc1C] |
| 2004 | 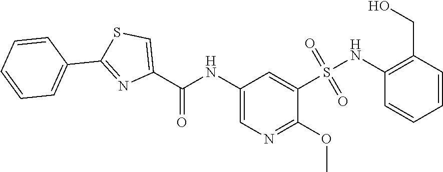 [SMILES: COc1ncc(NC(=O)c2csc(-c3ccccc3)n2)cc1S(=O)(=O)Nc1ccccc1CO] |
| 2005 | 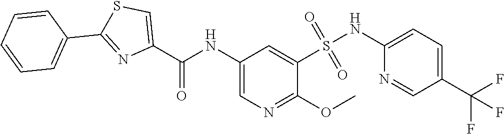 [SMILES: COc1ncc(NC(=O)c2csc(-c3ccccc3)n2)cc1S(=O)(=O)Nc1ccc(C(F)(F)F)cn1] |
| 2068 | 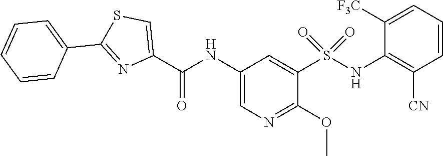 [SMILES: COc1ncc(NC(=O)c2csc(-c3ccccc3)n2)cc1S(=O)(=O)Nc1c(C#N)cccc1C(F)(F)F] |
| 2069 | 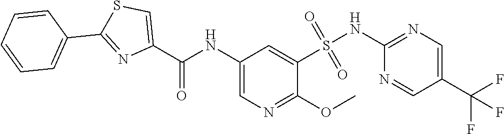 [SMILES: COc1ncc(NC(=O)c2csc(-c3ccccc3)n2)cc1S(=O)(=O)Nc1ncc(C(F)(F)F)cn1] |
| 2072 | 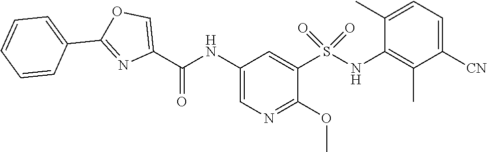 [SMILES: COc1ncc(NC(=O)c2coc(-c3ccccc3)n2)cc1S(=O)(=O)Nc1c(C)ccc(C#N)c1C] |
| 2088 | 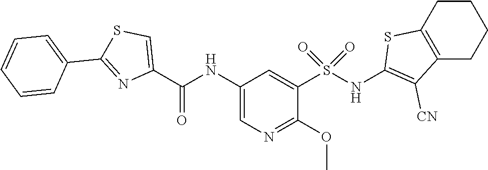 [SMILES: COc1ncc(NC(=O)c2csc(-c3ccccc3)n2)cc1S(=O)(=O)Nc1sc2c(c1C#N)CCCC2] |
| 2092 | 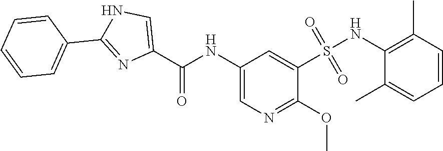 [SMILES: COc1ncc(NC(=O)c2c[nH]c(-c3ccccc3)n2)cc1S(=O)(=O)Nc1c(C)cccc1C] |
| 2119 | 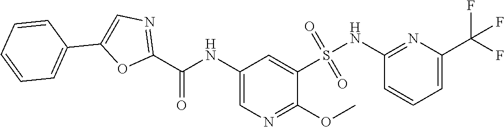 [SMILES: COc1ncc(NC(=O)c2ncc(-c3ccccc3)o2)cc1S(=O)(=O)Nc1cccc(C(F)(F)F)n1] |
| 2142 | 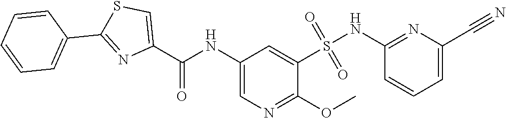 [SMILES: COc1ncc(NC(=O)c2csc(-c3ccccc3)n2)cc1S(=O)(=O)Nc1cccc(C#N)n1] |
| 2148 | 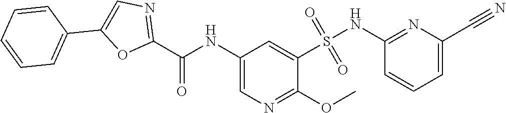 [SMILES: COc1ncc(NC(=O)c2ncc(-c3ccccc3)o2)cc1S(=O)(=O)Nc1cccc(C#N)n1] |
| 2151 | 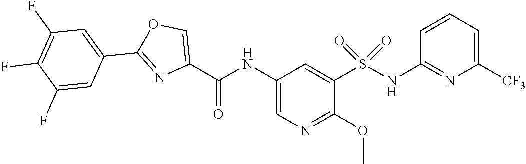 [SMILES: COc1ncc(NC(=O)c2coc(-c3cc(F)c(F)c(F)c3)n2)cc1S(=O)(=O)Nc1cccc(C(F)(F)F)n1] |
| 2155 | 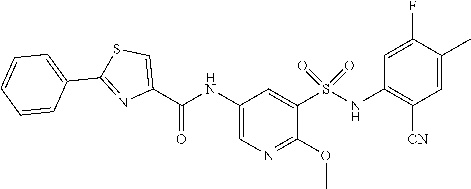 [SMILES: COc1ncc(NC(=O)c2csc(-c3ccccc3)n2)cc1S(=O)(=O)Nc1cc(F)c(C)cc1C#N] |
| 2156 | 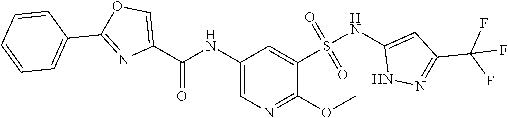 [SMILES: COc1ncc(NC(=O)c2coc(-c3ccccc3)n2)cc1S(=O)(=O)Nc1cc(C(F)(F)F)n[nH]1] |
| 2157 | 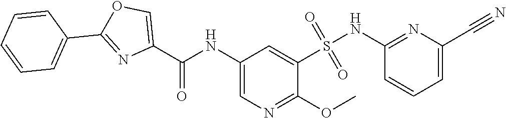 [SMILES: COc1ncc(NC(=O)c2coc(-c3ccccc3)n2)cc1S(=O)(=O)Nc1cccc(C#N)n1] |
| 2361 | 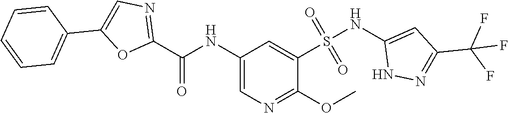 [SMILES: COc1ncc(NC(=O)c2ncc(-c3ccccc3)o2)cc1S(=O)(=O)Nc1cc(C(F)(F)F)n[nH]1] |
| 2365 | 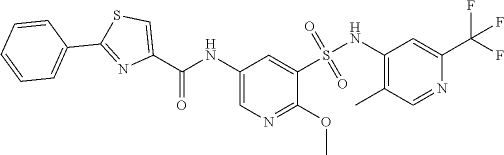 [SMILES: COc1ncc(NC(=O)c2csc(-c3ccccc3)n2)cc1S(=O)(=O)Nc1cc(C(F)(F)F)ncc1C] |
| 2366 | 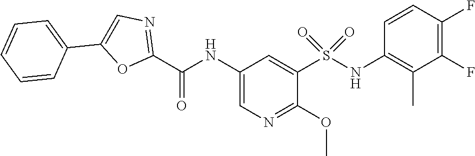 [SMILES: COc1ncc(NC(=O)c2ncc(-c3ccccc3)o2)cc1S(=O)(=O)Nc1ccc(F)c(F)c1C] |
| 2381 | 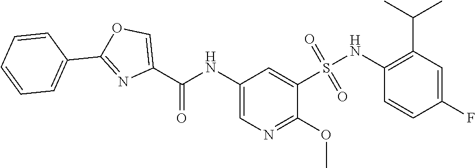 [SMILES: COc1ncc(NC(=O)c2coc(-c3ccccc3)n2)cc1S(=O)(=O)Nc1ccc(F)cc1C(C)C] |
| 2387 | 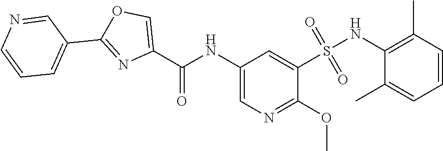 [SMILES: COc1ncc(NC(=O)c2coc(-c3cccnc3)n2)cc1S(=O)(=O)Nc1c(C)cccc1C] |
| 2390 | 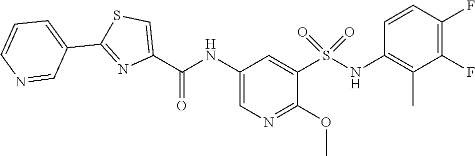 [SMILES: COc1ncc(NC(=O)c2csc(-c3cccnc3)n2)cc1S(=O)(=O)Nc1ccc(F)c(F)c1C] |
| 2396 | 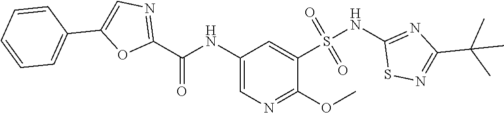 [SMILES: COc1ncc(NC(=O)c2ncc(-c3ccccc3)o2)cc1S(=O)(=O)Nc1nc(C(C)(C)C)ns1] |
| 2397 | 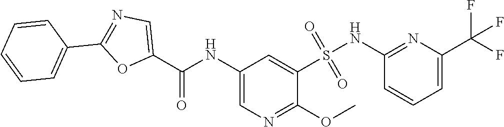 [SMILES: COc1ncc(NC(=O)c2cnc(-c3ccccc3)o2)cc1S(=O)(=O)Nc1cccc(C(F)(F)F)n1] |
| 2403 | 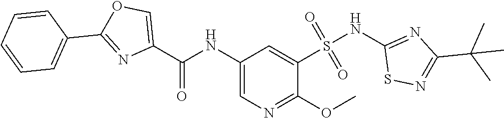 [SMILES: COc1ncc(NC(=O)c2coc(-c3ccccc3)n2)cc1S(=O)(=O)Nc1nc(C(C)(C)C)ns1] |
| 2407 | 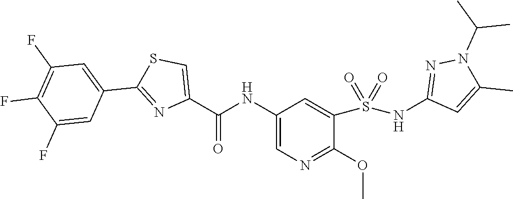 [SMILES: COc1ncc(NC(=O)c2csc(-c3cc(F)c(F)c(F)c3)n2)cc1S(=O)(=O)Nc1cc(C)n(C(C)C)n1] |
| 2418 | 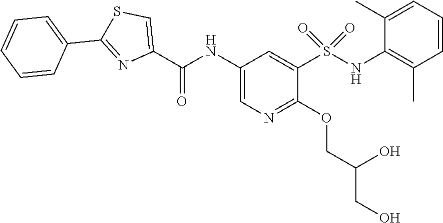 [SMILES: Cc1cccc(C)c1NS(=O)(=O)c1cc(NC(=O)c2csc(-c3ccccc3)n2)cnc1OCC(O)CO] |
| 2427 | 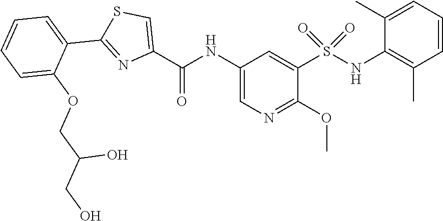 [SMILES: COc1ncc(NC(=O)c2csc(-c3ccccc3OCC(O)CO)n2)cc1S(=O)(=O)Nc1c(C)cccc1C] |
| 2431 | 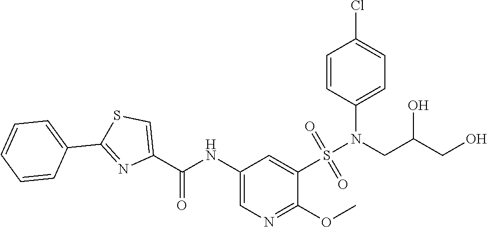 [SMILES: COc1ncc(NC(=O)c2csc(-c3ccccc3)n2)cc1S(=O)(=O)N(CC(O)CO)c1ccc(Cl)cc1] |
| 2445 | 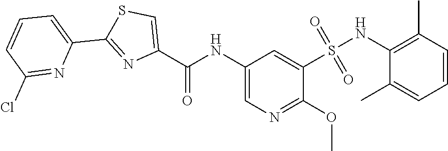 [SMILES: COc1ncc(NC(=O)c2csc(-c3cccc(Cl)n3)n2)cc1S(=O)(=O)Nc1c(C)cccc1C] |
| 2447 | 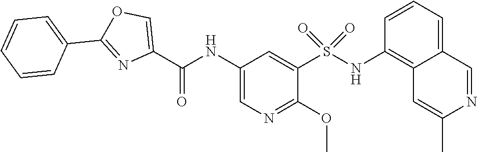 [SMILES: COc1ncc(NC(=O)c2coc(-c3ccccc3)n2)cc1S(=O)(=O)Nc1cccc2cnc(C)cc12] |
| 2454 | 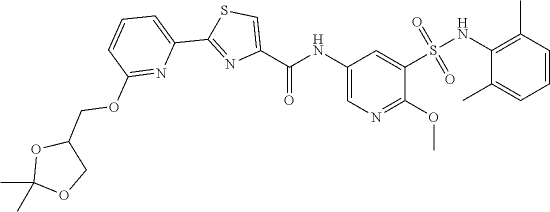 [SMILES: COc1ncc(NC(=O)c2csc(-c3cccc(OCC4COC(C)(C)O4)n3)n2)cc1S(=O)(=O)Nc1c(C)cccc1C] |
| 2463 | 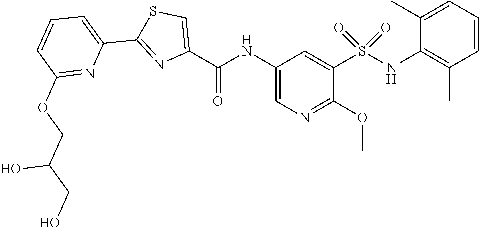 [SMILES: COc1ncc(NC(=O)c2csc(-c3cccc(OCC(O)CO)n3)n2)cc1S(=O)(=O)Nc1c(C)cccc1C] |
| 2466 | 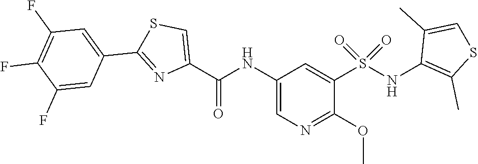 [SMILES: COc1ncc(NC(=O)c2csc(-c3cc(F)c(F)c(F)c3)n2)cc1S(=O)(=O)Nc1c(C)csc1C] |
| 2470 | 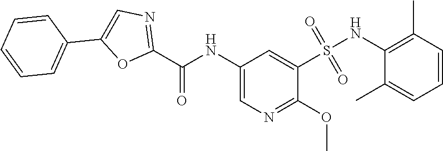 [SMILES: COc1ncc(NC(=O)c2ncc(-c3ccccc3)o2)cc1S(=O)(=O)Nc1c(C)cccc1C] |
| 2474 | 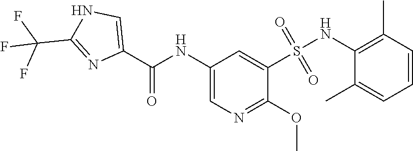 [SMILES: COc1ncc(NC(=O)c2c[nH]c(C(F)(F)F)n2)cc1S(=O)(=O)Nc1c(C)cccc1C] |
| 2487 | 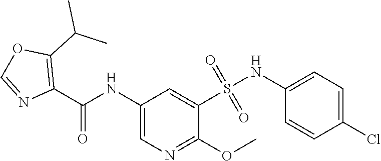 [SMILES: COc1ncc(NC(=O)c2ncoc2C(C)C)cc1S(=O)(=O)Nc1ccc(Cl)cc1] |
| 2492 | 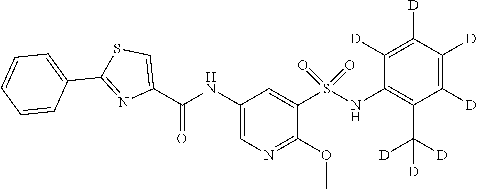 [SMILES: [2H]c1c([2H])c([2H])c(C([2H])([2H])[2H])c(NS(=O)(=O)c2cc(NC(=O)c3csc(-c4ccccc4)n3)cnc2OC)c1[2H]] |
| 2493 | 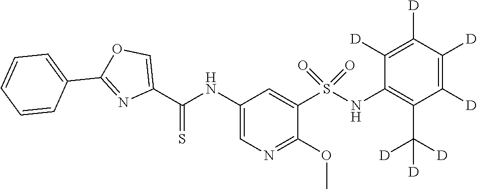 [SMILES: [2H]c1c([2H])c([2H])c(C([2H])([2H])[2H])c(NS(=O)(=O)c2cc(NC(=S)c3coc(-c4ccccc4)n3)cnc2OC)c1[2H]] |
| 2496 | 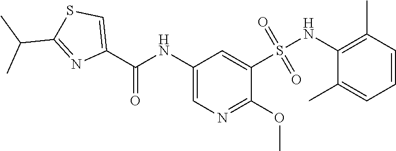 [SMILES: COc1ncc(NC(=O)c2csc(C(C)C)n2)cc1S(=O)(=O)Nc1c(C)cccc1C] |
| 2497 | 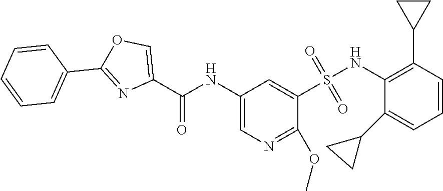 [SMILES: COc1ncc(NC(=O)c2coc(-c3ccccc3)n2)cc1S(=O)(=O)Nc1c(C2CC2)cccc1C1CC1] |
| 2498 | 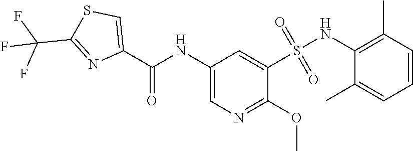 [SMILES: COc1ncc(NC(=O)c2csc(C(F)(F)F)n2)cc1S(=O)(=O)Nc1c(C)cccc1C] |
| 2499 | 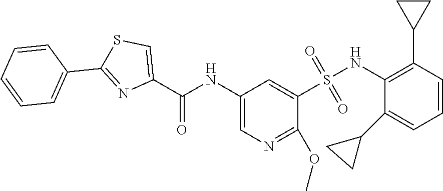 [SMILES: COc1ncc(NC(=O)c2csc(-c3ccccc3)n2)cc1S(=O)(=O)Nc1c(C2CC2)cccc1C1CC1] |
| 2500 | 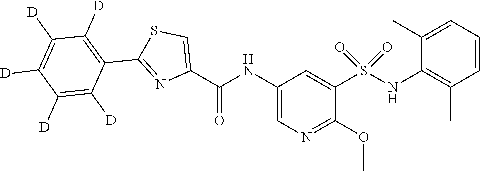 [SMILES: [2H]c1c([2H])c([2H])c(-c2nc(C(=O)Nc3cnc(OC)c(S(=O)(=O)Nc4c(C)cccc4C)c3)cs2)c([2H])c1[2H]] |
| 2501 | 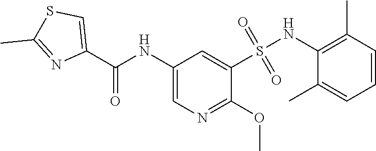 [SMILES: COc1ncc(NC(=O)c2csc(C)n2)cc1S(=O)(=O)Nc1c(C)cccc1C] |
| 2502 | 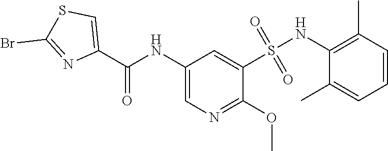 [SMILES: COc1ncc(NC(=O)c2csc(Br)n2)cc1S(=O)(=O)Nc1c(C)cccc1C] |
| 2506 | 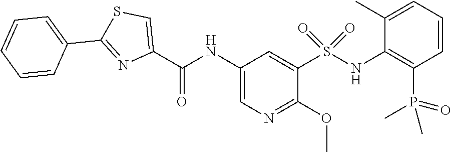 [SMILES: COc1ncc(NC(=O)c2csc(-c3ccccc3)n2)cc1S(=O)(=O)Nc1c(C)cccc1P(C)(C)=O] |
| 2512 | 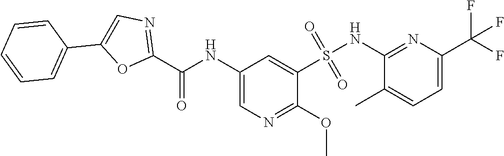 [SMILES: COc1ncc(NC(=O)c2ncc(-c3ccccc3)o2)cc1S(=O)(=O)Nc1nc(C(F)(F)F)ccc1C] |
| 2523 | 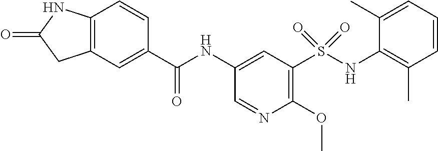 [SMILES: COc1ncc(NC(=O)c2ccc3c(c2)CC(=O)N3)cc1S(=O)(=O)Nc1c(C)cccc1C] |
| 2524 | 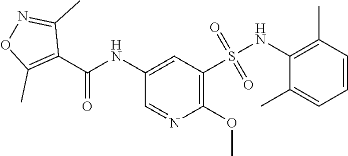 [SMILES: COc1ncc(NC(=O)c2c(C)noc2C)cc1S(=O)(=O)Nc1c(C)cccc1C] |
| 2525 | 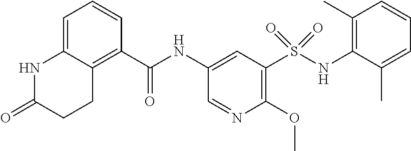 [SMILES: COc1ncc(NC(=O)c2cccc3c2CCC(=O)N3)cc1S(=O)(=O)Nc1c(C)cccc1C] |
| 2526 | 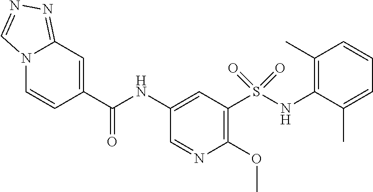 [SMILES: COc1ncc(NC(=O)c2ccn3cnnc3c2)cc1S(=O)(=O)Nc1c(C)cccc1C] |
| 2528 | 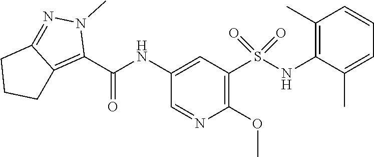 [SMILES: COc1ncc(NC(=O)c2c3c(nn2C)CCC3)cc1S(=O)(=O)Nc1c(C)cccc1C] |
| 2529 | 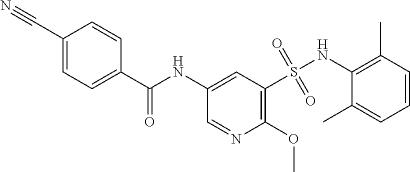 [SMILES: COc1ncc(NC(=O)c2ccc(C#N)cc2)cc1S(=O)(=O)Nc1c(C)cccc1C] |
| 2531 | 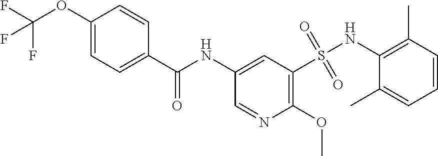 [SMILES: COc1ncc(NC(=O)c2ccc(OC(F)(F)F)cc2)cc1S(=O)(=O)Nc1c(C)cccc1C] |
| 2532 | 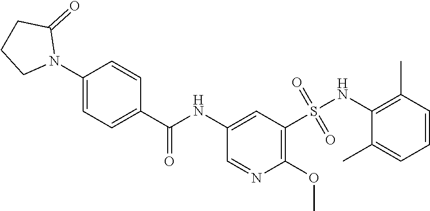 [SMILES: COc1ncc(NC(=O)c2ccc(N3CCCC3=O)cc2)cc1S(=O)(=O)Nc1c(C)cccc1C] |
| 2535 | 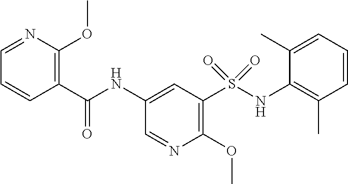 [SMILES: COc1ncccc1C(=O)Nc1cnc(OC)c(S(=O)(=O)Nc2c(C)cccc2C)c1] |
| 2536 | 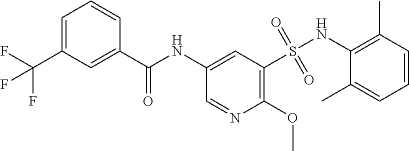 [SMILES: COc1ncc(NC(=O)c2cccc(C(F)(F)F)c2)cc1S(=O)(=O)Nc1c(C)cccc1C] |
| 2537 | 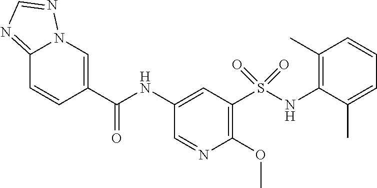 [SMILES: COc1ncc(NC(=O)c2ccc3ncnn3c2)cc1S(=O)(=O)Nc1c(C)cccc1C] |
| 2538 | 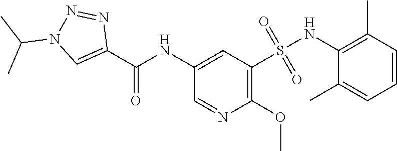 [SMILES: COc1ncc(NC(=O)c2cn(C(C)C)nn2)cc1S(=O)(=O)Nc1c(C)cccc1C] |
| 2539 | 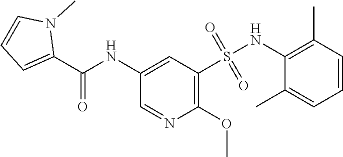 [SMILES: COc1ncc(NC(=O)c2cccn2C)cc1S(=O)(=O)Nc1c(C)cccc1C] |
| 2541 | 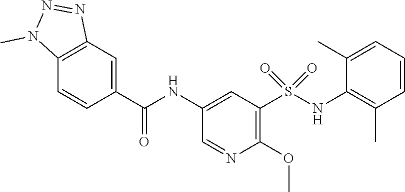 [SMILES: COc1ncc(NC(=O)c2ccc3c(c2)nnn3C)cc1S(=O)(=O)Nc1c(C)cccc1C] |
| 2542 | 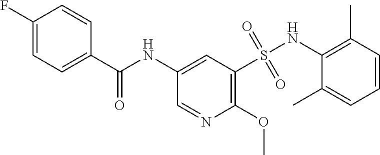 [SMILES: COc1ncc(NC(=O)c2ccc(F)cc2)cc1S(=O)(=O)Nc1c(C)cccc1C] |
| 2543 | 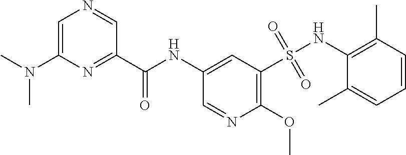 [SMILES: COc1ncc(NC(=O)c2cncc(N(C)C)n2)cc1S(=O)(=O)Nc1c(C)cccc1C] |
| 2544 | 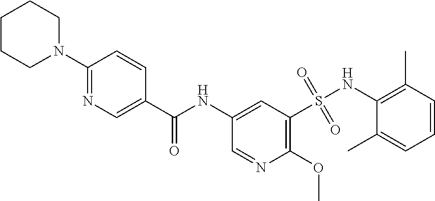 [SMILES: COc1ncc(NC(=O)c2ccc(N3CCCCC3)nc2)cc1S(=O)(=O)Nc1c(C)cccc1C] |
| 2545 | 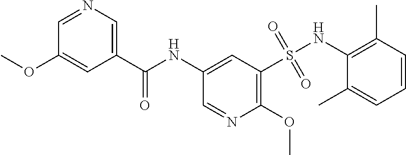 [SMILES: COc1cncc(C(=O)Nc2cnc(OC)c(S(=O)(=O)Nc3c(C)cccc3C)c2)c1] |
| 2546 | 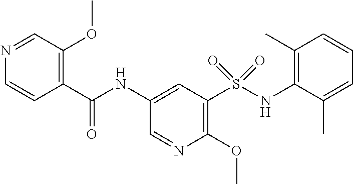 [SMILES: COc1cnccc1C(=O)Nc1cnc(OC)c(S(=O)(=O)Nc2c(C)cccc2C)c1] |
| 2547 | 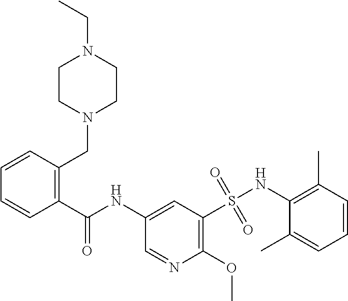 [SMILES: CCN1CCN(Cc2ccccc2C(=O)Nc2cnc(OC)c(S(=O)(=O)Nc3c(C)cccc3C)c2)CC1] |
| 2548 | 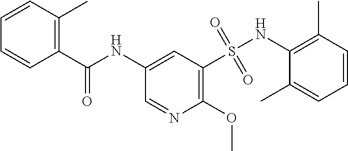 [SMILES: COc1ncc(NC(=O)c2ccccc2C)cc1S(=O)(=O)Nc1c(C)cccc1C] |
| 2549 | 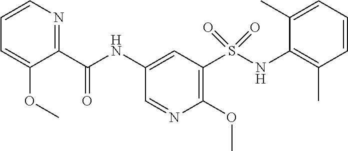 [SMILES: COc1cccnc1C(=O)Nc1cnc(OC)c(S(=O)(=O)Nc2c(C)cccc2C)c1] |
| 2550 | 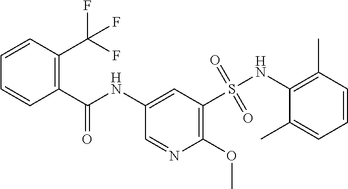 [SMILES: COc1ncc(NC(=O)c2ccccc2C(F)(F)F)cc1S(=O)(=O)Nc1c(C)cccc1C] |
| 2551 | 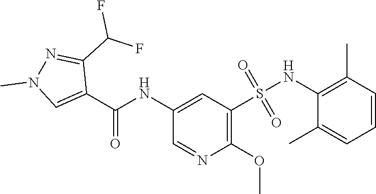 [SMILES: COc1ncc(NC(=O)c2cn(C)nc2C(F)F)cc1S(=O)(=O)Nc1c(C)cccc1C] |
| 2552 | 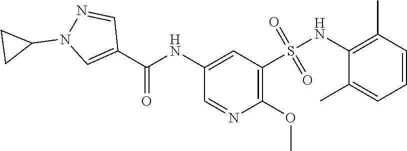 [SMILES: COc1ncc(NC(=O)c2cnn(C3CC3)c2)cc1S(=O)(=O)Nc1c(C)cccc1C] |
| 2553 | 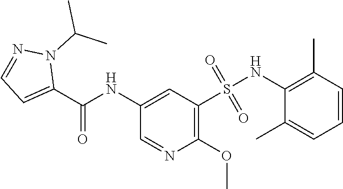 [SMILES: COc1ncc(NC(=O)c2ccnn2C(C)C)cc1S(=O)(=O)Nc1c(C)cccc1C] |
| 2554 | 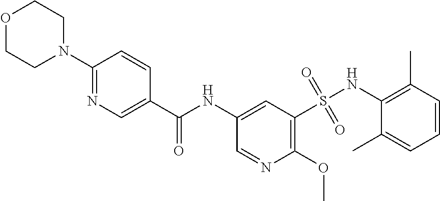 [SMILES: COc1ncc(NC(=O)c2ccc(N3CCOCC3)nc2)cc1S(=O)(=O)Nc1c(C)cccc1C] |
| 2555 | 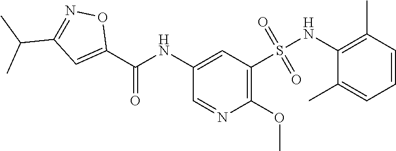 [SMILES: COc1ncc(NC(=O)c2cc(C(C)C)no2)cc1S(=O)(=O)Nc1c(C)cccc1C] |
| 2556 | 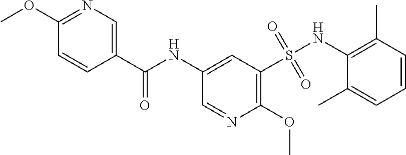 [SMILES: COc1ccc(C(=O)Nc2cnc(OC)c(S(=O)(=O)Nc3c(C)cccc3C)c2)cn1] |
| 2557 | 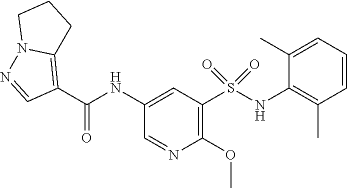 [SMILES: COc1ncc(NC(=O)c2cnn3c2CCC3)cc1S(=O)(=O)Nc1c(C)cccc1C] |
| 2558 | 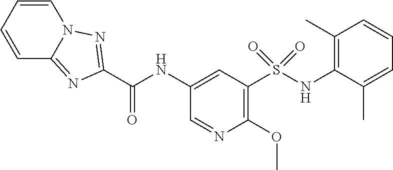 [SMILES: COc1ncc(NC(=O)c2nc3ccccn3n2)cc1S(=O)(=O)Nc1c(C)cccc1C] |
| 2559 | 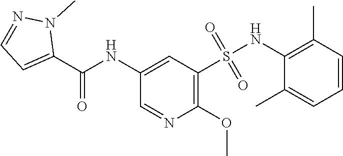 [SMILES: COc1ncc(NC(=O)c2ccnn2C)cc1S(=O)(=O)Nc1c(C)cccc1C] |
| 2561 | 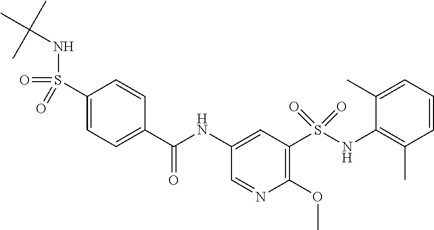 [SMILES: COc1ncc(NC(=O)c2ccc(S(=O)(=O)NC(C)(C)C)cc2)cc1S(=O)(=O)Nc1c(C)cccc1C] |
| 2562 | 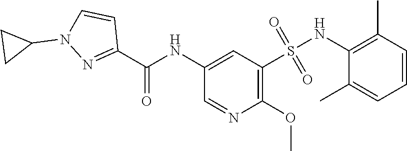 [SMILES: COc1ncc(NC(=O)c2ccn(C3CC3)n2)cc1S(=O)(=O)Nc1c(C)cccc1C] |
| 2564 | 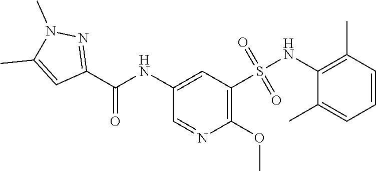 [SMILES: COc1ncc(NC(=O)c2cc(C)n(C)n2)cc1S(=O)(=O)Nc1c(C)cccc1C] |
| 2566 | 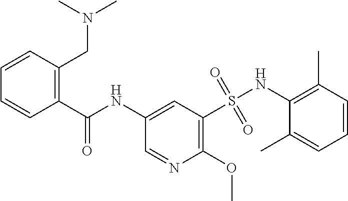 [SMILES: COc1ncc(NC(=O)c2ccccc2CN(C)C)cc1S(=O)(=O)Nc1c(C)cccc1C] |
| 2567 | 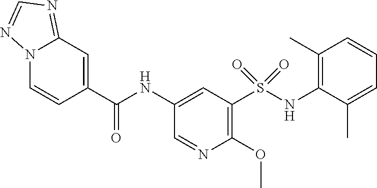 [SMILES: COc1ncc(NC(=O)c2ccn3ncnc3c2)cc1S(=O)(=O)Nc1c(C)cccc1C] |
| 2568 | 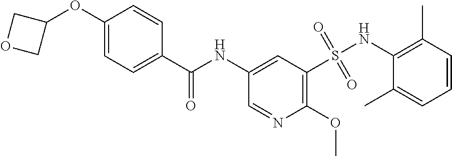 [SMILES: COc1ncc(NC(=O)c2ccc(OC3COC3)cc2)cc1S(=O)(=O)Nc1c(C)cccc1C] |
| 2569 | 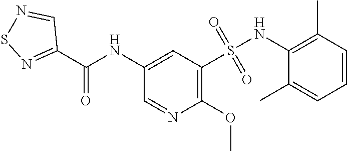 [SMILES: COc1ncc(NC(=O)c2cnsn2)cc1S(=O)(=O)Nc1c(C)cccc1C] |
| 2570 | 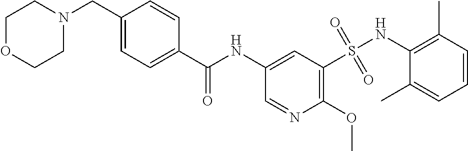 [SMILES: COc1ncc(NC(=O)c2ccc(CN3CCOCC3)cc2)cc1S(=O)(=O)Nc1c(C)cccc1C] |
| 2571 | 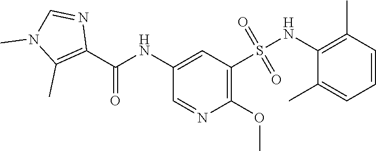 [SMILES: COc1ncc(NC(=O)c2ncn(C)c2C)cc1S(=O)(=O)Nc1c(C)cccc1C] |
| 2572 | 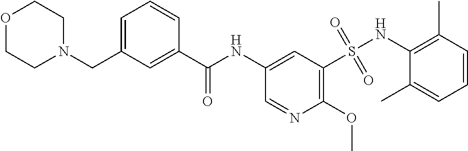 [SMILES: COc1ncc(NC(=O)c2cccc(CN3CCOCC3)c2)cc1S(=O)(=O)Nc1c(C)cccc1C] |
| 2573 | 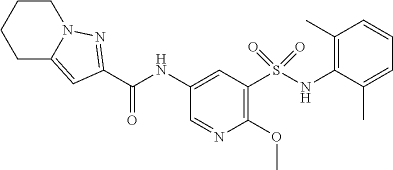 [SMILES: COc1ncc(NC(=O)c2cc3n(n2)CCCC3)cc1S(=O)(=O)Nc1c(C)cccc1C] |
| 2574 | 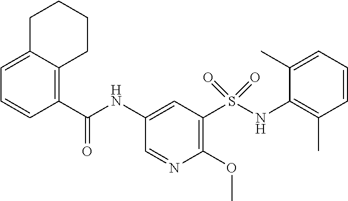 [SMILES: COc1ncc(NC(=O)c2cccc3c2CCCC3)cc1S(=O)(=O)Nc1c(C)cccc1C] |
| 2575 | 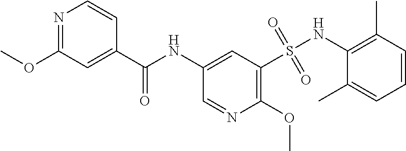 [SMILES: COc1cc(C(=O)Nc2cnc(OC)c(S(=O)(=O)Nc3c(C)cccc3C)c2)ccn1] |
| 2576 | 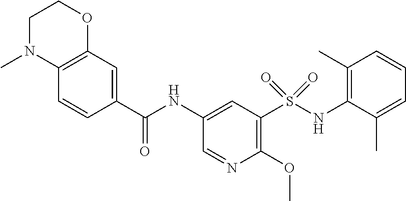 [SMILES: COc1ncc(NC(=O)c2ccc3c(c2)OCCN3C)cc1S(=O)(=O)Nc1c(C)cccc1C] |
| 2577 | 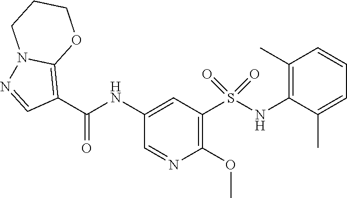 [SMILES: COc1ncc(NC(=O)c2cnn3c2OCCC3)cc1S(=O)(=O)Nc1c(C)cccc1C] |
| 2578 | 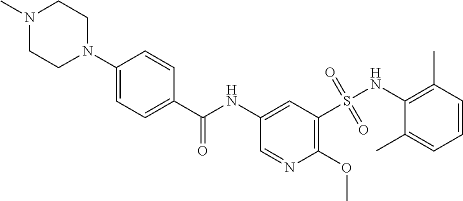 [SMILES: COc1ncc(NC(=O)c2ccc(N3CCN(C)CC3)cc2)cc1S(=O)(=O)Nc1c(C)cccc1C] |
| 2580 | 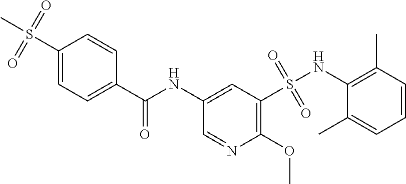 [SMILES: COc1ncc(NC(=O)c2ccc(S(C)(=O)=O)cc2)cc1S(=O)(=O)Nc1c(C)cccc1C] |
| 2581 | 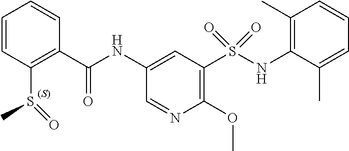 |
| 2582 | 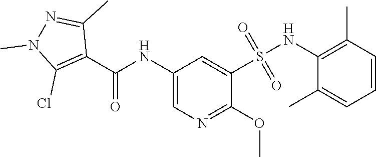 [SMILES: COc1ncc(NC(=O)c2c(C)nn(C)c2Cl)cc1S(=O)(=O)Nc1c(C)cccc1C] |
| 2583 | 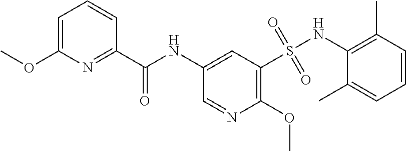 [SMILES: COc1cccc(C(=O)Nc2cnc(OC)c(S(=O)(=O)Nc3c(C)cccc3C)c2)n1] |
| 2584 | 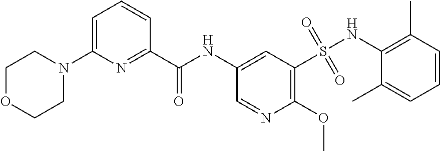 [SMILES: COc1ncc(NC(=O)c2cccc(N3CCOCC3)n2)cc1S(=O)(=O)Nc1c(C)cccc1C] |
| 2586 | 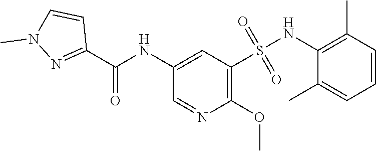 [SMILES: COc1ncc(NC(=O)c2ccn(C)n2)cc1S(=O)(=O)Nc1c(C)cccc1C] |
| 2587 | 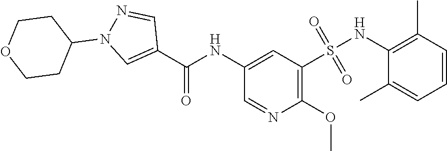 [SMILES: COc1ncc(NC(=O)c2cnn(C3CCOCC3)c2)cc1S(=O)(=O)Nc1c(C)cccc1C] |
| 2590 | 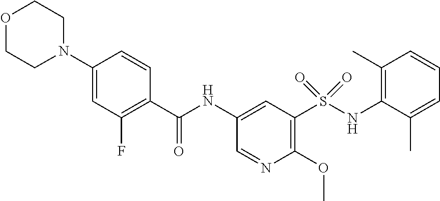 [SMILES: COc1ncc(NC(=O)c2ccc(N3CCOCC3)cc2F)cc1S(=O)(=O)Nc1c(C)cccc1C] |
| 2591 | 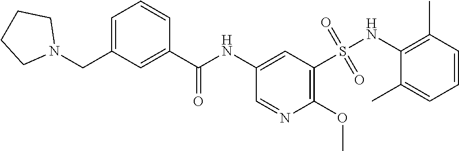 [SMILES: COc1ncc(NC(=O)c2cccc(CN3CCCC3)c2)cc1S(=O)(=O)Nc1c(C)cccc1C] |
| 2593 | 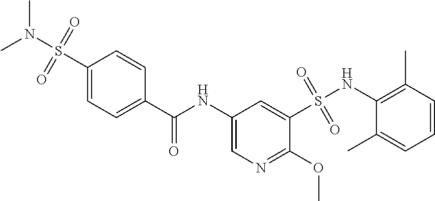 [SMILES: COc1ncc(NC(=O)c2ccc(S(=O)(=O)N(C)C)cc2)cc1S(=O)(=O)Nc1c(C)cccc1C] |
| 2594 | 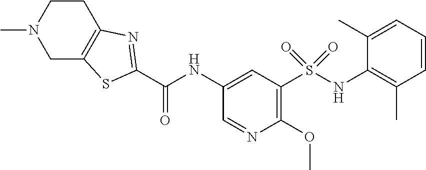 [SMILES: COc1ncc(NC(=O)c2nc3c(s2)CN(C)CC3)cc1S(=O)(=O)Nc1c(C)cccc1C] |
| 2595 | 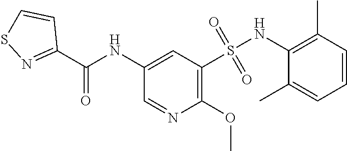 [SMILES: COc1ncc(NC(=O)c2ccsn2)cc1S(=O)(=O)Nc1c(C)cccc1C] |
| 2597 | 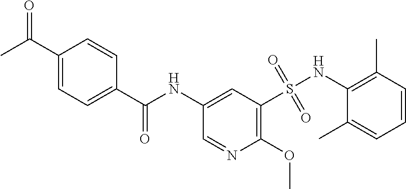 [SMILES: COc1ncc(NC(=O)c2ccc(C(C)=O)cc2)cc1S(=O)(=O)Nc1c(C)cccc1C] |
| 2599 | 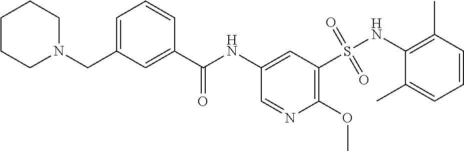 [SMILES: COc1ncc(NC(=O)c2cccc(CN3CCCCC3)c2)cc1S(=O)(=O)Nc1c(C)cccc1C] |
| 2600 | 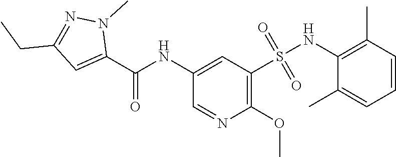 [SMILES: CCc1cc(C(=O)Nc2cnc(OC)c(S(=O)(=O)Nc3c(C)cccc3C)c2)n(C)n1] |
| 2601 | 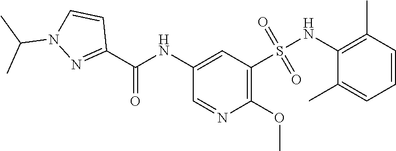 [SMILES: COc1ncc(NC(=O)c2ccn(C(C)C)n2)cc1S(=O)(=O)Nc1c(C)cccc1C] |
| 2602 | 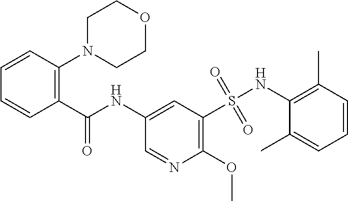 [SMILES: COc1ncc(NC(=O)c2ccccc2N2CCOCC2)cc1S(=O)(=O)Nc1c(C)cccc1C] |
| 2607 | 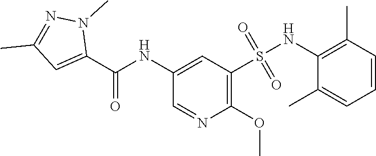 [SMILES: COc1ncc(NC(=O)c2cc(C)nn2C)cc1S(=O)(=O)Nc1c(C)cccc1C] |
| 2608 | 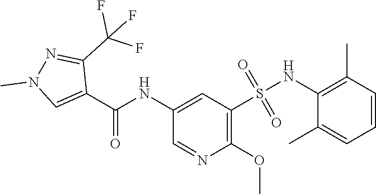 [SMILES: COc1ncc(NC(=O)c2cn(C)nc2C(F)(F)F)cc1S(=O)(=O)Nc1c(C)cccc1C] |
| 2609 | 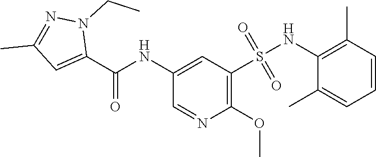 [SMILES: CCn1nc(C)cc1C(=O)Nc1cnc(OC)c(S(=O)(=O)Nc2c(C)cccc2C)c1] |
| 2611 | 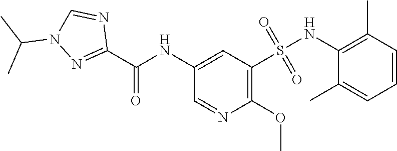 [SMILES: COc1ncc(NC(=O)c2ncn(C(C)C)n2)cc1S(=O)(=O)Nc1c(C)cccc1C] |
| 2612 | 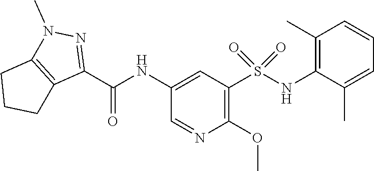 [SMILES: COc1ncc(NC(=O)c2nn(C)c3c2CCC3)cc1S(=O)(=O)Nc1c(C)cccc1C] |
| 2613 | 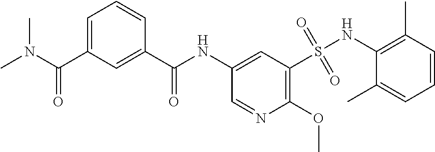 [SMILES: COc1ncc(NC(=O)c2cccc(C(=O)N(C)C)c2)cc1S(=O)(=O)Nc1c(C)cccc1C] |
| 2614 | 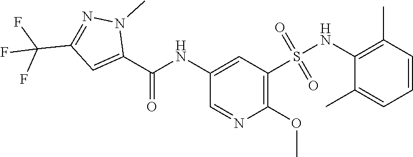 [SMILES: COc1ncc(NC(=O)c2cc(C(F)(F)F)nn2C)cc1S(=O)(=O)Nc1c(C)cccc1C] |
| 2615 | 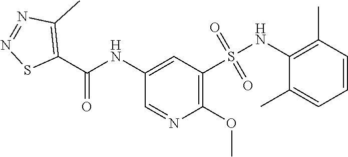 [SMILES: COc1ncc(NC(=O)c2snnc2C)cc1S(=O)(=O)Nc1c(C)cccc1C] |
| 2617 | 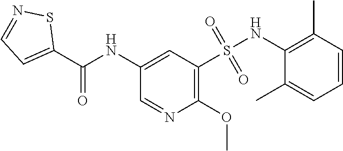 [SMILES: COc1ncc(NC(=O)c2ccns2)cc1S(=O)(=O)Nc1c(C)cccc1C] |
| 2618 | 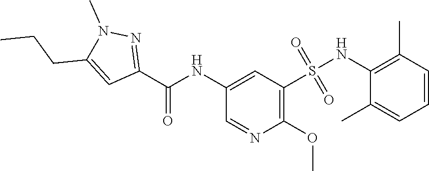 [SMILES: CCCc1cc(C(=O)Nc2cnc(OC)c(S(=O)(=O)Nc3c(C)cccc3C)c2)nn1C] |
| 2619 | 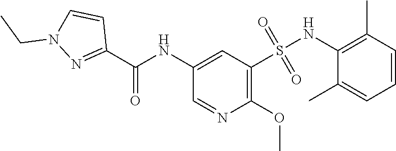 [SMILES: CCn1ccc(C(=O)Nc2cnc(OC)c(S(=O)(=O)Nc3c(C)cccc3C)c2)n1] |
| 2621 | 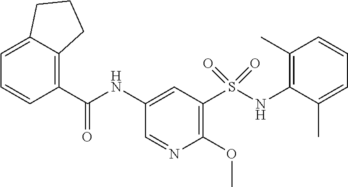 [SMILES: COc1ncc(NC(=O)c2cccc3c2CCC3)cc1S(=O)(=O)Nc1c(C)cccc1C] |
| 2622 | 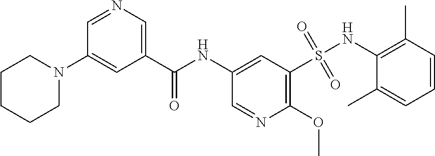 [SMILES: COc1ncc(NC(=O)c2cncc(N3CCCCC3)c2)cc1S(=O)(=O)Nc1c(C)cccc1C] |
| 2624 | 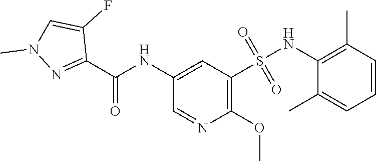 [SMILES: COc1ncc(NC(=O)c2nn(C)cc2F)cc1S(=O)(=O)Nc1c(C)cccc1C] |
| 2625 | 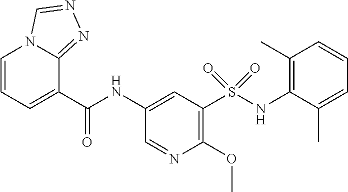 [SMILES: COc1ncc(NC(=O)c2cccn3cnnc23)cc1S(=O)(=O)Nc1c(C)cccc1C] |
| 2626 | 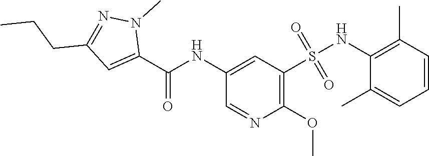 [SMILES: CCCc1cc(C(=O)Nc2cnc(OC)c(S(=O)(=O)Nc3c(C)cccc3C)c2)n(C)n1] |
| 2627 | 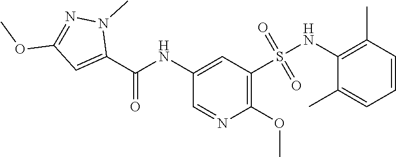 [SMILES: COc1cc(C(=O)Nc2cnc(OC)c(S(=O)(=O)Nc3c(C)cccc3C)c2)n(C)n1] |
| 2628 | 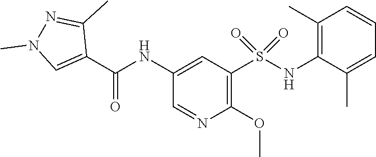 [SMILES: COc1ncc(NC(=O)c2cn(C)nc2C)cc1S(=O)(=O)Nc1c(C)cccc1C] |
| 2629 | 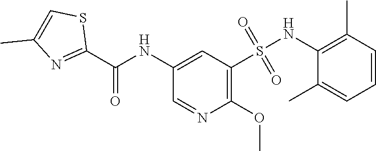 [SMILES: COc1ncc(NC(=O)c2nc(C)cs2)cc1S(=O)(=O)Nc1c(C)cccc1C] |
| 2630 | 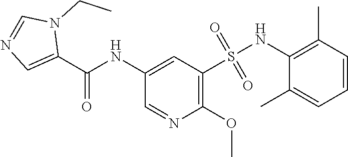 [SMILES: CCn1cncc1C(=O)Nc1cnc(OC)c(S(=O)(=O)Nc2c(C)cccc2C)c1] |
| 2631 | 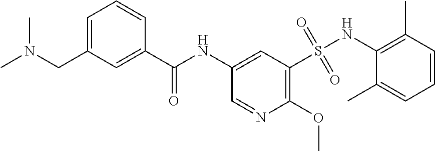 [SMILES: COc1ncc(NC(=O)c2cccc(CN(C)C)c2)cc1S(=O)(=O)Nc1c(C)cccc1C] |
| 2632 | 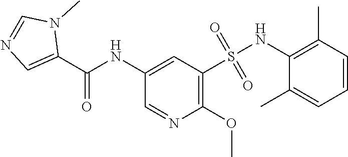 [SMILES: COc1ncc(NC(=O)c2cncn2C)cc1S(=O)(=O)Nc1c(C)cccc1C] |
| 2633 | 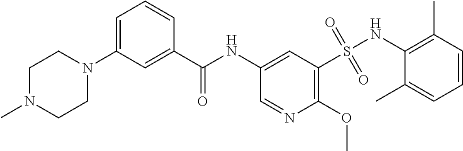 [SMILES: COc1ncc(NC(=O)c2cccc(N3CCN(C)CC3)c2)cc1S(=O)(=O)Nc1c(C)cccc1C] |
| 2634 | 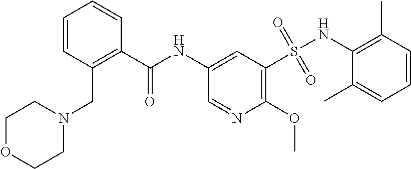 [SMILES: COc1ncc(NC(=O)c2ccccc2CN2CCOCC2)cc1S(=O)(=O)Nc1c(C)cccc1C] |
| 2635 | 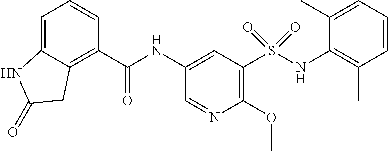 [SMILES: COc1ncc(NC(=O)c2cccc3c2CC(=O)N3)cc1S(=O)(=O)Nc1c(C)cccc1C] |
| 2636 | 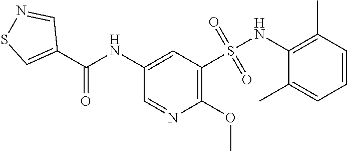 [SMILES: COc1ncc(NC(=O)c2cnsc2)cc1S(=O)(=O)Nc1c(C)cccc1C] |
| 2637 | 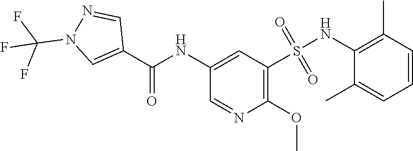 [SMILES: COc1ncc(NC(=O)c2cnn(C(F)(F)F)c2)cc1S(=O)(=O)Nc1c(C)cccc1C] |
| 2638 | 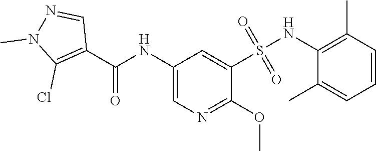 [SMILES: COc1ncc(NC(=O)c2cnn(C)c2Cl)cc1S(=O)(=O)Nc1c(C)cccc1C] |
| 2639 | 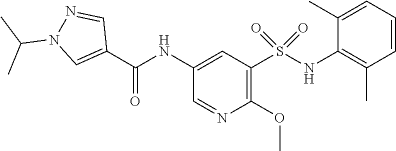 [SMILES: COc1ncc(NC(=O)c2cnn(C(C)C)c2)cc1S(=O)(=O)Nc1c(C)cccc1C] |
| 2640 | 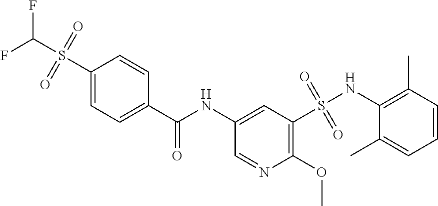 [SMILES: COc1ncc(NC(=O)c2ccc(S(=O)(=O)C(F)F)cc2)cc1S(=O)(=O)Nc1c(C)cccc1C] |
| 2641 | 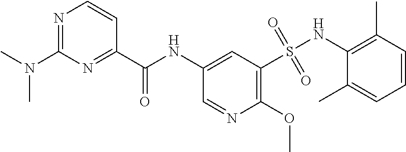 [SMILES: COc1ncc(NC(=O)c2ccnc(N(C)C)n2)cc1S(=O)(=O)Nc1c(C)cccc1C] |
| 2642 | 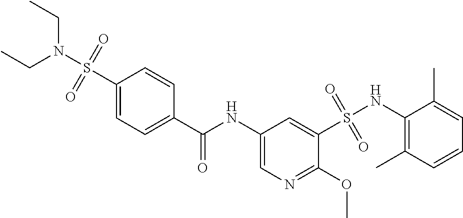 [SMILES: CCN(CC)S(=O)(=O)c1ccc(C(=O)Nc2cnc(OC)c(S(=O)(=O)Nc3c(C)cccc3C)c2)cc1] |
| 2643 | 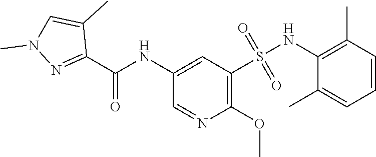 [SMILES: COc1ncc(NC(=O)c2nn(C)cc2C)cc1S(=O)(=O)Nc1c(C)cccc1C] |
| 2644 | 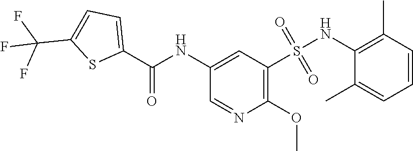 [SMILES: COc1ncc(NC(=O)c2ccc(C(F)(F)F)s2)cc1S(=O)(=O)Nc1c(C)cccc1C] |
| 2646 | 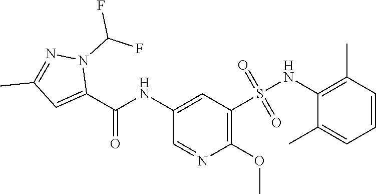 [SMILES: COc1ncc(NC(=O)c2cc(C)nn2C(F)F)cc1S(=O)(=O)Nc1c(C)cccc1C] |
| 2649 | 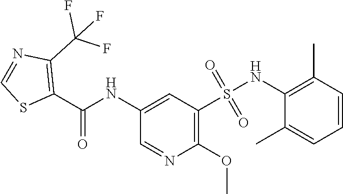 [SMILES: COc1ncc(NC(=O)c2scnc2C(F)(F)F)cc1S(=O)(=O)Nc1c(C)cccc1C] |
| 2650 | 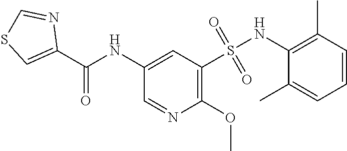 [SMILES: COc1ncc(NC(=O)c2cscn2)cc1S(=O)(=O)Nc1c(C)cccc1C] |
| 2651 | 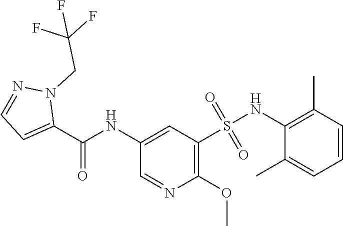 [SMILES: COc1ncc(NC(=O)c2ccnn2CC(F)(F)F)cc1S(=O)(=O)Nc1c(C)cccc1C] |
| 2653 | 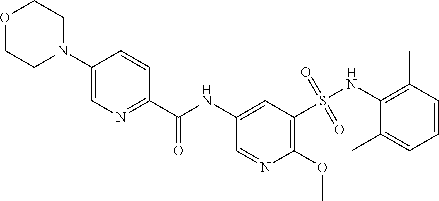 [SMILES: COc1ncc(NC(=O)c2ccc(N3CCOCC3)cn2)cc1S(=O)(=O)Nc1c(C)cccc1C] |
| 2654 | 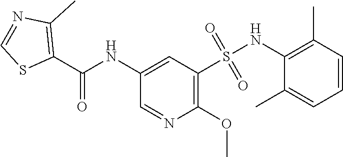 [SMILES: COc1ncc(NC(=O)c2scnc2C)cc1S(=O)(=O)Nc1c(C)cccc1C] |
| 2655 | 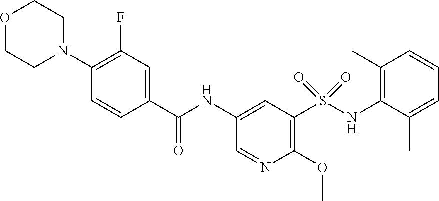 [SMILES: COc1ncc(NC(=O)c2ccc(N3CCOCC3)c(F)c2)cc1S(=O)(=O)Nc1c(C)cccc1C] |
| 2656 | 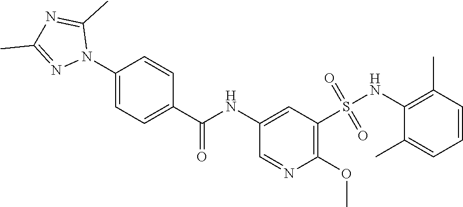 [SMILES: COc1ncc(NC(=O)c2ccc(-n3nc(C)nc3C)cc2)cc1S(=O)(=O)Nc1c(C)cccc1C] |
| 2657 | 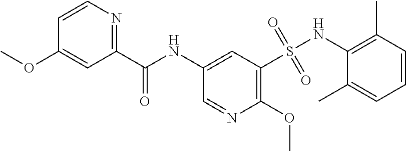 [SMILES: COc1ccnc(C(=O)Nc2cnc(OC)c(S(=O)(=O)Nc3c(C)cccc3C)c2)c1] |
| 2658 | 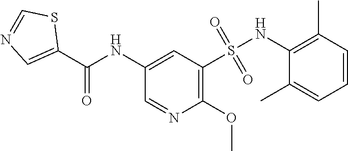 [SMILES: COc1ncc(NC(=O)c2cncs2)cc1S(=O)(=O)Nc1c(C)cccc1C] |
| 2659 | 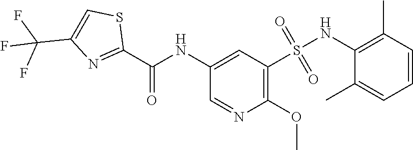 [SMILES: COc1ncc(NC(=O)c2nc(C(F)(F)F)cs2)cc1S(=O)(=O)Nc1c(C)cccc1C] |
| 2660 | 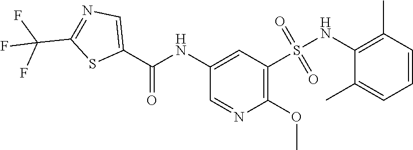 [SMILES: COc1ncc(NC(=O)c2cnc(C(F)(F)F)s2)cc1S(=O)(=O)Nc1c(C)cccc1C] |
| 2661 | 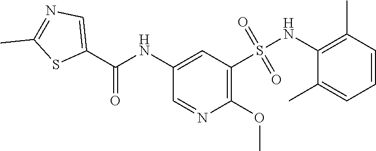 [SMILES: COc1ncc(NC(=O)c2cnc(C)s2)cc1S(=O)(=O)Nc1c(C)cccc1C] |
| 2662 | 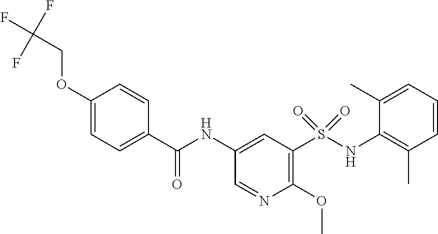 [SMILES: COc1ncc(NC(=O)c2ccc(OCC(F)(F)F)cc2)cc1S(=O)(=O)Nc1c(C)cccc1C] |
| 2663 | 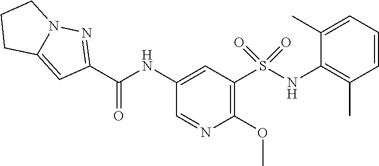 [SMILES: COc1ncc(NC(=O)c2cc3n(n2)CCC3)cc1S(=O)(=O)Nc1c(C)cccc1C] |
| 2665 | 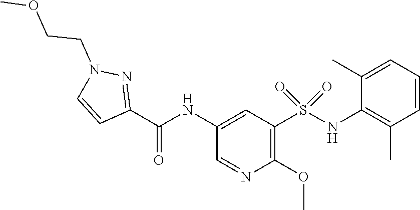 [SMILES: COCCn1ccc(C(=O)Nc2cnc(OC)c(S(=O)(=O)Nc3c(C)cccc3C)c2)n1] |
| 2666 | 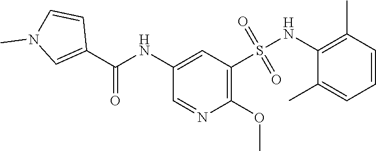 [SMILES: COc1ncc(NC(=O)c2ccn(C)c2)cc1S(=O)(=O)Nc1c(C)cccc1C] |
| 2667 | 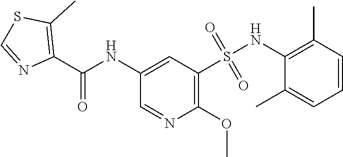 [SMILES: COc1ncc(NC(=O)c2ncsc2C)cc1S(=O)(=O)Nc1c(C)cccc1C] |
| 2669 | 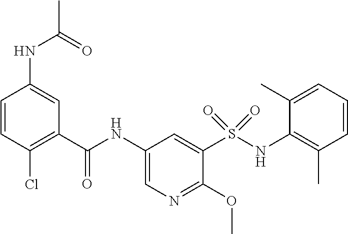 [SMILES: COc1ncc(NC(=O)c2cc(NC(C)=O)ccc2Cl)cc1S(=O)(=O)Nc1c(C)cccc1C] |
| 2670 | 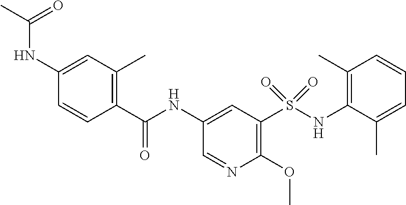 [SMILES: COc1ncc(NC(=O)c2ccc(NC(C)=O)cc2C)cc1S(=O)(=O)Nc1c(C)cccc1C] |
| 2671 | 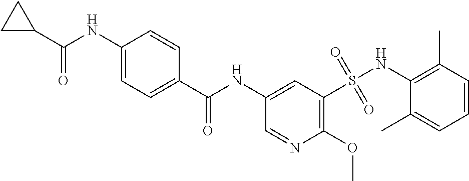 [SMILES: COc1ncc(NC(=O)c2ccc(NC(=O)C3CC3)cc2)cc1S(=O)(=O)Nc1c(C)cccc1C] |
| 2672 | 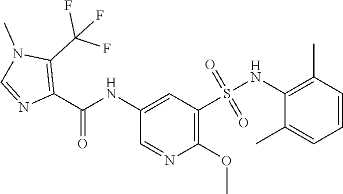 [SMILES: COc1ncc(NC(=O)c2ncn(C)c2C(F)(F)F)cc1S(=O)(=O)Nc1c(C)cccc1C] |
| 2673 | 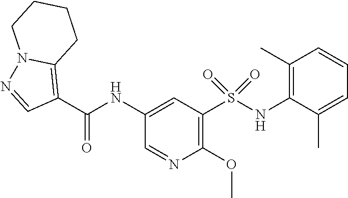 [SMILES: COc1ncc(NC(=O)c2cnn3c2CCCC3)cc1S(=O)(=O)Nc1c(C)cccc1C] |
| 2674 | 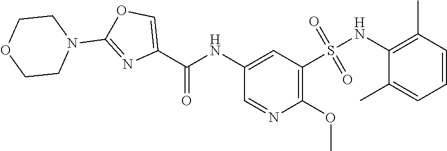 [SMILES: COc1ncc(NC(=O)c2coc(N3CCOCC3)n2)cc1S(=O)(=O)Nc1c(C)cccc1C] |
| 2675 | 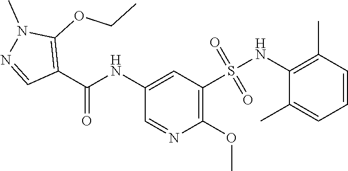 [SMILES: CCOc1c(C(=O)Nc2cnc(OC)c(S(=O)(=O)Nc3c(C)cccc3C)c2)cnn1C] |
| 2677 | 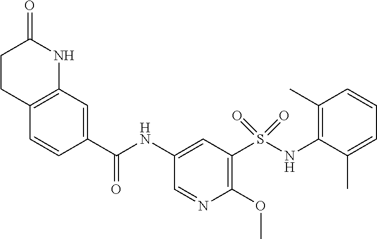 [SMILES: COc1ncc(NC(=O)c2ccc3c(c2)NC(=O)CC3)cc1S(=O)(=O)Nc1c(C)cccc1C] |
| 2678 | 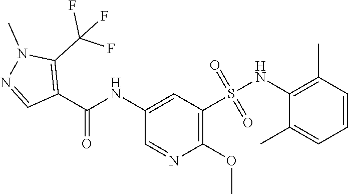 [SMILES: COc1ncc(NC(=O)c2cnn(C)c2C(F)(F)F)cc1S(=O)(=O)Nc1c(C)cccc1C] |
| 2679 | 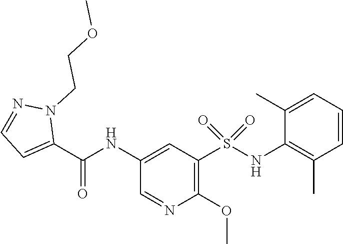 [SMILES: COCCn1nccc1C(=O)Nc1cnc(OC)c(S(=O)(=O)Nc2c(C)cccc2C)c1] |
| 2682 | 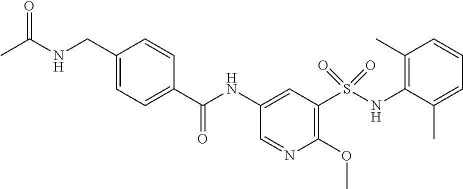 [SMILES: COc1ncc(NC(=O)c2ccc(CNC(C)=O)cc2)cc1S(=O)(=O)Nc1c(C)cccc1C] |
| 2683 | 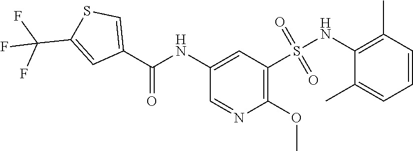 [SMILES: COc1ncc(NC(=O)c2csc(C(F)(F)F)c2)cc1S(=O)(=O)Nc1c(C)cccc1C] |
| 2684 | 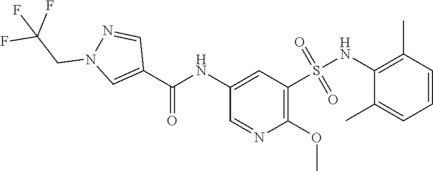 [SMILES: COc1ncc(NC(=O)c2cnn(CC(F)(F)F)c2)cc1S(=O)(=O)Nc1c(C)cccc1C] |
| 2687 | 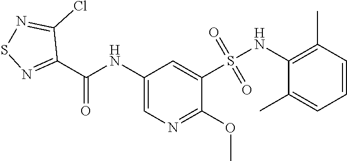 [SMILES: COc1ncc(NC(=O)c2nsnc2Cl)cc1S(=O)(=O)Nc1c(C)cccc1C] |
| 2688 | 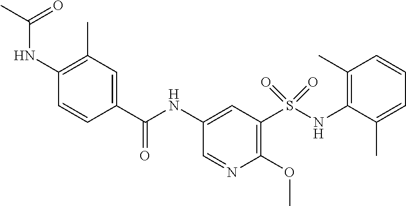 [SMILES: COc1ncc(NC(=O)c2ccc(NC(C)=O)c(C)c2)cc1S(=O)(=O)Nc1c(C)cccc1C] |
| 2689 | 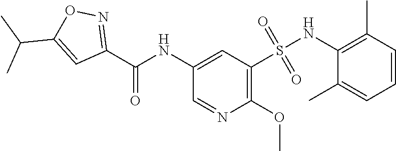 [SMILES: COc1ncc(NC(=O)c2cc(C(C)C)on2)cc1S(=O)(=O)Nc1c(C)cccc1C] |
| 2690 | 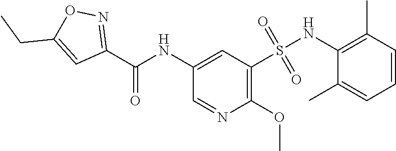 [SMILES: CCc1cc(C(=O)Nc2cnc(OC)c(S(=O)(=O)Nc3c(C)cccc3C)c2)no1] |
| 2691 | 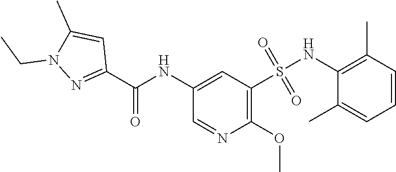 [SMILES: CCn1nc(C(=O)Nc2cnc(OC)c(S(=O)(=O)Nc3c(C)cccc3C)c2)cc1C] |
| 2692 | 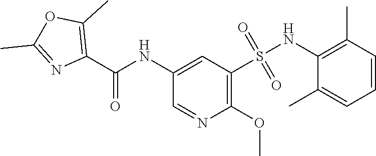 [SMILES: COc1ncc(NC(=O)c2nc(C)oc2C)cc1S(=O)(=O)Nc1c(C)cccc1C] |
| 2693 | 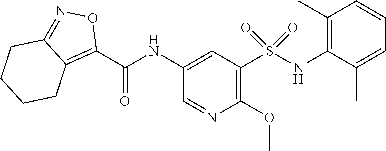 [SMILES: COc1ncc(NC(=O)c2onc3c2CCCC3)cc1S(=O)(=O)Nc1c(C)cccc1C] |
| 2694 | 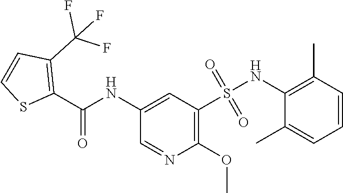 [SMILES: COc1ncc(NC(=O)c2sccc2C(F)(F)F)cc1S(=O)(=O)Nc1c(C)cccc1C] |
| 2695 | 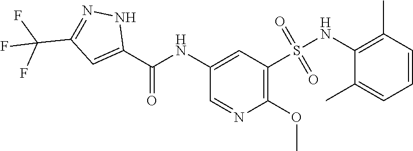 [SMILES: COc1ncc(NC(=O)c2cc(C(F)(F)F)n[nH]2)cc1S(=O)(=O)Nc1c(C)cccc1C] |
| 2696 | 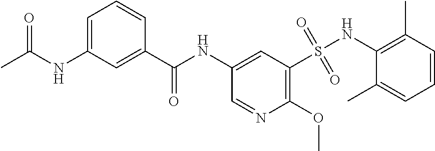 [SMILES: COc1ncc(NC(=O)c2cccc(NC(C)=O)c2)cc1S(=O)(=O)Nc1c(C)cccc1C] |
| 2697 | 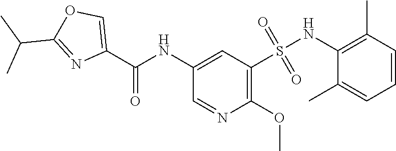 [SMILES: COc1ncc(NC(=O)c2coc(C(C)C)n2)cc1S(=O)(=O)Nc1c(C)cccc1C] |
| 2699 | 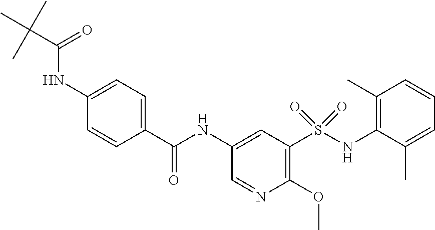 [SMILES: COc1ncc(NC(=O)c2ccc(NC(=O)C(C)(C)C)cc2)cc1S(=O)(=O)Nc1c(C)cccc1C] |
| 2700 | 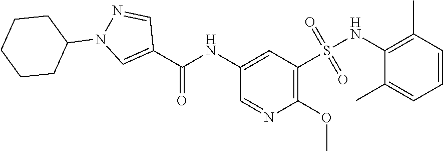 [SMILES: COc1ncc(NC(=O)c2cnn(C3CCCCC3)c2)cc1S(=O)(=O)Nc1c(C)cccc1C] |
| 2701 | 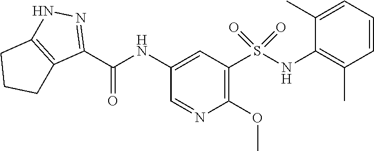 [SMILES: COc1ncc(NC(=O)c2n[nH]c3c2CCC3)cc1S(=O)(=O)Nc1c(C)cccc1C] |
| 2703 | 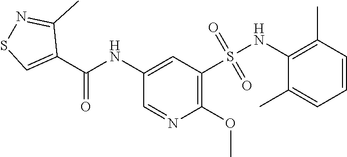 [SMILES: COc1ncc(NC(=O)c2csnc2C)cc1S(=O)(=O)Nc1c(C)cccc1C] |
| 2706 | 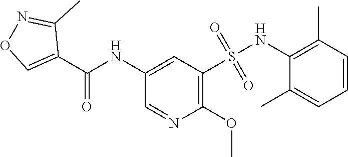 [SMILES: COc1ncc(NC(=O)c2conc2C)cc1S(=O)(=O)Nc1c(C)cccc1C] |
| 2708 | 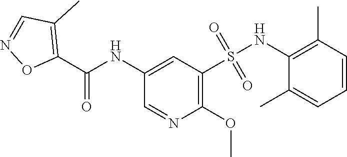 [SMILES: COc1ncc(NC(=O)c2oncc2C)cc1S(=O)(=O)Nc1c(C)cccc1C] |
| 2709 | 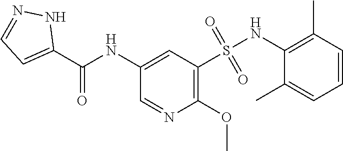 [SMILES: COc1ncc(NC(=O)c2ccn[nH]2)cc1S(=O)(=O)Nc1c(C)cccc1C] |
| 2710 | 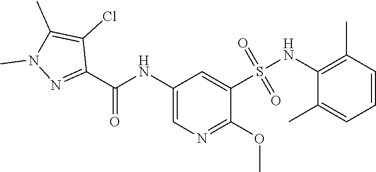 [SMILES: COc1ncc(NC(=O)c2nn(C)c(C)c2Cl)cc1S(=O)(=O)Nc1c(C)cccc1C] |
| 2711 | 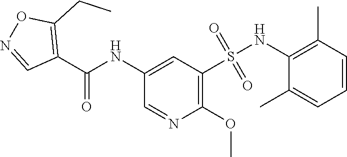 [SMILES: CCc1oncc1C(=O)Nc1cnc(OC)c(S(=O)(=O)Nc2c(C)cccc2C)c1] |
| 2712 | 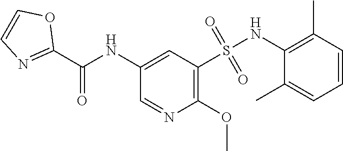 [SMILES: COc1ncc(NC(=O)c2ncco2)cc1S(=O)(=O)Nc1c(C)cccc1C] |
| 2713 | 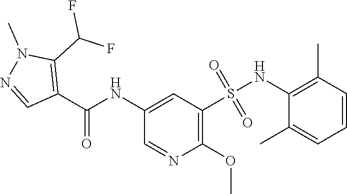 [SMILES: COc1ncc(NC(=O)c2cnn(C)c2C(F)F)cc1S(=O)(=O)Nc1c(C)cccc1C] |
| 2714 | 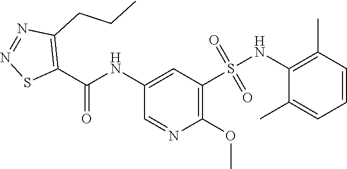 [SMILES: CCCc1nnsc1C(=O)Nc1cnc(OC)c(S(=O)(=O)Nc2c(C)cccc2C)c1] |
| 2715 | 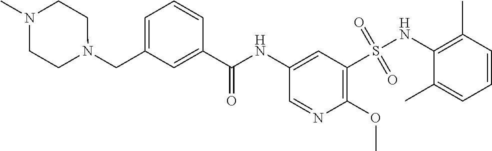 [SMILES: COc1ncc(NC(=O)c2cccc(CN3CCN(C)CC3)c2)cc1S(=O)(=O)Nc1c(C)cccc1C] |
| 2716 | 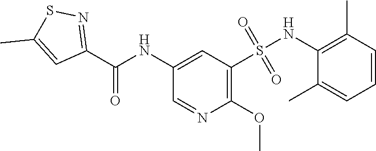 [SMILES: COc1ncc(NC(=O)c2cc(C)sn2)cc1S(=O)(=O)Nc1c(C)cccc1C] |
| 2718 | 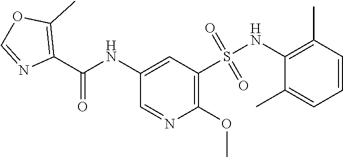 [SMILES: COc1ncc(NC(=O)c2ncoc2C)cc1S(=O)(=O)Nc1c(C)cccc1C] |
| 2719 | 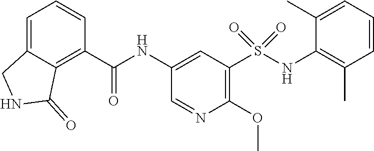 [SMILES: COc1ncc(NC(=O)c2cccc3c2C(=O)NC3)cc1S(=O)(=O)Nc1c(C)cccc1C] |
| 2720 | 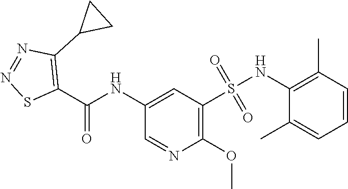 [SMILES: COc1ncc(NC(=O)c2snnc2C2CC2)cc1S(=O)(=O)Nc1c(C)cccc1C] |
| 2724 | 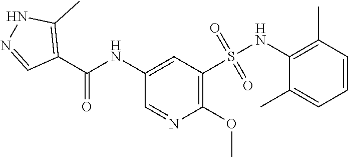 [SMILES: COc1ncc(NC(=O)c2cn[nH]c2C)cc1S(=O)(=O)Nc1c(C)cccc1C] |
| 2725 | 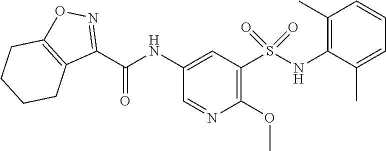 [SMILES: COc1ncc(NC(=O)c2noc3c2CCCC3)cc1S(=O)(=O)Nc1c(C)cccc1C] |
| 2726 | 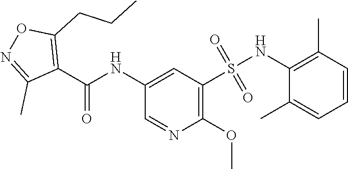 [SMILES: CCCc1onc(C)c1C(=O)Nc1cnc(OC)c(S(=O)(=O)Nc2c(C)cccc2C)c1] |
| 2727 | 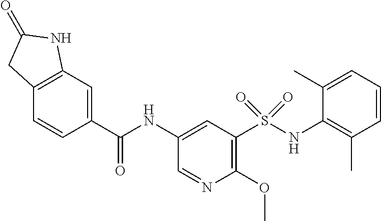 [SMILES: COc1ncc(NC(=O)c2ccc3c(c2)NC(=O)C3)cc1S(=O)(=O)Nc1c(C)cccc1C] |
| 2729 | 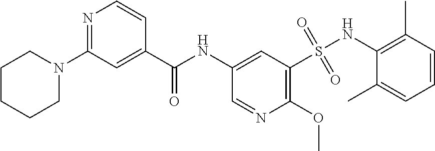 [SMILES: COc1ncc(NC(=O)c2ccnc(N3CCCCC3)c2)cc1S(=O)(=O)Nc1c(C)cccc1C] |
| 2730 | 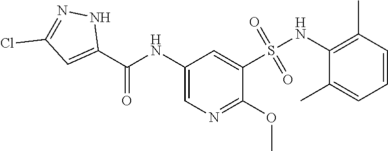 [SMILES: COc1ncc(NC(=O)c2cc(Cl)n[nH]2)cc1S(=O)(=O)Nc1c(C)cccc1C] |
| 2731 | 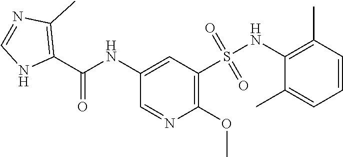 [SMILES: COc1ncc(NC(=O)c2[nH]cnc2C)cc1S(=O)(=O)Nc1c(C)cccc1C] |
| 2732 | 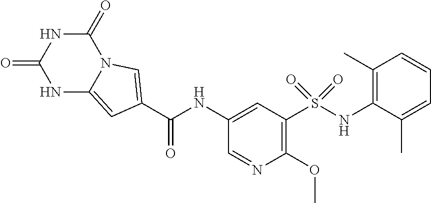 [SMILES: COc1ncc(NC(=O)c2cc3[nH]c(=O)[nH]c(=O)n3c2)cc1S(=O)(=O)Nc1c(C)cccc1C] |
| 2733 | 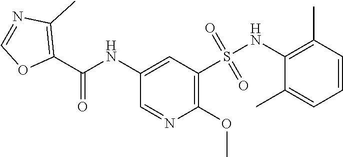 [SMILES: COc1ncc(NC(=O)c2ocnc2C)cc1S(=O)(=O)Nc1c(C)cccc1C] |
| 2734 | 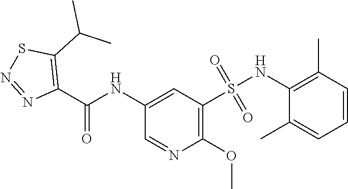 [SMILES: COc1ncc(NC(=O)c2nnsc2C(C)C)cc1S(=O)(=O)Nc1c(C)cccc1C] |
| 2735 | 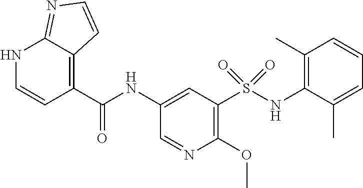 [SMILES: COc1ncc(NC(=O)c2cc[nH]c3nccc2-3)cc1S(=O)(=O)Nc1c(C)cccc1C] |
| 2736 | 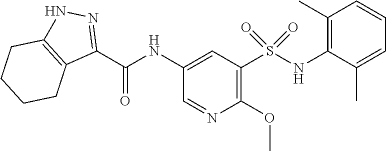 [SMILES: COc1ncc(NC(=O)c2n[nH]c3c2CCCC3)cc1S(=O)(=O)Nc1c(C)cccc1C] |
| 2737 | 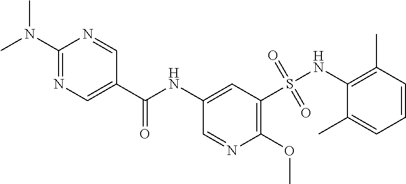 [SMILES: COc1ncc(NC(=O)c2cnc(N(C)C)nc2)cc1S(=O)(=O)Nc1c(C)cccc1C] |
| 2738 | 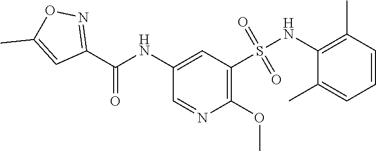 [SMILES: COc1ncc(NC(=O)c2cc(C)on2)cc1S(=O)(=O)Nc1c(C)cccc1C] |
| 2740 | 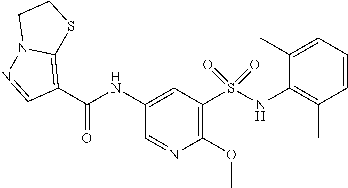 [SMILES: COc1ncc(NC(=O)c2cnn3c2SCC3)cc1S(=O)(=O)Nc1c(C)cccc1C] |
| 2741 | 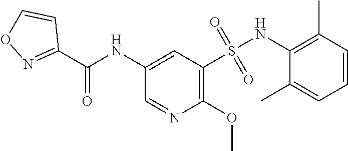 [SMILES: COc1ncc(NC(=O)c2ccon2)cc1S(=O)(=O)Nc1c(C)cccc1C] |
| 2742 | 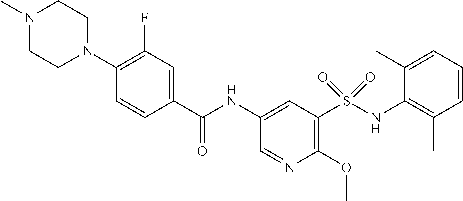 [SMILES: COc1ncc(NC(=O)c2ccc(N3CCN(C)CC3)c(F)c2)cc1S(=O)(=O)Nc1c(C)cccc1C] |
| 2743 | 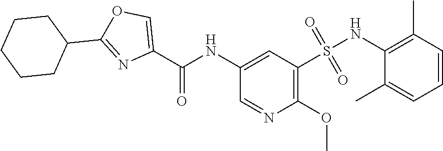 [SMILES: COc1ncc(NC(=O)c2coc(C3CCCCC3)n2)cc1S(=O)(=O)Nc1c(C)cccc1C] |
| 2744 | 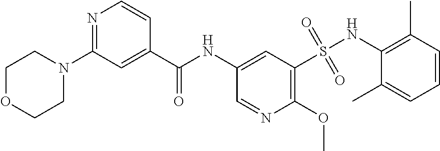 [SMILES: COc1ncc(NC(=O)c2ccnc(N3CCOCC3)c2)cc1S(=O)(=O)Nc1c(C)cccc1C] |
| 2745 | 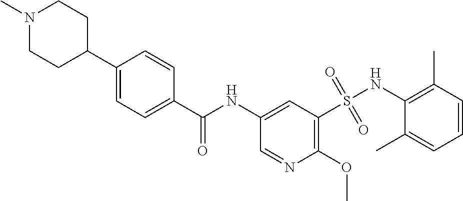 [SMILES: COc1ncc(NC(=O)c2ccc(C3CCN(C)CC3)cc2)cc1S(=O)(=O)Nc1c(C)cccc1C] |
| 2746 | 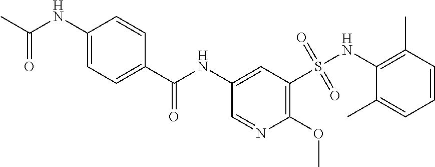 [SMILES: COc1ncc(NC(=O)c2ccc(NC(C)=O)cc2)cc1S(=O)(=O)Nc1c(C)cccc1C] |
| 2747 | 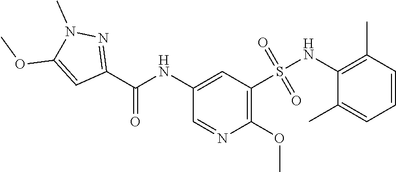 [SMILES: COc1ncc(NC(=O)c2cc(OC)n(C)n2)cc1S(=O)(=O)Nc1c(C)cccc1C] |
| 2749 | 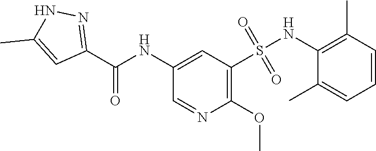 [SMILES: COc1ncc(NC(=O)c2cc(C)[nH]n2)cc1S(=O)(=O)Nc1c(C)cccc1C] |
| 2750 | 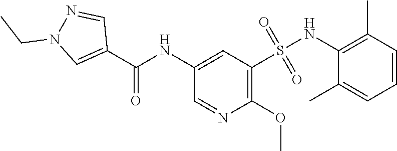 [SMILES: CCn1cc(C(=O)Nc2cnc(OC)c(S(=O)(=O)Nc3c(C)cccc3C)c2)cn1] |
| 2751 | 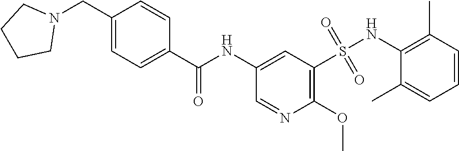 [SMILES: COc1ncc(NC(=O)c2ccc(CN3CCCC3)cc2)cc1S(=O)(=O)Nc1c(C)cccc1C] |
| 2752 | 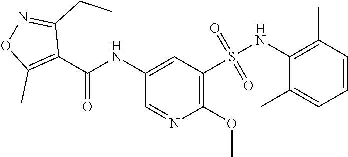 [SMILES: CCc1noc(C)c1C(=O)Nc1cnc(OC)c(S(=O)(=O)Nc2c(C)cccc2C)c1] |
| 2753 | 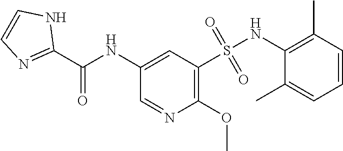 [SMILES: COc1ncc(NC(=O)c2ncc[nH]2)cc1S(=O)(=O)Nc1c(C)cccc1C] |
| 2754 | 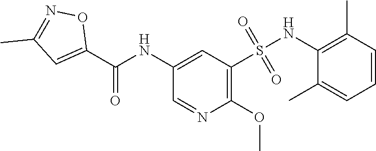 [SMILES: COc1ncc(NC(=O)c2cc(C)no2)cc1S(=O)(=O)Nc1c(C)cccc1C] |
| 2755 | 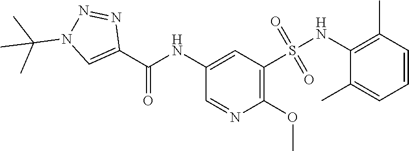 [SMILES: COc1ncc(NC(=O)c2cn(C(C)(C)C)nn2)cc1S(=O)(=O)Nc1c(C)cccc1C] |
| 2756 | 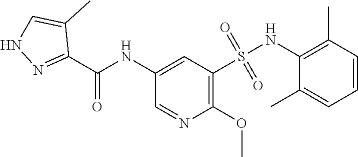 [SMILES: COc1ncc(NC(=O)c2n[nH]cc2C)cc1S(=O)(=O)Nc1c(C)cccc1C] |
| 2758 | 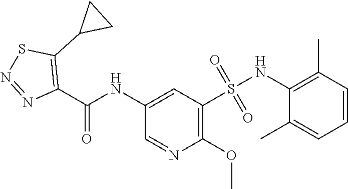 [SMILES: COc1ncc(NC(=O)c2nnsc2C2CC2)cc1S(=O)(=O)Nc1c(C)cccc1C] |
| 2759 | 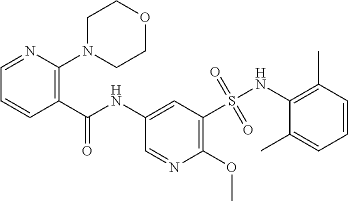 [SMILES: COc1ncc(NC(=O)c2cccnc2N2CCOCC2)cc1S(=O)(=O)Nc1c(C)cccc1C] |
| 2761 | 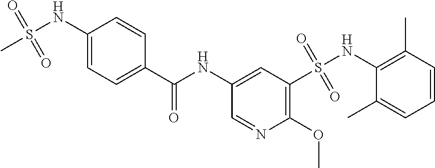 [SMILES: COc1ncc(NC(=O)c2ccc(NS(C)(=O)=O)cc2)cc1S(=O)(=O)Nc1c(C)cccc1C] |
| 2762 | 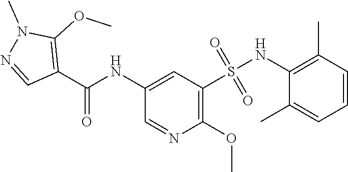 [SMILES: COc1ncc(NC(=O)c2cnn(C)c2OC)cc1S(=O)(=O)Nc1c(C)cccc1C] |
| 2764 | 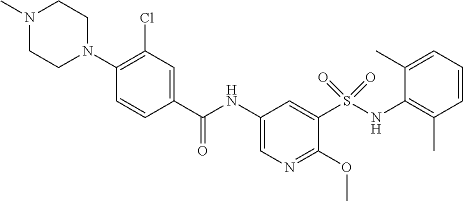 [SMILES: COc1ncc(NC(=O)c2ccc(N3CCN(C)CC3)c(Cl)c2)cc1S(=O)(=O)Nc1c(C)cccc1C] |
| 2766 | 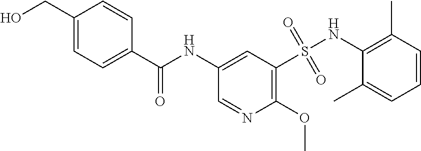 [SMILES: COc1ncc(NC(=O)c2ccc(CO)cc2)cc1S(=O)(=O)Nc1c(C)cccc1C] |
| 2767 | 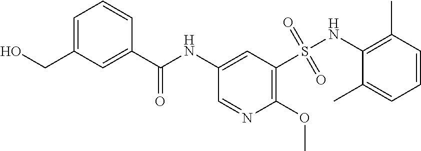 [SMILES: COc1ncc(NC(=O)c2cccc(CO)c2)cc1S(=O)(=O)Nc1c(C)cccc1C] |
| 2768 | 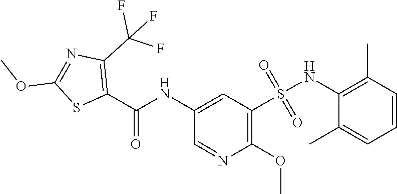 [SMILES: COc1nc(C(F)(F)F)c(C(=O)Nc2cnc(OC)c(S(=O)(=O)Nc3c(C)cccc3C)c2)s1] |
| 2769 | 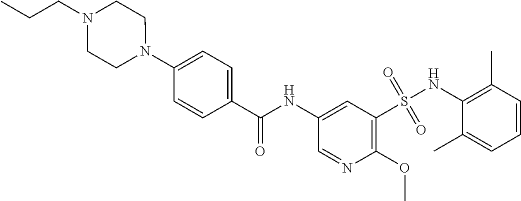 [SMILES: CCCN1CCN(c2ccc(C(=O)Nc3cnc(OC)c(S(=O)(=O)Nc4c(C)cccc4C)c3)cc2)CC1] |
| 2770 | 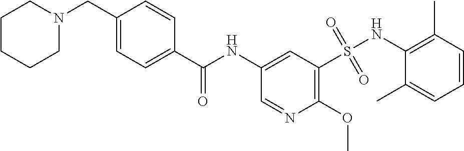 [SMILES: COc1ncc(NC(=O)c2ccc(CN3CCCCC3)cc2)cc1S(=O)(=O)Nc1c(C)cccc1C] |
| 2771 | 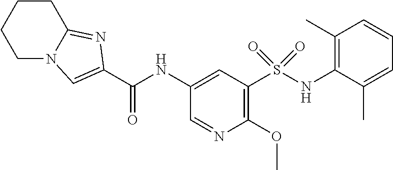 [SMILES: COc1ncc(NC(=O)c2cn3c(n2)CCCC3)cc1S(=O)(=O)Nc1c(C)cccc1C] |
| 2772 | 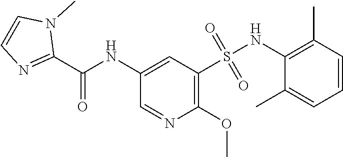 [SMILES: COc1ncc(NC(=O)c2nccn2C)cc1S(=O)(=O)Nc1c(C)cccc1C] |
| 2774 | 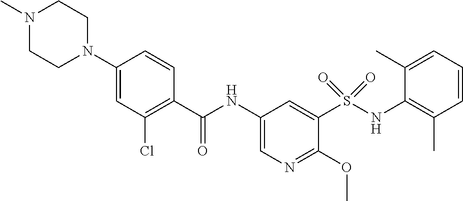 [SMILES: COc1ncc(NC(=O)c2ccc(N3CCN(C)CC3)cc2Cl)cc1S(=O)(=O)Nc1c(C)cccc1C] |
| 2775 | 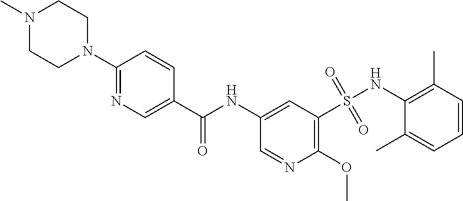 [SMILES: COc1ncc(NC(=O)c2ccc(N3CCN(C)CC3)nc2)cc1S(=O)(=O)Nc1c(C)cccc1C] |
| 2776 | 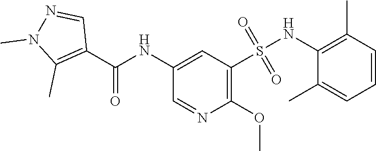 [SMILES: COc1ncc(NC(=O)c2cnn(C)c2C)cc1S(=O)(=O)Nc1c(C)cccc1C] |
| 2777 | 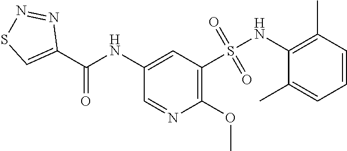 [SMILES: COc1ncc(NC(=O)c2csnn2)cc1S(=O)(=O)Nc1c(C)cccc1C] |
| 2779 | 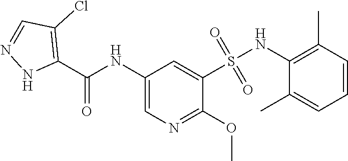 [SMILES: COc1ncc(NC(=O)c2[nH]ncc2Cl)cc1S(=O)(=O)Nc1c(C)cccc1C] |
| 2780 | 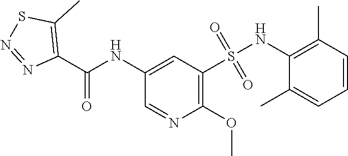 [SMILES: COc1ncc(NC(=O)c2nnsc2C)cc1S(=O)(=O)Nc1c(C)cccc1C] |
| 2781 | 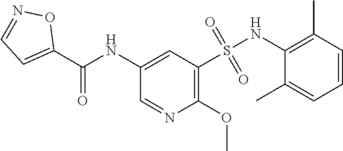 [SMILES: COc1ncc(NC(=O)c2ccno2)cc1S(=O)(=O)Nc1c(C)cccc1C] |
| 2782 | 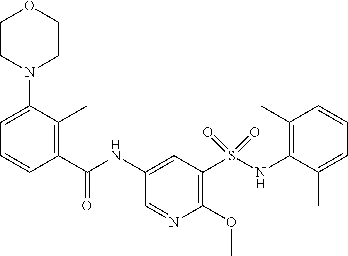 [SMILES: COc1ncc(NC(=O)c2cccc(N3CCOCC3)c2C)cc1S(=O)(=O)Nc1c(C)cccc1C] |
| 2784 | 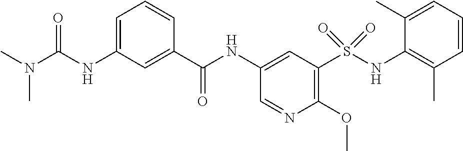 [SMILES: COc1ncc(NC(=O)c2cccc(NC(=O)N(C)C)c2)cc1S(=O)(=O)Nc1c(C)cccc1C] |
| 2785 | 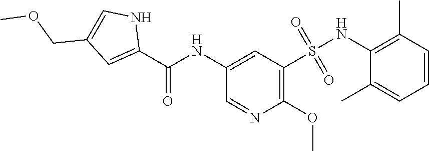 [SMILES: COCc1c[nH]c(C(=O)Nc2cnc(OC)c(S(=O)(=O)Nc3c(C)cccc3C)c2)c1] |
| 2786 | 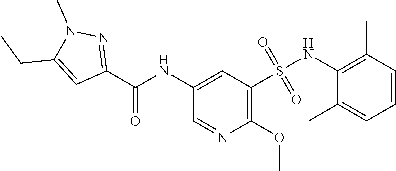 [SMILES: CCc1cc(C(=O)Nc2cnc(OC)c(S(=O)(=O)Nc3c(C)cccc3C)c2)nn1C] |
| 2787 | 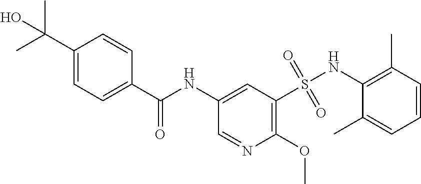 [SMILES: COc1ncc(NC(=O)c2ccc(C(C)(C)O)cc2)cc1S(=O)(=O)Nc1c(C)cccc1C] |
| 2788 | 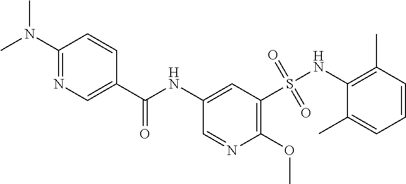 [SMILES: COc1ncc(NC(=O)c2ccc(N(C)C)nc2)cc1S(=O)(=O)Nc1c(C)cccc1C] |
| 2789 | 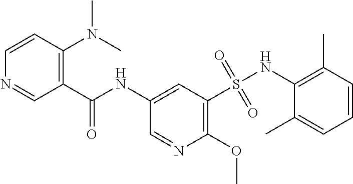 [SMILES: COc1ncc(NC(=O)c2cnccc2N(C)C)cc1S(=O)(=O)Nc1c(C)cccc1C] |
| 2790 | 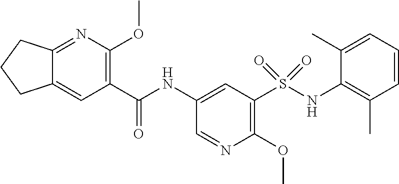 [SMILES: COc1nc2c(cc1C(=O)Nc1cnc(OC)c(S(=O)(=O)Nc3c(C)cccc3C)c1)CCC2] |
| 2791 | 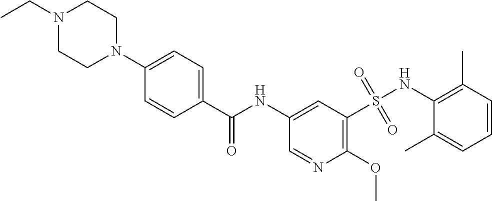 [SMILES: CCN1CCN(c2ccc(C(=O)Nc3cnc(OC)c(S(=O)(=O)Nc4c(C)cccc4C)c3)cc2)CC1] |
| 2792 | 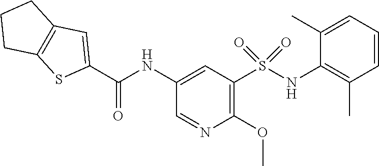 [SMILES: COc1ncc(NC(=O)c2cc3c(s2)CCC3)cc1S(=O)(=O)Nc1c(C)cccc1C] |
| 2793 | 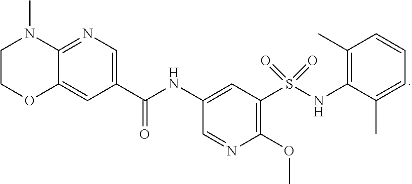 [SMILES: COc1ncc(NC(=O)c2cnc3c(c2)OCCN3C)cc1S(=O)(=O)Nc1c(C)cccc1C] |

or a pharmaceutically acceptable salt, solvate, hydrate, polymorph, co-crystal, tautomer, stereoisomer, isotopically labeled compound, or prodrug thereof (e.g., a pharmaceutically acceptable salt thereof).

In certain embodiments, the compound is Compound 1178, or a pharmaceutically acceptable salt, solvate, hydrate, polymorph, co-crystal, tautomer, stereoisomer, isotopically labeled compound, or prodrug thereof. In certain embodiments, the compound is Compound 1178, or a pharmaceutically acceptable salt thereof. In certain embodiments, the compound is Compound 1206, or a pharmaceutically acceptable salt, solvate, hydrate, polymorph, co-crystal, tautomer, stereoisomer, isotopically labeled compound, or prodrug thereof. In certain embodiments, the compound is Compound 1206, or a pharmaceutically acceptable salt thereof. In certain embodiments, the compound is Compound 1385, or a pharmaceutically acceptable salt, solvate, hydrate, polymorph, co-crystal, tautomer, stereoisomer, isotopically labeled compound, or prodrug thereof. In certain embodiments, the compound is Compound 1385, or a pharmaceutically acceptable salt thereof. In certain embodiments, the compound is Compound 1481, or a pharmaceutically acceptable salt, solvate, hydrate, polymorph, co-crystal, tautomer, stereoisomer, isotopically labeled compound, or prodrug thereof. In certain embodiments, the compound is Compound 1481, or a pharmaceutically acceptable salt thereof. In certain embodiments, the compound is Compound 2116, or a pharmaceutically acceptable salt, solvate, hydrate, polymorph, co-crystal, tautomer, stereoisomer, isotopically labeled compound, or prodrug thereof. In certain embodiments, the compound is Compound 2116, or a pharmaceutically acceptable salt thereof.

In certain embodiments, a provided compound (a compound of the present disclosure) is a compound of Formula (I), or a pharmaceutically acceptable salt, solvate, hydrate, polymorph, co-crystal, tautomer, stereoisomer, isotopically labeled compound, or prodrug thereof. In certain embodiments, a provided compound is a compound of Formula (I), or a pharmaceutically acceptable salt, solvate, hydrate, polymorph, co-crystal, tautomer, stereoisomer, or isotopically labeled compound thereof. In certain embodiments, a provided compound is a compound of Formula (I), or a pharmaceutically acceptable salt, tautomer, or stereoisomer thereof. In certain embodiments, a provided compound is a compound of Formula (I), or a pharmaceutically acceptable salt thereof. In certain embodiments, a provided compound is a mixture (e.g., a racemic mixture) of enantiomers and/or diastereomers.

In certain embodiments, a provided compound is electrically neutral. In certain embodiments, a provided compound further comprises one or more counterions so that the provided compound is electrically neutral.

In certain embodiments, the molecular weight of a provided compound that is not in the form of a salt, solvate, hydrate, co-crystal, or prodrug is lower than 1,000, lower than 800, lower than 600, lower than 500, or lower than 400 g/mol. In certain embodiments, the molecular weight of a provided compound that is not in the form of a salt, solvate, hydrate, co-crystal, or prodrug is lower than 600 g/mol. In certain embodiments, the molecular weight of a provided compound that is not in the form of a salt, solvate, hydrate, co-crystal, or prodrug is lower than 500 g/mol.

In certain embodiments, a provided compound is a TRAP1 modulator. In certain embodiments, a provided compound modulates the function of TRAP1. In certain embodiments, a provided compound is a TRAP1 activator. In certain embodiments, a provided compound increases the expression and/or activity of TRAP1. In certain embodiments, a provided compound increases the activity of TRAP1. In certain embodiments, the activity of TRAP1 is ATPase activity of TRAP1. In certain embodiments, a provided compound increases the expression and/or activity of TRAP1 in an in vitro assay (e.g., an in vitro assay described herein). In certain embodiments, a provided compound increases the expression and/or activity of TRAP1 in a cellular assay (e.g., a cellular assay described herein). In certain embodiments, a provided compound increases the expression and/or activity of TRAP1 (e.g., as measured by Eₘₐₓ (maximal percent activation)) by at least 10%, at least 20%, at least 30%, at least 50%, at least 70%, at least 100%, at least 300%, or at least 1,000%. In certain embodiments, the TRAP1 is a human TRAP1. In certain embodiments, the TRAP1 is a non-human mammal TRAP1. In certain embodiments, the TRAP1 is a wild type TRAP1. In certain embodiments, the TRAP1 is a mutant TRAP1. In certain embodiments, a provided compound is selective for increasing the expression and/or activity of TRAP1 over a different protein (e.g., a protein kinase or a heat shock protein (e.g., a HSP90 (e.g., HSP90B, GRP94))). In certain embodiments, the selectivity is at least 2-fold, at least 3-fold, at least 4-fold, at least 5-fold, at least 7-fold, at least 10-fold, at least 20-fold, at least 50-fold, at least 100-fold, at least 300-fold, or at least 1,000-fold. For example, if a provided compound's TRAP1 EC₅₀ is 1 μM, and the provided compound's EC₅₀ regarding a different protein is 10 μM, then the selectivity is (10 μM)/(1 μM)=10 folds. In certain embodiments, a provided compound reversibly binds to TRAP1.

It has also been reported that TRAP1 may protect against mitochondrial apoptosis (Altieri et al., Biochim Biophys Acta 2012, 1823: 767-73). In certain embodiments, a provided compound increases the quality, health, function, quantity, and/or activity of mitochondria by at least 10%, at least 20%, at least 30%, at least 50%, at least 70%, at least 100%, at least 300%, or at least 1,000%.

In certain embodiments, the increase is obtained with an assay described herein.

The provided compounds may be advantageous over known compounds. In certain embodiments, the provided compounds are more soluble and/or permeable than known compounds. In certain embodiments, the provided compounds show higher brain penetration than known compounds. In certain embodiments, the provided compounds show more desirable absorption, distribution, metabolism, excretion, and/or liberation than known compounds. In certain embodiments, the provided compounds show higher bioavailability than known compounds. In certain embodiments, the provided compounds are more physically, chemically, and/or metabolically stable than known compounds. In certain embodiments, the provided compounds are more potent than known compounds. In certain embodiments, the provided compounds show less frequent and/or less severe side effects than known compounds. In certain embodiments, the provided compounds show less frequent and/or less severe off-target effects than known compounds. In certain embodiments, the provided compounds are less toxic than known compounds. In certain embodiments, the provided compounds are more efficacious than known compounds. In certain embodiments, the provided compounds show wider therapeutic window than known compounds. In certain embodiments, the provided compounds show better subject (e.g., a human in need of treatment or prevention of a disease) compliance than known compounds.

### Pharmaceutical Compositions and Administration

In another aspect, the present disclosure provides pharmaceutical compositions comprising a provided compound and optionally a pharmaceutically acceptable excipient.

In certain embodiments, the pharmaceutical composition comprises an effective amount of the provided compound. In certain embodiments, an effective amount is an amount effective for increasing the expression of TRAP1 in a subject, biological sample, tissue, or cell. In certain embodiments, an effective amount is an amount effective for increasing the activity of TRAP1 in a subject, biological sample, tissue, or cell. In certain embodiments, an effective amount is an amount effective for increasing the health, quality, function, quantity, and/or activity of mitochondria in a subject, biological sample, tissue, or cell. In certain embodiments, the effective amount increases the expression and/or activity of TRAP1 in a subject, biological sample, tissue, or cell by at least 10%, at least 20%, at least 30%, at least 50%, at least 70%, at least 100%, at least 300%, or at least 1,000%. In certain embodiments, the effective amount increases the health, quality, function, quantity, and/or activity of mitochondria in a subject, biological sample, tissue, or cell by at least 10%, at least 20%, at least 30%, at least 50%, at least 70%, at least 100%, at least 300%, or at least 1,000%.

In certain embodiments, the effective amount is a therapeutically effective amount. In certain embodiments, the effective amount is effective in treating (e.g., therapeutically treating) a disease in a subject in need thereof. In certain embodiments, the effective amount is a prophylactically effective amount. In certain embodiments, the effective amount is effective in preventing a disease in a subject in need thereof.

In certain embodiments, the subject is an animal. The animal may be of either sex and may be at any stage of development. In certain embodiments, the subject is a human (e.g., an adult, juvenile, or child). In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the subject is a genetically engineered animal. In certain embodiments, the subject is a transgenic animal (e.g., transgenic mice, transgenic pigs). In certain embodiments, the subject is a fish or reptile.

In certain embodiments, the biological sample, tissue, or cell (e.g., the biological sample, tissue, or cell being contacted with a provided compound or pharmaceutical composition) is in vitro. In certain embodiments, the biological sample, tissue, or cell is in vivo. In certain embodiments, the biological sample, tissue, or cell is ex vivo. In certain embodiments, the cell is a neuron (e.g., dysfunctional neuron).

Pharmaceutical compositions can be prepared by any method known in the art of pharmacology. In general, such preparatory methods include bringing the provided compound (“active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition will vary, depending upon the identity, size, and/or condition of the subject treated and further depending upon the route by which the pharmaceutical composition is to be administered. The pharmaceutical composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol*), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip©, methylparaben, Germall© 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral pharmaceutical compositions can include adjuvants such as wetting agents, emulsifying and suspending agents, sweetening, flavoring, and perfuming agents. In certain embodiments for parenteral administration, the conjugates are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid pharmaceutical compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Pharmaceutical compositions for rectal or vaginal administration are typically suppositories which can be prepared by mixing the conjugates with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid pharmaceutical compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a pharmaceutical composition that they release the active ingredient(s) only, or preferentially, in a certain part of the intestinal tract, optionally, in a delayed manner. Examples of encapsulating pharmaceutical compositions which can be used include polymeric substances and waxes. Solid pharmaceutical compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a pharmaceutical composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a provided compound may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions include short needle devices. Intradermal pharmaceutical compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the provided compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients.

A pharmaceutical composition can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such pharmaceutical compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder pharmaceutical compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations as being useful for pulmonary delivery are useful for intranasal delivery of a pharmaceutical composition. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients. A pharmaceutical composition can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable pharmaceutical composition and, optionally, one or more of the additional ingredients. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients.

A pharmaceutical composition can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of the pharmaceutical compositions are principally directed to pharmaceutical compositions which are suitable for administration to humans, it will be understood by the skilled artisan that such pharmaceutical compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

The provided compounds are typically formulated in dosage unit form for ease of administration and uniformity of dosage. It will be understood, however, that the total daily usage of the pharmaceutical compositions will be decided by a physician within the scope of sound medical judgment. The specific therapeutically effective dose level for any particular subject or organism will depend upon a variety of factors including the disease being treated and the severity of the disorder; the activity of the specific active ingredient employed; the specific pharmaceutical composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The provided compounds and pharmaceutical compositions can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the provided compound or pharmaceutical composition is suitable for topical administration to the eye of a subject.

The exact amount of a provided compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a provided compound. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample, tissue, or cell is one dose per day. In certain embodiments, the frequency of administering the multiple doses to the subject or applying the multiple doses to the biological sample, tissue, or cell is two doses per day. In certain embodiments, the frequency of administering the multiple doses to the subject or applying the multiple doses to the biological sample, tissue, or cell is three doses per day. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject or cell. In certain embodiments, the duration between the first dose and last dose of the multiple doses is three months, six months, or one year. In certain embodiments, the duration between the first dose and last dose of the multiple doses is the lifetime of the subject or cell. In certain embodiments, a dose (e.g., a single dose, or any dose of multiple doses) includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a provided compound. In certain embodiments, a dose includes independently between 1 mg and 3 mg, inclusive, of a provided compound. In certain embodiments, a dose includes independently between 3 mg and 10 mg, inclusive, of a provided compound. In certain embodiments, a dose includes independently between 10 mg and 30 mg, inclusive, of a provided compound. In certain embodiments, a dose includes independently between 30 mg and 100 mg, inclusive, of a provided compound.

Dose ranges as provide guidance for the administration of provided pharmaceutical compositions to an adult. The amount to be administered to, for example, a child or an adolescent can be determined by a medical practitioner or person skilled in the art and can be lower or the same as that administered to an adult.

In certain embodiments, the pharmaceutical composition further comprises an additional pharmaceutical agent. The additional pharmaceutical agent is different from the provided compound. In certain embodiments, the additional pharmaceutical agent is an additional therapeutically active agent. In certain embodiments, the additional pharmaceutical agent is an additional prophylactically active agent. A provided compound or pharmaceutical composition can be administered in combination with one or more additional pharmaceutical agents. The provided compounds or pharmaceutical compositions can be administered in combination with additional pharmaceutical agents that improve their activity (e.g., activity (e.g., potency and/or efficacy) in treating a disease in a subject in need thereof, in preventing a disease in a subject in need thereof, improve bioavailability, improve safety, reduce drug resistance, reduce and/or modify metabolism, inhibit excretion, and/or modify distribution in a subject, biological sample, or cell. It will also be appreciated that the therapy employed may achieve a desired effect for the same disorder, and/or it may achieve different effects. In certain embodiments, a pharmaceutical composition including a provided compound and an additional pharmaceutical agent shows a synergistic effect that is absent in a pharmaceutical composition including one of the provided compound and the additional pharmaceutical agent, but not both.

The provided compound or pharmaceutical composition can be administered concurrently with, prior to, or subsequent to one or more additional pharmaceutical agents, which may be useful as, e.g., combination therapies. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease or premalignant condition. Each additional pharmaceutical agent may be administered at a dose and/or on a time schedule determined for that pharmaceutical agent. The additional pharmaceutical agents may also be administered together with each other and/or with a provided compound or pharmaceutical composition in a single dose or administered separately in different doses. The particular combination to employ in a regimen will take into account compatibility of the provided compound with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, cytotoxic chemotherapeutic agents, epigenetic modifiers, glucocorticoids, immunotherapeutic agents, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In some embodiments, the additional pharmaceutical agent is a topoisomerase inhibitor, a MCL1 inhibitor, a BCL-2 inhibitor, a BCL-xL inhibitor, a BRD4 inhibitor, a BRCA1 inhibitor, BRCA2 inhibitor, HER1 inhibitor, HER2 inhibitor, a CDK9 inhibitor, a Jumonji histone demethylase inhibitor, or a DNA damage inducer. In certain embodiments, the additional pharmaceutical agent is a binder or inhibitor of a kinase (e.g., tyrosine kinase). In certain embodiments, the additional pharmaceutical agent is an antibody or a fragment thereof (e.g., monoclonal antibody). In certain embodiments, the additional therapy is an immunotherapy (e.g., an immunotherapeutic monoclonal antibody). In certain embodiments, the additional pharmaceutical agent is an immunosuppressor. In certain embodiments, the additional pharmaceutical agent is an immunoactivator. In certain embodiments, the additional pharmaceutical agent is an immune checkpoint inhibitor. In certain embodiments, the additional pharmaceutical agent is a programmed cell death 1 protein (PD-1) inhibitor. In certain embodiments, the additional pharmaceutical agent is a programmed cell death 1 protein ligand 1 (PD-L1) inhibitor. In certain embodiments, the additional pharmaceutical agent is a cytotoxic T-lymphocyte-associated protein 4 (CTLA-4) inhibitor. In certain embodiments, the additional pharmaceutical agent is a T-cell immunoglobulin domain and mucin domain 3 (TIM3) inhibitor, lymphocyte activation gene-3 (LAG3) inhibitor, V-set domain-containing T-cell activation inhibitor 1 (VTCN1 or B7-H4) inhibitor, cluster of differentiation 276 (CD276 or B7-H3) inhibitor, B and T lymphocyte attenuator (BTLA) inhibitor, galectin-9 (GAL9) inhibitor, checkpoint kinase 1 (Chki) inhibitor, adenosine A2A receptor (A2AR) inhibitor, indoleamine 2,3-dioxygenase (IDO) inhibitor, killer-cell immunoglobulin-like receptor (KIR) inhibitor, or V-domain Ig suppressor of T cell activation (VISTA) inhibitor. In certain embodiments, the additional pharmaceutical agent is metformin. In certain embodiments, the additional pharmaceutical agent is approved for human and/or veterinarian administration by a regulatory agency, such as the U.S. Food and Drug Administration (FDA) or the European Agency for the Evaluation of Medicinal Products (EMA). In certain embodiments, the provided compounds or pharmaceutical compositions can be administered in combination with surgery, radiation therapy, and/or transplantation (e.g., stem cell transplantation, bone marrow transplantation).

### Kits

In another aspect, the present disclosure provides kits comprising a provided compound or pharmaceutical composition, and instructions for using the provided compound or pharmaceutical composition. In certain embodiments, the kit comprises a first container, wherein the first container comprises the provided compound or pharmaceutical composition. In some embodiments, the kit further comprises a second container. In certain embodiments, the second container includes an excipient (e.g., an excipient for dilution or suspension of the provided compound or pharmaceutical composition). In certain embodiments, the second container includes an additional pharmaceutical agent. In some embodiments, the kit further comprises a third container. In certain embodiments, the third container includes an additional pharmaceutical agent. In some embodiments, the provided compound or pharmaceutical composition included in the first container and the excipient or additional pharmaceutical agent included in the second container are combined to form one unit dosage form. In some embodiments, the provided compound or pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the first container, second container, and third container do not comprise the instructions. In certain embodiments, the instructions are for administering the provided compound or pharmaceutical composition to a subject (e.g., a subject in need of treatment or prevention of a disease). In certain embodiments, the instructions are for contacting a biological sample, tissue, or cell with the provided compound or pharmaceutical composition. In certain embodiments, the instructions comprise information required by a regulatory agency, such as the FDA or EMA. In certain embodiments, the instructions comprise prescribing information.

### Methods of Use and Uses

The present disclosure also provides methods of using the provided compounds and pharmaceutical compositions. In another aspect, the present disclosure provides methods of increasing the expression and/or activity of TRAP1 in a subject in need thereof, the methods comprising administering to the subject in need thereof an effective amount of a provided compound or pharmaceutical composition. In certain embodiments, the activity of TRAP1 is the ATPase activity of TRAP1.

In another aspect, the present disclosure provides methods of increasing the health, quality, function, quantity, and/or activity of mitochondria in a subject in need thereof, the methods comprising administering to the subject in need thereof an effective amount of a provided compound or pharmaceutical composition.

In another aspect, the present disclosure provides methods of increasing the expression and/or activity of TRAP1 in a cell, tissue, or biological sample, the methods comprising contacting the cell, tissue, or biological sample with an effective amount of a provided compound or pharmaceutical composition.

In another aspect, the present disclosure provides methods of increasing the health, quality, function, quantity, and/or activity of mitochondria in a cell, tissue, or biological sample, the methods comprising contacting the cell, tissue, or biological sample with an effective amount of a provided compound or pharmaceutical composition.

The provided compounds and pharmaceutical compositions may also be useful for treating or preventing a disease in a subject in need thereof. It has been reported that that PINK1 may protect against oxidative-stress-induced cell death by suppressing cytochrome c release from mitochondria, and this protective action of PINK1 may depend on its kinase activity to phosphorylate TRAP1 (Pridgeon et al., PLoS Biol., 2007, 5, e172). Moreover, the ability of PINK1 to promote TRAP1 phosphorylation and cell survival may be impaired by of Parkinson's disease linked PINK1 G309D, L347P, and W437X mutations. See, id. PINK1 may phosphorylate downstream effector TRAP1 to prevent oxidative-stress-induced apoptosis. See, id.

It has been reported that that TRAP1 may work downstream of PINK1 and in parallel with parkin in Drosophila, and that enhancing its function may ameliorate mitochondrial dysfunction and rescue neurodegeneration in Parkinson's disease (Costa et al., Cell Death and Disease (2013) 4, e467).

It has been reported that in certain human cell models, TRAP1 overexpression may be protective, rescuing HTRA2 and PINK1-associated mitochondrial dysfunction, that TRAP1 may act downstream of HTRA2 and PINK1, and that TRAP1 loss of function may lead to reduced control of energy metabolism, ultimately impacting mitochondrial membrane potential (Fitzgerald et al., Brain 2017: 140; 2444-2459).

It has been reported that that [A53T]α-Synuclein toxicity may be intimately connected to mitochondrial dysfunction, and that toxicity reduction in fly and rat primary neurons and human cell lines may be achieved using overexpression of the mitochondrial chaperone TRAP1 (Butler et al., PLoS Genetics, 2012, volume 8, issue 2, e1002488). α-Synuclein may be a causal factor in Parkinson's disease pathogenesis. See id.

It has been reported that mitochondria are intimately involved in the regulation of calcium homeostasis, stress response, and cell death pathways, that an impairment of mitochondrial function results in cellular damage and is linked to aging and neurodegeneration, that mitochondrial dysfunction plays a central role in the pathogenesis of Parkinson's disease, and that several Parkinson's disease-associated genes interface with pathways regulating mitochondrial function, morphology, and dynamics (Winklhofer et al., Biochimica et Biophysica Acta, 1802 (2010) 29-44).

It has been reported that there is overwhelming evidence of impaired mitochondrial function as a causative factor in neurodegenerative diseases, that evidence has emerged for impaired mitochondrial dynamics (e.g., shape, size, fission-fusion, distribution, movement etc.) in neurodegenerative diseases such as Parkinson's disease, Huntington's disease, amyotrophic lateral sclerosis, Alzheimer's disease, and Friedreich's ataxia.

It has been reported that mitochondrial dysfunction is not only observed in monogenic mitochondrial disorders but is also associated with more common pathologic conditions, such as Alzheimer's disease, Parkinson's disease, cancer, cardiac disease, diabetes, epilepsy, Huntington's disease, and obesity (Koopman et al., The New England Journal of Medicine, 2012, 366, 1132-1141).

It has been reported that some mitochondrial disorders only affect a single organ (e.g., the eye in Leber hereditary optic neuropathy [LHON]), many other mitochondrial disorders involve multiple organ systems and often present with prominent neurologic and myopathic features (Chinnery P F. Mitochondrial Disorders Overview. 2000 Jun. 8 [Updated 2014 Aug. 14]. In: Adam M P, Ardinger H H, Pagon R A, et al., editors. GeneReviews® [Internet]. Seattle (Wash.): University of Washington, Seattle; 1993-2020). Chinnery also discloses that many individuals with a mutation of mtDNA display a cluster of clinical features that fall into a discrete clinical syndrome, such as the Kearns-Sayre syndrome (KSS), chronic progressive external ophthalmoplegia (CPEO), mitochondrial encephalomyopathy with lactic acidosis and stroke-like episodes (MELAS), myoclonic epilepsy with ragged-red fibers fibers fibers (MERRF), neurogenic weakness with ataxia and retinitis pigmentosa (NARP), or Leigh syndrome (LS). Chinnery also discloses that considerable clinical variability exists, and many individuals do not fit neatly into one particular category, which is well-illustrated by the overlapping spectrum of disease phenotypes (including mitochondrial recessive ataxia syndrome (MIRAS)). Chinnery also discloses that common clinical features of mitochondrial disease—whether involving a mitochondrial or nuclear gene—include ptosis, external ophthalmoplegia, proximal myopathy and exercise intolerance, cardiomyopathy, sensorineural deafness, optic atrophy, pigmentary retinopathy, and diabetes mellitus. Common central nervous system findings are fluctuating encephalopathy, seizures, dementia, migraine, stroke-like episodes, ataxia, and spasticity.

Ng et al., J Neurol (2016) 263:179-191 discloses the genetics and management of mitochondrial diseases, e.g., mitochondrial encephalopathy, lactic acidosis, and stroke-like episodes (MELAS); myoclonic epilepsy with ragged red fibres (MERRF); mitochondrial neuro-gastrointestinal involvement and encephalopathy (MNGIE); neuropathy, ataxia, and retinitis pigmentosa (NARP); chronic progressive external ophthalmoplegia (CPEO); Alpers disease; Pearson syndrome; Leigh disease; Sengers syndrome; and Kearns-Sayre syndrome.

It has been reported that three very-highly conserved variants, p. Ile253Val, p.Glu192Lys, and p.Arg128His, in the ATPase domain of the TRAP1 gene may be associated with a statistically-significant, several-fold increased, prevalence of common chronic functional conditions, including at least pain, fatigue, and GI dysmotility (Boles et al., Mitochondrion 23 (2015) 64-70). These variants may be an important factor in the etiology of functional symptomatology. See, id.

It has been reported that mutations in TRAP1 may cause congenital abnormalities of the kidney and urinary tract (CAKUT) CAKUT or VACTERL association with CAKUT (Saisawat et al., Kidney International (2014) 85, 1310-1317).

In another aspect, the present disclosure provides methods of treating a disease in a subject in need thereof, the methods comprising administering to the subject in need thereof an effective amount of a provided compound or pharmaceutical composition.

In another aspect, the present disclosure provides methods of preventing a disease in a subject in need thereof, the methods comprising administering to the subject in need thereof an effective amount of a provided compound or pharmaceutical composition.

In certain embodiments, the disease is a disease described herein. In certain embodiments, the disease is associated with decreased expression and/or activity of TRAP1. In certain embodiments, the disease is associated with decreased activity of TRAP1. In certain embodiments, the disease is associated with a mutation in the gene encoding TRAP1. In certain embodiments, the effective amount is effective in increasing the expression and/or activity of TRAP1. In certain embodiments, the effective amount is effective in increasing the activity of TRAP1.

In certain embodiments, the disease is associated with decreased expression and/or activity of PTEN induced putative kinase 1 (PINK1). In certain embodiments, the disease is associated with a mutation in the gene encoding PINK1. In certain embodiments, the effective amount is effective in increasing the expression and/or activity of PINK1.

In certain embodiments, the disease is associated with increased production of reactive oxygen species.

In certain embodiments, the disease is associated with decreased health, quality, function, quantity, and/or activity of mitochondria. In certain embodiments, the effective amount is effective in increasing the health, quality, function, quantity, and/or activity of mitochondria.

In certain embodiments, the disease is a mitochondrial disease, disease associated with oxidative stress, neurodegenerative disease, or kidney disease.

In certain embodiments, the disease is a mitochondrial disease. In certain embodiments, the disease is Alpers-Huttenlocher syndrome, Barth syndrome, Friedreich ataxia, Kearns-Sayre syndrome, Leigh disease, mitochondrial encephalomyopathy (e.g., MELAS syndrome, MERRF syndrome, or MNGIE syndrome), mitochondrial injury, mitochondrial myopathy (e.g., Kearns-Sayre syndrome or mitochondrial encephalomyopathy (e.g., MELAS syndrome, MERRF syndrome, or MNGIE syndrome)), multiple symmetric lipomatosis, Pearson marrow-pancreas syndrome, or Sengers syndrome. In certain embodiments, the disease is diabetes mellitus and deafness (DAD); Leber's hereditary optic neuropathy (LHON); neuropathy, ataxia, retinitis pigmentosa, and ptosis (NARP); or mitochondrial DNA depletion syndrome. In certain embodiments, the disease is Leigh disease.

In certain embodiments, the disease is a neurological disease. In certain embodiments, the disease is a neurodegenerative disease. In certain embodiments, the disease is Parkinson's disease (e.g., Guamanian parkinsonism-dementia or X-linked dystonia parkinsonism). In certain embodiments, the disease is Parkinson's disease associated with a mutation in the gene encoding PINK1. In certain embodiments, the disease is Huntington's disease. In certain embodiments, the disease is Alzheimer's disease. In certain embodiments, the disease is dementia. In certain embodiments, the disease is frontotemporal dementia. In certain embodiments, the disease is amyotrophic lateral sclerosis. In certain embodiments, the disease is Friedreich's ataxia.

In certain embodiments, the disease is associated with increased protein misfolding and/or protein aggregation. In certain embodiments, the disease is a proteopathy. In certain embodiments, the disease is pathological protein aggregation (e.g., synucleinopathy (e.g., (Lewy body dementia, multiple system atrophy, or Parkinson's disease)); or trinucleotide repeat disorder (e.g., polyglutamine disease (e.g., dentatorubral-pallidoluysian atrophy, Huntington disease, Machado-Joseph disease, spinal-bulbar muscular atrophy, or spinocerebellar ataxia)).

In certain embodiments, the disease is a psychiatric disease. In certain embodiments, the disease is bipolar disorder. In certain embodiments, the disease is schizophrenia. In certain embodiments, the disease is anxiety disorder. In certain embodiments, the disease is a learning disability.

In certain embodiments, the disease is autonomic dysfunction.

In certain embodiments, the disease is a lysosomal storage disease. In certain embodiments, the disease is aspartylglucosaminuria; Danon disease; Farber disease; fucosidosis; galactosialidosis; Hermansky-Pudlak syndrome; mannosidase deficiency disorder (e.g., α-mannosidosis or β-mannosidosis); mucolipidosis; mucopolysaccharidosis (e.g., Di Ferrante syndrome, Hunter syndrome, Hurler syndrome, Hurler-Sheie syndrome, Maroteaux-Lamy syndrome, Morquio syndrome type A, Morquio syndrome type B, Natowicz syndrome, Sanfilippo syndrome, or Scheie syndrome); neuronal ceroid lipofuscinosis (e.g., adult neuronal ceroid lipofuscinosis, infantile neuronal ceroid lipofuscinosis (e.g., late infantile neuronal ceroid lipofuscinosis or variant late infantile neuronal ceroid lipofuscinosis), juvenile neuronal ceroid lipofuscinosis, or Northern epilepsy); Salla disease; Schindler disease; or sphingolipidosis (e.g., Fabry disease, gangliosidosis (e.g., GM1 gangliosidosis, GM2 gangliosidoses, GM2 gangliosidosis AB variant, Sandhoff disease, or Tay-Sachs disease), Gaucher's disease, Krabbe disease, metachromatic leukodystrophy, or Niemann-Pick disease. In certain embodiments, the disease is Niemann-Pick disease, Fabry disease, Farber disease, Wolman disease, Gaucher's disease, Krabbe disease, mucopolysaccharidosis type VII, neuronal ceroid lipofuscinosis type 2, or Pompe disease. In certain embodiments, the disease is (1) sphingolipidose (e.g., Fabry disease; Farber lipogranulomatosis; Gaucher disease types I, II, or III; Niemann-Pick disease types A or B; GM1 gangliosidosis; GM2-gangliosidosis (Sandhoff); GM2-gangliosidosis (Tay-Sachs); GM2-gangliosidosis (GM2-activator deficiency); GM3-gangliosidosis; metachromatic leukodystrophy; or sphingolipid-activator deficiency); (2) mucopolysaccharidose (e.g., MPS I (Schele, Hurler-Schele, or Hurler disease); MPS II (Hunter); MPS IIIA (Sanfilippo A); MPS IIIB (Sanfilippo B); MPS IIIC (Sanfilippo C); MPS IIID (Sanfilippo D); MPS IVA (Morquio syndrome A); MPS IVB (Morquio syndrome B); MPS VI (Maroteaux-Lamy); MPS VII (Sly disease); or MPS IX); (3) oligosaccharidose (e.g., α-mannosidosis; β-mannosidosis; fucosidosis; aspartylglucosaminuria; Schindler disease; sialidosis; galactosialidosis; mucolipidosis II (I-cell disease); or mucolipidosis III); (4) glycogen storage disease (e.g., Pompe disease); or (5) integral membrane protein disorder (e.g., cystinosis; Danon disease; action myoclonus-renal failure syndrome; Sailia disease; Niemann-Pick disease type C1; or Mucolipidosis IV). In certain embodiments, the disease is Haitia-Santavuori; Jansky-Bielschowsky; Spielmeyer-Sjogren; Parry; Hermansky-Pudlak diseases types 1-8; Griscelli 1, 2, or 3; or Chediak-Higashi disease. In certain embodiments, the disease is Tay-Sachs disease. In certain embodiments, the disease is Sandhoff disease. In certain embodiments, the disease is Niemann-Pick disease. In certain embodiments, the disease is Fabry disease. In certain embodiments, the disease is Gaucher's disease.

In certain embodiments, the disease is chronic pain, fatigue, gastrointestinal dysmotility, congenital abnormality of the kidney and urinary tract, VACTERL association, or cardiac hypertrophy. In certain embodiments, the disease is a painful condition (e.g., chronic pain). In certain embodiments, the disease is fatigue (e.g., chronic fatigue syndrome). In certain embodiments, the disease is a kidney disease. In certain embodiments, the disease is autoimmune kidney disease (e.g., autoimmune nephritis); Bartter syndrome; cardiorenal syndrome; chronic kidney disease (e.g., chronic nephritis, chronic obstructive nephropathy, or chronic renal failure); diabetes insipidus (e.g., genetic diabetes insipidus (e.g., X-linked nephrogenic diabetes insipidus), nephrogenic diabetes insipidus (e.g., X-linked nephrogenic diabetes insipidus), or neurogenic diabetes insipidus); diabetic nephropathy (e.g., diabetic glomerulopathy); Gitelman syndrome; glomerular kidney disease (e.g., diabetic glomerulopathy, glomerular hypertrophy, glomerular kidney injury, glomerulitis, glomerulonephritis (e.g., Heymann nephritis, mesangial proliferative glomerulonephritis, or minimal change glomerulonephritis), glomerulosclerosis (e.g., focal segmental glomerulosclerosis), Goodpasture syndrome, kidney mesangial injury, or renal glomerular thrombosis); HANAC syndrome; hemolytic-uremic syndrome; hemorrhagic fever with renal syndrome; hepatorenal syndrome; hydronephrosis; idiopathic membranous nephropathy; Kelley-Seegmiller syndrome; kidney cyst; kidney failure; kidney infection; kidney injury (e.g., acute kidney injury, glomerular kidney injury, kidney mesangial injury, or kidney tubule injury); kidney interstitial fibrosis; kidney ischemia; kidney lesion; kidney necrosis (e.g., kidney tubular necrosis); kidney neoplasm (e.g., nephroblastoma, renal cell carcinoma (e.g., papillary renal cell carcinoma, renal adenocarcinoma, or renal clear cell carcinoma), renal epithelioid leiomyoma, renal pelvis neoplasm, or Wilms tumor (e.g., Denys-Drash syndrome); kidney tubule disease (e.g., Dent disease, Fanconi syndrome, kidney tubular necrosis, kidney tubule injury, kidney tubulointerstitial disease, or renal tubular acidosis); Lesch-Nyhan syndrome; Lowe oculocerebrorenal syndrome; Mainzer-Saldino syndrome; nephritis (e.g., autoimmune nephritis, chronic nephritis, glomerulitis, immune complex nephritis (e.g., immune complex glomerulonephritis), interstitial nephritis, nephronophthisis (e.g., familial juvenile nephronophthisis), nephrotoxic nephritis, pyelitis, pyelonephritis, or Senior-Loken syndrome); nephrosclerosis; nephrotic syndrome; obstructive nephropathy; oligomeganephronic hypoplasia; polycystic kidney disease (e.g., autosomal dominant polycystic kidney disease or autosomal recessive polycystic kidney disease); primary hyperoxaluria type I; primary hyperoxaluria type II; renal amyloidosis; renal anemia; renal artery spasm; renal calculi; renal embolism; renal hypertrophy; renal infarction; renal-coloboma syndrome; Schinzel-Giedion midface retraction syndrome; ureteropelvic junction obstruction; or Zellweger syndrome. In certain embodiments, the disease is polycystic kidney disease. In certain embodiments, the disease is autosomal dominant polycystic kidney disease. In certain embodiments, the disease is autosomal recessive polycystic kidney disease. Polycystic kidney disease is an inherited genetic disorder that is characterized by the formation of renal cysts that block normal tubular function and thereby cause a progressive decline in kidney function with age, typically leading to end-stage renal disease (ESRD) by the sixth decade of life. See, e.g., Booij et al., SLAS Discovery, 2017, Vol. 22(8), 974-984.

In certain embodiments, the disease is a heart disease.

In certain embodiments, the disease is a gastrointestinal disease.

In certain embodiments, the disease is a liver disease.

In certain embodiments, the disease is a respiratory disease.

In certain embodiments, the disease is a cardiovascular disease.

In certain embodiments, the disease is sarcopenia.

In certain embodiments, the disease is a central nervous system (CNS) disease. In certain embodiments, the disease is a brain disease.

In certain embodiments, the disease is a hematological disease.

In certain embodiments, the disease is a metabolic disease. In certain embodiments, the disease is diabetes. In certain embodiments, the disease is obesity.

In certain embodiments, the disease is a genetic disease.

In certain embodiments, the disease is an inflammatory disease.

In certain embodiments, the disease is an autoimmune disease.

In certain embodiments, the disease is an autoinflammatory disease.

In certain embodiments, the disease is a proliferative disease. In certain embodiments, the disease is a cancer.

In certain embodiments, the effective amount, subject, biological sample, tissue, and cell are as described in the present disclosure.

In certain embodiments, the provided method further comprises administering to the subject in need thereof an additional therapy. In certain embodiments, the additional therapy is an additional pharmaceutical agent. In certain embodiments, the additional pharmaceutical agent is as described in the present disclosure. In certain embodiments, a provided method that further comprises administering to the subject in need thereof the additional therapy is synergistic as compared to a provided method that does not comprise administering to the subject in need thereof the additional therapy and as compared to a method comprises administering to the subject in need thereof the additional therapy as the only active therapy. In certain embodiments, the subject is a subject that has been administered the additional therapy. In certain embodiments, the subject is resistant to the additional therapy. In certain embodiments, the effective amount of a provided compound or pharmaceutical composition is effective in decreasing the resistance to the additional therapy. The additional therapy may be administered to the subject concurrently with, prior to, or subsequent to the administration of the provided compound or pharmaceutical composition.

In another aspect, the present disclosure provides compounds and pharmaceutical compositions for use in increasing the expression and/or activity of TRAP1 in a subject in need thereof.

In another aspect, the present disclosure provides compounds and pharmaceutical compositions for use in increasing the health, quality, function, quantity, and/or activity of mitochondria in a subject in need thereof.

In another aspect, the present disclosure provides compounds and pharmaceutical compositions for use in increasing the expression and/or activity of TRAP1 in a cell, tissue, or biological sample.

In another aspect, the present disclosure provides compounds and pharmaceutical compositions for use in increasing the health, quality, function, quantity, and/or activity of mitochondria in a cell, tissue, or biological sample.

In another aspect, the present disclosure provides compounds and pharmaceutical compositions for use in the treatment of a disease.

In another aspect, the present disclosure provides compounds and pharmaceutical compositions for use in the prevention of a disease.

In another aspect, the present disclosure provides use of compounds and pharmaceutical compositions in increasing the expression and/or activity of TRAP1 in a subject in need thereof.

In another aspect, the present disclosure provides use of compounds and pharmaceutical compositions in increasing the health, quality, function, quantity, and/or activity of mitochondria in a subject in need thereof.

In another aspect, the present disclosure provides use of compounds and pharmaceutical compositions in increasing the expression and/or activity of TRAP1 in a cell, tissue, or biological sample.

In another aspect, the present disclosure provides use of compounds and pharmaceutical compositions in increasing the health, quality, function, quantity, and/or activity of mitochondria in a cell, tissue, or biological sample.

In another aspect, the present disclosure provides use of compounds and pharmaceutical compositions in the treatment of a disease.

In another aspect, the present disclosure provides use of compounds and pharmaceutical compositions in the prevention of a disease.

In another aspect, the present disclosure provides use of compounds and pharmaceutical compositions in the manufacture of a medicament for increasing the expression and/or activity of TRAP1 in a subject in need thereof.

In another aspect, the present disclosure provides use of compounds and pharmaceutical compositions in the manufacture of a medicament for increasing the health, quality, function, quantity, and/or activity of mitochondria in a subject in need thereof.

In another aspect, the present disclosure provides use of compounds and pharmaceutical compositions in the manufacture of a medicament for increasing the expression and/or activity of TRAP1 in a cell, tissue, or biological sample.

In another aspect, the present disclosure provides use of compounds and pharmaceutical compositions in the manufacture of a medicament for increasing the health, quality, function, quantity, and/or activity of mitochondria in a cell, tissue, or biological sample.

In another aspect, the present disclosure provides use of compounds and pharmaceutical compositions in the manufacture of a medicament for the treatment of a disease.

In another aspect, the present disclosure provides use of compounds and pharmaceutical compositions in the manufacture of a medicament for the prevention of a disease.

### EXAMPLES

In order that the invention described herein may be more fully understood, the following examples are set forth. The synthetic and biological examples described in this application are offered to illustrate the methods and uses provided herein and are not to be construed in any way as limiting their scope. Certain known compounds are disclosed in U.S. patent application publication No. US 2018/0044286, which is incorporated by reference in its entirety.

### Example 1. Preparation of Exemplary Compounds of the Present Disclosure

### Methods and Materials

### Pyridinyl Synthetic Preparations

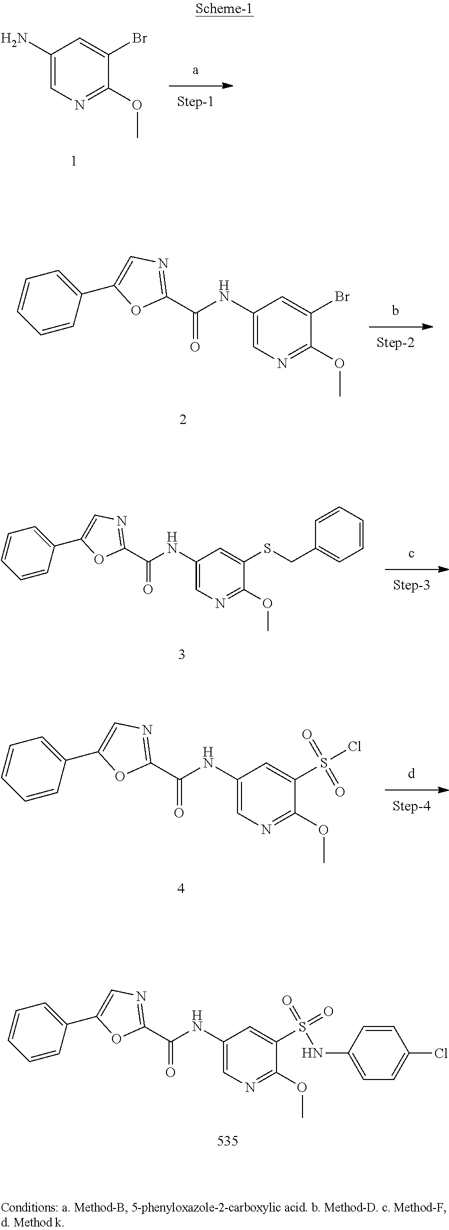

### General Experimental Procedures for Scheme 1, Step-1:

Method-A: To a stirred solution of corresponding amine (1.0 eq.) in DMF were added corresponding acid (1.5 eq.), EDC.HCl (2.5 eq.), HOBt (2.5 eq.) and DIPEA (4.0 eq.) at room temperature. The resulting reaction mixture was stirred at same temperature for 16 h. After completion of reaction (monitored by TLC), reaction mixture was poured on ice cold water. Solid separated was filtered, washed with water and dried. The crude product was purified by combi-flash to obtain corresponding amide.

Method-B: To a stirred solution of corresponding acid (1.0 eq.) in DMF were added DIPEA (3.0 eq.) and HATU (1.5 eq.) at 0° C. and stirred for 15 min, then corresponding amine (1.0 eq.) was added to the above mixture. The resulting reaction mixture was stirred at room temperature for 12 h. After completion of reaction (monitored by TLC), reaction mixture was poured on ice cold water, solid separated was filtered and dried under reduced pressure. Crude compound was purified by combi-flash to obtain corresponding amide.

Method-C: To a stirred solution of corresponding amine (1.0 eq.) and corresponding ester (2.0 eq.) in THF at 0° C. was added LiHMDS (3.0 eq.) and allowed to stir at room temperature for 16 h. After completion of reaction (monitored by TLC), the reaction mixture was diluted with water and extracted with EtOAc. The organic layer was dried over Na₂SO₄, filtered and concentrated under reduced pressure. The crude product was purified by combi-flash to obtain corresponding amide.

### General Experimental Procedures for Scheme 1, Step-2:

Method-D: To a stirred solution of N-(5-bromo-6-methoxypyridin-3-yl)-5-phenyloxazole-2-carboxamide (2, 1.80 g, 4.81 mmol, 1.0 eq.) and phenylmethanethiol (895.0 mg, 7.21 mmol, 1.5 eq.) in 1,4-dioxane was added Pd₂(dba)₃ (220 mg, 0.24 mmol, 0.05 eq.), Xantphos (278 mg, 0.48 mmol, 0.1 eq.) and N,N-Diisopropyl-ethylamine (1.77 mL, 9.62 mmol, 2.0 eq.) under nitrogen atmosphere. The resulting reaction mixture was stirred at 100° C. for 1 h. The reaction was monitored by TLC. The reaction mixture was quenched with water and extracted with EtOAc. The organic layer was washed with saturated NaHCO₃ solution, brine solution and dried over anhydrous Na₂SO₄ and concentrated under reduced pressure. The crude compound was purified by Combi-Flash chromatography by using 0-50% EtOAc in hexanes to obtain (3, 1.4 g, 3.35 mmol, 69% yield) as a yellow solid. LC-MS: m/z 418.0 ([M+H]⁺).

Method-E: To DMF (75 mL) in a sealed tube, was added phenylmethanethiol (18.2 mL, 0.015 mol, 2.0 eq.) followed by NaO^{t}Bu (16.44 g, 0.171 mol, 2.2 eq.) at room temperature and stirred for 30 min. Then 4-chloropyridin-2-amine (32, 10.0 g, 0.077 mol, 1.0 eq.) was added in portions at room temperature, followed by KF (9.03 g, 0.15 mol, 2.0 eq.). The reaction was sealed and heated at 90° C. for 16 h. After completion of the reaction (monitored by TLC), it was poured into ice-cold water (350 mL) and stirred for 30 min. The solid precipitated was filtered, washed with water and dried in vacuum. The isolated solid was stirred in hexanes (350 mL) for 30 min, solid was collected by filtration and dried in vacuum to obtain (33, 9.2 g, 0.0425 mol, 55% yield) as a white solid.

### General Experimental Procedures for Scheme 1, Step-3:

Method-F: To a stirred solution of N-(5-(benzylthio)-6-methoxypyridin-3-yl)-5-phenyloxazole-2-carboxamide (3, 600 mg, 1.43 mmol, 1.0 eq.) in CHCl₃ (15 mL) was added conc. HCl (15 mL) and 10% NaOCl (6.41 mL, 9.48 mmol, 6.6 eq.) dropwise at 0° C. The resulting reaction mixture was stirred 0° C. for 1 h. The reaction was monitored by TLC. The reaction mixture was quenched with ice and extracted with CHCl₃ (2×20 mL), the organic layer was dried over anhydrous Na₂SO₄ and concentrated under reduced pressure to obtain 4 (400 mg crude) as a pale yellow solid. LC-MS: m/z 394.0 ([M+H]⁺).

Method-G: A stirred solution of tert-butyl (4-(benzylthio)pyridin-2-yl)carbamate (34, 3.6 g, 0.011 mol, 1.0 eq.) in dichloromethane (90 mL) and water (30 mL) was purged with Cl₂ gas (generated from KMnO₄+Conc. HCl) for 1 h at 0° C. After reaction completion, nitrogen gas was purged into the reaction mixture for 20 min. The reaction mixture was then diluted with water (100 mL). The reaction mixture was extracted with dichloromethane thrice. The combined organic portion was washed with brine solution, dried over anhydrous Na₂SO₄ and concentrated.

### General Experimental Procedures for Scheme 1, Step-4:

Method-H: 4-fluoro-2,6-dimethylaniline (0.141 g, 0.99 mmol, 1.3 eq.) in pyridine (3 mL) was stirred for 15 min at 0° C. To the above solution, 2-methoxy-5-(2-phenyloxazole-4-carboxamido)-pyridine-3-sulfonyl chloride (7, 0.3 g, 0.76 mmol, 1.0 eq.) was added portion wise. The resulting reaction mixture was gradually warmed and stirred at room temperature for 1 h. After completion of reaction (monitored by TLC), reaction mixture was diluted with water and extracted with dichloromethane (2×30 mL). The combined organic extracts were washed with 2N HCl (2×30 mL) and water (30 mL) followed by brine (30 mL). The organic layer was dried over anhydrous Na₂SO₄ and concentrated under reduced pressure to obtain crude product (1964, 0.18 g).

Method-I: A solution of 4-amino-3,5-dimethylbenzonitrile (0.048 g, 0.32 mmol, 1.3 eq.), 4A molecular sieves (0.048 g) and 2-methoxy-5-(2-phenyloxazole-4-carboxamido)pyridine-3-sulfonyl chloride (7, 0.2 g, 0.253 mmol, 1.0 eq.) in 2,2,2-trifluoroethanol (3.0 mL) were stirred at 70° C. for 16 h. Reaction was monitored by TLC, after completion of reaction, mixture was diluted with water (2×30 mL) and extracted with EtOAc (2×30 mL). The combined organic layers were dried over Na₂SO₄, filtered and concentrated under reduced pressure to obtain crude product which was purified by combi-flash eluting with (40% EtOAc/hexanes). Clean fractions were concentrated and dried to obtain pure compound 1965 (0.09 g, 0.178 mmol, yield 35%) as an off-white solid.

Method-J: To a stirred solution of NaH (0.030 g, 0.75 mmol 3.0 eq.) in DMF (1.5 mL) was added 2-amino-3-(trifluoromethyl)benzonitrile (0.061 g, 0.33 mmol, 1.3 eq.) portion wise at 0° C. and the mixture stirred for 5 min. To the above solution, 2-methoxy-5-(5-phenyloxazole-2-carboxamido)pyridine-3-sulfonyl chloride (4, 0.1 g, 0.25 mmol, 1.0 eq.) in DMF (1.0 mL) was added slowly at 0° C. The reaction mixture was stirred at 0° C. for 10 minutes. After completion of the reaction (monitored by TLC), it was quenched with aq. ammonium chloride solution (20 mL) and extracted with EtOAc (2×20 mL) and the organic layer was washed with water (25 mL), brine solution (25 mL) and dried under reduced pressure to yield crude product. The crude product was purified by combi-flash by eluting with (EtOAc: hexanes/40:60) to obtain 1358 (0.012 g, 0.02 mmol, 9% yield) as a yellow solid.

Method-K: To a stirred solution of 4-chloroaniline (130 mg, 1.01 mmol, 1.0 eq.), Pyridine (0.26 mL, 3.03 mmol, 3.0 eq.) and DMAP (12 mg, 0.10 mmol, 0.1 eq.) in dichloromethane (3.0 mL) was added 2-methoxy-5-(5-phenyloxazole-2-carboxamido)pyridine-3-sulfonyl chloride (4, 400 mg, 1.01 mmol, 1.0 eq.) at 0° C. The resulting reaction mixture was stirred at room temperature for 16 h. The reaction was monitored by TLC; the reaction mixture was quenched with water and extracted with dichloromethane (2×30 mL). The organic layer was washed with 1N HCl (20 mL), dried over anhydrous sodium sulphate, and the solvent concentrated under reduced pressure to yield crude compound. The crude compound was purified by combi-flash column chromatography by using 0-50% EtOAc in hexanes as eluent to obtain 535 (150 mg, 0.30 mmol, 30% yield) as an off-white solid.

### Experimental Procedures for Scheme-1:

### Step-1: Preparation of N-(5-bromo-6-methoxypyridin-3-yl)-5-phenyloxazole-2-carboxamide (2)

To a stirred solution of 5-phenyloxazole-2-carboxylic acid (45, 1.39 g, 7.38 mmol, 1.5 eq.) and N,N-Diisopropylethylamine (2.72 mL, 14.76 mmol, 3.0 eq.) in N,N-dimethylformamide was added HATU (3.74 g, 9.84 mmol, 2.0 eq.) at 0° C. and the mixture stirred for 5 minutes. To this reaction mixture, 5-bromo-6-methoxypyridin-3-amine (1, 1.0 g, 4.92 mmol, 1.0 eq.) was added. The resulting reaction mixture was stirred at room temperature for 16 h. After completion of the reaction (monitored by TLC), the mixture was quenched with water and the resulting solid was filtered and dried to obtain 2 (1.80 g, 4.81 mmol, 97% yield) as a light brown solid. LC-MS: m/z 376.0 ([M+2H]⁺).

### Step-2: Preparation of N-(5-(benzylthio)-6-methoxypyridin-3-yl)-5-phenyloxazole-2-carboxamide (3)

To a stirred solution of N-(5-bromo-6-methoxypyridin-3-yl)-5-phenyloxazole-2-carboxamide (2, 1.80 g, 4.81 mmol, 1.0 eq.) and phenylmethanethiol (895 mg, 7.21 mmol, 1.5 eq.) in 1,4-dioxane was added Pd₂(dba)₃ (220 mg, 0.24 mmol, 0.05 eq.), Xantphos (278 mg, 0.48 mmol, 0.1 eq.) and N,N-diisopropyl ethylamine (1.77 mL, 9.62 mmol, 2.0 eq.) under nitrogen atmosphere. The resulting reaction mixture was stirred at 100° C. for 1 h. The reaction was monitored by TLC. The reaction mixture was quenched with water and extracted with EtOAc. The organic layer was washed with saturated NaHCO₃ solution, brine solution, and then dried over anhydrous Na₂SO₄. The solvent was concentrated under reduced pressure and the resulting crude compound was purified by Combi-Flash chromatography using 0-50% EtOAc in hexanes to obtain 3 (1.4 g, 3.35 mmol, 69% yield) as a yellow solid. LC-MS: m/z 418.0 ([M+H]⁺).

### Step-3: 2-methoxy-5-(5-phenyloxazole-2-carboxamido) pyridine-3-sulfonyl chloride (4)

To a stirred solution of N-(5-(benzylthio)-6-methoxypyridin-3-yl)-5-phenyloxazole-2-carboxamide (3, 600 mg, 1.43 mmol, 1.0 eq.) in CHCl₃ (15.0 mL) was added conc. HCl (15.0 mL) and 10% NaOCl (6.41 mL, 9.48 mmol, 6.6 eq.) dropwise at 0° C. The resulting reaction mixture was stirred at 0° C. for 1 h and monitored by TLC. The reaction mixture was quenched with ice and extracted with CHCl₃ (2×20 mL), the organic layer dried over anhydrous Na₂SO₄, and then concentrated under reduced pressure to obtain 4 (400 mg crude) as a pale yellow solid. LC-MS: m/z 394.0 ([M+H]⁺).

### Step-4: N-(5-(N-(4-chlorophenyl) sulfamoyl)-6-methoxypyridin-3-yl)-5-phenyloxazole-2-carboxamide (535)

To a stirred solution of 4-chloroaniline (130 mg, 1.01 mmol, 1.0 eq.), pyridine (0.26 mL, 3.03 mmol, 3.0 eq.) and DMAP (12 mg, 0.10 mmol, 0.1 eq.) in dichloromethane (3.0 mL) was added 2-methoxy-5-(5-phenyloxazole-2-carboxamido)pyridine-3-sulfonyl chloride (4, 400 mg, 1.01 mmol, 1.0 eq.) at 0° C. The resulting reaction mixture was stirred at room temperature for 16 h and monitored by TLC. The reaction mixture was quenched with water and extracted with dichloromethane (2×30 mL). The organic layer was washed with 1N HCl (20 mL), dried over anhydrous Na₂SO₄, and the solvent concentrated under reduced pressure to yield crude product. The crude compound was purified by Combi-flash column chromatography using 0-50% EtOAc in hexanes as eluent to obtain 535 (150 mg, 0.30 mmol, 30% yield) as an off-white solid. ¹H NMR (400 MHz, DMSO-d₆): δ 11.21 (s, 1H), 10.58 (s, 1H), 8.77 (d, J=2.4 Hz, 1H), 8.69 (d, J=2.8 Hz, 1H), 8.04 (s, 1H), 7.88 (d, J=7.2 Hz, 2H), 7.57-7.53 (m, 2H), 7.49-7.47 (m, 1H), 7.31 (d, J=8.8 Hz, 2H), 7.13 (d, J=8.8 Hz, 2H), 3.96 (s, 3H). LC-MS: m/z 485.1 ([M+H]⁺).

### Below Compounds were Prepared Using Methods Analogous to Those Used in Scheme-1

### Preparation of N-(6-methoxy-5-(N-(4-(trifluoromethyl) phenyl)sulfamoyl)pyridin-3-yl)-5-phenyloxazole-2-carboxamide (594)

Prepared by Method-K by reacting 2-methoxy-5-(5-phenyloxazole-2-carboxamido)pyridine-3-sulfonyl chloride (4) with 4-(trifluoromethyl)-aniline, to afford an off-white solid (37% yield). ¹H NMR (400 MHz, DMSO-d₆): δ 11.20 (s, 1H), 10.99 (s, 1H), 8.78 (s, 2H), 8.05 (s, 1H), 7.89 (d, J=12 Hz, 2H), 7.62 (d, J=8.0 Hz, 2H), 7.57 (t, J=4.0 Hz, 2H), 7.49-7.47 (m, 1H), 7.31 (d, J=8.0 Hz, 2H), 3.93 (s, 3H). LC-MS: m/z 519.1 ([M+H]⁺).

### Preparation of N-(5-(N-(2-chloro-6-(trifluoromethyl) phenyl) sulfamoyl)-6-methoxypyridin-3-yl)-5-phenyloxazole-2-carboxamide (1303)

Prepared by Method-K by reacting 2-methoxy-5-(5-phenyloxazole-2-carboxamido)pyridine-3-sulfonyl chloride (4) with 2-chloro-6-(trifluoromethyl)aniline, to afford an off-white solid (66% yield). ¹H NMR (400 MHz, DMSO-d₆): δ 11.19 (s, 1H), 10.20 (s, 1H), 8.83 (s, 1H), 8.49 (s, 1H), 7.87-7.85 (m, 4H), 7.75-7.73 (m, 1H), 7.56-7.44 (m, 3H), 4.00 (s, 3H). LC-MS: m/z 552.9 ([M+H]⁺).

### Preparation of N-(5-(N-(2,6-dichlorophenyl)sulfamoyl)-6-methoxypyridin-3-yl)-5-phenyloxazole-2-carboxamide (670)

Prepared by Method-K by reacting 2-methoxy-5-(2-phenyloxazole-5-carboxamido) pyridine-3-sulfonyl chloride (4) with 2, 6-dichloroaniline, to afford a white solid (48% yield). ¹H NMR (400 MHz, DMSO-d₆+D₂O): δ 8.77 (d, J=2.0 Hz, 1H), 8.49 (d, J=2.4 Hz, 1H), 7.98 (s, 1H), 7.87 (d, J=7.2 Hz, 2H), 7.57-7.53 (m, 2H), 7.49-7.44 (m, 3H), 7.26-7.22 (m, 1H), 3.93 (s, 3H). LC-MS: m/z 517.0 ([M−2H]⁻).

### Preparation of N-(5-(N-(2-cyano-6-(trifluoromethyl) phenyl) sulfamoyl)-6-methoxypyridin-3-yl)-5-phenyloxazole-2-carboxamide (1358)

Prepared by Method-J by reacting 2-methoxy-5-(2-phenyloxazole-5-carboxamido) pyridine-3-sulfonyl chloride (4) with 2-amino-3-(trifluoromethyl)benzonitrile, to afford a yellow solid (8.6% yield). ¹H NMR (400 MHz, DMSO-d₆): δ 11.14 (bs, 1H), 10.85 (bs, 1H), 8.83 (bs, 1H), 8.53 (bs, 1H), 8.22 (d, J=7.6 Hz, 1H), 8.09-8.02 (m, 2H), 7.87-7.75 (m, 3H), 7.56-7.46 (m, 3H), 3.95 (bs, 3H). LC-MS: m/z 542.1 ([M−H]⁻).

### Preparation of N-(5-(N-(2-fluoro-6-(trifluoromethyl) phenyl) sulfamoyl)-6-methoxypyridin-3-yl)-5-phenyloxazole-2-carboxamide (1377)

Prepared by Method-J by reacting 2-methoxy-5-(2-phenyloxazole-5-carboxamido) pyridine-3-sulfonyl chloride (4) with 2-fluoro-6-(trifluoromethyl) aniline to afford a white solid (50% yield). ¹H NMR (400 MHz, DMSO-d₆): δ 11.11 (bs, 1H), 10.09 (bs, 1H), 8.81 (bs, 1H), 8.50 (s, 1H), 8.02 (s, 1H), 7.87 (d, J=7.2 Hz, 2H), 7.56-7.44 (m, 6H), 3.98 (s, 3H). LC-MS: m/z 534.95 ([M−2H]⁻).

### Preparation of N-(5-bromo-6-methoxypyridin-3-yl)-2-phenyloxazole-5-carboxamide (5)

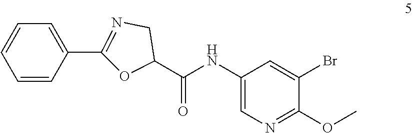 [SMILES: COc1ncc(NC(=O)C2CN=C(c3ccccc3)O2)cc1Br]

Prepared by Method-B by reacting 5-bromo-6-methoxypyridin-3-amine with 2-phenyloxazole-5-carboxylic acid to give a light brown solid (98% yield). LC-MS: m/z 376.0 ([M+2H]⁺).

### Preparation of N-(5-(benzylthio)-6-methoxypyridin-3-yl)-2-phenyloxazole-5-carboxamide (6)

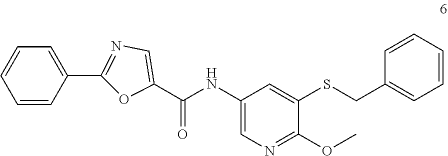 [SMILES: COc1ncc(NC(=O)c2cnc(-c3ccccc3)o2)cc1SCc1ccccc1]

Prepared by Method-D by reacting N-(5-bromo-6-methoxypyridin-3-yl)-2-phenyloxazole-5-carboxamide (11) with phenylmethanethiol to yield a yellow solid (74% yield). LC-MS: m/z 418.1 ([M+H]⁺).

### Preparation of 2-methoxy-5-(2-phenyloxazole-5-carboxamido) pyridine-3-sulfonyl chloride (7)

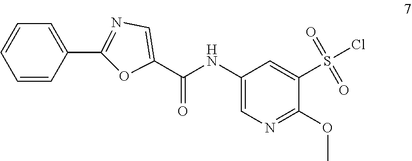 [SMILES: COc1ncc(NC(=O)c2cnc(-c3ccccc3)o2)cc1S(=O)(=O)Cl]

Prepared by Method-F starting with N-(5-(benzylthio)-6-methoxypyridin-3-yl)-2-phenyloxazole-5-carboxamide to give a pale yellow solid (crude). LC-MS: m/z 391.9 ([M−H]⁻).

### Preparation of N-(6-methoxy-5-(N-(4-(trifluoromethyl) phenyl)sulfamoyl)pyridin-3-yl)-2-phenyloxazole-5-carboxamide (604)

Prepared by Method-K by reacting 2-methoxy-5-(2-phenyloxazole-5-carboxamido)pyridine-3-sulfonyl chloride (7) with 4-(trifluoromethyl)aniline, to give an off-white solid (32% yield). ¹H NMR (400 MHz, DMSO-d₆): δ 10.99 (s, 1H), 10.65 (s, 1H), 8.75-8.74 (m, 1H), 8.63-8.63 (m, 1H), 8.16-8.14 (m, 2H), 8.08 (s, 1H), 7.61-7.59 (m, 5H), 7.30 (d, J=8.4 Hz, 2H), 3.93 (s, 3H). LC-MS: m/z 519.0 ([M+H]⁺).

### Preparation of 3-(benzylthio)-2-methoxy-5-nitropyridine (8)

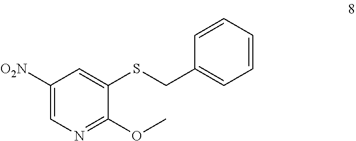 [SMILES: COc1ncc([N+](=O)[O-])cc1SCc1ccccc1]

Prepared by Method-D by reacting 3-bromo-2-methoxy-5-nitropyridine with phenylmethanethiol, to yield a yellow solid (65% yield). LC-MS: m/z 275.0 ([M−H]⁻).

### Preparation of 5-(benzylthio)-6-methoxypyridin-3-amine (9)

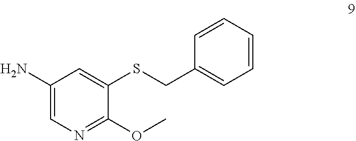 [SMILES: COc1ncc(N)cc1SCc1ccccc1]

To a stirred solution of 3-(benzylthio)-2-methoxy-5-nitropyridine (8, 3.0 g, 0.0108 mol, 1.0 eq.) in EtOH (30 mL) and water (6 mL) were added NH₄Cl (5.76 g, 0.108 mol, 10.0 eq.) and iron powder (3.0 g, 0.054 mol, 5.0 eq.) at room temperature. The resulting mixture was stirred at 90° C. for 1 h. After completion of the reaction (monitored by TLC), the reaction mixture was cooled to room temperature diluted with EtOAc (100 mL), and filtered through a celite bed. The filtrate was transferred to a separatory funnel and washed with water (20 mL) and brine (20 mL). The organic layer was dried over anhydrous Na₂SO₄ and concentrated under reduced pressure. The crude product was purified by Combi-flash eluting with hexanes/EtOAc (2:3) to obtain 9 (2.5 g, 0.010 mol, 93% yield) as a brown solid. LC-MS: m/z 247.1 ([M−H]⁻).

### Preparation of N-(5-(benzylthio)-6-methoxypyridin-3-yl)-2-phenyloxazole-4-carboxamide (10)

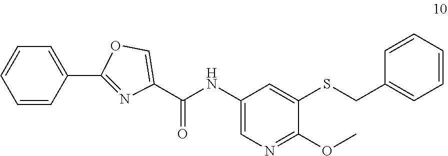 [SMILES: COc1ncc(NC(=O)c2coc(-c3ccccc3)n2)cc1SCc1ccccc1]

Prepared by Method-B by reacting 5-(benzylthio)-6-methoxypyridin-3-amine (9) with 2-phenyloxazole-4-carboxylic acid to afford a brown solid (91% yield). LC-MS: m/z 418.05 ([M+H]⁺).

### Preparation of 2-methoxy-5-(2-phenyloxazole-4-carboxamido) pyridine-3-sulfonyl chloride (11)

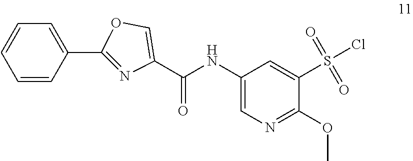 [SMILES: COc1ncc(NC(=O)c2coc(-c3ccccc3)n2)cc1S(=O)(=O)Cl]

To a stirred solution of N-(5-(benzylthio)-6-methoxypyridin-3-yl)-2-phenyloxazole-4-carboxamide (10, 3.0 g, 7.1 mmol, 1.0 eq.) in AcOH:H₂O (4:1) (30 mL) was added NCS (1.9 g, 0.0143 mol, 2.0 eq.) at 0° C. The resulting reaction mixture was stirred at 0° C. for 2 h. After completion of the reaction (monitored by TLC), the mixture was diluted with ice cold water then filtered and dried to obtain crude 11 (2.4 g, 6.09 mmol, 85% yield) as an off-white solid.

### Preparation of N-(5-(N-(2-(difluoromethyl) phenyl)sulfamoyl)-6-methoxypyridin-3-yl)-2-phenyloxazole-5-carboxamide (1991)

Prepared by Method-H by reacting 2-methoxy-5-(2-phenyloxazole-4-carboxamido)pyridine-3-sulfonyl chloride (11) with 2-(difluoromethyl)aniline, to afford an off-white solid (20% yield). ¹H NMR (400 MHz, DMSO-d₆): δ 10.45 (bs, 1H), 10.25 (bs, 1H), 8.87 (m, 2H), 8.51 (d, J=2.4 Hz, 1H), 8.07 (d, J=3.2 Hz, 2H), 7.60-7.59 (m, 4H), 7.47 (d, J=7.2 Hz, 1H), 7.39 (t, J=7.6 Hz, 1H), 7.23-7.09 (m, 2H), 3.98 (s, 3H). LC-MS: m/z 501.0 ([M+H]⁺).

### Preparation of N-(5-(N-(2-cyclopropyl-6-methylphenyl) sulfamoyl)-6-methoxypyridin-3-yl)-2-phenyloxazole-4-carboxamide (1986)

Prepared by Method-J by reacting 2-methoxy-5-(2-phenyloxazole-4-carboxamido)pyridine-3-sulfonyl chloride (11) with 2-cyclopropyl-6-methylaniline, to afford a yellow solid (48% yield). ¹H NMR (400 MHz, DMSO-d₆): δ 10.49 (s, 1H), 9.59 (s, 1H), 8.87-8.83 (m, 2H), 8.46 (s, 1H), 8.07 (s, 2H), 7.60 (s, 3H), 7.07-6.56 (m, 3H) 3.98 (s, 3H), 2.16 (s, 3H), 1.92 (s, 1H), 0.52-0.42 (m, 4H). LC-MS: m/z 505.1 ([M+H]⁺).

### Preparation of N-(5-(N-(5-chloro-3-methylpyridin-2-yl) sulfamoyl)-6-methoxypyridin-3-yl)-2-phenyloxazole-4-carboxamide (1966)

Prepared by Method-H by reacting 2-methoxy-5-(2-phenyloxazole-4-carboxamido)pyridine-3-sulfonyl chloride (11) with 5-chloro-3-methylpyridin-2-amine, to afford an off-white solid (12% yield). ¹H NMR (400 MHz, DMSO-d₆): δ 10.57 (bs, 1H), 10.53 (bs, 1H), 8.91 (s, 1H), 8.83 (d, J=2.0 Hz, 1H), 8.71 (d, J=2.0 Hz, 1H), 8.10 (d, J=3.2 Hz, 2H), 7.98 (s, 1H), 7.76 (s, 1H), 7.62-7.60 (m, 3H), 3.92 (s, 3H), 2.32 (s, 3H). LC-MS: m/z 500.1 ([M]⁺).

### Preparation of N-(5-(N-(5-chloro-3-methylpyridin-2-yl) sulfamoyl)-6-methoxypyridin-3-yl)-2-phenyloxazole-4-carboxamide (1964)

Prepared by Method-H substituting 2-methoxy-5-(2-phenyloxazole-4-carboxamido) pyridine-3-sulfonyl chloride (11) in scheme-1, step-4 with 4-fluoro-2, 6-dimethylaniline, as an off-white solid (34% yield). ¹H NMR (400 MHz, DMSO-d₆): δ 10.55 (bs, 1H), 9.48 (bs, 1H), 8.87 (s, 2H), 8.47 (d, J=2.4 Hz, 1H), 8.09-8.07 (m, 2H), 7.60-7.59 (m, 3H), 6.89 (d, J=9.2 Hz, 2H), 4.01 (s, 3H), 2.03 (s, 6H). LC-MS: m/z 497.05 ([M+H]⁺).

### Preparation of N-(5-(N-(4-cyano-2,6-dimethylphenyl)sulfamoyl)-6-methoxypyridin-3-yl)-2-phenyloxazole-4-carboxamide (1965)

Prepared by Method-I by reacting 2-methoxy-5-(2-phenyloxazole-4-carboxamido) pyridine-3-sulfonyl chloride (11) with 4-amino-3, 5-dimethylbenzonitrile to give an off-white solid (35% yield). ¹H NMR (400 MHz, DMSO-d₆): δ 10.55 (bs, 1H), 9.83 (bs, 1H), 8.84 (s, 2H), 8.51 (d, J=1.6 Hz, 1H), 8.08 (d, J=3.6 Hz, 2H) 7.60-7.56 (m, 5H), 3.98 (s, 3H), 2.09 (s, 6H). LC-MS: m/z 504.0 ([M+H]⁺).

### Preparation of N-(5-(N-(2-chloro-6-(trifluoromethyl) phenyl) sulfamoyl)-6-methoxypyridin-3-yl)-2-phenyloxazole-4-carboxamide (1962)

Prepared by Method-K by reacting 2-methoxy-5-(2-phenyloxazole-4-carboxamido)pyridine-3-sulfonyl chloride (11) with 2-chloro-6-(trifluoromethyl)aniline, to afford an off-white solid (12% yield). ¹H NMR (400 MHz, DMSO-d₆): δ 10.25 (s, 1H), 10.1 (s, 1H), 8.86-8.83 (m, 2H), 8.47 (s, 1H), 8.07-8.06 (m, 2H), 7.56-7.84 (m, 1H), 7.73-7.71 (m, 1H), 7.58-7.53 (m, 4H), 4.0 (s, 3H). LC-MS: m/z 554.0 ([M+H]⁺).

### Preparation of N-(5-(N-(4-fluoro-2-methylphenyl) sulfamoyl)-6-methoxypyridin-3-yl)-2-phenyloxazole-4-carboxamide (1950)

Prepared by Method-K by reacting 2-methoxy-5-(2-phenyloxazole-4-carboxamido) pyridine-3-sulfonyl chloride (11) with 4-fluoro-2-methylaniline to afford a light brown solid (69% yield). ¹H NMR (400 MHz, DMSO-d₆): δ 10.50 (bs, 1H), 9.7 (bs, 1H), 8.86-8.88 (m, 2H), 8.50 (d, J=2.4 Hz, 1H), 8.00-7.98 (m, 2H), 7.60-7.65 (m, 3H), 7.05-6.94 (m, 3H), 3.98 (s, 3H), 2.14 (s, 3H). LC-MS: m/z 483.1 ([M+H]⁺).

### Preparation of N-(5-(N-(2-chloro-6-methylphenyl) sulfamoyl)-6-methoxypyridin-3-yl)-2-phenyloxazole-4-carboxamide (1941)

Prepared by Method-H by reacting 2-methoxy-5-(2-phenyloxazole-4-carboxamido) pyridine-3-sulfonyl chloride (11) with 2-chloro-6-methylaniline to afford a white solid (2% yield). ¹H NMR (400 MHz, DMSO-d₆): δ 10.50 (bs, 1H), 9.79 (bs, 1H), 8.87-8.85 (m, 2H), 8.49 (d, J=2.0 Hz, 1H), 8.08 (d, J=3.2 Hz, 2H), 7.60 (d, J=2.4 Hz, 3H), 7.25-7.19 (m, 3H), 3.98 (s, 3H), 2.25 (s, 3H). LC-MS: m/z 498.95 ([M+H]⁺).

### Preparation of N-(5-(N-(2-fluoro-6-methylphenyl) sulfamoyl)-6-methoxypyridin-3-yl)-2-phenyloxazole-4-carboxamide (1485)

Prepared by Method-H by reacting 2-methoxy-5-(2-phenyloxazole-4-carboxamido)pyridine-3-sulfonyl chloride (11) with 2-fluoro-6-methylaniline to afford a white solid (8% yield). ¹H NMR (400 MHz, DMSO-d₆): δ 10.47 (bs, 1H), 8.87 (s, 2H), 8.47 (s, 1H), 8.08 (d, J=2.8 Hz, 2H), 7.60 (s, 3H), 7.14-6.94 (m, 3H), 3.96 (s, 3H), 2.22 (s, 3H). LC-MS: m/z 483.0 ([M+H]⁺).

### Preparation of N-(6-methoxy-5-(N-(2-methoxy-6-methylphenyl) sulfamoyl) pyridin-3-yl)-2-phenyloxazole-4-carboxamide (1484)

Prepared by Method-H by reacting 2-methoxy-5-(2-phenyloxazole-4-carboxamido)pyridine-3-sulfonyl chloride (11) with 2-methoxy-6-methylaniline, as an off-white solid (45% yield). ¹H NMR (400 MHz, DMSO-d₆): δ 10.44 (bs, 1H), 9.07 (bs, 1H), 8.85 (s, 1H), 8.82 (d, J=2.4 Hz, 1H), 8.35 (d, J=2.4 Hz, 1H), 8.09-8.06 (m, 2H), 7.60-7.59 (m, 3H), 7.10-7.08 (m, 1H), 6.83-6.81 (m, 1H), 6.70-6.68 (m, 1H), 4.02 (s, 3H), 3.20 (s, 3H), 2.32 (s, 3H). LC-MS: m/z 495.0 ([M+H]⁺).

### Preparation of N-(5-(N-(5-chloro-3-fluoropyridin-2-yl) sulfamoyl)-6-methoxypyridin-3-yl)-2-phenyloxazole-4-carboxamide (1977)

Prepared by Method-I by reacting 2-methoxy-5-(2-phenyloxazole-4-carboxamido) pyridine-3-sulfonyl chloride (11) with 5-chloro-3-fluoropyridin-2-amine to afford an off-white solid (12% yield). ¹H NMR (400 MHz, DMSO-d₆): δ 11.45 (bs, 1H), 10.58 (s, 1H), 8.92 (s, 1H), 8.84 (s, 1H), 8.75 (s, 1H), 8.11-8.08 (m, 4H), 7.62-7.60 (m, 3H) 3.90 (s, 3H). LC-MS: m/z 504.0 ([M+H]⁺),

### Preparation of N-(5-bromo-6-methoxypyridin-3-yl)-2-phenylthiazole-4-carboxamide (12)

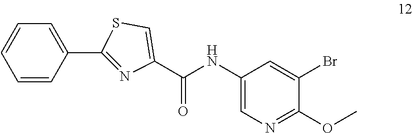 [SMILES: COc1ncc(NC(=O)c2csc(-c3ccccc3)n2)cc1Br]

Prepared by Method-B by reacting 5-bromo-6-methoxypyridin-3-amine with 2-phenylthiazole-4-carboxylic acid to give a light brown solid (41% yield). LC-MS: m/z 389.9 ([M−H]⁻),

### Preparation of N-(5-(benzylthio)-6-methoxypyridin-3-yl)-2-phenylthiazole-4-carboxamide (13)

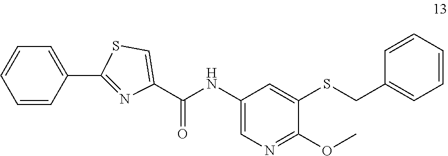 [SMILES: COc1ncc(NC(=O)c2csc(-c3ccccc3)n2)cc1SCc1ccccc1]

Prepared by Method-D by reacting N-(5-bromo-6-methoxypyridin-3-yl)-2-phenylthiazole-4-carboxamide (12) with phenylmethanethiol to give a yellow solid (90% yield).

### Preparation of 2-methoxy-5-(2-phenylthiazole-4-carboxamido) pyridine-3-sulfonyl chloride (14)

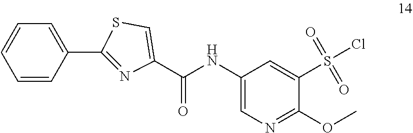 [SMILES: COc1ncc(NC(=O)c2csc(-c3ccccc3)n2)cc1S(=O)(=O)Cl]

Prepared by Method-F starting with N-(5-(benzylthio)-6-methoxypyridin-3-yl)-2-phenylthiazole-4-carboxamide (13) to give crude product as a yellow solid. LC-MS: m/z 408.0 ([M−H]⁻).

### Preparation of N-(3-(N-(2,6-dimethylphenyl)sulfamoyl)-4-methoxyphenyl)-2-phenylthiazole-4-carboxamide (1178)

Prepared by Method-K by reacting 2-methoxy-5-(2-phenylthiazole-4-carboxamido) pyridine-3-sulfonyl chloride (14) with 2,6-dimethylaniline to afford an off-white solid (12% yield). ¹H NMR (400 MHz, DMSO-d₆): δ 10.55 (bs, 1H), 9.47 (bs, 1H), 8.93 (d, J=2.4 Hz, 1H), 8.48-8.46 (m, 2H), 8.14-8.12 (m, 2H), 7.55-7.54 (m, 3H), 7.06-7.01 (m, 3H), 4.03 (s, 3H), 2.04 (s, 6H). LC-MS: m/z 495.0 ([M+H]⁺).

### Preparation of N-(6-methoxy-5-(N-(o-tolyl) sulfamoyl) pyridin-3-yl)-2-phenylthiazole-4-carboxamide (1350)

Prepared by Method-K by reacting 2-methoxy-5-(2-phenylthiazole-4-carboxamido) pyridine-3-sulfonyl chloride (14) with o-toluidine to afford an off-white solid (59% yield). ¹H NMR (400 MHz, DMSO-d₆): δ 10.58 (s, 1H), 9.67 (s, 1H), 8.90-8.90 (d, J=2.4 Hz, 1H), 8.51-8.48 (m, 2H), 8.14-8.12 (m, 2H), 7.56-7.54 (m, 3H), 7.09-7.07 (m, 4H), 3.99 (s, 3H), 2.16 (s, 3H). LC-MS: m/z 481.1 ([M+H]⁺).

### Preparation of N-(5-(N-(2,4-dimethylthiophen-3-yl)sulfamoyl)-6-methoxypyridin-3-yl)-2-phenylthiazole-4-carboxamide (1985)

Prepared by Method-K by reacting 2-methoxy-5-(2-phenylthiazole-4-carboxamido) pyridine-3-sulfonyl chloride (14) with 2,4-dimethylthiophen-3-amine to afford a pale yellow solid (58% yield). ¹H NMR (400 MHz, DMSO-d₆): δ 10.56 (bs, 1H), δ 9.49 (bs, 1H), 8.94 (d, J=2.4 Hz, 1H), 8.48-8.46 (m, 2H), 8.15-8.12 (m, 2H), 7.56-7.54 (m, 3H), 6.83 (s, 1H), 4.02 (s, 3H), 2.03 (s, 3H), 1.84 (s, 3H). LC-MS: m/z 501.0 ([M+H]⁺).

### Preparation of N-(5-(N-(2,6-bis(trifluoromethyl)phenyl)sulfamoyl)-6-methoxypyridin-3-yl)-2-phenylthiazole-4-carboxamide (1970)

Prepared by Method-K by reacting 2-methoxy-5-(2-phenylthiazole-4-carboxamido)pyridine-3-sulfonyl chloride (14) with 2,6-bis(trifluoromethyl)aniline to afford a white solid (50% yield). ¹H NMR (400 MHz, DMSO-d₆): δ 10.54 (bs, 1H), 10.16 (bs, 1H), 8.89 (d, J=2.0 Hz, 1H), 8.48 (bs, 2H), 8.15-8.09 (m, 4H), 7.81 (d, J=8.0 Hz, 1H), 7.56-7.54 (m, 3H), 4.04 (s, 3H). LC-MS: m/z 602.9 ([M+H]⁺).

### Preparation of N-(6-methoxy-5-(N-(3-methylthiophen-2-yl) sulfamoyl) pyridin-3-yl)-2-phenylthiazole-4-carboxamide (1954)

Prepared by Method-K by reacting 2-methoxy-5-(2-phenylthiazole-4-carboxamido) pyridine-3-sulfonyl chloride (14) with 3-methylthiophen-2-amine to afford an off-white solid (50% yield). ¹H NMR (400 MHz, DMSO-d₆): δ 10.53 (s, 1H), 10.12 (s, 1H), 8.94 (d, J=2.0 Hz, 1H), 8.51-8.48 (m, 2H), 8.13 (d, J=4.0 Hz, 2H), 7.56-7.54 (m, 3H), 7.13 (d, J=5.2 Hz, 1H), 6.73 (d, J=5.2 Hz, 1H), 4.04 (s, 3H), 1.98 (s, 3H). LC-MS: m/z 486.90 ([M+H]⁺).

### Preparation of N-(5-(N-(2-hydroxy-6-methylphenyl) sulfamoyl)-6-methoxypyridin-3-yl)-2-phenylthiazole-4-carboxamide (1953)

Prepared by Method-K by reacting 2-methoxy-5-(2-phenylthiazole-4-carboxamido)pyridine-3-sulfonyl chloride (14) with 2-((tert-butyldimethylsilyl)oxy)-6-methylaniline which was deprotected with TBAF to afford a white solid (49% yield). ¹H NMR (400 MHz, DMSO-d₆): δ 10.50 (s, 1H), 9.17 (s, 1H), 8.92-8.88 (m, 2H), 8.47-8.40 (m, 2H), 8.13 (d, J=3.2 Hz, 2H) 7.55-7.54 (m, 3H), 6.91 (t, J=7.6 Hz, 1H), 6.63 (d, J=7.6 Hz, 1H), 6.52 (d, J=7.6 Hz, 1H), 3.99 (s, 3H), 2.27 (s, 3H). LC-MS: m/z 497.1 ([M+H]⁺).

### Preparation of N-(6-methoxy-5-(N-(1, 3, 5-trimethyl-1H-pyrazol-4-yl) sulfamoyl)pyridin-3-yl)-2-phenylthiazole-4-carboxamide (1417)

Prepared by Method-K by reacting 2-methoxy-5-(2-phenylthiazole-4-carboxamido) pyridine-3-sulfonyl chloride (14) with 1,3,5-trimethyl-1H-pyrazol-4-amine to afford a white solid (66 yield %). ¹H NMR (400 MHz, DMSO-d₆): δ 10.55 (bs, 1H), 9.27 (bs, 1H), 8.95 (d, J=2.4 Hz, 1H), 8.48 (s, 1H), 8.41 (d, J=2.4 Hz, 1H), 8.15-8.13 (m, 2H), 7.56-7.54 (m, 3H), 4.05 (s, 3H), 3.53 (s, 3H), 1.91 (s, 3H), 1.70 (s, 3H). LC-MS: m/z 499.1 ([M+H]⁺).

### Preparation of N-(5-(N-(3-cyano-2-methylphenyl) sulfamoyl)-6-methoxypyridin-3-yl)-2-phenylthiazole-4-carboxamide (1406)

Prepared by Method-K by reacting 2-methoxy-5-(2-phenylthiazole-4-carboxamido) pyridine-3-sulfonyl chloride (14) with 3-amino-2-methylbenzonitrile to afford an off-white solid (40 yield %). ¹H NMR (400 MHz, DMSO-d₆): δ 10.57 (s, 1H), 10.15 (s, 1H), 8.92-8.91 (m, 1H), 8.57-8.56 (m, 1H), 8.50 (s, 1H), 8.14-8.12 (m, 2H), 7.65-7.64 (m, 1H), 7.57-7.55 (m, 3H), 7.38-7.34 (m, 2H), 3.94 (s, 3H), 2.35 (s, 3H). LC-MS: m/z 506.0 ([M+H]⁺).

### Preparation of N-(5-(N-(4-fluoro-2, 6-dimethylphenyl)sulfamoyl)-6-methoxypyridin-3-yl)-2-phenylthiazole-4-carboxamide (1389)

Prepared by Method-K by reacting 2-methoxy-5-(2-phenylthiazole-4-carboxamido)pyridine-3-sulfonyl chloride (14) with 4-fluoro-2,6-dimethylaniline, as an off-white solid (47% yield). ¹H NMR (400 MHz, DMSO-d₆): δ 10.49 (s, 1H), 9.49 (s, 1H), 8.93-8.93 (m, 1H), 8.48-8.47 (m, 2H), 8.14-8.13 (m, 2H), 7.56-7.54 (m, 3H), 7.90-6.88 (m, 2H), 4.03 (s, 3H), 2.04 (s, 6H). LC-MS: m/z 513.1 ([M+H]⁺).

### Preparation of N-(5-(N-(5-chloropyrimidin-2-yl) sulfamoyl)-6-methoxypyridin-3-yl)-2-phenylthiazole-4-carboxamide (1383)

Prepared by Method-J by reacting 2-methoxy-5-(2-phenylthiazole-4-carboxamido)pyridine-3-sulfonyl chloride (14) with 5-chloropyrimidin-2-amine to afford an off-white solid (7% yield). ¹H NMR (400 MHz, DMSO-d₆): δ 12.22 (s, 1H), 10.51 (s, 1H), 8.89-8.82 (m, 2H), 8.55-8.51 (m, 3H), 8.16-8.14 (m, 2H), 7.56-7.54 (m, 3H), 3.90 (s, 3H). LC-MS: m/z 503.0 ([M+H]⁺).

### Preparation of N-(5-(N-(2,6-dimethoxyphenyl)sulfamoyl)-6-methoxypyridin-3-yl)-2-phenylthiazole-4-carboxamide (1386)

Prepared by Method-J by reacting 2-methoxy-5-(2-phenylthiazole-4-carboxamido)pyridine-3-sulfonyl chloride (14) with 2,6-dimethoxyaniline to afford an off-white solid (37% yield). ¹H NMR (400 MHz, DMSO-d₆): δ 10.57 (s, 1H), 8.85 (d, J=2.8 Hz, 2H), 8.47 (s, 1H), 8.38 (d, J=2.0 Hz, 1H), 8.15-8.13 (m, 2H), 7.55 (t, J=5.6 Hz, 3H), 7.16 (s, 1H), 6.57 (d, J=8.8 Hz, 2H), 4.02 (s, 3H), 3.48 (s, 6H). LC-MS: m/z 527.05 ([M+H]⁺).

### Preparation of N-(5-(N-(2-ethyl-6-methylphenyl) sulfamoyl)-6-methoxypyridin-3-yl)-2-phenylthiazole-4-carboxamide (1393)

Prepared by Method-J by reacting 2-methoxy-5-(2-phenylthiazole-4-carboxamido) pyridine-3-sulfonyl chloride (14) with 2-ethyl-6-methylaniline to afford a pink solid (36% yield). ¹H NMR (400 MHz, DMSO-d₆): δ 10.54 (s, 1H), 9.46 (s, 1H), 8.92 (s, 1H), 8.48 (s, 2H), 8.13-8.12 (m, 2H), 7.54 (s, 3H), 7.12-7.02 (m, 3H), 4.04 (s, 3H), 2.50-7.43 (m, 2H), 2.06 (s, 3H), 0.96 (t, J=7.6 Hz, 3H). LC-MS: m/z 509.0 ([M+H]⁺).

### Preparation of N-(5-(N-(4-chloro-2,6-dimethylphenyl)sulfamoyl)-6-methoxypyridin-3-yl)-2-phenylthiazole-4-carboxamide (1388)

Prepared by Method-J by reacting 2-methoxy-5-(2-phenylthiazole-4-carboxamido) pyridine-3-sulfonyl chloride (14) with 4-chloro-2,6-dimethylaniline to afford an off-white solid (28% yield). ¹H NMR (400 MHz, DMSO-d₆): δ 10.54 (s, 1H), 9.46 (s, 1H), 8.92 (s, 1H), 8.48 (s, 2H), 8.13-8.12 (m, 2H), 7.54 (s, 3H), 7.12-7.02 (m, 3H), 4.04 (s, 3H), 2.06 (s, 6H). LC-MS: m/z 529.1 ([M+H]⁺).

### Preparation of N-(5-(N-(5-chloropyridin-2-yl) sulfamoyl)-6-methoxypyridin-3-yl)-2-phenylthiazole-4-carboxamide (1385)

Prepared by Method-J by reacting 2-methoxy-5-(2-phenylthiazole-4-carboxamido) pyridine-3-sulfonyl chloride (14) with 5-chloropyridin-2-amine to afford a white solid (21% yield). ¹H NMR (400 MHz, DMSO-d₆): δ 11.39 (bs, 1H), 10.65 (bs, 1H), 8.88 (d, J=2.4 Hz, 1H), 8.83 (d, J=2.8 Hz, 1H), 8.54 (s, 1H). 8.18-8.15 (m, 3H), 7.82 (dd, J₁=8.8, J₂=2.8 Hz, 1H), 7.59-7.57 (m, 3H), 7.08 (d, J=8.0 Hz, 1H), 3.88 (s, 3H). LC-MS: m/z 501.95 ([M+H]⁺).

### Preparation of N-(5-(N-(6-chloro-2-methylpyridin-3-yl) sulfamoyl)-6-methoxypyridin-3-yl)-2-phenylthiazole-4-carboxamide (1382)

Prepared by Method-J by reacting 2-methoxy-5-(2-phenylthiazole-4-carboxamido) pyridine-3-sulfonyl chloride (14) with 6-chloro-2-methylpyridin-3-amine to afford an off-white solid (60% yield). ¹H NMR (400 MHz, DMSO-d₆): δ 10.60 (bs, 1H), 10.20 (bs, 1H), 8.91 (d, J=2.4 Hz, 1H), 8.57 (d, J=2.0 Hz, 1H), 8.50 (s, 1H), 8.14-8.13 (m, 2H), 7.56-7.54 (m, 4H), 7.31 (d, J=8.4 Hz, 1H), 3.94 (s, 3H), 2.30 (s, 3H). LC-MS: m/z 513.95 ([M−2H]⁻).

### Preparation of N-(5-(N-(2, 6-diethylphenyl) sulfamoyl)-6-methoxypyridin-3-yl)-2-phenylthiazole-4-carboxamide (1418)

Prepared by Method-J by reacting 2-methoxy-5-(2-phenylthiazole-4-carboxamido) pyridine-3-sulfonyl chloride (14) with 2,6-diethylaniline to afford a pale yellow solid (57% yield). ¹H NMR (400 MHz, DMSO-d₆): δ 10.60 (bs, 1H), 10.20 (bs, 1H) 8.83 (d, J=2.4 Hz, 1H), 8.48 (d, J=2.8 Hz, 1H), 8.30 (s, 1H), 8.11-8.08 (m, 2H), 7.51-7.49 (m, 3H), 7.18-7.15 (m, 1H), 7.06 (d, J=7.6 Hz, 2H), 4.11 (d, J=15.6 Hz, 3H), 2.56 (dd, J₁=14.8 Hz, J₂=7.6 Hz, 4H), 1.06 (t, J=7.6 Hz, 6H). LC-MS: m/z 523.05 ([M+H]⁺).

### Preparation of N-(5-bromopyridin-3-yl)-2-phenyl-1H-imidazole-5-carboxamide (15)

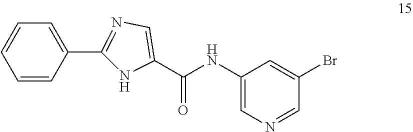 [SMILES: O=C(Nc1cncc(Br)c1)c1cnc(-c2ccccc2)[nH]1]

Prepared by Method-B, by reacting 5-bromo-pyridin-3-amine with sodium 2-phenyl-1H-imidazole-5-carboxylate (46), to give crude product as a light brown solid. LC-MS: m/z 345.0 ([M+2H]⁺).

### Preparation of N-(5-(benzylthio)pyridin-3-yl)-2-phenyl-1H-imidazole-5-carboxamide (16)

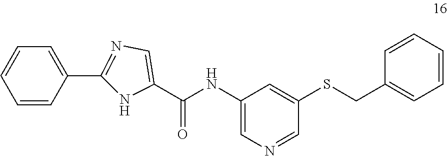 [SMILES: O=C(Nc1cncc(SCc2ccccc2)c1)c1cnc(-c2ccccc2)[nH]1]

Prepared by Method-D by reacting N-(5-bromopyridin-3-yl)-2-phenyl-1H-imidazole-5-carboxamide (15) with phenylmethanethiol to afford a light yellow solid (47% yield). LC-MS: m/z 387.02 ([M+H]⁺).

### Preparation of 5-(2-phenyl-1H-imidazole-5-carboxamido)pyridine-3-sulfonyl chloride (17)

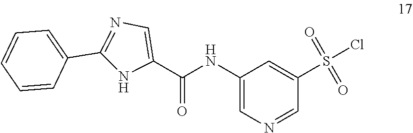 [SMILES: O=C(Nc1cncc(S(=O)(=O)Cl)c1)c1cnc(-c2ccccc2)[nH]1]

Prepared by Method-F starting with N-(5-(benzylthio)pyridin-3-yl)-2-phenyl-1H-imidazole-5-carboxamide (16) to give crude product as a light yellow solid. LC-MS: m/z 363.1 ([M+H]⁺).

### Preparation of N-(5-(N-(2,6-dimethylphenyl)sulfamoyl)pyridin-3-yl)-2-phenyl-1H-imidazole-5-carboxamide (592)

Prepared by Method-K by reacting 5-(2-phenyl-1H-imidazole-5-carboxamido)pyridine-3-sulfonyl chloride (17) with 2, 6-dimethylaniline to afford an off-white solid (6% yield). ¹H NMR (400 MHz, DMSO-d₆): δ 13.21 (s, 1H), 10.42 (s, 1H), 9.65 (s, 1H), 9.25 (s, 1H), 8.76 (s, 1H), 8.48 (s, 1H), 8.09 (d, J=7.2 Hz, 2H), 8.01 (s, 1H), 7.51 (t, J=7.2 Hz, 2H), 7.44 (t, J=7.2 Hz, 1H), 7.11-7.03 (m, 3H), 2.00 (s, 6H). LC-MS: m/z 448.2 ([M+H]⁺).

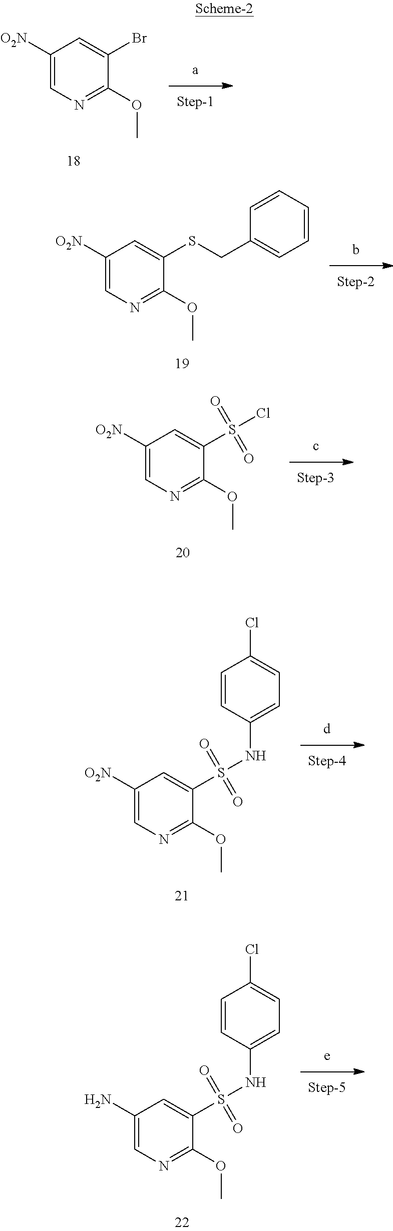 [SMILES: COc1ncc([N+](=O)[O-])cc1Br ; COc1ncc([N+](=O)[O-])cc1SCc1ccccc1 ; COc1ncc([N+](=O)[O-])cc1S(=O)(=O)Cl ; COc1ncc([N+](=O)[O-])cc1S(=O)(=O)Nc1ccc(Cl)cc1 ; COc1ncc(N)cc1S(=O)(=O)Nc1ccc(Cl)cc1]

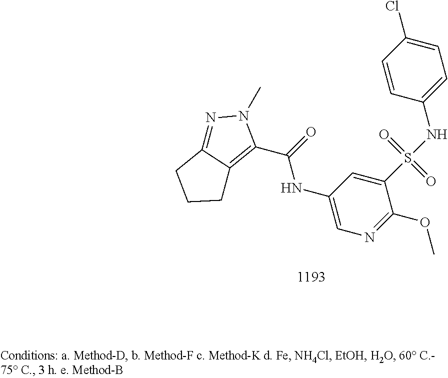 [SMILES: COc1ncc(NC(=O)c2c3c(nn2C)CCC3)cc1S(=O)(=O)Nc1ccc(Cl)cc1]

### Experimental Procedures for Scheme-2

### Scheme-2, Step-1: Preparation of 3-(benzylthio)-2-methoxy-5-nitropyridine (19)

Prepared by Method-D by reacting 3-bromo-2-methoxy-5-nitropyridine with phenylmethanethiol to give crude product as a brown sticky solid.

### Scheme-2, Step-2: Preparation of 2-methoxy-5-nitropyridine-3-sulfonyl chloride (20)

Prepared by Method-F starting with 3-(benzylthio)-2-methoxy-5-nitropyridine (19) to afford crude product as a white solid.

### Scheme-2, Step-3: Preparation of N-(4-chlorophenyl)-2-methoxy-5-nitropyridine-3-sulfonamide (21)

Prepared by Method-K by reacting 2-methoxy-5-nitropyridine-3-sulfonyl chloride (20) with 4-chloroaniline to afford a white solid (64% yield).

### Scheme-2, Step-4: Preparation of 5-amino-N-(4-chlorophenyl)-2-methoxypyridine-3-sulfonamide (22)

Was prepared by following compound 9 preparation procedure, to give an off-white solid (73% yield).

### Scheme-2, Step-5: Preparation of N-(5-(N-(4-chlorophenyl)sulfamoyl)-6-methoxypyridin-3-yl)-2-methyl-2,4,5,6-tetrahydrocyclopenta[c]pyrazole-3-carboxamide (1193)

Prepared by Method-B by reacting 5-amino-N-(4-chlorophenyl)-2-methoxypyridine-3-sulfonamide (22) with 2-methyl-2,4,5,6-tetrahydrocyclopenta[c]pyrazole-3-carboxylic acid to give an off-white solid (50% yield). ¹H NMR (400 MHz, DMSO-d₆): δ 10.48 (s, 1H), 10.32 (s, 1H), 8.76 (d, J=2.4 Hz, 1H), 8.69 (d, J=2.4 Hz, 1H), 7.30-7.27 (m, 2H), 7.12-7.09 (m, 2H), 3.92 (s, 3H), 3.81 (s, 3H), 3.77-3.33 (m, 2H), 2.72-2.69 (m, 2H), 2.52-2.49 (m, 2H). LC-MS: m/z 462.1 ([M+H]⁺).

The Following Compounds were Prepared According to Methods Analogous to Those Used in Scheme 2.

### Preparation of N-(5-(N-(4-chlorophenyl)sulfamoyl)-6-methoxypyridin-3-yl)-3-(2-oxocyclopentyl)benzamide (1201)

Prepared by Method-B by reacting 5-amino-N-(4-chlorophenyl)-2-methoxypyridine-3-sulfonamide (22) with 3-(2-oxocyclopentyl)benzoic acid to afford a white solid (48% yield). ¹H NMR (400 MHz, DMSO-d₆): δ 10.50 (s, 1H), 8.75 (d, J=2.4 Hz, 1H), 8.62 (d, J=2.4 Hz, 1H), 8.13 (bs, 1H), 7.95-7.93 (m, 1H), 7.54 (t, J=7.6 Hz, 1H), 7.31-7.11 (m, 4H), 3.95 (s, 3H), 3.92-3.89 (m, 2H), 2.55-2.50 (m, 3H), 2.11-2.08 (m, 2H). LC-MS: m/z 501.1 ([M+H]⁺).

### Preparation of N-(5-(N-(4-chlorophenyl) sulfamoyl)-6-methoxypyridin-3-yl)-2-(1-(4-methoxybenzyl)-1H-pyrazol-3-yl) acetamide (1200)

Prepared by Method-B by reacting 5-amino-N-(4-chlorophenyl)-2-methoxypyridine-3-sulfonamide (22) with 2-(1-(4-methoxybenzyl)-1H-pyrazol-3-yl)acetic acid to afford a white solid (28% yield). ¹H NMR (400 MHz, DMSO-d₆): δ 10.46 (s, 1H), 10.29 (s, 1H), 8.48 (s, 1H), 7.65 (s, 1H), 7.35-7.07 (m, 7H), 6.89 (d, J=8.8 Hz, 2H), 5.18 (s, 2H), 3.91 (s, 3H), 3.72 (s, 3H), 3.45 (s, 2H). LC-MS: m/z 542.2 ([M+H]⁺).

### Preparation of N-(5-(N-(4-chlorophenyl) sulfamoyl)-6-methoxypyridin-3-yl)-3-(1H-1,2,4-triazol-1-yl)benzamide (1204)

Prepared by Method-B by reacting 5-amino-N-(4-chlorophenyl)-2-methoxypyridine-3-sulfonamide (22) with 3-(1H-1,2,4-triazol-1-yl)benzoic acid to afford a white solid (48% yield.) ¹H NMR (400 MHz, DMSO-d₆): δ 10.67 (s, 1H), 10.52 (s, 1H), 9.40 (s, 1H), 8.77 (d, J=2.4 Hz, 1H), 8.64 (d, J=2.4 Hz, 1H), 8.44 (s, 1H), 8.30 (s, 1H), 8.12-7.99 (m, 2H), 7.75 (t, J=8.4 Hz, 1H), 7.30 (d, J=2.6 Hz), 7.13 (d, J=2.7 Hz, 1H), 3.97 (s, 3H). LC-MS: m/z 485.1 ([M+H]⁺).

### Preparation of 1-benzyl-N-(5-(N-(4-chlorophenyl) sulfamoyl)-6-methoxypyridin-3-yl)-1H-pyrazole-3-carboxamide (1199)

Prepared by Method-B by reacting 5-amino-N-(4-chlorophenyl)-2-methoxypyridine-3-sulfonamide (22) with 1-benzyl-1H-pyrazole-3-carboxylic acid to afford a white solid (15% yield). ¹H NMR (400 MHz, DMSO-d₆): δ 10.25 (s, 1H), 10.1 (s, 1H), 8.7 (s, 1H), 8.5 (s, 1H), 8.4 (s, 1H), 8.0 (s, 1H), 7.39-7.27 (m, 7H), 7.1-7.09 (m, 2H), 5.40 (s, 2H), 3.93 (s, 3H). LC-MS: m/z 499 ([M+H]⁺).

### Preparation of N-(5-(N-(4-chlorophenyl) sulfamoyl)-6-methoxypyridin-3-yl)-1, 3-dimethyl-2-oxo-2,3-dihydro-1H-benzo[d]imidazole-5-carboxamide (1206)

Prepared by Method-B by reacting 5-amino-N-(4-chlorophenyl)-2-methoxypyridine-3-sulfonamide (22) with 1,3-dimethyl-2-oxo-2,3-dihydro-1H-benzo[d]imidazole-5-carboxylic acid, to afford a white solid (31% yield). ¹H NMR (400 MHz, DMSO-d₆): δ 10.50 (s, 1H), 10.38 (s, 1H), 8.77 (d, J=2.8 Hz, 1H), 8.62 (d, J=2.4 Hz, 1H), 7.80-7.77 (m, 2H), 7.30 (d, J=8.8 Hz, 3H), 7.12 (d, J=9.2 Hz, 2H), 3.96 (s, 3H), 3.40 (s, 3H), 3.38 (s, 3H). LC-MS: m/z 500.0 ([M−H]⁻).

### Preparation of N-(5-(N-(4-chlorophenyl) sulfamoyl)-6-methoxypyridin-3-yl)-2-methylquinoline-6-carboxamide (1202)

Prepared by Method-B by reacting 5-amino-N-(4-chlorophenyl)-2-methoxypyridine-3-sulfonamide (22) with 2-methylquinoline-6-carboxylic acid to afford a white solid (39% yield). ¹H NMR (400 MHz, DMSO-d₆): δ 10.25 (s, 1H), 10.1 (s, 1H), 8.7 (s, 1H), 8.5 (s, 1H), 8.4 (s, 1H), 8.0 (s, 1H), 7.39-7.27 (m, 7H), 7.1-7.09 (m, 2H), 5.4 (s, 3H), 3.93 (s, 3H). LC-MS: m/z 483.1 ([M−H]⁻).

### Preparation of 3-benzoyl-N-(5-(N-(4-chlorophenyl) sulfamoyl)-6-methoxypyridin-3-yl)picolinamide (1195)

Prepared by Method-B by reacting 5-amino-N-(4-chlorophenyl)-2-methoxypyridine-3-sulfonamide (22) with 3-benzoylpicolinic acid to afford a white solid (26% yield). ¹H NMR (400 MHz, DMSO-d₆): δ 10.85 (bs, 1H), 10.13 (bs, 1H), 8.83 (dd, J₁=17.2 Hz, J₂=3.6 Hz, 1H), 8.57-8.53 (m, 1H), 8.33 (dd, J₁=20.8 Hz, J₂=2.4 Hz, 1H), 7.96 (d, J=8.0 Hz, 2H), 7.67-6.99 (m, 8H), 3.59 (d, J=5.2 Hz, 3H). LC-MS: m/z 523.1 ([M+H]⁺).

### Preparation of N-(5-(N-(4-chlorophenyl) sulfamoyl)-6-methoxypyridin-3-yl) quinoxaline-6-carboxamide (1198)

Prepared by Method-B by reacting 5-amino-N-(4-chlorophenyl)-2-methoxypyridine-3-sulfonamide (22) with quinoxaline-6-carboxylic acid to afford a white solid (49% yield). ¹H NMR (400 MHz, DMSO-d₆): δ 10.87 (bs, 1H), 10.53 (bs, 1H), 9.08-9.07 (t, J=1.6 Hz, 2H), 8.82-8.70 (m, 4H), 8.26 (dd, J₁=8.8 Hz, J₂=2.0 Hz, 1H), 8.33 (d, J=8.8 Hz, 2H), 7.14 (d, J=8.8 Hz, 2H), 3.98 (s, 3H). LC-MS: m/z 470.1 ([M+H]⁺).

### Preparation of (4-bromo-1H-pyrazol-1-yl)-N-(5-(N-(4-chlorophenyl)sulfamoyl)-6-methoxypyridin-3-yl)propanamide (1213)

Prepared by Method-B by reacting 5-amino-N-(4-chlorophenyl)-2-methoxypyridine (22) with 2-(4-bromo-1H-pyrazol-1-yl) propanoic acid (47) to afford an off-white solid (24% yield). ¹H NMR (400 MHz, DMSO-d₆): δ 10.56 (bs, 1H), 10.48 (bs, 1H), 8.50 (s, 1H), 8.44 (s, 1H), 8.14 (s, 1H), 7.58 (s, 1H), 7.29 (d, J=8.4 Hz, 1H), 7.09 (d, J=8.8 Hz, 2H), 5.16 (d, J=6.8 Hz, 1H), 3.92 (s, 3H), 1.69 (d, J=6.8 Hz, 1H). LC-MS: m/z 514.0 ([M+H]⁺).

### Preparation of N-(5-(N-(4-chlorophenyl) sulfamoyl)-6-methoxypyridin-3-yl)-4,5,6,7-tetrahydrobenzo[d]isoxazole-3-carboxamide (1194)

Prepared by Method-B by reacting 5-amino-N-(4-chlorophenyl)-2-methoxypyridine (22) with 4,5,6,7-tetrahydrobenzo[d]isoxazole-3-carboxylic acid to afford an off-white solid (17% yield). ¹H NMR (400 MHz, DMSO-d₆): δ 10.94 (bs, 1H), 10.51 (bs, 1H), 8.69 (bs, 2H), 7.29 (d, J=8.8 Hz, 2H), 7.11 (d, J=8.8 Hz, 2H), 3.94 (s, 3H), 2.77-2.74 (m, 2H), 2.61-2.58 (m, 2H), 1.82-1.80 (m, 2H), 1.72-1.70 (m, 2H). LC-MS: m/z 461.1 ([M−2H]⁻).

### Preparation of N-(5-(N-(4-chlorophenyl) sulfamoyl)-6-methoxypyridin-3-yl)-4-(1H-imidazol-1-yl)picolinamide (1203)

Prepared by Method-B by reacting 5-amino-N-(4-chlorophenyl)-2-methoxypyridine-3-sulfonamide (22) with 4-(1H-imidazol-1-yl)picolinic acid to afford an off-white solid (13% yield). ¹H NMR (400 MHz, DMSO-d₆): δ 11.14 (bs, 1H), 10.52 (bs, 1H), 8.86-8.80 (m, 3H), 8.69 (s, 1H), 8.43 (d, J=1.2 Hz, 1H), 8.12 (s, 1H), 8.06 (dd, J₁=5.2 Hz, J₂=2.0 Hz, 1H), 7.29 (d, J=8.8 Hz, 2H), 7.21 (s, 1H), 7.13 (d, J=8.8 Hz, 2H), 3.96 (s, 3H). LC-MS: m/z 483.1 ([M−2H]⁻).

### Preparation of N-(5-(N-(4-chlorophenyl) sulfamoyl)-6-methoxypyridin-3-yl)-1-methyl-1H-pyrrolo [2,3-b]pyridine-3-carboxamide (1359)

Prepared by Method-B by reacting 5-amino-N-(4-chlorophenyl)-2-methoxypyridine-3-sulfonamide (22) with 1-methyl-1H-pyrrolo[2,3-b]pyridine-3-carboxylic acid to afford an off-white solid (27% yield). ¹H NMR (400 MHz, DMSO-d₆): δ 10.49 (s, 1H), 10.93 (s, 1H), 8.73 (s, 1H), 8.61 (s, 1H), 8.50 (d, J=6.8 Hz, 1H), 8.39 (d, J=8.8 Hz, 2H), 7.30 (d, J=8.0 Hz, 3H), 7.13 (d, J=7.2 Hz, 2H), 3.93 (d, J=14.6 Hz, 1H). LC-MS: m/z 472.1 ([M+H]⁺).

### Preparation of 2-benzoyl-N-(5-(N-(4-chlorophenyl) sulfamoyl)-6-methoxypyridin-3-yl)benzamide (1197)

Prepared by Method-B by reacting 5-amino-N-(4-chlorophenyl)-2-methoxypyridine-3-sulfonamide (22) with 2-benzoylbenzoic acid to afford an off-white solid (40% yield). ¹H NMR (400 MHz, DMSO-d₆): δ 10.50 (bs, 1H), 8.42 (d, J=2.4 Hz, 1H), 8.34 (d, J=2.4 Hz, 1H), 7.86 (d, J=7.6 Hz, 1H) 7.80 (s, 1H), 7.67-7.58 (m, 2H), 7.31-7.21 (m, 8H), 6.97 (d, J=8.4 Hz, 2H), 3.89 (s, 3H). LC-MS: m/z 520.1 ([M−H]⁻).

### Preparation of N-(5-(N-(4-chlorophenyl)sulfamoyl)-6-methoxypyridin-3-yl)-5-methyloxazole-4-carboxamide (1959)

Prepared by Method-B by reacting 5-amino-N-(4-chlorophenyl)-2-methoxypyridine-3-sulfonamide (22) with 5-methyloxazole-4-carboxylic acid to afford an off-white solid (37% yield). ¹H-NMR (400 MHz, DMSO-d₆): δ 10.50-10.46 (m, 2H), 8.74-8.70 (m, 2H), 8.46 (s, 1H), 7.30-7.28 (m, 2H), 7.12-7.09 (m, 2H), 3.93 (s, 3H), 2.63 (m, 3H). LC-MS: m/z 423.0 ([M+H]⁺).

### Preparation of N-(5-(N-(4-chlorophenyl) sulfamoyl)-6-methoxypyridin-3-yl)-2-phenyl-1H-imidazole-5-carboxamide (1481)

Prepared by Method-B by reacting 5-amino-N-(4-chlorophenyl)-2-methoxypyridine-3-sulfonamide (22) with sodium 2-phenyl-1H-imidazole-5-carboxylate (46) to afford an off-white solid (48% yield). ¹H NMR (400 MHz, DMSO-d₆): δ 10.76 (s, 1H), 9.33 (s, 1H), 9.14 (s, 1H), 8.54 (d, J=5.2 Hz, 1H), 8.34 (s, 1H), 8.11-7.95 (m, 3H), 7.30 (d, J=9.2 Hz, 1H), 7.05-7.00 (m, 3H), 3.93 (s, 3H), 2.04 (s, 6H). LC-MS: m/z 468.0 ([M+H]⁺).

### Preparation of N-(2,6-dimethylphenyl)-2-methoxy-5-nitropyridine-3-sulfonamide (23)

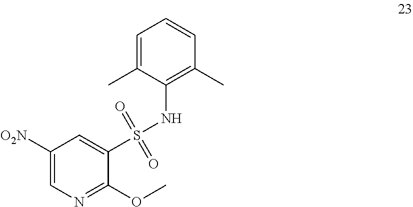 [SMILES: COc1ncc([N+](=O)[O-])cc1S(=O)(=O)Nc1c(C)cccc1C]

Prepared by reacting 2-methoxy-5-nitropyridine-3-sulfonyl chloride (20) with 2,6-dimethylaniline as in Method-K to give product as a yellow solid (60% yield). LC-MS: m/z 336.1 ([M−H]⁻).

### Preparation of 5-amino-N-(2,6-dimethylphenyl)-2-methoxypyridine-3-sulfonamide (24)

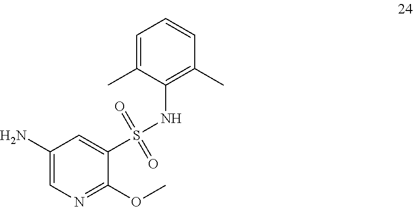 [SMILES: COc1ncc(N)cc1S(=O)(=O)Nc1c(C)cccc1C]

Prepared by following compound 9 preparation procedure to give a light yellow solid (90% yield). LC-MS: m/z 308.1 ([M+H]⁺).

### Preparation of N-(5-(N-(2,6-dimethylphenyl)sulfamoyl)-6-methoxypyridin-3-yl)-2-(pyridin-4-yl)thiazole-4-carboxamide (1351)

Prepared by Method-B by reacting 5-amino-N-(2, 6-dimethylphenyl)-2-methoxypyridine-3-sulfonamide (24) with 2-(pyridin-4-yl) thiazole-4-carboxylic acid to give an off-white solid (25% yield). ¹H NMR (400 MHz, DMSO-d₆): δ 10.85 (s, 1H), 9.5 (s, 1H), 8.92-8.93 (m, 1H), 8.76-8.78 (m, 2H), 8.64 (s, 1H), 8.44-8.55 (m, 1H), 8.07-8.11 (m, 2H), 7.01-7.09 (m, 3H), 4.03 (s, 3H), 2.04 (s, 6H). LC-MS: m/z 496.2 ([M+H]⁺).

### Preparation of N-(5-(N-(2,6-dimethylphenyl)sulfamoyl)-6-methoxypyridin-3-yl)-3,4-dihydro-2H-pyrano[3,2-b]pyridine-8-carboxamide (1364)

Prepared by Method-B by reacting 5-amino-N-(2,6-dimethylphenyl)-2-methoxypyridine-3-sulfonamide (24) with 3,4-dihydro-2H-pyrano[3,2-b]pyridine-8-carboxylic acid to give an off-white solid (27% yield). ¹H NMR (400 MHz, DMSO-d₆): δ 10.46 (s, 1H), 9.5 (s, 1H), 8.71 (d, J=2.8 Hz, 1H), 8.36 (d, J=2.4 Hz, 1H), 8.13 (d, J=4.8 Hz, 1H), 7.35-7.34 (m, 1H), 7.07-7.03 (m, 3H), 4.27-4.25 (m, 2H), 4.0 (s, 3H), 2.94-2.89 (m, 2H), 2.07-2.03 (m, 8H). LC-MS: m/z 469.2 ([M+H]⁺).

### Preparation of N-(5-(N-(2,6-dimethylphenyl)sulfamoyl)-6-methoxypyridin-3-yl)-2-(pyridin-4-yl)thiazole-4-carboxamide (1365)

Prepared by Method-B by reacting 5-amino-N-(2, 6-dimethylphenyl)-2-methoxypyridine-3-sulfonamide (24) with 2-(pyridin-3-yl)thiazole-4-carboxylic acid to give an off-white solid (10% yield). ¹H NMR (400 MHz, DMSO-d₆): δ 10.59 (s, 1H), 9.5 (s, 1H), 9.37-9.36 (m, 1H), 8.92-8.93 (m, 1H), 8.72-8.71 (m, 1H), 8.57 (s, 1H), 8.49-8.5 (m, 2H), 7.61-7.57 (m, 1H), 7.06-7.0 (m, 3H), 4.03 (s, 3H), 2.04 (s, 6H). LC-MS: m/z 496.2 ([M+H]⁺).

### Preparation of N-(5-(N-(2,6-dimethylphenyl)sulfamoyl)-6-methoxypyridin-3-yl)-2-phenyloxazole-4-carboxamide (1312)

Prepared by Method-B by reacting 5-amino-N-(2,6-dimethylphenyl)-2-methoxypyridine-3-sulfonamide (24) with 2-phenyloxazole-4-carboxylic acid to give an off-white solid (51% yield). ¹H NMR (400 MHz, DMSO-d₆): δ 10.45 (s, 1H), 9.46 (m, 1H), 8.87-8.65 (m, 2H), 8.46-8.46 (m, 1H), 8.09-8.06 (m, 2H), 7.60-7.59 (m, 3H), 7.05-7.01 (m, 3H), 4.01 (s, 3H), 2.07 (s, 6H). LC-MS: m/z 479.0 ([M+H]⁺).

### Preparation of N-(5-(N-(2,6-dimethylphenyl)sulfamoyl)-6-methoxypyridin-3-yl)benzo[d]oxazole-7-carboxamide (1367)

Prepared by Method-B by reacting 5-amino-N-(2,6-dimethylphenyl)-2-methoxypyridine-3-sulfonamide (24) with benzo[d]oxazole-7-carboxylic acid to give an off-white solid (24% yield). ¹H NMR (400 MHz, DMSO-d₆): δ 10.63 (bs, 1H), 9.50 (s, 1H), 8.90 (s, 1H), 8.83 (d, J=2.4 Hz, 1H), 8.38 (d, J=2.4 Hz, 1H), 8.03-8.01 (m, 1H), 7.86 (d, J=7.2 Hz, 1H), 7.56-7.24 (m, 1H), 7.09-7.01 (m, 3H), 4.02 (s, 3H), 2.04 (s, 6H). LC-MS: m/z 453.05 ([M+H]⁺).

### Preparation of N-(5-(N-(2,6-dimethylphenyl)sulfamoyl)-6-methoxypyridin-3-yl)benzo[d]oxazole-4-carboxamide (1366)

Prepared by Method-B by reacting 5-amino-N-(2, 6-dimethylphenyl)-2-methoxypyridine-3-sulfonamide (24) with benzo[d]oxazole-4-carboxylic acid to give an off-white solid (22% yield). ¹H NMR (400 MHz, DMSO-d₆): δ 10.63 (bs, 1H), 9.50 (s, 1H), 8.90 (s, 1H), 8.83 (d, J=2.4 Hz, 1H), 8.38 (d, J=2.4 Hz, 1H), 8.03-8.01 (m, 1H), 7.86 (d, J=7.2 Hz, 1H), 7.56-7.24 (m, 1H), 7.09-7.01 (m, 3H), 4.02 (s, 3H), 2.04 (s, 6H). LC-MS: m/z 453.05 ([M+H]⁺).

### Preparation of N-(5-(N-(2,6-dimethylphenyl)sulfamoyl)-6-methoxypyridin-3-yl)-5-fluoro-2-methoxybenzamide (1990)

Prepared by Method-B by reacting 5-amino-N-(2,6-dimethylphenyl)-2-methoxypyridine-3-sulfonamide (24) with 5-fluoro-2-methoxybenzoic acid to give an off-white solid (23% yield). ¹H NMR (400 MHz, DMSO-d₆): δ 10.37 (s, 1H), 9.48 (s, 1H), 8.70-8.70 (d, J=2.4 Hz, 1H), 8.40-8.39 (d, J=2.8 Hz, 1H), 7.47-7.44 (m, 1H), 7.38-7.33 (m, 1H), 7.20-7.17 (m, 1H), 7.07-7.01 (m, 3H), 4.00 (s, 3H), 3.85 (s, 3H), 2.03 (s, 6H). LC-MS: m/z 460.1 ([M+H]⁺).

### Preparation of N-(5-(N-(2, 6-dimethylphenyl) sulfamoyl)-6-methoxypyridin-3-yl)-4,5,6,7-tetrahydrothieno[3,2-c]pyridine-3-carboxamide (1972)

Prepared by Method-B by reacting 5-amino-N-(2,6-dimethylphenyl)-2-methoxypyridine-3-sulfonamide (24) with 6-(tert-butoxycarbonyl)-4,5,6,7-tetrahydrothieno[2,3-c]pyridine-3-carboxylic acid (49), and then deprotecting with dioxane-HCl to give an off-white solid (32% yield). ¹H NMR (400 MHz, DMSO-d₆): δ 10.2 (s, 1H), 9.484 (s, 1H), 9.28 (m, 2H), 8.76-8.76 (m, 1H), 8.37-8.30 (m, 2H), 7.06-7.01 (m, 3H), 4.37 (s, 2H), 4.00 (s, 3H), 3.07 (m, 2H), 2.03 (s, 6H). LC-MS: m/z 473 ([M+H]⁺).

### Preparation of N-(5-(N-(2, 6-dimethylphenyl) sulfamoyl)-6-methoxypyridin-3-yl)-5-methyl-4,5,6,7-tetrahydrothieno[3,2-c]pyridine-3-carboxamide (1956)

Prepared by method-C by reacting 5-amino-N-(2,6-dimethylphenyl)-2-methoxypyridine-3-sulfonamide in (24) with ethyl 6-methyl-4,5,6,7-tetrahydrothieno[2,3-c]pyridine-3-carboxylate (52) to give an off-white solid (25% yield). ¹H NMR (400 MHz, DMSO-d₆): δ 8.85 (s, 1H), 8.05 (s, 1H), 7.61 (s, 1H), 7.52 (m, 1H), 7.10-7.05 (m, 3H), 6.56 (s, 1H), 4.17 (s, 3H), 3.64 (s, 2H), 3.02 (m, 2H), 2.75-2.73 (m, 2H), 2.49 (s, 3H), 2.15 (s, 6H). LC-MS: m/z 487 ([M+H]⁺).

### Preparation of tert-butyl 3-((5-(N-(2,6-dimethylphenyl)sulfamoyl)-6-methoxypyridin-3-yl)carbamoyl)-6,7-dihydrothieno[3,2-c]pyridine-5(4H)-carboxylate (1949)

Prepared by Method-B by reacting 5-amino-N-(2,6-dimethylphenyl)-2-methoxypyridine-3-sulfonamide (24) with 6-(tert-butoxycarbonyl)-4,5,6,7-tetrahydrothieno[2,3-c]pyridine-3-carboxylic acid (49) to yield an off-white solid (22% yield). ¹H NMR (400 MHz, DMSO-d₆): δ 10.2 (s, 1H), 9.392 (m, 1H), 8.732 (s, 1H), 8.327 (s, 1H), 8.103 (s, 1H), 7.06-6.99 (m, 3H), 4.55 (s, 2H), 3.97 (s, 3H), 3.53 (m, 3H), 2.81 (m, 2H), 2.05-2.01 (m, 6H), 1.40 (s, 9H). LC-MS: m/z 573 ([M+H]⁺).

### Preparation of N-(5-(N-(2,6-dimethylphenyl)sulfamoyl)-6-methoxypyridin-3-yl)-5,6,7,8-tetrahydroimidazo[1,2-a]pyridine-2-carboxamide (1943)

Prepared by Method-B by reacting 5-amino-N-(2,6-dimethylphenyl)-2-methoxypyridine-3-sulfonamide (24) with 5,6,7,8-tetrahydroimidazo[1,2-a]pyridine-2-carboxylic acid to afford a white solid (67% yield). ¹H NMR (400 MHz, DMSO-d₆): δ 10.58 (bs, 1H), 9.40 (bs, 1H), 8.74 (d, J=2.4 Hz, 1H), 8.58 (d, J=2.4 Hz, 1H), 7.06-6.88 (m, 4H), 4.34 (t, J=5.6 Hz, 2H), 3.98 (s, 3H), 2.78 (t, J=6.9 Hz, 2H), 2.02 (s, 6H), 1.88-1.86 (m, 2H), 1.72 (t, J=6 Hz, 2H). LC-MS: 456.1 ([M+H]⁺).

### Preparation of N-(5-(N-(2,6-dimethylphenyl)sulfamoyl)-6-methoxypyridin-3-yl)-3-(1-methyl-1H-pyrazol-5-yl)benzamide (1942)

Prepared by Method-B by reacting 5-amino-N-(2, 6-dimethylphenyl)-2-methoxypyridine-3-sulfonamide (24) with 3-(1-methyl-1H-pyrazol-5-yl) benzoic acid to give a light-brown solid (86% yield). ¹H NMR (400 MHz, DMSO-d₆): δ 10.50 (bs, 1H), 9.49 (bs, 1H), 8.86 (d, J=4.0 Hz, 1H), 8.38 (d, J=2.4 Hz, 1H), 8.08 (s, 1H), 8.0 (d, J=8.0 Hz, 1H), 7.76 (d, J=8.0 Hz, 1H), 7.66 (d, J=8.0 Hz, 1H), 7.50 (s, 1H), 7.05-7.01 (m, 3H), 5.50 (d, J=4.0 Hz, 1H), 4.02 (s, 3H), 3.87 (s, 3H), 2.04 (s, 6H). LC-MS: m/z 492.0 ([M+H]⁺).

### Preparation of 1,2-bromo-N-(5-(N-(2,6-dimethylphenyl)sulfamoyl)-6-methoxypyridin-3-yl)isonicotinamide (1935)

Prepared by Method-B by reacting 5-amino-N-(2,6-dimethylphenyl)-2-methoxypyridine-3-sulfonamide (24) with 2-bromoisonicotinic acid to give an off-white solid (56% yield). ¹H NMR (400 MHz, DMSO-d₆): δ 10.76 (bs, 1H), 9.51 (bs, 1H), 8.82 (d, J=2.4 Hz, 1H), 8.59 (d, J=5.6 Hz, 1H), 8.35 (d, J=2.4 Hz, 1H), 8.12 (s, 1H), 7.88 (d, J=5.2 Hz, 1H), 7.08-7.01 (m, 3H), 4.02 (s, 3H), 2.02 (s, 6H). LC-MS: m/z 493.1 ([M−2H]⁻).

### Preparation of N-(5-(N-(2,6-dimethylphenyl)sulfamoyl)-6-methoxypyridin-3-yl)-2-phenylnicotinamide (1483)

Prepared by Method-B by reacting 5-amino-N-(2,6-dimethylphenyl)-2-methoxypyridine-3-sulfonamide (24) with 2-phenylnicotinic acid to give a light yellow solid (86% yield). ¹H NMR (400 MHz, DMSO-d₆): δ 10.76 (bs, 1H), 9.55 (bs, 1H), 8.87 (d, J=4.0 Hz, 2H), 8.41-8.38 (m, 2H), 8.16 (d, J=8.0 Hz, 2H), 7.80 (d, J=4.0 Hz, 1H), 7.56-7.48 (m, 3H), 7.08-7.02 (m, 3H), 4.03 (s, 3H), 2.04 (s, 6H). LC-MS: m/z 489.1 ([M+H]⁺).

### Preparation of N-(5-(N-(2,6-dimethylphenyl)sulfamoyl)-6-methoxypyridin-3-yl)-1-methyl-4,5,6,7-tetrahydro-1H-indazole-3-carboxamide (1424)

Prepared by Method-B by reacting 5-amino-N-(2, 6-dimethylphenyl)-2-methoxypyridine-3-sulfonamide (24) with 1-methyl-4,5,6,7-tetrahydro-1H-indazole-3-carboxylic acid to give an off-white solid (46% yield). ¹H NMR (400 MHz, DMSO-d₆): δ 10.22 (s, 1H), 9.38 (s, 1H), 8.74-8.74 (m, 1H), 8.56-8.56 (m, 1H), 7.04-7.00 (m, 3H), 3.97 (s, 3H), 3.76 (s, 3H), 2.65-2.58 (m, 4H), 2.02 (s, 6H), 1.74-1.63 (m, 4H). LC-MS: m/z 470.1 ([M+H]⁺).

### Preparation of N-(5-(N-(2, 6-dimethylphenyl) sulfamoyl)-6-methoxypyridin-3-yl)-1-methyl-1H-indazole-3-carboxamide (1423)

Prepared by Method-B by reacting 5-amino-N-(2,6-dimethylphenyl)-2-methoxypyridine-3-sulfonamide (24) with 1-methyl-1H-indazole-3-carboxylic acid to give an off-white solid (43% yield). ¹H NMR (400 MHz, DMSO-d₆): δ 10.71 (s, 1H), 9.43 (s, 1H), 8.85 (d, J=2.4 Hz, 1H), 8.63 (d, J=2.0 Hz, 1H), 8.19 (d, J=8.4 Hz, 1H), 7.78 (d, J=8.4 Hz, 1H), 7.50 (t, J=7.6 Hz, 1H), 7.31 (t, J=8.0 Hz, 1H), 7.07-7.01 (m, 3H), 4.19 (s, 3H), 4.00 (s, 3H), 2.04 (s, 6H). LC-MS: m/z 466.2 ([M+H]⁺).

### Preparation of N-(5-(N-(4-chlorophenyl) sulfamoyl)-6-methoxypyridin-3-yl)-4-(4-propylpiperazin-1-yl) benzamide (1422)

Prepared by Method-B by reacting 5-amino-N-(2, 6-dimethylphenyl)-2-methoxypyridine-3-sulfonamide (24) with 5-phenyl-1H-pyrazole-3-carboxylic acid to give a yellow solid (56% yield). ¹H NMR (400 MHz, DMSO-d₆): δ 8.50 (d, J=2.8 Hz, 1H), 8.42 (d, J=2.4 Hz, 1H), 7.55 (d, J=8.8 Hz, 2H), 7.11-7.09 (m, 2H), 7.04-7.01 (m, 2H), 6.91 (d, J=2.4 Hz, 2H), 3.94 (s, 3H), 3.28-3.24 (m, 4H), 2.56-2.53 (m, 4H), 2.32-2.28 (m, 2H), 1.51-1.48 (m, 2H), 0.87-0.84 (t, J=7.2 Hz, 3H). LC-MS: m/z 478.05 ([M+H]⁺).

### Preparation of N-(5-(N-(2, 6-dimethylphenyl) sulfamoyl)-6-methoxypyridin-3-yl) benzo[d]isoxazole-3-carboxamide (1421)

Prepared by Method-B by reacting 5-amino-N-(2, 6-dimethylphenyl)-2-methoxypyridine-3-sulfonamide (24) with benzo[d]isoxazole-3-carboxylic acid to give an off-white solid (36% yield). ¹H NMR (400 MHz, DMSO-d₆): δ 11.3 (bs, 1H), 9.50 (bs, 1H), 8.84 (d, J=2.4 Hz, 1H), 8.57 (d, J=2.4 Hz, 1H), 8.16 (d, J=8.0 Hz, 1H), 7.91 (d, J=8.4 Hz, 1H), 7.76 (t, J=8.0 Hz, 1H), 7.55-7.51 (m, 1H), 7.08-7.02 (m, 3H), 4.02 (s, 3H), 2.04 (s, 6H). LC-MS: m/z 453.1 ([M+H]⁺).

### Preparation of N-(5-(N-(2, 6-dimethylphenyl)sulfamoyl)-6-methoxypyridin-3-yl)-2-(4-fluorophenyl)thiazole-4-carboxamide (1413)

Prepared by Method-B by reacting 5-amino-N-(2, 6-dimethylphenyl)-2-methoxypyridine-3-sulfonamide (24) with 2-(4-fluorophenyl)thiazole-4-carboxylic acid to give an off-white solid (45% yield). ¹H NMR (400 MHz, DMSO-d₆): δ 10.55 (bs, 1H), 9.47 (s, 1H), 8.92 (d, J=2.0 Hz, 1H), 8.47-8.45 (m, 2H), 8.21-8.17 (m, 2H), 7.42-7.38 (m, 2H), 7.05-7.01 (m, 3H), 4.03 (s, 3H), 2.41 (s, 6H). LC-MS: m/z 513.1 ([M+H]⁺).

### Preparation of N-(5-(N-(2, 6-dimethylphenyl) sulfamoyl)-6-methoxypyridin-3-yl)-5,6,7,8-tetrahydroimidazo[1,2-a]pyridine-3-carboxamide (1404)

Prepared by Method-B by reacting 5-amino-N-(2,6-dimethylphenyl)-2-methoxypyridine-3-sulfonamide (24) with 5,6,7,8-tetrahydroimidazo[1,2-a]pyridine-3-carboxylic acid hydrochloride to give an off-white solid (27% yield). ¹H NMR (400 MHz, DMSO-d₆): δ 10.10 (bs, 1H), 9.48 (bs, 1H), 8.74 (d, J=2.4 Hz, 1H), 8.31 (d, J=2.8 Hz, 1H), 7.73 (s, 1H), 7.07-7.01 (m, 3H), 4.21 (t, J=6.0 Hz, 2H), 3.99 (s, 3H), 2.78 (t, J=6.0 Hz, 2H), 2.02 (s, 6H), 1.88-1.81 (m, 4H). LC-MS: m/z 456.5 ([M−2H]⁻).

### Preparation of N-(5-(N-(2,6-dimethylphenyl)sulfamoyl)-6-methoxypyridin-3-yl)benzo[d]isoxazole-3-carboxamide (1403)

Prepared by Method-B by reacting 5-amino-N-(2,6-dimethylphenyl)-2-methoxypyridine-3-sulfonamide (24) with 3-(pyridin-3-yl)benzoic acid to give a light yellow solid (37% yield). ¹H NMR (400 MHz, DMSO-d₆): δ 10.76 (bs, 1H), 9.55 (bs, 1H), 8.87 (dd, J=4.0 Hz, 2H), 8.41-8.38 (m, 2H), 8.16 (d, J=8.0 Hz, 2H), 7.80 (d, J=4.0 Hz, 1H), 7.56-7.48 (m, 3H), 7.08-7.02 (m, 3H), 4.03 (s, 3H), 2.04 (s, 6H). LC-MS: m/z 489.1 ([M+H]⁺).

### Preparation of 4-((4-chloro-1H-pyrazol-1-yl) methyl)-N-(5(N (2,6dimethylphenyl)sulfamoyl)-6-methoxypyridin-3-yl)benzamide (1402)

Prepared by Method-B by reacting 5-amino-N-(2,6-dimethylphenyl)-2-methoxypyridine-3-sulfonamide (24) with 4-((4-chloro-1H-pyrazol-1-yl)methyl)benzoic acid to give an off-white solid (58% yield). ¹H NMR (400 MHz, DMSO-d₆): δ 10.43 (bs, 1H), 9.49 (bs, 1H), 8.83 (s, 1H), 8.37 (s, 1H), 8.13 (s, 1H), 7.91 (d, J=8.0 Hz, 2H), 7.59 (s, 1H), 7.34 (d, J=8.4 Hz, 2H), 7.06-7.01 (m, 3H), 5.38 (s, 2H), 4.00 (s, 3H), 2.02 (s, 6H). LC-MS: m/z 524.1 ([M−2H]⁻).

### Preparation of 4-((4-chloro-1H-pyrazol-1-yl) methyl)-N-(5(N (2,6dimethylphenyl)sulfamoyl)-6-methoxypyridin-3-yl)benzamide (1398)

Prepared by Method-B by reacting 5-amino-N-(2,6-dimethylphenyl)-2-methoxypyridine-3-sulfonamide (24) with 3-chloro-4-methylthiophene-2-carboxylic acid to give an off-white solid (60% yield). ¹H NMR (400 MHz, DMSO-d₆): δ 10.40 (bs, 1H), 9.48 (bs, 1H), 8.71 (s, 1H), 8.30 (s, 1H), 7.65 (s, 1H), 7.08-7.01 (m, 3H), 4.00 (s, 3H), 2.19 (s, 3H), 2.03 (s, 6H). LC-MS: m/z 463.90 ([M−2H]⁻).

### Preparation of N-(5-(N-(2,6-dimethylphenyl)sulfamoyl)-6-methoxypyridin-3-yl)-3-(5-methyl-1,2,4-oxadiazol-3-yl)benzamide (1401)

Prepared by Method-B by reacting 5-amino-N-(2, 6-dimethylphenyl)-2-methoxypyridine-3-sulfonamide (24) with 3-(5-methyl-1,2,4-oxadiazol-3-yl)benzoic acid to give an off-white solid (33% yield). ¹H NMR (400 MHz, DMSO-d₆): δ 10.68 (bs, 1H), 9.50 (bs, 1H), 8.86 (d, J=2.4 Hz, 1H), 8.59 (s, 1H), 8.40 (d, J=2.8 Hz, 1H), 8.21-8.15 (m, 2H), 7.73 (t, J=8 Hz, 1H), 7.07-7.01 (m, 3H), 4.02 (s, 3H), 2.69 (s, 3H), 2.04 (s, 3H), 1.99 (s, 3H). LC-MS: m/z 494.2 ([M+H]⁺).

### Preparation of N-(5-(N-(2, 6-dimethylphenyl) sulfamoyl)-6-methoxypyridin-3-yl)-2-methylbenzo[d]thiazole-6-carboxamide (1400)

Prepared by Method-B by reacting 5-amino-N-(2, 6-dimethylphenyl)-2-methoxypyridine-3-sulfonamide (24) with 2-methylbenzo[d]thiazole-6-carboxylic acid to give a light yellow solid (38% yield). ¹H NMR (400 MHz, DMSO-d₆): δ 10.56 (bs, 1H), 9.48 (bs, 1H), 8.86 (d, J=2.4 Hz, 1H), 8.64 (s, 1H), 8.41 (d, J=2.4 Hz, 1H), 8.03 (s, 2H), 7.05-7.01 (m, 3H), 4.01 (s, 3H), 2.85 (s, 3H), 2.04 (s, 6H). LC-MS: 483.0 ([M+H]⁺).

### Preparation of N-(5-(N-(2, 6-dimethylphenyl) sulfamoyl)-6-methoxypyridin-3-yl)-2-fluoro-6-(trifluoromethoxy) benzamide (1396)

Prepared by Method-B by reacting 5-amino-N-(2,6-dimethylphenyl)-2-methoxypyridine-3-sulfonamide (24) with 2-fluoro-6-(trifluoromethoxy)benzoic acid to give an off-white solid (55% yield). ¹H NMR (400 MHz, DMSO-d₆): δ 10.8 (bs, 1H), 9.49 (bs, 1H), 8.68-8.67 (m, 1H), 8.34-8.33 (m, 1H), 7.71-7.68 (m, 1H), 7.60-7.52 (m, 2H), 7.01-7.09 (m, 3H), 4.03 (s, 3H), 2.03 (s, 6H). LC-MS: m/z 514.1 ([M+H]⁺).

### Preparation of N-(5-(N-(2,6-dimethylphenyl)sulfamoyl)-6-methoxypyridin-3-yl)-1-methyl-1H-benzo[d]imidazole-7-carboxamide (1394)

Prepared by Method-B by reacting 5-amino-N-(2,6-dimethylphenyl)-2-methoxypyridine-3-sulfonamide (24) with 1-methyl-1H-benzo[d]imidazole-7-carboxylic acid to give a white solid (66% yield). ¹H NMR (400 MHz, DMSO-d₆): δ 10.83 (bs, 1H), 9.49 (bs, 1H), 8.98 (d, J=2.4 Hz, 1H), 8.46 (d, J=2.4 Hz, 1H), 8.26 (bs, 1H), 7.83-7.81 (m, 1H), 7.53-7.51 (m, 1H), 7.31-7.02 (m, 4H). LC-MS: m/z 466.0 ([M+H]⁺).

### Preparation of N-(5-(N-(2,6-dimethylphenyl)sulfamoyl)-6-methoxypyridin-3-yl)-1-methyl-1H-pyrrolo[2,3-b]pyridine-3-carboxamide (1395)

Prepared by Method-B by reacting 5-amino-N-(2, 6-dimethylphenyl)-2-methoxypyridine-3-sulfonamide in (24) with 1-methyl-1H-pyrrolo[2, 3-b]pyridine-3-carboxylic acid to give a brown solid (40% yield). ¹H NMR (400 MHz, DMSO-d₆): δ 10.09 (bs, 1H), 9.48 (bs, 1H), 8.84 (d, J=2.4 Hz, 1H), 8.49-8.46 (m, 1H), 8.38-8.35 (m, 3H), 7.27-7.24 (m, 1H), 7.09-7.01 (m, 3H), 4.01 (s, 3H), 3.89 (s, 3H), 2.04 (s, 6H). LC-MS: m/z 466.2 ([M+H]⁺).

### Preparation of N-(5-(N-(2, 6-dimethylphenyl) sulfamoyl)-6-methoxypyridin-3-yl)-1,3-dimethyl-1H-thieno[2,3-c]pyrazole-5-carboxamide (1380)

Prepared by Method-B by reacting 5-amino-N-(2,6-dimethylphenyl)-2-methoxypyridine-3-sulfonamide (24) with 1,3-dimethyl-1H-thieno[2,3-c]pyrazole-5-carboxylic acid to give an off-white solid (41% yield). ¹H NMR (400 MHz, DMSO-d₆): δ 10.44 (bs, 1H), 9.48 (bs, 1H), 8.80 (bs, 1H), 7.84 (d, J=7.2 Hz, 1H), 7.67 (d, J=6.8 Hz, 1H), 7.38-7.26 (m, 2H), 7.07-7.01 (m, 4H), 3.92 (s, 3H), 2.05 (s, 6H). LC-MS: m/z 486.05 ([M+H]⁺).

### Preparation of N-(5-(N-(2, 6-dimethylphenyl) sulfamoyl)-6-methoxypyridin-3-yl)-2,2-dimethyl-2,3-dihydrobenzofuran-7-carboxamide (1379)

Prepared by Method-B by reacting 5-amino-N-(2, 6-dimethylphenyl)-2-methoxypyridine-3-sulfonamide (24) with 2, 2-dimethyl-2,3-dihydrobenzofuran-7-carboxylic acid to give an off-white solid (16% yield). ¹H NMR (400 MHz, DMSO-d₆): δ 9.83 (s, 1H), 9.46 (s, 1H), 8.65 (d, J=2.4 Hz, 1H), 8.42 (d, J=2.0 Hz, 1H), 7.57 (d, J=7.2 Hz, 1H), 7.39 (d, J=7.2 Hz, 1H), 7.09-6.92 (m, 4H), 4.00 (s, 3H), 3.07 (s, 2H), 2.04 (s, 6H), 1.51 (s, 6H). LC-MS: m/z 480.05 ([M−H]⁻).

### Preparation of N-(4-fluoro-2, 6-dimethylphenyl)-2-methoxy-5-nitropyridine-3-sulfonamide (25)

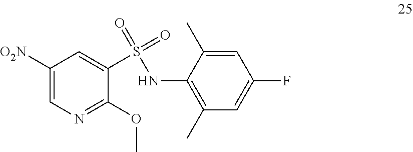 [SMILES: COc1ncc([N+](=O)[O-])cc1S(=O)(=O)Nc1c(C)cc(F)cc1C]

Prepared by Method-K by reacting 2-methoxy-5-nitropyridine-3-sulfonyl chloride (20) with 4-fluoro-2,6-dimethylaniline to give a yellow solid (85% yield).

### Step-4: Preparation of 5-amino-N-(4-fluoro-2, 6-dimethylphenyl)-2-methoxypyridine-3-sulfonamide (26)

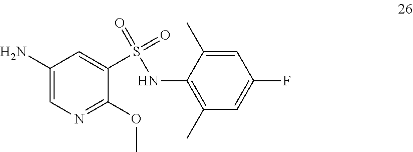 [SMILES: COc1ncc(N)cc1S(=O)(=O)Nc1c(C)cc(F)cc1C]

Prepared by following compound 9 preparation procedure to give an off-white solid (83% yield). ¹H NMR (400 MHz, DMSO-d₆): δ 9.21 (bs, 1H), 7.71 (d, J=2.4 Hz, 1H), 7.29 (d, J=2.4 Hz, 1H), 6.86 (d, J=9.6 Hz, 2H), 5.11 (bs, 2H), 3.86 (s, 3H), 2.01 (s, 6H). LC-MS: m/z 326.1 ([M+H]⁺).

### Preparation of N-(5-(N-(4-fluoro-2, 6-dimethylphenyl) sulfamoyl)-6-methoxypyridin-3-yl)-2-(4-fluorophenyl) oxazole-4-carboxamide (1937)

Prepared by Method-B by reacting 5-amino-N-(4-fluoro-2, 6-dimethylphenyl)-2-methoxypyridine-3-sulfonamide (26) with 2-(4-fluorophenyl)oxazole-4-carboxylic acid to give a white fluffy solid (61% yield). ¹H NMR (400 MHz, DMSO-d₆): δ 10.50 (bs, 1H), 9.49 (bs, 1H), 8.87 (d, J=4.0 Hz, 2H), 8.46 (d, J=2.0 Hz, 1H), 8.12 (dd, J₁=8.4 Hz, J₂=5.6 Hz, 2H), 7.45 (s, 2H), 6.89 (d, J=9.6 Hz, 2H), 4.01 (s, 3H), 2.03 (s, 6H). LC-MS: m/z 515.0 ([M+H]⁺).

### Preparation of N-(5-(N-(2,6-dimethylphenyl)sulfamoyl)-6-methoxypyridin-3-yl)-5,6-dihydro-4H-4,7-ethanothieno[2,3-b]pyridine-3-carboxamide (1946)

Prepared by method-C by reacting 5-amino-N-(2,6-dimethylphenyl)-2-methoxypyridine-3-sulfonamide (24) with ethyl 5,6-dihydro-4H-4,7-ethanothieno[2,3-b]pyridine-3-carboxylate (48) to give an off-white solid (49% yield). ¹H NMR (400 MHz, DMSO-d₆): δ 10.25 (bs, 1H), 9.46 (bs, 1H), 8.79 (d, J=2.4 Hz, 1H), 8.34 (d, J=2.4 Hz, 1H), 7.89 (s, 1H), 7.06-7.01 (m, 3H), 4.00 (s, 3H), 3.86 (bs, 1H), 3.35-3.30 (m, 2H), 3.13-3.11 (m, 2H), 2.03 (s, 6H), 1.72-7.79 (m, 2H), 1.28-1.38 (m, 2H). LC-MS: m/z 499.4 ([M+H]⁺).

### Preparation of N-(5-(N-(4-chlorophenyl) sulfamoyl)-6-methoxypyridin-3-yl)-5,6-dihydro-4H-4,7-ethanothieno[2,3-b]pyridine-3-carboxamide (1944)

Prepared by method-C by reacting 5-amino-N-(4-chlorophenyl)-2-methoxypyridine-3-sulfonamide (22) with ethyl 5,6-dihydro-4H-4,7-ethanothieno[2,3-b]pyridine-3-carboxylate (48) to give an off-white solid (8% yield). ¹H NMR (400 MHz, CD₃OD): δ 8.62 (d, J=2.8 Hz, 1H), 8.53 (d, J=2.8 Hz, 1H), 7.80 (s, 1H), 7.22-7.20 (m, 2H), 7.14-7.12 (m, 2H), 4.06 (s, 3H), 3.95 (t, J=2.8 Hz, 1H), 3.28-3.25 (m, 2H), 2.66-2.22 (m, 2H), 1.95-1.85 (m, 2H), 1.55-1.48 (m, 2H). LC-MS: m/z 503.0 ([M−2H]⁻).

### Preparation of N-(5-(N-(2, 6-dimethylphenyl) sulfamoyl)-6-methoxypyridin-3-yl)-[2,3′-bipyridine]-4-carboxamide (1940)

Prepared by method-C by reacting 5-amino-N-(2, 6-dimethylphenyl)-2-methoxypyridine-3-sulfonamide (24) with methyl [2,3′-bipyridine]-4-carboxylate (50) to give an off-white solid (42% yield). ¹H NMR (400 MHz, DMSO-d₆): δ 10.76 (bs, 1H), 9.54 (bs, 1H), 9.33 (s, 1H), 8.91-8.88 (m, 2H), 8.68 (s, 1H), 8.51-8.50 (m, 2H), 8.37 (d, J=2.4 Hz, 1H), 7.85 (dd, J₁=5.2 Hz, J₂=1.6 Hz, 1H), 7.57 (dd, J₁=7.6 Hz, J₂=4.8 Hz, 1H), 7.09-7.02 (m, 3H), 4.04 (s, 3H), 2.04 (s, 6H). LC-MS: m/z 490.2 ([M+H]⁺).

### Preparation of N-(5-(N-(2, 6-dimethylphenyl) sulfamoyl)-6-methoxypyridin-3-yl)-5, 6, 7, 8-tetrahydro-4H-cyclohepta[b]thiophene-3-carboxamide (1486)

Prepared by method-C by reacting 5-amino-N-(2, 6-dimethylphenyl)-2-methoxypyridine-3-sulfonamide (24) with ethyl 5,6,7,8-tetrahydro-4H-cyclohepta[b]thiophene-3-carboxylate (53) to give an off-white solid (38% yield). ¹H NMR (400 MHz, DMSO-d₆): δ 10.33 (s, 1H), 9.44 (s, 1H), 8.73 (s, 1H), 8.37 (s, 1H), 7.68 (s, 1H), 7.06-7.01 (m, 3H), 3.99 (s, 3H), 2.88-2.80 (m, 4H), 2.03 (s, 6H), 1.82-1.52 (m, 6H). LC-MS: m/z 484.2 ([M−H]⁻).

### Preparation of N-(5-(N-(2,6-dimethylphenyl)sulfamoyl)-6-methoxypyridin-3-yl)-5,6-dihydro-4H-cyclopenta[b]thiophene-3-carboxamide (1425)

Prepared by method-C by reacting 5-amino-N-(2, 6-dimethylphenyl)-2-methoxypyridine-3-sulfonamide (24) with ethyl 5,6-dihydro-4H-cyclopenta[b]thiophene-3-carboxylate (51) to give an off-white solid (42% yield). ¹H NMR (400 MHz, DMSO-d₆): δ 10.16 (bs, 1H), 9.46 (bs, 1H), 8.76 (d, J=2.4 Hz, 1H), 8.34 (d, J=2.4 Hz, 1H), 8.13 (s, 1H), 7.06-7.01 (m, 3H), 4.00 (s, 3H), 2.86-2.81 (m, 4H), 2.44-2.38 (m, 2H), 2.02 (s, 6H). LC-MS: m/z 458.0 ([M+H]⁺).

### Preparation of 6-bromo-N-(4-chlorophenyl) pyridine-2-sulfonamide (27)

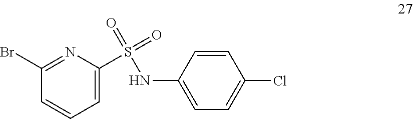 [SMILES: O=S(=O)(Nc1ccc(Cl)cc1)c1cccc(Br)n1]

Prepared by reacting 6-bromopyridine-2-sulfonyl chloride (Method-K) with 4-chloro-aniline to give a brown solid (92% yield). LC-MS: m/z 348.7 ([M+2H]⁺).

### Preparation of 6-amino-N-(4-chlorophenyl) pyridine-2-sulfonamide (28)

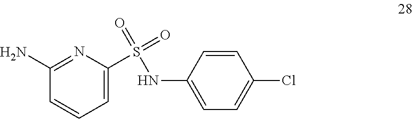 [SMILES: Nc1cccc(S(=O)(=O)Nc2ccc(Cl)cc2)n1]

A solution of 6-bromo-N-(4-chlorophenyl)pyridine-2-sulfonamide (27, 0.200 g, 0.578 mmol, 1.0 eq.) in NH₄OH (5.0 mL) at room temperature was added CuSO₄.5H₂O (0.092 g, 0.578 mmol, 1.0 eq.) and allowed to heat at 100° C. for 16 h in a steel bomb. After completion of reaction (monitored by TLC), cooled to room temperature, the reaction mixture was diluted with EtOAc (100 mL), organic layer was transferred to a separatory funnel and washed with water (2×100 mL) and brine solution (100 mL). The organic layer was dried over anhydrous Na₂SO₄ and concentrated under reduced pressure. The crude product was purified by Combi-flash by using 50-60% of EtOAc/hexanes to obtain 28 (0.100 g, 0.353 mmol, 61% yield) as a brown solid. LC-MS: m/z 281.95 ([M−H]⁻).

### Preparation of N-(6-(N-(4-chlorophenyl) sulfamoyl) pyridin-2-yl)-2-phenyl-1H-imidazole-5-carboxamide (612)

Prepared by method-C by reacting 6-amino-N-(4-chlorophenyl) pyridine-2-sulfonamide (28) with methyl 2-phenyl-1H-imidazole-5-carboxylate (54) to give a brown solid (17% yield). ¹H NMR (400 MHz, DMSO-d₆): δ 13.7 (bs, 1H), 10.68 (bs, 1H), 9.67 (bs, 1H), 8.42 (d, J=8.8 Hz, 1H), 8.14-8.03 (m, 4H), 7.56-7.31 (m, 5H), 7.21-7.19 (m, 2H). LC-MS: m/z 453.9 ([M+H]⁺).

### Preparation of 5-bromopyridine-3-sulfonic acid (29)

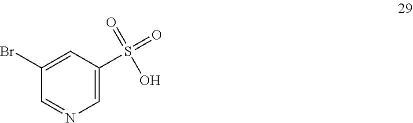 [SMILES: O=S(=O)(O)c1cncc(Br)c1]

A solution of pyridine-3-sulfonyl chloride (12.5 g, 70.38 mmol, 1.0 eq.) in bromine (4.2 mL, 84.46 mmol, 1.2 eq.) at room temperature was heated to 130° C. and reacted for 12 h. After completion of reaction (monitored by TLC), the mixture was cooled to room temperature, water was added (150 mL) and then heated at 90° C. for 2 h. The mixture was concentrated to a small volume and diluted with acetone to obtain solids which were filtered and dried under vacuum to afford 29 (7.50 g, 31.65 mmol, 45% yield) as a brown solid.

### Preparation of 5-bromopyridine-3-sulfonyl chloride (30)

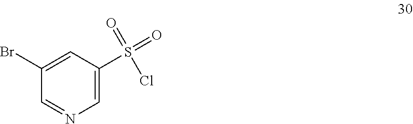 [SMILES: O=S(=O)(Cl)c1cncc(Br)c1]

To a solution of 5-bromopyridine-3-sulfonic acid (29, 15.0 g, 63.29 mmol, 1.0 eq.) in POCl₃ (450 mL) at room temperature, was added PCl₅ (43.79 g, 126.6 mmol, 1.0 eq.) and the reaction was heated at reflux for 16 h. After completion of reaction (monitored by TLC), the mixture was cooled to room temperature, concentrated under reduced pressure and poured into ice cold water and the formed solids were filtered and dried under vacuum to afford 30 (12.0 g, 47.06 mmol, 74% yield) as a brown solid.

### Preparation of 5-bromo-N-(2-(trifluoromethyl) phenyl)pyridine-3-sulfonamide (31)

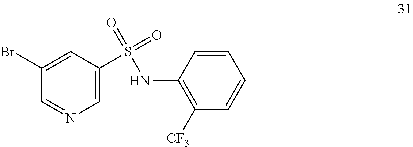 [SMILES: O=S(=O)(Nc1ccccc1C(F)(F)F)c1cncc(Br)c1]

Prepared by reacting 5-bromopyridine-3-sulfonyl chloride (30) (Method-K) with 2-(trifluoromethyl) aniline to give a yellow solid (33% yield). LC-MS: m/z 380.9 ([M+H]⁺).

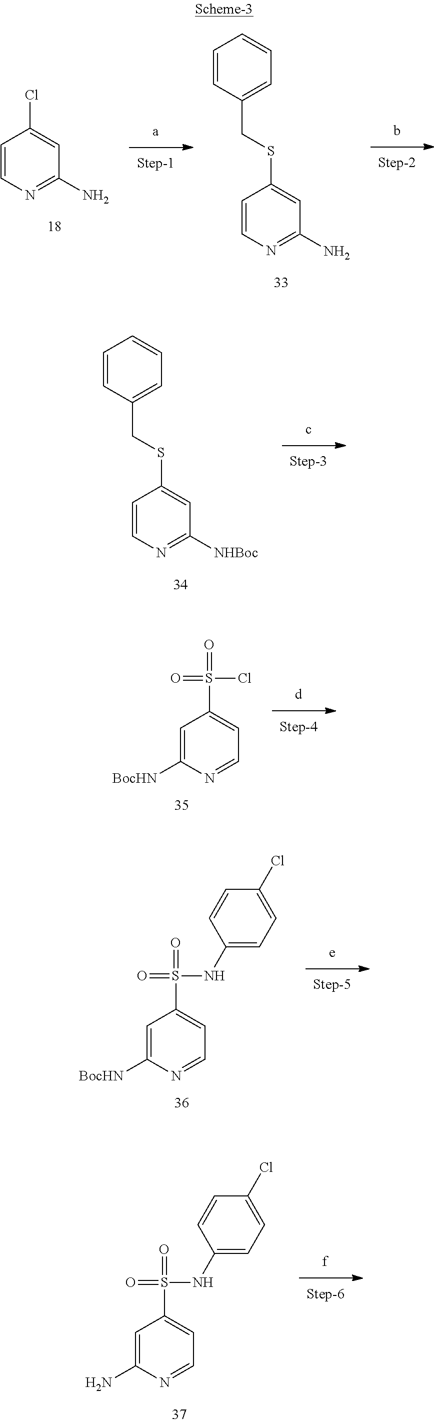 [SMILES: Nc1cc(Cl)ccn1 ; Nc1cc(SCc2ccccc2)ccn1 ; CC(C)(C)OC(=O)Nc1cc(SCc2ccccc2)ccn1 ; CC(C)(C)OC(=O)Nc1cc(S(=O)(=O)Cl)ccn1 ; CC(C)(C)OC(=O)Nc1cc(S(=O)(=O)Nc2ccc(Cl)cc2)ccn1 ; Nc1cc(S(=O)(=O)Nc2ccc(Cl)cc2)ccn1]

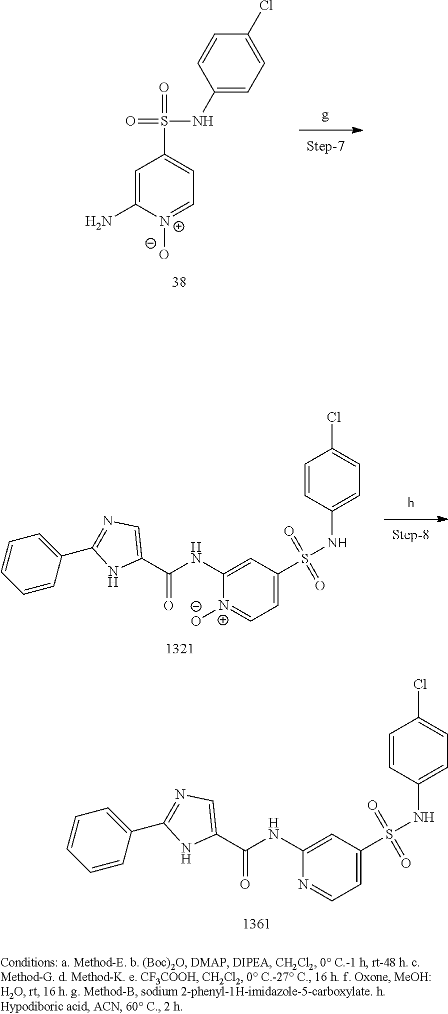 [SMILES: Nc1cc(S(=O)(=O)Nc2ccc(Cl)cc2)cc[n+]1[O-] ; O=C(Nc1cc(S(=O)(=O)Nc2ccc(Cl)cc2)cc[n+]1[O-])c1cnc(-c2ccccc2)[nH]1 ; O=C(Nc1cc(S(=O)(=O)Nc2ccc(Cl)cc2)ccn1)c1cnc(-c2ccccc2)[nH]1]

### Experimental Procedures for Scheme-3

### Scheme-3, Step-1: Preparation of 4-(benzylthio) pyridin-2-amine (33)

Prepared by Method-E by reacting 4-chloropyridin-2-amine (32) with phenylmethanethiol to give a white solid (55% yield).

### Scheme-3, Step-2: Preparation of tert-butyl (4-(benzylthio) pyridin-2-yl)carbamate (34)

To a stirred solution of 4-(benzylthio)pyridin-2-amine (33, 6.0 g, 0.028 mol, 1.0 eq.) in dichloromethane (200 mL) was added DMAP (0.338 g, 0.0027 mol, 0.1 eq.) followed by DIPEA (5.8 mL, 0.0332 mol, 1.2 eq.) and the reaction was stirred at 0° C. To this solution was added Boc anhydride (8.2 mL, 0.036 mol, 1.3 eq.) in dichloromethane (100 mL) dropwise over 1 h, and the reaction mixture was allowed to warm to room temperature over 48 h. After completion of reaction (monitored by TLC), the reaction mixture was washed with water (300 mL), the organic layer was separated and washed with brine (150 mL) and dried over anhydrous Na₂SO₄. The mixture was concentrated under reduced pressure to afford crude product which was stirred with diethyl ether (100 mL) for 20 min. The resulting solid was collected by filtration to obtain 34 (7.6 g) as a pale brown solid.

### Scheme-3, Step-3: Preparation of tert-butyl (4-(benzylthio) pyridin-2-yl)carbamate (35)

Prepared by Method-G in Scheme-1, step-3. Crude product was taken for the next step directly.

### Scheme-3, Step-4: Preparation of tert-butyl (4-(chlorosulfonyl) pyridin-2-yl)carbamate (36)

Prepared by Method-K by reacting tert-butyl (4-(chlorosulfonyl) pyridin-2-yl) carbamate (35) with 4-chloroaniline to give a yellow solid (36% yield). LC-MS: m/z 381.9 [(M−2H)⁻].

### Scheme-3, Step-5: Preparation of 2-amino-N-(4-chlorophenyl) pyridine-4-sulfonamide (37)

To a stirred solution of tert-butyl (4-(N-(4-chlorophenyl) sulfamoyl) pyridin-2-yl) carbamate (36, 1.4 g, 3.64 mmol, 1.0 eq.) in CH₂Cl₂ (30 mL) was added CF₃COOH (5 mL) at 0° C. The reaction mixture was stirred at 27° C. for 16 h. The reaction mixture was diluted with CH₂Cl₂ (250 mL) and was washed with aq. NaHCO₃ solution (150 mL), then dried over sodium sulphate and concentrated under reduced pressure. Crude compound was purified by Combi-flash by eluting with (EtOAc: hexanes/7:3) to obtain 37 (0.45 g, 1.59 mmol, 44% yield), as a pale brown solid. LC-MS: m/z 281.95 ([M−H]⁻).

### Scheme-3, Step-6: Preparation of 2-amino-4-(N-(4-chlorophenyl) sulfamoyl) pyridine 1-oxide (38)

To a stirred solution of 2-amino-N-(4-chlorophenyl)-pyridine-4-sulfonamide (37, 0.45 g, 1.58 mmol, 1.0 eq.) in MeOH:H₂O (3:1, 20 mL) was added Oxone (1.46 g, 4.76 mmol, 3.0 eq.) at 0° C. and stirred at room temperature for 6 h. The reaction mixture was diluted with methanol (40 mL) and filtered through celite. The filtrate was concentrated under reduced pressure and the crude product was purified by combi-flash eluting with (MeOH: CH₂Cl₂/10:90) to obtain 38 as a yellow sticky solid. LC-MS: m/z 300.0 ([M+H]⁺).

### Scheme-3, Step-7: Preparation of 4-(N-(4-chlorophenyl) sulfamoyl)-2-(2-phenyl-1H-imidazole-5-carboxamido) pyridine 1-oxide (1321)

Prepared by Method-B by reacting 2-amino-4-(N-(4-chlorophenyl) sulfamoyl) pyridine 1-oxide (38) with sodium 2-phenyl-1H-imidazole-5-carboxylate (46) to give a white solid (20% yield). ¹H NMR (400 MHz, DMSO-d₆): δ 13.45 (bs, 1H), 11.25 (s, 1H), 10.80 (bs, 1H), 8.80 (d, J=2.4 Hz, 1H), 8.54 (d, J=6.8 Hz, 1H), 8.19 (s, 1H), 8.03-8.01 (m, 2H), 7.55-7.45 (m, 3H), 7.34-7.31 (m, 3H), 7.12 (d, J=8.4 Hz, 2H). LC-MS: m/z 467.9 [(M−2H)⁻].

### Scheme-3, Step-8: Preparation of N-(4-(N-(4-chlorophenyl) sulfamoyl) pyridin-2-yl)-2-phenyl-1H-imidazole-5-carboxamide (1361)

To a stirred solution of 4-(N-(4-chlorophenyl) sulfamoyl)-2-(2-phenyl-1H-imidazole-5-carboxamido) pyridine 1-oxide (1321, 0.1 g, 0.213 mmol, 1.0 eq.) in ACN (5 mL) was added hypodiboric acid (0.038 g, 0.426 mmol, 2.0 eq.) at room temperature. The reaction mixture was stirred at 70° C. for 1 h. After completion of the reaction (monitored by TLC), acetonitrile was removed under reduced pressure and the obtained residue was diluted with EtOAc (20 mL). The organics were washed with water (10 mL) and brine solution (10 mL) and the organic layer was dried over Na₂SO₄ and concentrated under reduced pressure. The obtained solid was washed with methanol (5 mL) and pentane (20 mL) and dried under reduced pressure to obtain 1361 (0.025 g, 0.055 mmol, 26% yield) as a yellow solid. ¹H NMR (400 MHz, DMSO-d₆): δ 13.34 (bs, 1H), 10.86 (bs, 1H), 9.95 (bs, 1H), 8.63 (s, 1H), 8.57 (d, J=5.2 Hz, 1H), 8.14 (d, J=2.4 Hz, 1H), 8.06-8.04 (m, 2H), 7.51-7.34 (m, 6H), 7.16-7.14 (m, 2H). LC-MS: m/z 454.1 ([M+H]⁺).

### Preparation of 4-(N-(4-chlorophenyl)sulfamoyl)-2-(4-methyloxazole-5-carboxamido)pyridine 1-oxide (1294)

Prepared by Method-B by reacting 2-amino-4-(N-(4-chlorophenyl) sulfamoyl) pyridine 1-oxide (38) with 4-methyloxazole-5-carboxylic acid to give a white solid (50% yield). ¹H NMR (400 MHz, CD₃OD): δ 8.74 (d, J=2.4 Hz, 1H), 8.34 (d, J=6.8 Hz, 1H), 8.23 (s, 1H), 7.36-7.34 (m, 1H), 7.17-7.15 (m, 3H), 7.05-7.03 (m, 2H), 2.46 (s, 3H). LC-MS: m/z 406.9 ([M−2H]⁻).

### Preparation of 4-(benzylthio)-5-methylpyridin-2-amine (39)

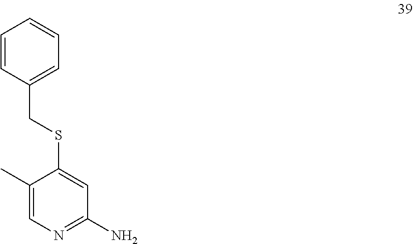 [SMILES: Cc1cnc(N)cc1SCc1ccccc1]

Prepared by Method-E by reacting 4-bromo-5-methylpyridin-2-amine with phenylmethanethiol to give a white solid (86% yield).

### Preparation of tert-butyl (4-(benzylthio)-5-methylpyridin-2-yl) carbamate (40)

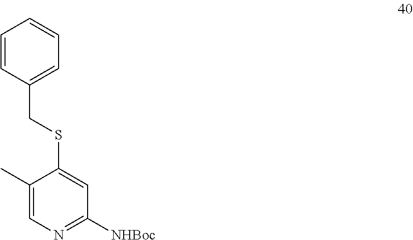 [SMILES: Cc1cnc(NC(=O)OC(C)(C)C)cc1SCc1ccccc1]

Prepared as per Scheme-3, step-2, to give a white solid (66% yield).

### Preparation of tert-butyl (4-(chlorosulfonyl)-5-methylpyridin-2-yl) carbamate (41)

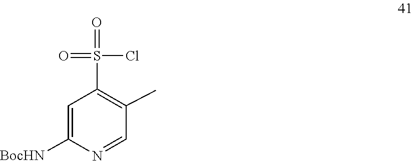 [SMILES: Cc1cnc(NC(=O)OC(C)(C)C)cc1S(=O)(=O)Cl]

Prepared by Method-G to afford product as a yellow sticky solid. LC-MS: m/z 287.0 ([M−H]⁻) (Sulfonic acid).

### Preparation of tert-butyl (4-(N-(4-chlorophenyl) sulfamoyl)-5-methylpyridin-2-yl) carbamate (42)

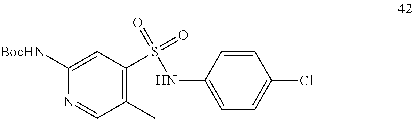 [SMILES: Cc1cnc(NC(=O)OC(C)(C)C)cc1S(=O)(=O)Nc1ccc(Cl)cc1]

Prepared by Method-K by reacting tert-butyl (4-(chlorosulfonyl)-5-methylpyridin-2-yl) carbamate (41) with 4-chloroaniline to give an orange red solid (37% yield). LC-MS: m/z 395.9 ([M−2H]⁻).

### Preparation of 2-amino-N-(4-chlorophenyl)-5-methylpyridine-4-sulfonamide (43)

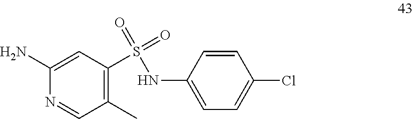 [SMILES: Cc1cnc(N)cc1S(=O)(=O)Nc1ccc(Cl)cc1]

Prepared by reacting tert-butyl (4-(N-(4-chlorophenyl) sulfamoyl)-5-methylpyridin-2-yl) carbamate (42) as per Scheme-3/step-5 to give a pale brown solid (37% yield). LC-MS: m/z 295.9 ([M−2H]⁻).

### Preparation of 2-amino-4-(N-(4-chlorophenyl) sulfamoyl)-5-methylpyridine 1-oxide (44)

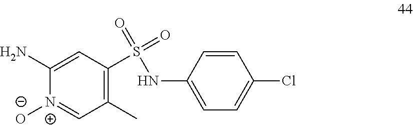 [SMILES: Cc1c[n+]([O-])c(N)cc1S(=O)(=O)Nc1ccc(Cl)cc1]

To a stirred solution of 2-amino-N-(4-chlorophenyl)-5-methylpyridine-4-sulfonamide (43, 0.5 g, 1.67 mmol, 1.0 eq.) in CH₂Cl₂ (20 mL) was added m-CPBA (0.86 g, 5.03 mmol, 3.0 eq.) at 0° C. and then stirred at room temperature for 2 h. The reaction mixture was concentrated under vacuum at which time a solid precipitated. The solid was washed with NaHCO₃ solution (15 mL) and the remaining solid was filtered and washed with water (4 mL) and pentane (50 mL) to afford 44 (0.3 g, 0.956 mmol, 57% yield) as a pale brown solid. LC-MS: m/z 311.9 ([M−2H]⁻).

### Preparation of 4-(N-(4-chlorophenyl) sulfamoyl)-5-methyl-2-(5-phenyloxazole-2-carboxamido) pyridine 1-oxide (1420)

Prepared by Method-B, by reacting 2-amino-4-(N-(4-chlorophenyl) sulfamoyl) pyridine 1-oxide (44) with 5-phenyloxazole-2-carboxylic acid (45) to give a yellow solid (16% yield). ¹H NMR (400 MHz, DMSO-d₆): δ 10.97 (bs, 1H), 10.95 (bs, 1H), 8.76 (s, 1H), 8.61 (s, 1H), 8.08 (s, 1H), 7.86 (d, J=7.2 Hz, 2H), 7.57-7.55 (m, 3H), 7.33 (d, J=7.2 Hz, 2H), 7.12 (d, J=8.4 Hz, 2H), 2.32 (s, 3H). LC-MS: m/z 485.1 ([M+H]⁺).

### Preparation of N-(4-(N-(4-chlorophenyl) sulfamoyl)-5-methylpyridin-2-yl)-5-phenyloxazole-2-carboxamide (1390)

Prepared by following compound 1361 preparation procedure to give a yellow solid (64% yield). ¹H NMR (400 MHz, DMSO-d₆): δ 11.17 (bs, 1H), 10.96 (bs, 1H), 8.59 (bs, 1H), 8.50 (bs, 1H), 8.03 (bs, 1H), 7.94 (d, J=7.6 Hz, 2H), 7.57-7.47 (m, 3H), 7.33 (d, J=8.8 Hz, 2H), 7.13 (d, J=8.4 Hz, 2H), 2.50 (bs, 3H). LC-MS: m/z 466.9 ([M−2H]⁻).

### Synthesis of Intermediates

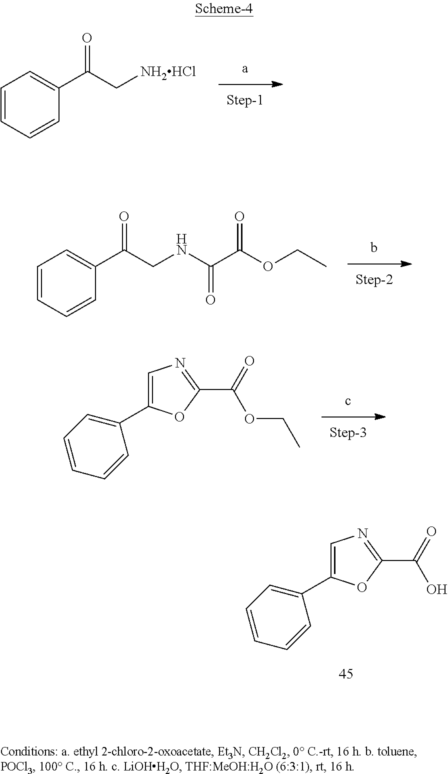

### Scheme-4, Step-1: Preparation of ethyl 2-oxo-2-((2-oxo-2-phenylethyl) amino) acetate

To a stirred solution of 2-amino-1-phenylethan-1-one hydrochloride (5.0 g, 0.0291 mol, 1.0 eq.) in dichloromethane (50 mL) were added Et₃N (12.0 mL, 0.0873 mol, 3.0 eq.) at 0° C. The reaction mixture was stirred at same temperature for 30 min, ethyl 2-chloro-2-oxoacetate (3.3 mL, 0.029 mol, 1.0 eq.) was added at room temperature. The resulting reaction mixture was stirred at same temperature for 16 h. The reaction mixture was diluted with EtOAc (100 mL). The organic layer was transferred to a separatory funnel and washed with water (50 mL) and brine (50 mL). The organic layer was dried over anhydrous Na₂SO₄ and concentrated under reduced pressure. The crude product was purified by Combi-flash by eluting with EtOAc/hexanes (3:7) to obtain product (5.0 g, 0.0212 mol, 73% yield) as a brown solid.

### Scheme-4, Step-2: Preparation of ethyl 5-phenyloxazole-2-carboxylate

To a stirred solution of ethyl 2-oxo-2-((2-oxo-2-phenylethyl amino) acetate (3.0 g, 0.0127 mol, 1.0 eq.) in toluene (30 mL) was added POCl₃ (4.5 mL, 0.0510 mol, 4.5 eq.) at room temperature. The reaction mixture was stirred at same temperature for 30 min. The resulting reaction mixture was stirred at 100° C. for 16 h. The reaction mixture was concentrated, quenched with ice cold water and diluted with EtOAc (100 mL). The organic layer was transferred to a separatory funnel and washed with water (50 mL) and brine (50 mL). The organic layer was dried over anhydrous Na₂SO₄ and concentrated under reduced pressure. The crude product was purified by Combi-flash by eluting with EtOAc/hexane (1:9) to obtain product (2.2 g, 0.0101 mol, 79% yield) as a brown solid.

### Scheme-4, Step-3: Preparation of 5-phenyloxazole-2-carboxylic acid (45)

To a stirred solution of ethyl 5-phenyloxazole-2-carboxylate (1.0 g, 0.0046 mol, 1.0 eq.) in THF:MeOH:H₂O (6:3:1) (25 mL) was added LiOH.H₂O (580 mg, 0.0138 mol, 3.0 eq.) at room temperature. The reaction mixture was stirred at same temperature for 16 h. The reaction mixture was diluted with EtOAc (100 mL) and acidified with 2N HCl. The organic layer was transferred to a separatory funnel and washed with water (50 mL) and brine (50 mL). The organic layer was dried over anhydrous Na₂SO₄ and concentrated under reduced pressure to obtain 45 (0.6 g, 0.0031 mol, 69% yield) as an off-white solid.

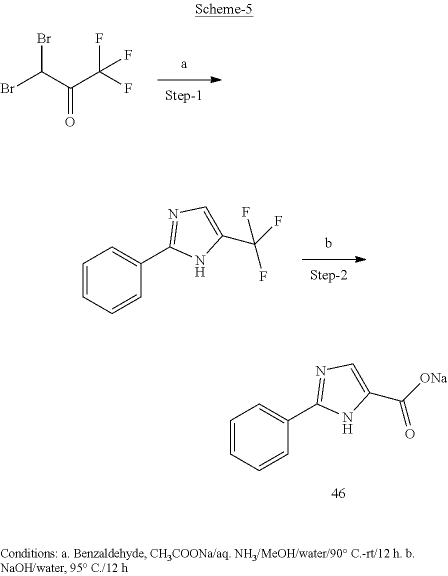

### Scheme-5, Step-1: Preparation of 2-phenyl-5-(trifluoromethyl)-1H-imidazole

1, 1-dibromo-3,3,3-trifluoro acetone (15.5 g, 0.057 mol, 1.11 eq.) was dissolved in aq. CH₃COONa solution (8.5 g, 0.0104 mol, 1.0 eq.) in 26 mL of water. It was stirred at 90° C. for 30 min and then cooled to room temperature. Benzaldehyde (5.5 g, 0.052 mol, 1 eq.) dissolved in a mixture of 160 mL methanol and 53 mL of aq. NH₃ was added. The reaction mixture was stirred overnight at room temperature. After completion of the reaction (monitored by TLC), the excess methanol was evaporated under reduced pressure, and ice water was added to the remaining aqueous phase. The solid which separated was filtered, washed with water (20 mL) and dried to obtain 2-phenyl-5-(trifluoromethyl)-1H-imidazole (9.0 g, 0.0424 mol, 74% yield) as an off-white solid. LC-MS: m/z 212.7 ([M+H]⁺).

### Scheme-5, Step-2: Preparation of sodium 2-phenyl-1H-imidazole-5-carboxylate (46)

To a solution of 2-phenyl-5-(trifluoromethyl)-1H-imidazole (9.0 g, 0.0424 mol, 1.0 eq.) in water (25 mL) was added NaOH (2.3 g, 0.0572 mol, 1.35 eq.) at 0° C. The resulting reaction mixture was gradually warmed to room temperature and then heated to 95° C. for 12 h. The reaction mixture was cooled to room temperature and diluted with water (10 mL). The contents were transferred to a separatory funnel, washed with dichloromethane (2×30 mL) and the layers were separated. The aqueous layer was neutralized with concentrated HCl. The aqueous solvent was removed by lyophilisation for 16 h to obtain the crude sodium 2-phenyl-1H-imidazole-5-carboxylate 46 (6.5 g crude) as a pale yellow solid. LC-MS: m/z 186.9 ([M−H]⁻).

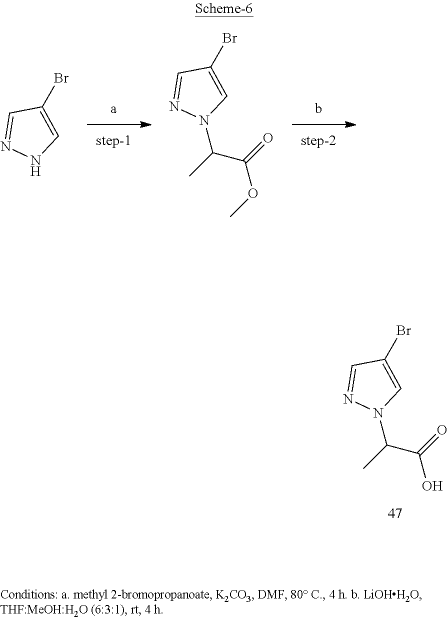

### Scheme-6, Step-1: Preparation of methyl 2-(4-bromo-1H-pyrazol-1-yl)propanoate

To a stirred solution of 4-bromo-1H-pyrazole (1.0 g, 6.80 mmol, 1.0 eq.) in DMF (10 mL) at room temperature was added K₂CO₃ (1.87 g, 13.61 mmol, 3.0 eq.) and methyl 2-bromopropanoate (1.70 g, 10.21 mmol, 1.5 eq.). The resulting reaction mixture was stirred at 80° C. for 4 h. After completion of the reaction (monitored by TLC), the mixture was diluted with water and extracted with EtOAc (150 mL). The combined organic extracts were washed with water (100 mL) followed by brine (100 mL). The organic layer was dried over anhydrous Na₂SO₄ and concentrated under reduced pressure. The crude product was purified by Combi-flash using EtOAc/hexanes as eluent to afford methyl 2-(4-bromo-1H-pyrazol-1-yl)propanoate (0.700 g, 3.017 mmol, 44% yield) as an off-white solid. LC-MS: m/z 234.85 ([M+2H]⁺).

### Scheme-6, Step-2: Preparation of 2-(4-bromo-1H-pyrazol-1-yl)propanoic acid (47)

Prepared by following compound 45 preparation procedure, to give an off-white solid (76% yield). LC-MS: m/z 216.95 ([M−H]⁻).

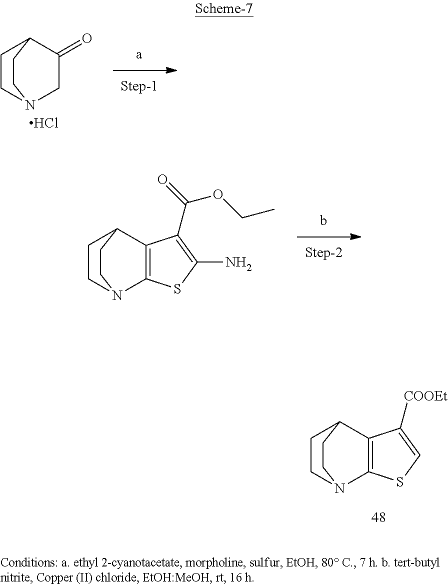 [SMILES: O=C1CN2CCC1CC2 ; Cl ; CCOC(=O)c1c(N)sc2c1C1CCN2CC1 ; CCOC(=O)c1csc2c1C1CCN2CC1]

### Scheme-7, Step-1: Preparation of ethyl 2-amino-5, 6-dihydro-4H-4,7-ethanothieno[2,3-b]pyridine-3-carboxylate

To a mixture of quinuclidin-3-one hydrochloride (1.5 g, 0.0093 mol, 1.0 eq.) and sulphur (0.328 g, 0.0102 mol, 1.1 eq.) in EtOH (30 mL) were added ethyl 2-cyanoacetate (1.154 g, 0.0102 mol, 1.1 eq.) and morpholine (1.62 g, 0.0186 mol, 2.0 eq.). The mixture was heated to 80° C. and stirred for 7 h. The reaction mixture was cooled to room temperature, diluted with water (100 mL) and extracted with EtOAc (2×250 mL), then the organic layer was washed with brine solution (100 mL) and dried under vacuum to afford crude product. The crude was purified by combi-flash by eluting with (EtOAc:hexanes/50:50) to obtain ethyl 2-amino-5, 6-dihydro-4H-4,7-ethanothieno[2,3-b]pyridine-3-carboxylate (1.8 g) as a yellow solid.

### Scheme-7, Step-2: Preparation of ethyl 5,6-dihydro-4H-4,7-ethanothieno[2,3-b]pyridine-3-carboxylate (48)

To a solution of copper (II) chloride (1.92 g, 0.0142 mol, 2.0 eq.) and tert-butyl nitrite (0.848 mL, 0.00713 mol, 1.0 eq.) in EtOH:MeOH (42 mL) was added ethyl 2-amino-5,6-dihydro-4H-4,7-ethanothieno[2,3-b]pyridine-3-carboxylate (1.8 g, 0.00713 mol, 1.0 eq.) at room temperature. After stirring for 16 h, the reaction mixture was diluted with water (50 mL) and EtOAc (250 mL). The organic layer was washed with water (75 mL), brine solution (75 mL) and then dried under vacuum to give crude product. The crude was purified by Combi-flash by eluting with (EtOAc:hexanes/35:65) to obtain ethyl 5,6-dihydro-4H-4,7-ethanothieno[2,3-b]pyridine-3-carboxylate 48 (1.0 g, 0.0042 mol, 59% yield) as a brown oil.

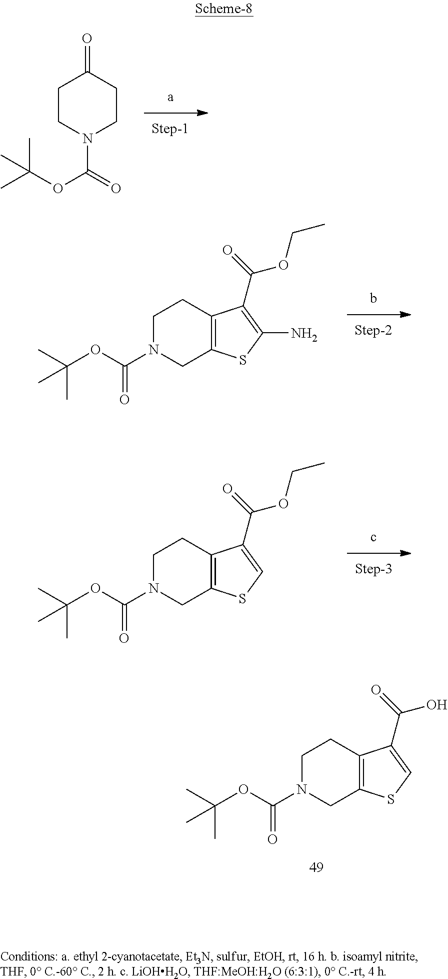

### Scheme-8, Step-1: Preparation of 6-(tert-butyl) 3-ethyl 2-amino-4,7-dihydrothieno[2,3-c]pyridine-3,6(5H)-dicarboxylate

To a stirred solution of ethyl 2-cyanoacetate (1.7 g, 15.06 mmol, 1.0 eq.) in Ethanol (25 mL) were added Et₃N (1.52 g, 15.06 mmol, 1.0 eq.) and sulfur (482 mg, 15.06 mmol, 1.0 eq.) at room temperature. The mixture was stirred for 15 min at the same temperature. To the above solution, tert-butyl 4-oxopiperidine-1-carboxylate (3.0 g, 15.06 mmol, 1.0 eq.) was added portion-wise and the resulting reaction mixture was stirred for 16 h at room temperature. After completion of the reaction (monitored by TLC), the reaction solid was filtered and washed with ethanol to obtain 6-(tert-butyl) 3-ethyl 2-amino-4,7-dihydrothieno[2,3-c]pyridine-3,6(5H)-dicarboxylate (3.0 g, 9.19 mmol, 61% yield) as a white solid.

### Scheme-8, Step-2: Preparation of 6-(tert-butyl) 3-ethyl 4,7-dihydrothieno[2,3-c]pyridine-3,6(5H)-dicarboxylate

To a stirred solution of 6-(tert-butyl)-3-ethyl-2-amino-4,7-dihydrothieno[2,3-c]pyridine-3,6(5H)-dicarboxylate (2.0 g, 6.12 mmol, 1.0 eq.) in THF (20 mL) was added isoamyl nitrite (2.44 g, 18.36 mmol, 3.0 eq.) at 0° C. and the resulting reaction mixture was stirred at 60° C. for 2 h. The reaction mixture was quenched with ice cold water (100 mL) and extracted with EtOAc. The crude product was purified by combi-flash by eluting with EtOAc/n-hexanes (3:7) to obtain 6-(tert-butyl)-3-ethyl-4,7-dihydrothieno[2,3-c]pyridine-3,6(5H)-dicarboxylate (700 mg, 2.247 mmol, 37% yield) as a yellow liquid. Structure was confirmed by ¹H NMR.

### Scheme-8, Step-3: Preparation of 6-(tert-butoxycarbonyl)-4,5,6,7-tetrahydrothieno[2,3-c]pyridine-3-carboxylic acid (49)

Was prepared by following compound 45 preparation procedure to give a white solid (66% yield). LC-MS: m/z 284 ([M+H]⁺).

### Preparation of methyl [2, 3′-bipyridine]-4-carboxylate (50)

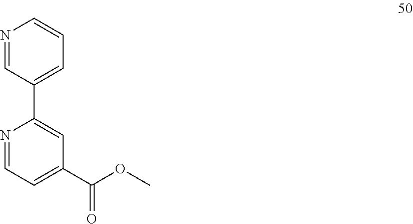 [SMILES: COC(=O)c1ccnc(-c2cccnc2)c1]

A stirred solution of methyl 2-bromoisonicotinate (2.0 g, 9.25 mmol, 1.0 eq.) and pyridin-3-ylboronic acid (1.13 g, 9.25 mmol, 1.0 eq.), K₂CO₃ (1.53 g, 11.1 mmol, 1.2 eq.) in ACN (20 mL), H₂O (4 mL) was degassed and then Pd(PPh₃)₄ (0.534 g, 0.46 mmol, 0.05 eq.) was added. The mixture was stirred for 24 h at 80° C. in nitrogen atmosphere. The reaction was monitored by TLC and after completion, the reaction was added to water and extracted with EtOAc (2×50 mL). The organic layer was separated, dried over Na₂SO₄, filtered and concentrated under vacuum. The crude compound was purified by Combi-flash eluting with (4:6/EtOAc:hexanes), fractions were collected and concentrated under reduced pressure to obtain methyl [2, 3′-bipyridine]-4-carboxylate 50 (0.5 g, 2.33 mmol, 25% yield) as a light yellow solid. LC-MS: m/z 215.05 ([M+H]⁺).

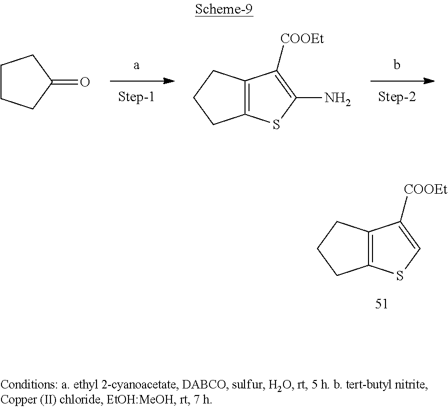 [SMILES: O=C1CCCC1 ; CCOC(=O)c1c(N)sc2c1CCC2 ; CCOC(=O)c1csc2c1CCC2]

### Scheme-9, Step-1: Preparation of ethyl 2-amino-5,6-dihydro-4H-cyclopenta[b]thiophene-3-carboxylate

A mixture of cyclopentanone (5.0 g, 0.0595 mol, 1.0 eq.) and ethyl 2-cyanoacetate (6.73 g, 0.0595 mol, 1.0 eq.) in H₂O (20 mL) and DABCO (6.674 g, 0.0595 mol, 1.0 eq.) was stirred at room temperature for 10 minutes. Sulfur (1.91 g, 0.0595 mol, 1.0 eq.) was added to this mixture and stirring was continued at room temperature for 5 h. The reaction mixture was diluted with water (100 mL) and extracted with EtOAc (2×250 mL). The organic layer was washed with brine solution (100 mL) and dried under vacuum to give crude product. The crude compound was purified by Combi-flash by eluting with (EtOAc:hexanes/5:95) to obtain 3.0 g of ethyl 2-amino-5,6-dihydro-4H-cyclopenta[b]thiophene-3-carboxylate as a yellow solid. LC-MS: m/z 212.05 ([M+H]⁺).

### Scheme-9, Step-2: Preparation of ethyl 5,6-dihydro-4H-cyclopenta[b]thiophene-3-carboxylate (51)

Was prepared by following compound 48 preparation procedure, to afford product as a colourless oil (11% yield). LC-MS: m/z 197.10 ([M+H]⁺).

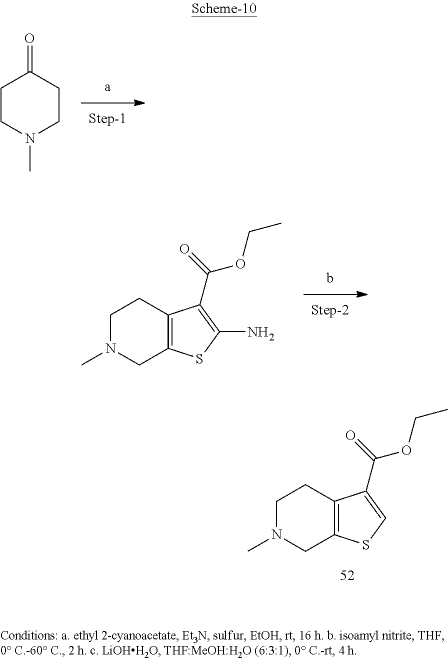

### Scheme-10, Step-1: Preparation of ethyl 2-amino-6-methyl-4,5,6,7-tetrahydrothieno[2,3-c]pyridine-3-carboxylate

To a stirred solution of ethyl 2-cyanoacetate (3.0 g, 26.51 mmol, 1.0 eq.) in ethanol (25 mL) were added Et₃N (2.67 g, 26.51 mmol, 1.0 eq.) and sulfur (850 mg, 26.51 mmol, 1.0 eq.) and the mixture was stirred for 15 min at room temperature. To the above solution, 1-methylpiperidin-4-one (3.0 g, 26.51 mmol, 1.0 eq.) was added portion-wise. The resulting reaction mixture was stirred for 16 h at room temperature. After completion of reaction (monitored by TLC), the mixture was filtered and the collected solid washed with ethanol to obtain ethyl 2-amino-6-methyl-4,5,6,7-tetrahydrothieno[2,3-c]pyridine-3-carboxylate (2.0 g, 8.32 mmol, 31% yield) as a white solid.

### Scheme-10, Step-2: Preparation of ethyl 6-methyl-4,5,6,7-tetrahydrothieno[2,3-c]pyridine-3-carboxylate (52)

To a stirred solution of ethyl 2-amino-6-methyl-4,5,6,7-tetrahydrothieno[2,3-c]pyridine-3-carboxylate (2.0 g, 8.32 mmol, 1.0 eq.) in THF (20 mL) was added isomyl nitrite (3.32 g, 24.96 mmol, 3.0 eq.) at 0° C. and the resulting reaction mixture was stirred at 60° C. for 2 h. The reaction mixture was quenched with ice cold water (100 mL) and extracted with EtOAc. The crude product was purified by Combi-flash eluting with EtOAc/n-hexanes (3:7) to obtain ethyl 6-methyl-4,5,6,7-tetrahydrothieno[2,3-c]pyridine-3-carboxylate 52 (500 mg, 2.22 mmol, 27% yield) as a yellow liquid.

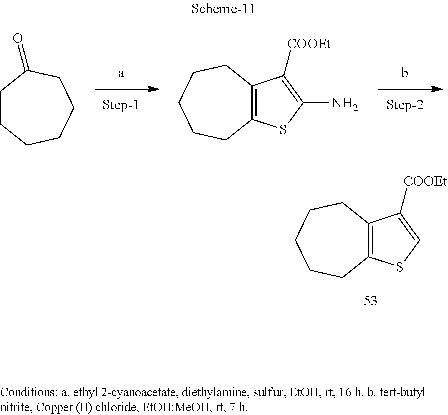 [SMILES: O=C1CCCCCC1 ; CCOC(=O)c1c(N)sc2c1CCCCC2 ; CCOC(=O)c1csc2c1CCCCC2]

### Scheme-11, Step-1: Preparation of ethyl 2-amino-5,6,7,8-tetrahydro-4H-cyclohepta[b]thiophene-3-carboxylate

To a stirred solution of cycloheptanone (2.50 g, 22.32 mmol, 1.0 eq.) in ethanol (10 mL) were added ethylcyanoacetate (2.52 g, 22.32 mmol, 1.0 eq.) and sulfur (0.714 g, 22.32 mmol, 1.0 eq.) at room temperature, followed by addition of diethylamine (5.82 mL, 55.80 mmol, 2.5 eq.) at room temperature. The resulting reaction mixture was stirred at the same temperature for 16 h. Following completion of the reaction (monitored by TLC) the mixture was diluted with water and extracted with EtOAc. The organic layer was dried and concentrated under reduced pressure, crude compound was purified by Combi-flash eluting with EtOAc/hexanes to obtain ethyl 2-amino-5,6,7,8-tetrahydro-4H-cyclohepta[b]thiophene-3-carboxylate (2.50 g, 10.46 mmol, 47% yield) as a yellow solid. LC-MS: m/z 240.1 ([M+H]⁺).

### Scheme-11, Step-2: Preparation of ethyl 5,6,7,8-tetrahydro-4H-cyclohepta[b]thiophene-3-carboxylate (53)

Prepared by following compound 48 preparation procedure to afford a colorless oil (53% yield). LC-MS: m/z 225.1 ([M+H]⁺).

### Preparation of methyl 2-phenyl-1H-imidazole-5-carboxylate (54)

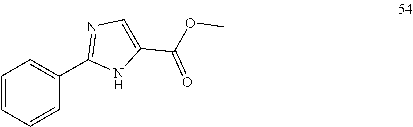

To a solution of sodium 2-phenyl-1H-imidazole-5-carboxylate (1.0 g, 4.761 mmol, 1.0 eq.) in methanol (15 mL) at room temperature was added H₂SO₄ (0.2 g, 2.38 mmol, 0.5 eq.) and the reaction heated at 60° C. for 16 h. After completion of reaction (monitored by TLC) the mixture was cooled to room temperature and concentrated under reduced pressure. The crude product was diluted with saturated NaHCO₃ solution and the solid filtered to obtain methyl 2-phenyl-1H-imidazole-5-carboxylate 54 (200 mg, 0.99 mmol, 20% yield) as a brown solid.

### Preparation of 5-phenyloxazole-2-carboxamide (55)

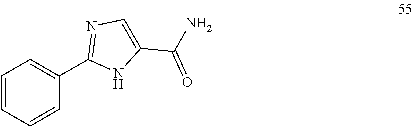 [SMILES: NC(=O)c1cnc(-c2ccccc2)[nH]1]

Was prepared by method-A by reacting 5-phenyloxazole-2-carboxylic acid (45) with aqueous ammonia, to afford an off-white solid (40% yield).

### Additional General Procedures to Prepare Right-Hand Side Variants

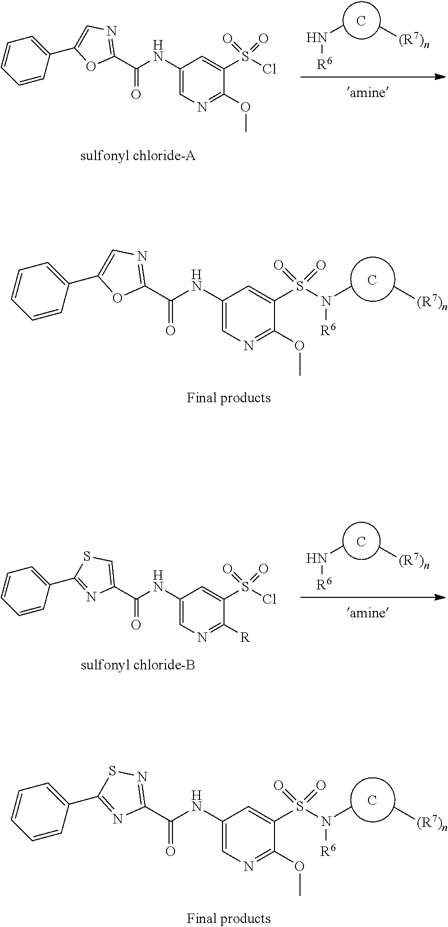

### General Procedure 1

To a solution of ‘amine’ (1.5 eq.) in DCM (1 mL) was added pyridine (4 eq.). Sulfonyl chloride (23-28 mg, 60 μmol, 1 eq.) was added and the resulting mixture stirred at 30° C. for 16 h. Progress of the reaction was monitored by LC-MS. The solvent was removed by Speedvac to give crude product which was purified by prep-HPLC to afford final product.

### General Procedure 2

For amines in HCl salt form: To a solution of ‘amine’ (1.5 eq.) in DCM (1 mL) was added pyridine (4 eq.) and TEA (4 eq.). Sulfonyl chloride (23-28 mg, 60 μmol, 1 eq.) was added to the mixture which was stirred at 30° C. for 16 h. The solvent was removed by Speedvac to give crude product which was purified by prep-HPLC to afford final product.

### General Procedure 3

To a solution of ‘amine’ (1.5 eq.) in pyridine (1 mL) was added the sulfonyl chloride (23-28 mg, 60 μmol, 1 eq.). The mixture was stirred at 80° C. for 16 h. The progress of the reaction was monitored by LC-MS. The solvent was removed by Speedvac to give crude product which was purified by prep-HPLC to afford final product.

### General Procedure 4

To a solution of ‘amine’ (1.5 eq.) in THF (1 mL) was added NaH (4 eq.) under N₂ atmosphere. The mixture was stirred at 30° C. for 1 h. The sulfonyl chloride (23-28 mg, 60 μmol, 1 eq.) was added to the mixture which was stirred at 30° C. for 16 h. The reaction was monitored by LC-MS. Upon completion, HCOOH (15 μl) was added to the reaction mixture to adjust the pH to 7. The solvent was removed by Speedvac to give crude product which was purified by prep-HPLC to afford final product.

### General Procedure 5

For amines in HCl salt form: To a solution of ‘amine’ (1.5 eq.) in DMF/THF (V/V=1:1, 1 mL), TEA (4 eq.) and NaH (4 eq.) were added under N₂ atmosphere. The mixture was stirred at 30° C. for 1 h. Sulfonyl chloride (23-28 mg, 60 μmol, 1 eq.) was added to the mixture which was stirred at 30° C. for 16 h. The reaction was monitored by LC-MS. Upon completion, HCOOH (15 μl) was added to the reaction mixture to adjust the pH to 7. The solvent was removed by Speedvac to give crude product which was purified by prep-HPLC to afford final product.

### General Procedure 6

To a solution of the ‘amine’ (1.5 eq.) in DCM (1 mL) was added DABCO (3 eq.). The sulfonyl chloride (23-28 mg, 60 μmol, 1 eq.) was added to the mixture which was then stirred at 30° C. for 16 h. The reaction was monitored by LC-MS. Upon completion, the solvent was removed by Speedvac to give crude product which was purified by prep-HPLC to give final product.

The compounds shown in Table 1 were prepared using the above general procedures by reacting sulfonyl chlorides-A and/or -B with a variety of amines. All products gave mass spectral and/or ¹H-NMR data consistent with the structure.

TABLE 1
Exemplary general procedures for
preparing select compounds

| | General |
|---|---|
| Compound | Procedure |
| No. | No. |
| 1417 | 3 |
| 1503 | 1 |
| 1511 | 3 |
| 1513 | 1 |
| 1514 | 1 |
| 1519 | 4 |
| 1525 | 1 |
| 1538 | 1 |
| 1552 | 4 |
| 1567 | 3 |
| 1568 | 3 |
| 1571 | 3 |
| 1624 | 1 |
| 1640 | 6 |
| 1648 | 6 |
| 1661 | 3 |
| 1706 | 1 |
| 1729 | 3 |
| 1746 | 3 |
| 1754 | 3 |
| 1760 | 6 |
| 1768 | 1 |
| 1773 | 6 |
| 1779 | 6 |
| 1780 | 1 |
| 1784 | 6 |
| 1785 | 1 |
| 1792 | 1 |
| 1808 | 1 |
| 1824 | 3 |
| 1839 | 4 |
| 1878 | 6 |
| 1895 | 6 |
| 1898 | 1 |
| 1908 | 6 |
| 1911 | 3 |
| 1912 | 6 |
| 1926 | 1 |

### Example 2. ADP Glo Assay

Potency of test compounds to modulate the ATPase activity of TRAP1 enzyme was evaluated using ADP Glo assay. The assay is based on quantification of the ADP generated from ATP in the ATPase reaction. The assay is performed in two steps; first, after the ATPase reaction an equal volume of ADP-Glo™ Reagent is added to terminate the ATPase reaction and deplete the remaining ATP. Second, the Kinase Detection Reagent is added to simultaneously convert ADP to ATP and allow the newly synthesized ATP to be measured using a luciferase/luciferin reaction.

All the assays were carried out in a 384-well Proxiplate. The buffer used was 25 mM Pipes pH-7.5, 40 mM KCl, 0.05 mM MgCl₂, 0.025% BSA, 0.316 mM EGTA, 0.003% Empigen, 0.01% Igepal. 150 nM Trap1 enzyme was incubated with various concentrations of the test compound (0.79 μM-100 μM) at 37° C. for 30 mins (except in blank wells). 20 μM ATP was added to each well to start the reaction. The plate was spun for 1 min at 1200 rpm and then incubated at 37° C. for 60 mins. 5 μl of ADP Glo reagent was added to all the wells and spun the plate for 1 min at 1200 rpm and kept it on a shaker at ambient temperature for 60 mins. 10 μl of kinase detection mix was added to all the wells and the plate was read for Luminescence at 495 nm. The amount of ADP released was estimated from ATP/ADP standard curve generated at the same experimental conditions. Eₘₐₓ (Maximal percent activation) and EC₅₀ (concentration for half maximal activation) were estimated by fitting the dose response data to a sigmoidal curve fitting equation using Graphpad Prism software V. 8. Exemplary results are shown in Table 2. Exemplary percent activation values at a compound concentration of 50 μM are shown in Table 2A.

TABLE 2A
Exemplary percent activation values
at a compound concentration of
50 μM in the ADP-Glo assay

| | ADP-Glo |
|---|---|
| | Percent |
| Compound | Activation |
| No. | at 50 μM |
| 2523 | C |
| 2524 | B |
| 2525 | B |
| 2526 | A |
| 2528 | C |
| 2529 | C |
| 2531 | A |
| 2532 | B |
| 2535 | B |
| 2536 | B |
| 2537 | A |
| 2538 | A |
| 2539 | C |
| 2541 | A |
| 2542 | B |
| 2543 | C |
| 2544 | C |
| 2545 | B |
| 2546 | A |
| 2547 | A |
| 2548 | C |
| 2549 | A |
| 2550 | B |
| 2551 | A |
| 2552 | B |
| 2553 | A |
| 2554 | C |
| 2555 | C |
| 2556 | B |
| 2557 | B |
| 2558 | B |
| 2559 | A |
| 2561 | C |
| 2562 | C |
| 2564 | A |
| 2566 | A |
| 2567 | A |
| 2568 | C |
| 2569 | A |
| 2570 | C |
| 2571 | B |
| 2572 | C |
| 2573 | B |
| 2574 | B |
| 2575 | B |
| 2576 | C |
| 2577 | A |
| 2578 | C |
| 2580 | A |
| 2581 | B |
| 2582 | A |
| 2583 | C |
| 2584 | C |
| 2586 | A |
| 2587 | A |
| 2590 | C |
| 2591 | B |
| 2593 | B |
| 2594 | B |
| 2595 | A |
| 2597 | C |
| 2599 | C |
| 2600 | B |
| 2601 | C |
| 2602 | B |
| 2607 | A |
| 2608 | B |
| 2609 | C |
| 2611 | A |
| 2612 | B |
| 2613 | B |
| 2614 | A |
| 2615 | A |
| 2617 | A |
| 2618 | C |
| 2619 | A |
| 2621 | B |
| 2622 | A |
| 2624 | B |
| 2625 | C |
| 2626 | C |
| 2627 | B |
| 2628 | A |
| 2629 | C |
| 2630 | A |
| 2631 | B |
| 2632 | A |
| 2633 | C |
| 2634 | C |
| 2635 | C |
| 2636 | A |
| 2637 | A |
| 2638 | B |
| 2639 | B |
| 2640 | B |
| 2641 | C |
| 2642 | B |
| 2643 | C |
| 2644 | B |
| 2646 | B |
| 2649 | B |
| 2650 | A |
| 2651 | C |
| 2653 | B |
| 2654 | A |
| 2655 | C |
| 2656 | C |
| 2657 | C |
| 2658 | A |
| 2659 | B |
| 2660 | A |
| 2661 | B |
| 2662 | A |
| 2663 | B |
| 2665 | A |
| 2666 | A |
| 2667 | C |
| 2669 | B |
| 2670 | B |
| 2671 | A |
| 2672 | A |
| 2673 | C |
| 2674 | B |
| 2675 | A |
| 2677 | A |
| 2678 | B |
| 2679 | A |
| 2682 | B |
| 2683 | A |
| 2684 | A |
| 2687 | B |
| 2688 | B |
| 2689 | C |
| 2690 | B |
| 2691 | C |
| 2692 | A |
| 2693 | A |
| 2694 | C |
| 2695 | B |
| 2696 | C |
| 2697 | C |
| 2699 | B |
| 2700 | C |
| 2701 | C |
| 2703 | A |
| 2706 | A |
| 2708 | A |
| 2709 | A |
| 2710 | C |
| 2711 | A |
| 2712 | A |
| 2713 | C |
| 2714 | C |
| 2715 | A |
| 2716 | C |
| 2718 | B |
| 2719 | B |
| 2720 | C |
| 2724 | A |
| 2725 | C |
| 2726 | C |
| 2727 | C |
| 2729 | C |
| 2730 | C |
| 2731 | A |
| 2732 | B |
| 2733 | B |
| 2734 | C |
| 2735 | C |
| 2736 | C |
| 2737 | A |
| 2738 | B |
| 2740 | C |
| 2741 | A |
| 2742 | C |
| 2743 | B |
| 2744 | C |
| 2745 | B |
| 2746 | C |
| 2747 | B |
| 2749 | B |
| 2750 | A |
| 2751 | A |
| 2752 | A |
| 2753 | A |
| 2754 | A |
| 2755 | B |
| 2756 | B |
| 2758 | B |
| 2759 | C |
| 2761 | C |
| 2762 | A |
| 2764 | C |
| 2766 | B |
| 2767 | C |
| 2768 | C |
| 2769 | C |
| 2770 | B |
| 2771 | B |
| 2772 | A |
| 2774 | C |
| 2775 | C |
| 2776 | A |
| 2777 | A |
| 2779 | B |
| 2780 | B |
| 2781 | A |
| 2782 | C |
| 2784 | C |
| 2785 | A |
| 2786 | C |
| 2787 | B |
| 2788 | C |
| 2789 | A |
| 2790 | C |
| 2791 | C |
| 2792 | A |
| 2793 | C |

In Table 2A, “A” refers to 120-150%, inclusive; “B” refers to 151-180%, inclusive; and “C” refers to 181-352%, inclusive.

### Example 3. Malachite Green Phosphate Assay

The Potency of test compounds to modulate the ATPase activity of TRAP1 enzyme was evaluated using malachite green phosphatase assay. The assay is based on quantification of the green complex formed between Malachite Green, molybdate and inorganic phosphate generated in the phosphatase reaction. Assays were performed using a Malachite Green assay kit. All the assays were carried out in a 96-well transparent plate. The buffer used was 50 mM HEPES (pH 7.5), 20 mM KCl, 4 mM MgCl₂ and 0.05% BSA. 150 nM Trap1 enzyme was incubated with various concentrations of the test compound (0.78 μM-100 μM) at 37° C. for 30 mins. 20 μM ATP was added to each well (except blank wells) to start the reaction and the plate was then incubated at 37° C. for 180 mins. 10 μl of the reaction volume of each sample was then transferred to a white 384-well proxiplate in quadruplets. 2.5 μl of Malachite Green reagent was added to all the wells and the plate was equilibrated for 15 mins. The reaction was stopped by adding 12% H₂SO₄ and the plate was read for fluorescence at 620 nm (λₑₓ=573 nm). The amount of phosphate released was estimated from a phosphate standard curve generated at the same experimental conditions. Eₘₐₓ (Maximal percent activation) and EC₅₀ (concentration of test compound for half maximal activation) were estimated by fitting the dose response data to a sigmoidal curve fitting equation using Graphpad Prism software V.7.0. Exemplary results are shown in Table 2.

TABLE 2
Exemplary biochemical data of select compounds

| | ADP | | Malachite | |
|---|---|---|---|---|
| | GLO | ADP | Green | |
| | DRC: | GLO | Fluor DRC: | Malachite |
| | GeoMean | DRC: | GeoMean | Green |
| Compound | EC₅₀ | Mean | EC₅₀ | Fluor DRC: |
| No. | (μM) | Eₘₐₓ | (μM) | Mean Eₘₐₓ |
| 535 | | | B | C |
| 592 | | | C | C |
| 594 | | | C | D |
| 604 | | | C | C |
| 612 | | | C | B |
| 670 | C | C | C | B |
| 1178 | A | B | | |
| 1193 | B | C | | |
| 1194 | C | D | | |
| 1195 | B | C | | |
| 1197 | A | C | | |
| 1198 | B | C | | |
| 1199 | A | D | | |
| 1200 | B | D | | |
| 1201 | B | C | | |
| 1202 | A | C | | |
| 1203 | B | C | | |
| 1204 | B | C | | |
| 1206 | A | D | | |
| 1213 | C | D | | |
| 1294 | C | D | | |
| 1303 | B | C | | |
| 1312 | A | B | | |
| 1321 | B | C | | |
| 1350 | A | D | | |
| 1351 | B | D | | |
| 1358 | C | B | | |
| 1359 | C | D | | |
| 1361 | B | B | | |
| 1364 | C | A | | |
| 1365 | A | A | | |
| 1366 | A | B | | |
| 1367 | C | A | | |
| 1377 | C | C | | |
| 1379 | C | B | | |
| 1380 | A | A | | |
| 1382 | C | B | | |
| 1383 | C | A | | |
| 1385 | B | C | | |
| 1386 | A | B | | |
| 1388 | B | D | | |
| 1389 | A | D | | |
| 1390 | B | C | | |
| 1393 | A | D | | |
| 1394 | C | C | | |
| 1395 | C | B | | |
| 1396 | B | C | | |
| 1398 | B | C | | |
| 1400 | B | B | | |
| 1401 | B | C | | |
| 1402 | B | C | | |
| 1403 | A | B | | |
| 1404 | C | A | | |
| 1406 | A | D | | |
| 1413 | A | D | | |
| 1417 | C | B | | |
| 1418 | A | D | | |
| 1420 | B | D | | |
| 1421 | A | B | | |
| 1422 | A | C | | |
| 1423 | A | C | | |
| 1424 | B | B | | |
| 1425 | A | C | | |
| 1481 | B | C | | |
| 1483 | A | C | | |
| 1484 | A | C | | |
| 1485 | A | D | | |
| 1486 | A | C | | |
| 1503 | C | C | | |
| 1511 | C | D | | |
| 1513 | A | C | | |
| 1514 | A | C | | |
| 1519 | B | C | | |
| 1525 | C | B | | |
| 1538 | C | D | | |
| 1552 | C | D | | |
| 1567 | C | B | | |
| 1568 | C | B | | |
| 1571 | B | C | | |
| 1624 | B | C | | |
| 1640 | B | C | | |
| 1648 | C | D | | |
| 1661 | C | C | | |
| 1706 | B | D | | |
| 1729 | C | D | | |
| 1746 | C | B | | |
| 1754 | B | C | | |
| 1760 | C | D | | |
| 1768 | C | B | | |
| 1773 | C | D | | |
| 1779 | C | D | | |
| 1780 | C | D | | |
| 1784 | C | D | | |
| 1785 | C | B | | |
| 1792 | C | D | | |
| 1808 | B | B | | |
| 1824 | C | C | | |
| 1839 | B | B | | |
| 1878 | C | D | | |
| 1895 | C | C | | |
| 1898 | C | B | | |
| 1908 | C | D | | |
| 1911 | C | D | | |
| 1912 | C | B | | |
| 1926 | C | D | | |
| 1935 | C | B | | |
| 1937 | A | D | | |
| 1940 | B | A | | |
| 1941 | A | C | | |
| 1942 | B | A | | |
| 1943 | A | A | | |
| 1944 | B | B | | |
| 1946 | A | C | | |
| 1949 | A | B | | |
| 1950 | A | D | | |
| 1953 | C | D | | |
| 1954 | C | D | | |
| 1956 | C | B | | |
| 1959 | B | C | | |
| 1962 | C | C | | |
| 1964 | A | D | | |
| 1965 | A | D | | |
| 1966 | C | C | | |
| 1970 | B | B | | |
| 1972 | C | C | | |
| 1977 | C | B | | |
| 1985 | A | C | | |
| 1986 | A | B | | |
| 1990 | B | B | | |
| 1991 | B | C | | |
| 2000 | B | B | | |
| 2002 | B | C | | |
| 2004 | B | C | | |
| 2005 | C | D | | |
| 2068 | C | B | | |
| 2069 | C | B | | |
| 2072 | A | C | | |
| 2088 | C | B | | |
| 2092 | A | B | | |
| 2119 | C | B | | |
| 2142 | C | B | | |
| 2148 | C | C | | |
| 2151 | C | B | | |
| 2155 | B | B | | |
| 2156 | C | B | | |
| 2157 | C | C | | |
| 2361 | C | D | | |
| 2365 | C | B | | |
| 2366 | A | C | | |
| 2381 | A | B | | |
| 2387 | C | B | | |
| 2390 | B | B | | |
| 2396 | C | C | | |
| 2397 | C | D | | |
| 2403 | C | B | | |
| 2407 | B | D | | |
| 2418 | A | C | | |
| 2427 | A | B | | |
| 2431 | A | C | | |
| 2445 | A | C | | |
| 2447 | B | C | | |
| 2454 | A | D | | |
| 2463 | A | B | | |
| 2466 | A | C | | |
| 2470 | A | C | | |
| 2474 | C | C | | |
| 2487 | B | C | | |
| 2492 | A | D | | |
| 2493 | C | C | | |
| 2496 | B | C | | |
| 2497 | A | C | | |
| 2498 | B | C | | |
| 2499 | A | C | | |
| 2500 | A | D | | |
| 2501 | C | C | | |
| 2502 | B | D | | |
| 2506 | C | B | | |
| 2512 | B | D | | |

In Table 2, for EC₅₀: “A” refers to <10 μM; “B” refers to 10-20 μM, inclusive; and “C” refers to 20-100 μM, exclusive; and for Eₘₐₓ: “A” refers to >300%; “B” refers to 200-300%, inclusive; “C” refers to 160-199%, inclusive; and “D” refers to 119-159%, inclusive.

### Example 4. In-Cell Target Engagement Assay

In-cell target engagement assays were performed as described in Robers M. B. et al. (2019) Quantitative, Real-Time Measurements of Intracellular Target Engagement Using Energy Transfer. In: Ziegler S., Waldmann H. (eds) Systems Chemical Biology. Methods in Molecular Biology, vol 1888. Humana Press, New York, N.Y.; Machleidt et al., ACS Chem. Biol. 2015, 10, 8, 1797-1804; or Vasta et al., Cell Chemical Biology 25, 206-214, Feb. 15, 2018; or as described herein.

Transient Transfection of SHSY5Y Cells with NanoLuc® Fusions

Below is a protocol for transient transfection of SHSY5Y cells with NanoLuc® fusions.
1. Cultivate SHSY5Y cells appropriately prior to assay in MEM w NEAA supplemented with 10% FBS.
2. Remove medium from cell flask via aspiration, trypsinize, and allow cells to dissociate from the flask.
3. Neutralize trypsin using growth medium and pellet cells via centrifugation at 200 g for 5 minutes.
4. Aspirate medium and re-suspend cells into a single cell suspension using complete cell culture medium.
5. Adjust the cell density to 300K/mL in cell culture medium in a sterile, conical tube.
6. Prepare lipid:DNA complexes as follows (this covers two assay plates, adjust numbers for more plates)
   a. Prepare a 10 μg/mL solution of DNA in Opti-MEM without serum. This solution should contain the following ratios of carrier DNA and DNA encoding NanoLuc® fusion.
      i. 5.0 μg/mL of Transfection Carrier DNA (pGEM)
      ii. 5.0 pg/mL of NanoLuc® fusion DNA
      iii. 1 mL of Opti-MEM without phenol red
   b. Mix thoroughly.
   c. Add 30 μL of FuGENE® HD into each mL of DNA mixture to form lipid:DNA complex and mix by inversion.
   d. Incubate at room temperature for 20 minutes to allow complexes to form, prepare cells while waiting for complex formation
7. Mix 1 part (e.g. 1 mL) of lipid:DNA complex with 20 parts (e.g. 20 mL) of SHSY5Y cells in suspension at 300K/mL. Mix gently by inversion 5 times in a sterile, conical tube (Again, this covers two plates, adjust for more plates) Note: Larger or smaller bulk transfections should be scaled accordingly, using this ratio.
8. Dispense cells+lipid:DNA complex into 96-well white tissue culture treated plates (0.1 mL (30K cells)/well)
9. Incubate at 37° C. in 5% CO₂ at least 20 hours to allow expression to occur.

NanoBRET™ Target Engagement Assay Protocol

Below is a preparation of 20× NanoBRET tracer in tracer dilution buffer:
1) Prepare a 100× solution of serially diluted NanoBRET Tracer in 100% DMSO. Note: For displacement test, prepare ten 2-fold serial dilutions of Tracer from the top concentration 200 μM. For competition test, prepare 100 μM concentration of Tracer.
2) Add 1 part of 100× tracer to 4 parts NanoBRET Tracer Dilution Buffer to generate 20× NanoBRET Tracer Dilution Buffer.

Below is a preparation of unlabeled ‘cold’ parental compound:
1) Prepare unlabeled parental compound at 1000× final concentration in 100% DMSO. Note: For tracer dose response, prepare 1000× (20 mM) concentration in 100% DMSO. For matrix experiments, prepare 1000× serial dilutions in 100% DMSO and then dilute to 10× in Opti-MEM without serum or phenol red.
2) Dilute it to 10× final concentration in Opti-MEM without serum or phenol red.

Below is a preparation of permeabilizing reagent:
1) Prepare 10× final concentration 500 μg/mL Digitonin in OptiMEM without serum or phenol red

Below is an assay procedure:
Prior additions of tracer and compound remove growth media and add 0.1 mL/well Opti-MEM without serum or phenol red.
1) Remove Opti-MEM without serum or phenol red and add new 85 μL (testing in live cells) or 75 μL (testing in permeabilized cells) Opti-MEM
2) Add 5 μL/well 10×Digitonin (only if testing in permeabilized cells), mix 15 sec at 700 rpm
3) Add 5 μL/well 20× Tracer in NanoBret Dilution Buffer, mix 15 sec at 700 rpm
4) Add 10 μL/well 1% DMSO in Opti-MEM or 10×‘cold’ compound, mix 15 sec at 700 rpm
5) Incubate 120 min in tissue culture incubator

Below is a protocol for the NanoBRET™ measurement:
1) Immediately prior to BRET measurements, prepare 3× Complete NanoBRET™ Nano-Glo® Substrate in OptiMEM without serum or phenol red. This solution consists of a 1:166 dilution of NanoBRET™ Nano-Glo® Substrate plus a 1:500 dilution of Extracellular NanoLuc Inhibitor in OptiMEM without serum or phenol red. Mix gently by inversion 5-10 times in a conical tube.
   Note: The final concentration of Extracellular NanoLuc inhibitor in the 3× solution is 60 μM, for a working concentration of 20 μM.
   Note: 3× solutions should be used within 2 hours of preparation.
   Note: Do not add inhibitor to permeabilized assays.
2) Add 50 μL per well of 3× Complete NanoBRET™ Nano-Glo® Substrate for a 96-well plate. Incubate 2-3 minutes at RT.
   Note: For optimal performance, BRET measurements should occur within 10 minutes of substrate addition.
3) Following addition of NanoBRET™ Nano-Glo® Substrate, measure donor emission (e.g. 450 nm) and acceptor emission (e.g. 610 nm or 630 nm) using a NanoBRET™-compatible luminometer.

Below is a protocol for BRET ratio determinations and data processing:
1) To generate raw BRET ratio values, divide the acceptor emission value (e.g. 610 nm) by the donor emission value (e.g. 450 nm) for each sample.
2) Convert raw BRET units to milli-BRET units (mBBET) by multiplying each raw BRET value by 1000.

NanoBRET equation, including optional background correction:

[(Acceptorₛₐₘₚₗₑ/Donorₛₐₘₚₗₑ)]×1000.

### NanoBRET™ Target Engagement Assay Formats

Displacement was performed by titration of Tracer+/−20 μM compound.

Competition was performed by using 1 μM Tracer+/−titration of compound.

### Example 5. Mouse Cell Derived Cyst Assay

Mouse cell derived cyst assays were performed as described in Booij, Tijmen H et al., SLAS discovery: advancing life sciences R & D vol. 22.8 (2017): 974-984, or as described below.

Murine inner medullary collecting duct cell line (mIMCD3 Pkd1^{-/-} cells)
mIMCD3 Pkd1 KO cells were seeded in 384 wells plates in extracellular matrix.
4 days after seeding, cultures were co-exposed to treatments and stimuli (2.5 μM forskolin);
After 72 h, cultures were fixed, stained for nuclei and actin cytoskeleton and imaged with 4× magnification; and
Image analysis of the 3D stack was done using Ominer™ image analysis software.

### Example 6. Effects of Compounds on ROS Production in Primary Rat Dopaminergic TH-Positive Neurons

### Primary Culture of Mesencephalic Neurons

All experiments were carried out in accordance with the National Institutes of Health Guide for the Care and Use of Laboratory Animals and followed current European Union regulations (Directive 2010/63/EU). Agreement number: A1301310.

Rat dopaminergic neurons were cultured as described by Visanji et al., 2008. Briefly, pregnant female rat of 15 days of gestation are sacrificed using a deep anesthesia with CO₂ chamber and a cervical dislocation. The midbrains obtained from 15-day-old rat embryos are dissected under a microscope. The embryonic midbrains are removed and placed in ice-cold medium of Leibovitz (L15) containing 2% of Penicillin-Streptomycin (PS) and 1% of bovine serum albumin (BSA). The ventral portion of the mesencephalic flexure, a region of the developing brain rich in dopaminergic neurons, is used for the cell preparations.

The midbrains are dissociated by trypsinisation for 20 min at 37° C. (solution at a final concentration of 0.05% trypsin and 0.02% EDTA). The reaction is stopped by the addition of Dulbecco's modified Eagle's medium (DMEM) containing DNAase I grade II (0.5 mg/mL) and 10% of foetal calf serum (FCS). Cells are then mechanically dissociated by 3 passages through a 10 mL pipette. Cells are then centrifuged at 180×g for 10 min at +4° C. on a layer of BSA (3.5%) in L15 medium. The supernatant is discarded and the cell pellets re-suspended in a defined culture medium consisting of Neurobasal supplemented with B27 (2%), L-glutamine (2 mM) and 2% of PS solution and 10 ng/mL of Brain-derived neurotrophic factor (BDNF) and 1 ng/mL of Glial-Derived Neurotrophic Factor (GDNF). Viable cells are counted in a Neubauer cytometer using the trypan blue exclusion test. The cells are seeded at a density of 40,000 cells/well in 96 well-plates (pre-coated with poly-L-lysine) and maintained in a humidified incubator at 37° C. in 5% C_{02/95}% air atmosphere. Half of the medium is changed every 2 days with fresh medium. To avoid any edge effect, the first and last columns as well as first and last lines of culture plates are not used in the study. Briefly, on day 4 of culture, the medium is removed, and fresh medium added, without or with compounds, as well as ferulic acid. On day 6 the toxin is added for 4 h or 24 h diluted in control medium, in presence of the compounds. Six wells per condition are assessed.

### Test Compounds and 6OHDA and MPP+ Exposure

Pre-incubation: On day 4 of culture, the compounds and the reference compound (ferulic acid) are dissolved in culture medium and then pre-incubated with mesencephalic neurons for 48 hours before the toxin application (plate 1 and plate 2). Ferulic acid (10 PM) is used as a reference positive control for its anti-oxidative properties. Injury: 48 hours after the application of the test compounds (on day 6), 6OHDA (20 μM) is diluted in culture medium, in presence of the compounds and added to the culture for 4 hours (plate 1) or 24 hours (plate 2). For plate 1, the culture is stopped after the 4-hour injury. For plate 2, the culture medium is removed after 24 hours and replaced with fresh medium, without 6-OHDA and without the compounds, for an additional 24 hours.

### End Point Evaluation

Immunostaining: TH and ROS—plate 1

4 hours (plate 1) after injury, the cell culture supernatant is removed, and the live cells are incubated with CellROX green reagent (marker of ROS production) for 30 min at 37° C. The CellROX reagent is cell-penetrant and will become fluorescent once oxidized by ROS. Then, cells are fixed by a solution of 4% paraformaldehyde in PBS, pH=7.3 for 20 min at room temperature. The cells are washed twice in PBS, and then are permeabilized and non-specific sites are blocked with a solution of PBS containing 0.1% of saponin and 1% FCS for 15 min at room temperature. Then, the cultures are incubated with a monoclonal Anti-Tyrosine Hydroxylase (TH) antibody produced in mouse at dilution of 1/10000 in PBS containing 1% FCS, 0.1% saponin, for 2 hours at room temperature. This antibody will be revealed with Alexa Fluor 568 goat anti-mouse IgG at the dilution 1/800 in PBS containing 1% FCS, 0.1% saponin, for 1 h at room temperature.

### Immunostaining: TH and Caspase 3—Plate 2

48 hours (24 h injury+24 h recovery) after the injury, the cell culture supernatant is removed, and cells are fixed by a solution of 4% paraformaldehyde in PBS, pH=7.3 for 20 min at room temperature. The cells are washed twice in PBS, and then are permeabilized and non-specific sites blocked with a solution of PBS containing 0.1% of saponin and 1% FCS for 15 min at room temperature. Then, the cultures are incubated with i) a monoclonal Anti-Tyrosine Hydroxylase (TH) antibody produced in mouse at dilution of 1/10000 and ii) a rabbit polyclonal antibody anti-activated caspase 3 at dilution of 1/500 in PBS containing 1% fetal calf serum and 0.1% of saponin, for 2 hours at room temperature. These antibodies will be revealed with Alexa Fluor 488 goat anti-mouse IgG and Alexa fluor 568 goat anti-rabbit IgG at the dilution 1/400 in PBS containing 1% FCS, 0.1% saponin, for 1 hour at room temperature.

### Automatic Computer Analysis

For each condition, 20 pictures at 10× magnification (plate 1) or 30 pictures at 20× magnification (representing the whole well area) were automatically taken using ImageXpress®. All images were generated by ImageXpress® using the same acquisition parameters. From images, analyses were directly and automatically performed by Custom Module Editor®. The following read-outs were measured: Analysis of total number of TH neurons (TH positive neuron number), Analysis of the ROS into TH positive neurons, Analysis of caspase 3 positive TH neurons, and Analysis of total neurite network of TH positive neurons (in μm).

### Statistical Analysis

All values are expressed as mean+/−SEM (standard error of the mean). Statistical analysis was performed by one-way ANOVA, followed by a Dunnett's or a PLSD Fisher's test. p<0.05 is considered significant.

### Example 7. Compound Profiling in an α-Synuclein Aggregation Assay

Assay measuring α-synuclein expression and aggregation in differentiated neuronal ReNcell VM cells transduced with human α-synuclein-WT were used to assess the α-synuclein modulation characteristics of compounds. To this purpose, ReNcell VM cells in growth medium (containing EGF and bFGF) were seeded in 96-well laminin-coated plates.

Three (3) days post-seeding, on DO, the cells were refreshed with differentiation medium (devoid of EGF and bFGF, supplemented with cAMP and GDNF) and transduced with α-synuclein-encoding adenoviruses. On D1, after overnight transduction, the adenoviral medium was removed and test compound CRCs diluted in differentiation medium were added to the cells in biological duplicates (i.e. in wells located on separate assay plates processed on the same day). The assay includes a compound refreshment on D4. On each plate tested, up to six (6) vehicle control samples (0.1% DMSO) and up to six (6) positive control samples (10 μM KU 0063794) were included. Furthermore, each assay run includes an 8-point CRC of the reference compound KU 0063794. After six (6) days of compound treatment (on D7), the cells were fixed using 3.7% formaldehyde.

Assay readouts were aggregate-selective (MJFR-14) and α/β-synuclein (Syn205) immunoreactivity, quantified using high-content analysis with algorithms for Density×Area (D×A). Co-staining of cell nuclei with DAPI is performed to quantify cell number as a measure of potential toxicity. Synuclein immunoreactivity (D×A) was normalized for the number of nuclei (nuc) per imaged well (D×A/nuc). Compound performance was evaluated by efficacy (percentage inhibition (PIN) relative to positive control) and potency (pIC₅₀, when calculable) determination.

Assay quality was confirmed using the following pre-set criteria: Toxicity: ≤25% nuclei loss compared to average DMSO control, Coefficient of variation (CV): ≤20% CV for DMSO control, Assay window: ≥2.0 for Syn205, and Assay window: ≥1.5 for MJF-14.

TABLE 3
Exemplary α-synuclein aggregation assay results of compound 1178

| | Syn205 | | | | | |
|---|---|---|---|---|---|---|
| | | Max | Max | | CV Blank | |
| Compound | Potency | efficacy | efficacy | Assay | (0.1% | |
| No. | (pIC₅₀) | (PIN) | (PE) | Window | DMSO) | |
| 1178 | 5.6 | 64.7 | 44.9 | 3.3 | 9.9 | |
| | MJFR-14 | | | Toxicity | | |
| | | Max | Max | | CV Blank | Cell |
| Compound | Potency | efficacy | efficacy | Assay | (0.1% | loss |
| No. | (pIC₅₀) | (PIN) | (PE) | Window | DMSO) | (>25%) |
| 1178 | 5.6 | 52.1 | 36.2 | 3.3 | 8.3 | N.D. |

N.D. = not detectable.

### Example 8. Effects of Compound 1206 in a Mouse Model of Polycystic Kidney Disease

Compound 1206 (10 mg/kg po, QD dosing for 14d, n=13/group) was assessed in a tamoxifen-induced polycystic kidney disease model (Ksp-TamCre×PkdlLox; Human Molecular Genetics, 2007, Vol. 16, No. 24, 3188-3196). A negative control group induced with tamoxifen (Vehicle) and a negative control group not induced with tamoxifen (No Tam) were included. A positive control group was dosed with everolimus. Mice were dosed orally on both PND 10, 11 with 25 mg/kg tamoxifen. Oral dosage of vehicle, compound 1206, and everolimus started from PND 14 onwards to PND 27. The readouts of this study were body weight, kidney weight, BUN levels and histopathology assessment including cyst index.

The fixated kidney half was processed for paraffin embedding and further pathological assessment after hematoxylin-eosin staining. One piece of each H-E stained kidney half was observed by a board certified pathologist, who read a cross section of the kidney. For each kidney one section was evaluated. From this section an overview photo was taken (2X) and used to calculate the cystic index. The cystic areas were identified by a color thresholding method using an image analysis system (Image software, public domain software, NIH, Bethesda, Md.). The threshold area was calculated within a defined ROI (region of interest) representing the total area analyzed. This area represents at least 70-80% of the entire kidney section. The cystic index (CI) is defined as the percentage of the area of cysts lumen (in pixels) over the total kidney area in the selected ROI (in pixels).

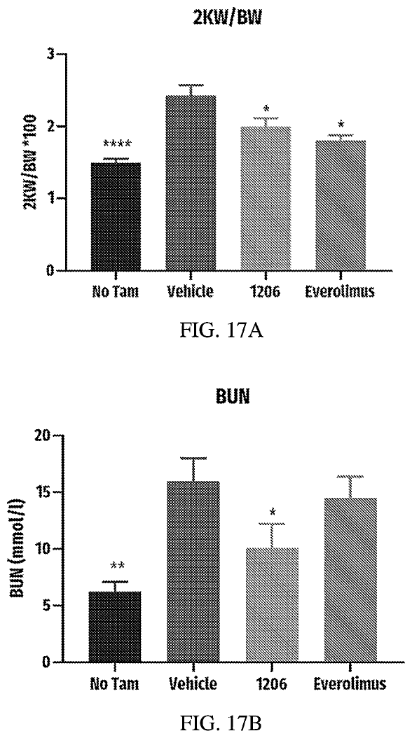

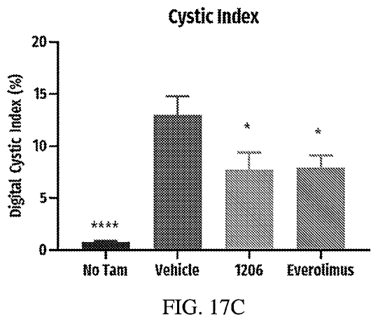

Exemplary results are shown in FIGS. 17A to 17C. FIG. 17A shows that, compared to the tamoxifen-induced vehicle control group, the 2 KW/BW ratio was significantly lower in the everolimus (positive control) group and in the compound 1206 group. FIG. 17B shows that the mice treated with compound 1206 had significantly lower urea levels (BUN) as compared with the mice in the tamoxifen-induced vehicle control group. In general, the mice in the no-tamoxifen induced control group (No Tam) had urea levels within the normal physiological range. FIG. 17C shows that the mice in the compound 1206 group had a significantly lower cystic index than the mice in the tamoxifen-induced vehicle control group. The same was true for the everolimus (positive control) group.

List of abbreviations

| | |
|---|---|
| ACN | acetonitrile |
| AcOH | acetic acid |
| aq. | aqueous |
| Boc | tert-Butoxycarbonyl |
| CH₂Cl₂ | Dichloromethane |
| CHCl₃ | Chloroform |
| CD₃OD | Deuterated methanol |
| d | doublet |
| DABCO | 1,4-diazabicyclo[2.2.2]octane |
| dd | Doublet of doublets |
| DIPEA | N,N-Diisopropylethylamine |
| DMAP | 4-(dimethylamino)pyridine |
| DIPEA | N,N-Diisopropylethylamine |
| DMF | N,N-dimethylformamide |
| DMSO | Dimethyl sulfoxide |
| DMSO-d₆ | Deuterated DMSO |
| EDC•HCl | N-Ethyl-N′-(3-dimethylaminopropyl)carbodiimide |
| | hydrochloride |
| eq. | equivalent |
| EtOAc | Ethyl acetate |
| Et₃N | triethylamine |
| EtOH | Ethanol |
| g | gram |
| h | hour |
| HOBt | Hydroxybenzotriazole |
| Hz | Hertz |
| HATU | (1-[Bis(dimethylamino)methylene]-1H-1,2,3-triazolo |
| | [4,5-b]pyridinium 3-oxide hexafluorophosphate |
| J | Coupling constant |
| LC-MS | Liquid chromatography-mass spectrometry |
| LiHMDS | Lithium bis(trimethylsilyl)amide |
| m | multiplet |
| mg | milligram |
| MHz | Mega Hertz |
| mL | millilitre |
| min | Minute(s) |
| mmol | Millimole |
| MeOH | Methanol |
| m/z | Mass-to-charge ratio |
| NMR | Nuclear magnetic resonance |
| Pd₂(dba)₃ | Tris(dibenzylideneacetone)dipalladium(0) |
| py | pyridine |
| q | quartet |
| s | singlet |
| t | triplet |
| THF | Tetrahydrofuran |
| TLC | Thin layer chromatography |

### REFERENCES

1. Abou-Sleiman P M, Muqit M M & Wood N W (2006) Expanding insights of mitochondrial dysfunction in Parkinson's disease. Nat Rev Neurosci, 7, 207-19.
2. Bayir H, Kapralov A A, Jiang J, Huang Z, Tyurina Y Y, Tyurin V A, Zhao Q, Belikova N A, Vlasova I I, Maeda A, Zhu J, Na H M, Mastroberardino P G, Sparvero L J, Amoscato A A, Chu C T, Greenamyre J T & Kagan V E (2009) Peroxidase mechanism of lipid-dependent cross-linking of synuclein with cytochrome C: protection against apoptosis versus delayed oxidative stress in Parkinson disease. J Biol Chem, 284, 15951-69.
3. Giordano S, Lee J, Darley-Usmar V M & Zhang J (2012) Distinct effects of rotenone, 1-methyl-4-phenylpyridinium and 6-hydroxydopamine on cellular bioenergetics and cell death. PLoS One, 7, e44610.
4. Jankovic J (2008) Parkinson's disease: clinical features and diagnosis. J Neurol Neurosurg Psychiatry, 79, 368-76.
5. Martin I, Dawson V L, & Dawson™ (2010) The impact of genetic research on our understanding of Parkinson's disease. Prog Brain Res, 183, 21-41.
6. Vila, M. & Przedborski, S. (2004). Genetic clues to the pathogenesis of Parkinson's disease. Nat. Med., 10 Suppl, S58-S62.
7. Visanji N P, Orsi A, Johnston T H, Howson P A, Dixon K, Callizot N, Brotchie J M and Rees D D. PYM50028, a novel, orally active, nonpeptide neurotrophic factor inducer, prevents and reverses neuronal damage induced by MPP+ in mesencephalic neurons and by MPTP in a mouse model of Parkinson's disease. FASEB J. 2008; 22(7):2488-97.
8. Vogiatzi T, Xilouri M, Vekrellis K & Stefanis L (2008) Wild type alpha-synuclein is degraded by chaperone-mediated autophagy and macroautophagy in neuronal cells. J Biol Chem, 283, 23542-56.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 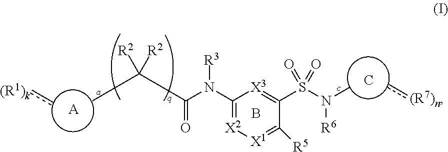
or a pharmaceutically acceptable salt, solvate, hydrate, polymorph, co-crystal, tautomer, stereoisomer, isotopically labeled compound, or prodrug thereof, wherein: 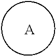
is aryl or heteroaryl;
each [symbol] is independently a single or double bond, as valency permits;
when attached to a carbon atom, each R¹ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)₂R^{a}, —NR^{a}S(═O)₂OR^{a}, —NR^{a}S(═O)₂N(R^{a})₂, —OS(═O)₂R^{a}, —OS(═O)₂OR^{a}, —OS(═O)₂N(R^{a})₂, —S(═O)₂R^{a}, —S(═O)₂OR^{a}, —S(═O)₂N(R^{a})₂, —P(═O)(R^{a})₂, or ═O, as valency permits;
when attached to a nitrogen atom, each R¹ is independently substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —S(═O)₂R^{a}, —S(═O)₂OR^{a}, —S(═O)₂N(R^{a})₂, —P(═O)(R^{a})₂, a nitrogen protecting group, or ═O, as valency permits;
or one R¹ and R³ are joined with their intervening atoms to form substituted or unsubstituted heterocyclyl;
each R^{a} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on a nitrogen atom are joined with the nitrogen atom to form substituted or unsubstituted heterocyclyl or substituted or unsubstituted heteroaryl;
k is 0 or an integer between 1 and 13, inclusive, as valency permits;
each R² is independently hydrogen, halogen, or substituted or unsubstituted alkyl;
q is 0 or 1;
R³ is hydrogen, substituted or unsubstituted alkyl, or a nitrogen protecting group;
one of X¹, X², and X³ is N or N(O);
each of the other two of X¹, X², and X³ is CR⁴;
each R⁴ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)₂R^{a}, —NR^{a}S(═O)₂OR^{a}, —NR^{a}S(═O)₂N(R^{a})₂, —OS(═O)₂R^{a}, —OS(═O)₂OR^{a}, —OS(═O)₂N(R^{a})₂, —S(═O)₂R^{a}, —S(═O)₂OR^{a}, or —S(═O)₂N(R^{a})₂;
R⁵ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)₂R^{a}, —NR^{a}S(═O)₂OR^{a}, —NR^{a}S(═O)₂N(R^{a})₂, —OS(═O)₂R^{a}, —OS(═O)₂OR^{a}, —OS(═O)₂N(R^{a})₂, —S(═O)₂R^{a}, —S(═O)₂OR^{a}, or —S(═O)₂N(R^{a})₂;
R⁶ is hydrogen, substituted or unsubstituted alkyl, or a nitrogen protecting group;
or R⁶ and one R⁷ are joined with their intervening atoms to form substituted or unsubstituted heterocyclyl; 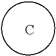
is aryl or heteroaryl;
when attached to a carbon atom, each R⁷ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)₂R^{a}, —NR^{a}S(═O)₂OR^{a}, —NR^{a}S(═O)₂N(R^{a})₂, —OS(═O)₂R^{a}, —OS(═O)₂OR^{a}, —OS(═O)₂N(R^{a})₂, —S(═O)₂R^{a}, —S(═O)₂OR^{a}, —S(═O)₂N(R^{a})₂, —P(═O)(R^{a})₂, or ═O, as valency permits;
when attached to a nitrogen atom, each R⁷ is independently substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{a}, —C(═O)OR^{a}, —C(═O)N(R^{a})₂, —C(═NR^{a})R^{a}, —C(═NR^{a})OR^{a}, —C(═NR^{a})N(R^{a})₂, —S(═O)₂R^{a}, —S(═O)₂OR^{a}, —S(═O)₂N(R^{a})₂, —P(═O)(R^{a})₂, a nitrogen protecting group, or ═O, as valency permits; and
n is 0 or an integer between 1 and 13, inclusive, as valency permits;
provided that the compound is not of the formula:

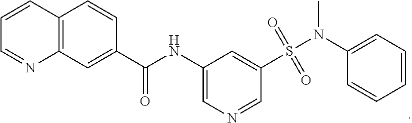 [SMILES: CN(c1ccccc1)S(=O)(=O)c1cncc(NC(=O)c2ccc3cccnc3c2)c1]

2. The compound of claim 1, or a pharmaceutically acceptable salt, solvate, hydrate, polymorph, co-crystal, tautomer, stereoisomer, isotopically labeled compound, or prodrug thereof, wherein 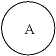
is phenyl.

3. The compound of claim 1, or a pharmaceutically acceptable salt, solvate, hydrate, polymorph, co-crystal, tautomer, stereoisomer, isotopically labeled compound, or prodrug thereof, wherein 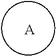
is 5- or 6-membered monocyclic heteroaryl.

4. The compound of claim 1, or a pharmaceutically acceptable salt, solvate, hydrate, polymorph, co-crystal, tautomer, stereoisomer, isotopically labeled compound, or prodrug thereof, wherein

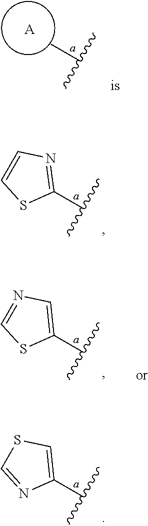

5. The compound of claim 1, or a pharmaceutically acceptable salt, solvate, hydrate, polymorph, co-crystal, tautomer, stereoisomer, isotopically labeled compound, or prodrug thereof, wherein

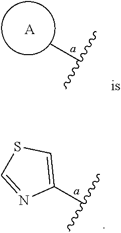

6. The compound of claim 1, or a pharmaceutically acceptable salt, solvate, hydrate, polymorph, co-crystal, tautomer, stereoisomer, isotopically labeled compound, or prodrug thereof, wherein

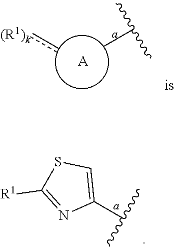

7. The compound of claim 1, or a pharmaceutically acceptable salt, solvate, hydrate, polymorph, co-crystal, tautomer, stereoisomer, isotopically labeled compound, or prodrug thereof, wherein

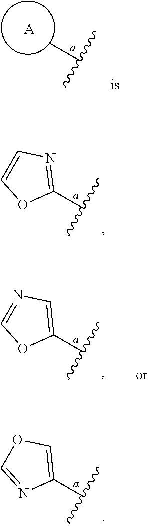

8. The compound of claim 1, or a pharmaceutically acceptable salt, solvate, hydrate, polymorph, co-crystal, tautomer, stereoisomer, isotopically labeled compound, or prodrug thereof, wherein

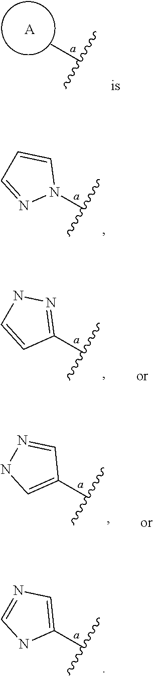

9. The compound of claim 1, or a pharmaceutically acceptable salt, solvate, hydrate, polymorph, co-crystal, tautomer, stereoisomer, isotopically labeled compound, or prodrug thereof, wherein

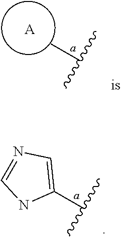

10. The compound of claim 1, or a pharmaceutically acceptable salt, solvate, hydrate, polymorph, co-crystal, tautomer, stereoisomer, isotopically labeled compound, or prodrug thereof, wherein

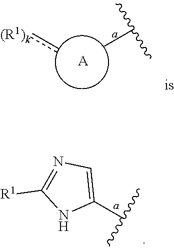

11. The compound of claim 1, or a pharmaceutically acceptable salt, solvate, hydrate, polymorph, co-crystal, tautomer, stereoisomer, isotopically labeled compound, or prodrug thereof, wherein

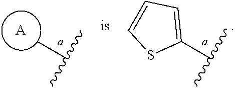

12. The compound of claim 1, or a pharmaceutically acceptable salt, solvate, hydrate, polymorph, co-crystal, tautomer, stereoisomer, isotopically labeled compound, or prodrug thereof, wherein

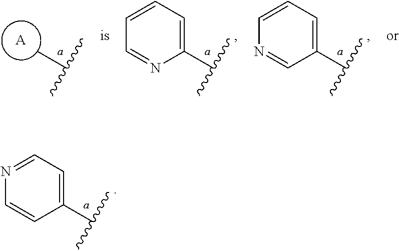

13. The compound of claim 1, or a pharmaceutically acceptable salt, solvate, hydrate, polymorph, co-crystal, tautomer, stereoisomer, isotopically labeled compound, or prodrug thereof, wherein 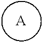
is phenyl fused with monocyclic or bicyclic, carbocyclyl, heterocyclyl, aryl, or heteroaryl, wherein bond a is attached to the phenyl.

14. The compound of claim 1, or a pharmaceutically acceptable salt, solvate, hydrate, polymorph, co-crystal, tautomer, stereoisomer, isotopically labeled compound, or prodrug thereof, wherein

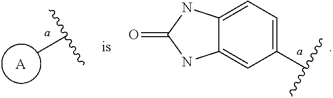

15. The compound of claim 1, or a pharmaceutically acceptable salt, solvate, hydrate, polymorph, co-crystal, tautomer, stereoisomer, isotopically labeled compound, or prodrug thereof, wherein

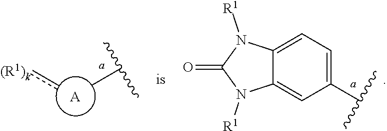

16. The compound of claim 1, or a pharmaceutically acceptable salt, solvate, hydrate, polymorph, co-crystal, tautomer, stereoisomer, isotopically labeled compound, or prodrug thereof, wherein

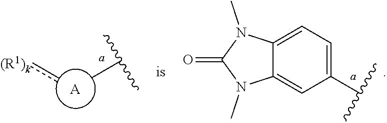

17. The compound of claim 1, or a pharmaceutically acceptable salt, solvate, hydrate, polymorph, co-crystal, tautomer, stereoisomer, isotopically labeled compound, or prodrug thereof, wherein

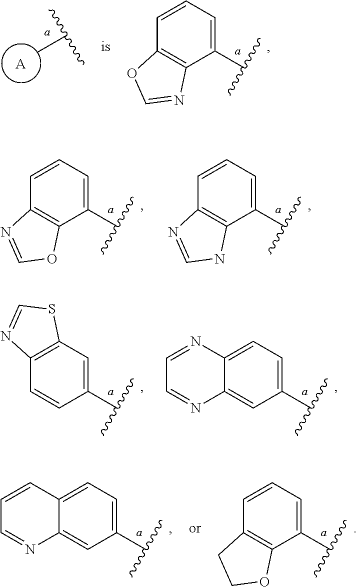

18. The compound of claim 1, or a pharmaceutically acceptable salt, solvate, hydrate, polymorph, co-crystal, tautomer, stereoisomer, isotopically labeled compound, or prodrug thereof, wherein 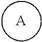
is 5- or 6-membered monocyclic heteroaryl fused with monocyclic or bicyclic, carbocyclyl, heterocyclyl, aryl, or heteroaryl, wherein bond a is attached to the 5- or 6-membered monocyclic heteroaryl.

19. The compound of claim 1, or a pharmaceutically acceptable salt, solvate, hydrate, polymorph, co-crystal, tautomer, stereoisomer, isotopically labeled compound, or prodrug thereof, wherein

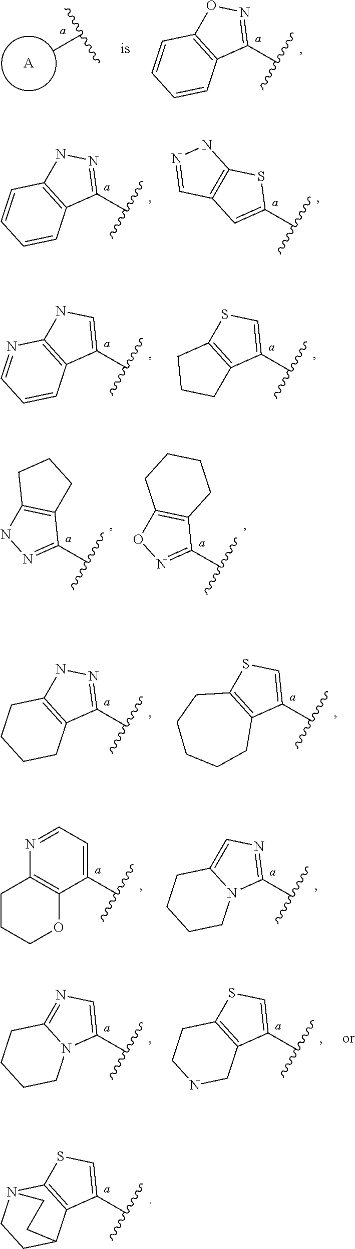

20. The compound of any one of claims 1-19, or a pharmaceutically acceptable salt, solvate, hydrate, polymorph, co-crystal, tautomer, stereoisomer, isotopically labeled compound, or prodrug thereof, wherein X¹ is N.

21. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

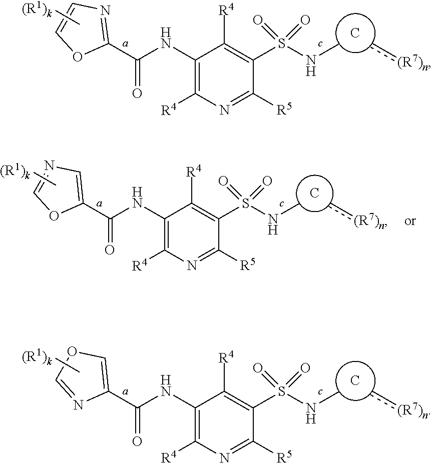

22. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

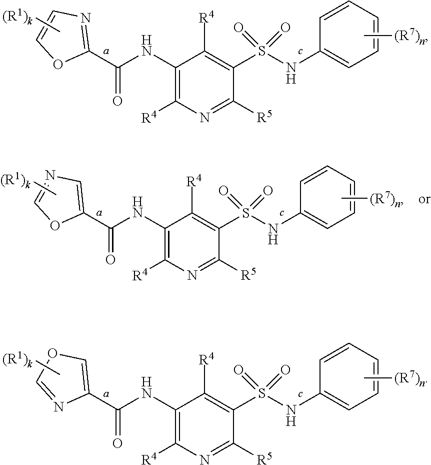

23. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

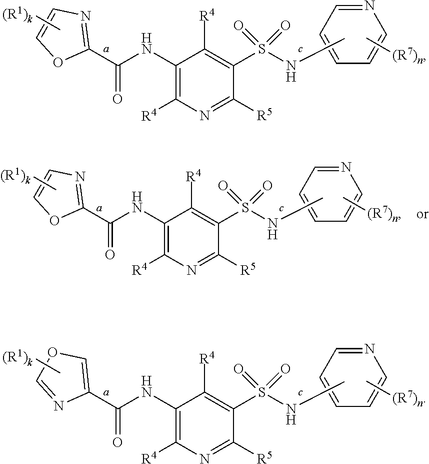

24. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

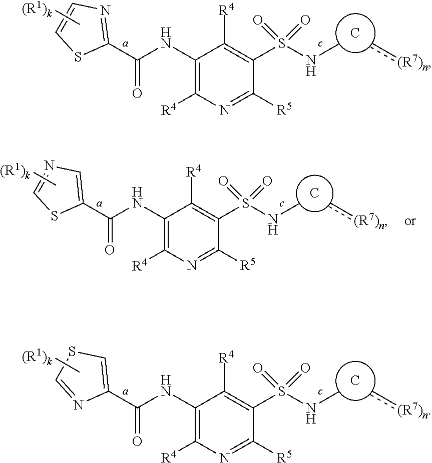

25. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

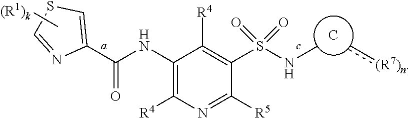

26. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

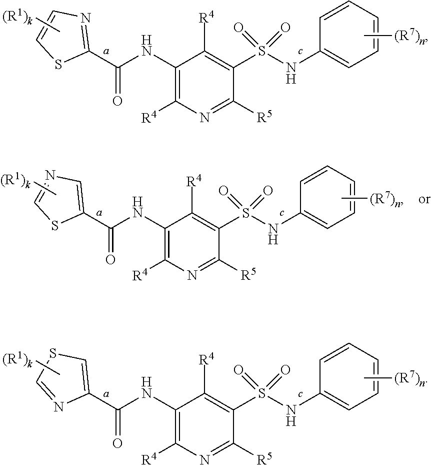

27. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

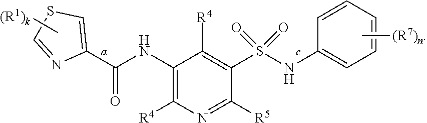

28. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

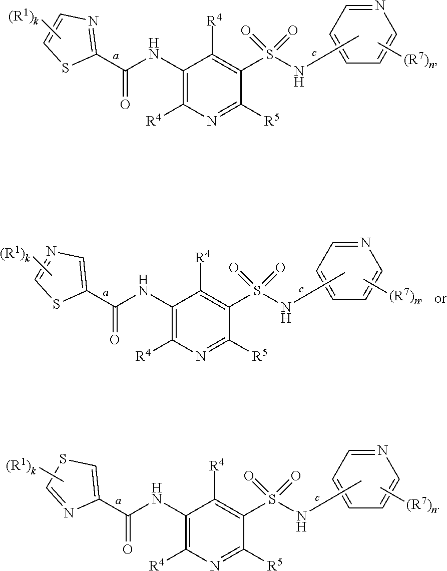

29. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

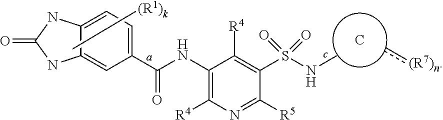

30. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

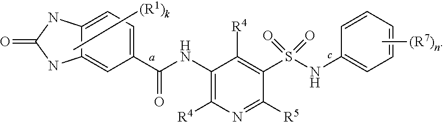

31. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

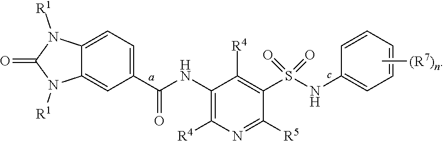

32. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

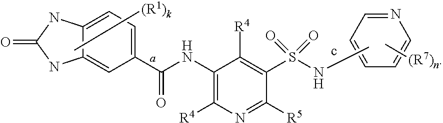

33. The compound of any one of claims 1-32, or a pharmaceutically acceptable salt, solvate, hydrate, polymorph, co-crystal, tautomer, stereoisomer, isotopically labeled compound, or prodrug thereof, wherein the molecular weight of each R¹ is less than 150 g/mol.

34. The compound of any one of claims 1-33, or a pharmaceutically acceptable salt, solvate, hydrate, polymorph, co-crystal, tautomer, stereoisomer, isotopically labeled compound, or prodrug thereof, wherein at least one instance of R¹ attached to a carbon atom is halogen, substituted or unsubstituted alkyl, —OR^{a}, —C(═O)R^{a}, —C(═O)OR^{a}, substituted or unsubstituted, 3- to 7-membered, monocyclic heterocyclyl, substituted or unsubstituted phenyl, or substituted or unsubstituted, 5- to 6-membered, monocyclic heteroaryl.

35. The compound of any one of claims 1-33, or a pharmaceutically acceptable salt, solvate, hydrate, polymorph, co-crystal, tautomer, stereoisomer, isotopically labeled compound, or prodrug thereof, wherein at least one instance of R¹ is substituted or unsubstituted phenyl.

36. The compound of any one of claims 1-5, 7-9, 11-14, 17-30, and 32-35, or a pharmaceutically acceptable salt, solvate, hydrate, polymorph, co-crystal, tautomer, stereoisomer, isotopically labeled compound, or prodrug thereof, wherein k is 0, 1, or 2.

37. The compound of any one of claims 1-20 and 33-36, or a pharmaceutically acceptable salt, solvate, hydrate, polymorph, co-crystal, tautomer, stereoisomer, isotopically labeled compound, or prodrug thereof, wherein q is 0.

38. The compound of any one of claims 1-20 and 33-37, or a pharmaceutically acceptable salt, solvate, hydrate, polymorph, co-crystal, tautomer, stereoisomer, isotopically labeled compound, or prodrug thereof, wherein R³ is hydrogen.

39. The compound of any one of claims 1-38, or a pharmaceutically acceptable salt, solvate, hydrate, polymorph, co-crystal, tautomer, stereoisomer, isotopically labeled compound, or prodrug thereof, wherein each instance of R⁴ is hydrogen.

40. The compound of any one of claims 1-39, or a pharmaceutically acceptable salt, solvate, hydrate, polymorph, co-crystal, tautomer, stereoisomer, isotopically labeled compound, or prodrug thereof, wherein R⁵ is —OR^{a} or substituted or unsubstituted alkyl.

41. The compound of claim 40, or a pharmaceutically acceptable salt, solvate, hydrate, polymorph, co-crystal, tautomer, stereoisomer, isotopically labeled compound, or prodrug thereof, wherein R⁵ is —OCH₃.

42. The compound of claim 40, or a pharmaceutically acceptable salt, solvate, hydrate, polymorph, co-crystal, tautomer, stereoisomer, isotopically labeled compound, or prodrug thereof, wherein R⁵ is —CH₃.

43. The compound of any one of claims 1-39, or a pharmaceutically acceptable salt, solvate, hydrate, polymorph, co-crystal, tautomer, stereoisomer, isotopically labeled compound, or prodrug thereof, wherein R⁵ is hydrogen.

44. The compound of any one of claims 1-20 and 33-43, or a pharmaceutically acceptable salt, solvate, hydrate, polymorph, co-crystal, tautomer, stereoisomer, isotopically labeled compound, or prodrug thereof, wherein R⁶ is hydrogen.

45. The compound of any one of claims 1-21, 24, 25, 29, and 33-44, or a pharmaceutically acceptable salt, solvate, hydrate, polymorph, co-crystal, tautomer, stereoisomer, isotopically labeled compound, or prodrug thereof, wherein 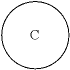
is phenyl.

46. The compound of claim 45, or a pharmaceutically acceptable salt, solvate, hydrate, polymorph, co-crystal, tautomer, stereoisomer, isotopically labeled compound, or prodrug thereof, wherein

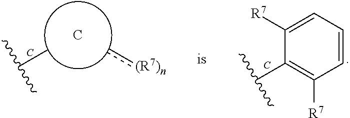

47. The compound of claim 45, or a pharmaceutically acceptable salt, solvate, hydrate, polymorph, co-crystal, tautomer, stereoisomer, isotopically labeled compound, or prodrug thereof, wherein

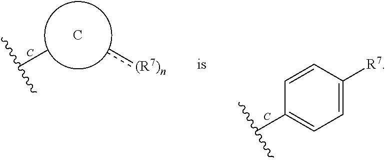

48. The compound of claim 45, or a pharmaceutically acceptable salt, solvate, hydrate, polymorph, co-crystal, tautomer, stereoisomer, isotopically labeled compound, or prodrug thereof, wherein

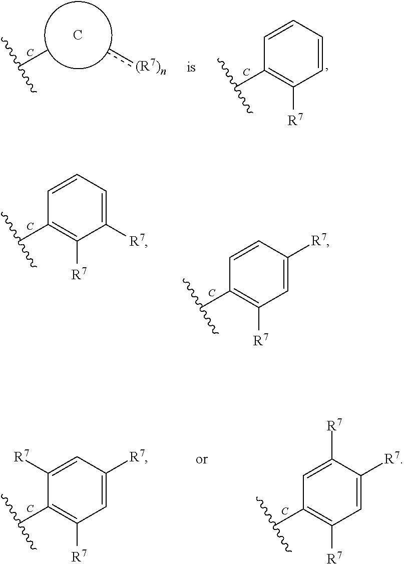

49. The compound of any one of claims 1-21, 24, 25, 29, and 33-44, or a pharmaceutically acceptable salt, solvate, hydrate, polymorph, co-crystal, tautomer, stereoisomer, isotopically labeled compound, or prodrug thereof, wherein 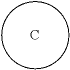
is 5- or 6-membered monocyclic heteroaryl.

50. The compound of claim 49, or a pharmaceutically acceptable salt, solvate, hydrate, polymorph, co-crystal, tautomer, stereoisomer, isotopically labeled compound, or prodrug thereof, wherein 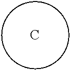
is pyridinyl.

51. The compound of claim 49, or a pharmaceutically acceptable salt, solvate, hydrate, polymorph, co-crystal, tautomer, stereoisomer, isotopically labeled compound, or prodrug thereof, wherein

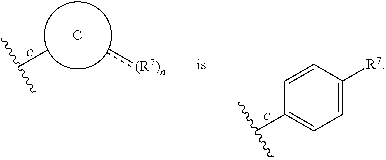

52. The compound of claim 49, or a pharmaceutically acceptable salt, solvate, hydrate, polymorph, co-crystal, tautomer, stereoisomer, isotopically labeled compound, or prodrug thereof, wherein 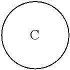
is pyrimidinyl, thienyl, pyrazolyl, tetrazolyl, isoxazolyl, or

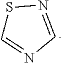

53. The compound of any one of claims 1-21, 24, 25, 29, and 33-44, or a pharmaceutically acceptable salt, solvate, hydrate, polymorph, co-crystal, tautomer, stereoisomer, isotopically labeled compound, or prodrug thereof, wherein 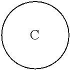
is phenyl fused with monocyclic or bicyclic, carbocyclyl, heterocyclyl, aryl, or heteroaryl, wherein bond c is attached to the phenyl; or 5- or 6-membered monocyclic heteroaryl fused with monocyclic or bicyclic, carbocyclyl, heterocyclyl, aryl, or heteroaryl, wherein bond c is attached to the 5- or 6-membered monocyclic heteroaryl.

54. The compound of claim 53, or a pharmaceutically acceptable salt, solvate, hydrate, polymorph, co-crystal, tautomer, stereoisomer, isotopically labeled compound, or prodrug thereof, wherein

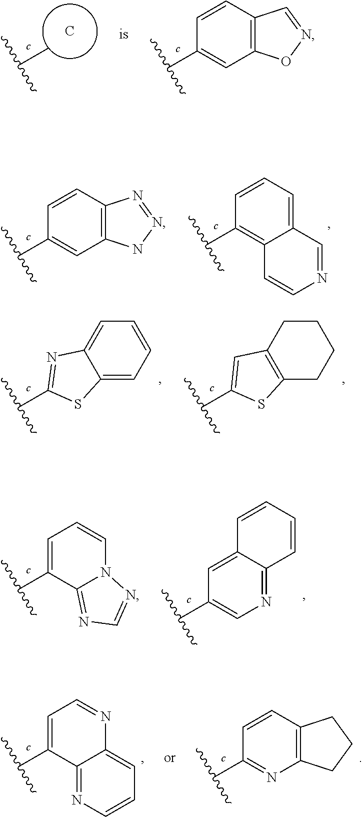

55. The compound of any one of claims 1-45, 49-50, and 52-54, or a pharmaceutically acceptable salt, solvate, hydrate, polymorph, co-crystal, tautomer, stereoisomer, isotopically labeled compound, or prodrug thereof, wherein n is 0, 1, or 2.

56. The compound of any one of claims 1-55, or a pharmaceutically acceptable salt, solvate, hydrate, polymorph, co-crystal, tautomer, stereoisomer, isotopically labeled compound, or prodrug thereof, provided that:
R¹ and R³ are not joined with their intervening atoms to form substituted or unsubstituted heterocyclyl; and
R⁶ and R⁷ are not joined with their intervening atoms to form substituted or unsubstituted heterocyclyl.

57. The compound of any one of claims 1-56, or a pharmaceutically acceptable salt, solvate, hydrate, polymorph, co-crystal, tautomer, stereoisomer, isotopically labeled compound, or prodrug thereof, wherein the molecular weight of each R⁷ is less than 150 g/mol.

58. The compound of any one of claims 1-57, or a pharmaceutically acceptable salt, solvate, hydrate, polymorph, co-crystal, tautomer, stereoisomer, isotopically labeled compound, or prodrug thereof, wherein at least one R⁷ attached to a carbon atom is halogen, substituted or unsubstituted alkyl, substituted or unsubstituted, 3- to 7-membered, monocyclic carbocyclyl, substituted or unsubstituted phenyl, —OR^{a}, —CN, or —N(R^{a})₂.

59. The compound of any one of claims 1-57, or a pharmaceutically acceptable salt, solvate, hydrate, polymorph, co-crystal, tautomer, stereoisomer, isotopically labeled compound, or prodrug thereof, wherein at least one R⁷ attached to a carbon atom is chloro, fluoro, —CH₃, —CF₃, unsubstituted benzyl, unsubstituted C₂₋₆ alkyl, —OCH₃, —O(unsubstituted C₂₋₆ alkyl), —OCH₂CH₂OCH₃, —CN, —NHCH₃, or —N(CH₃)₂.

60. The compound of any one of claims 1-57, or a pharmaceutically acceptable salt, solvate, hydrate, polymorph, co-crystal, tautomer, stereoisomer, isotopically labeled compound, or prodrug thereof, wherein each R⁷ attached to a carbon atom is independently halogen or substituted or unsubstituted alkyl.

61. The compound of claim 60, or a pharmaceutically acceptable salt, solvate, hydrate, polymorph, co-crystal, tautomer, stereoisomer, isotopically labeled compound, or prodrug thereof, wherein each R⁷ attached to a carbon atom is independently chloro or —CH₃.

62. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

| No. | Formula |
|---|---|
| 535 | 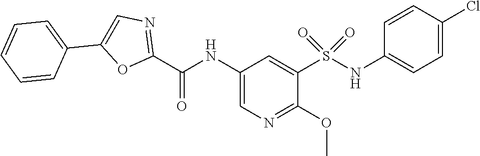 [SMILES: COc1ncc(NC(=O)c2ncc(-c3ccccc3)o2)cc1S(=O)(=O)Nc1ccc(Cl)cc1] |
| 592 | 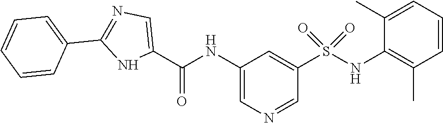 [SMILES: Cc1cccc(C)c1NS(=O)(=O)c1cncc(NC(=O)c2cnc(-c3ccccc3)[nH]2)c1] |
| 594 | 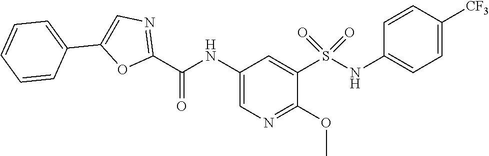 [SMILES: COc1ncc(NC(=O)c2ncc(-c3ccccc3)o2)cc1S(=O)(=O)Nc1ccc(C(F)(F)F)cc1] |
| 604 | 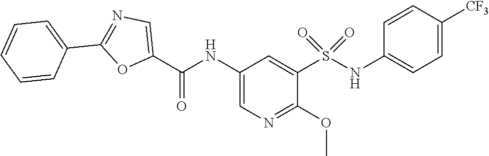 [SMILES: COc1ncc(NC(=O)c2cnc(-c3ccccc3)o2)cc1S(=O)(=O)Nc1ccc(C(F)(F)F)cc1] |
| 612 | 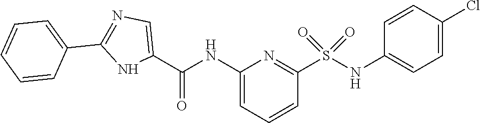 [SMILES: O=C(Nc1cccc(S(=O)(=O)Nc2ccc(Cl)cc2)n1)c1cnc(-c2ccccc2)[nH]1] |
| 670 | 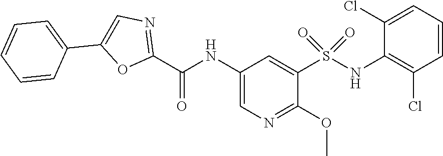 [SMILES: COc1ncc(NC(=O)c2ncc(-c3ccccc3)o2)cc1S(=O)(=O)Nc1c(Cl)cccc1Cl] |
| 1178 | 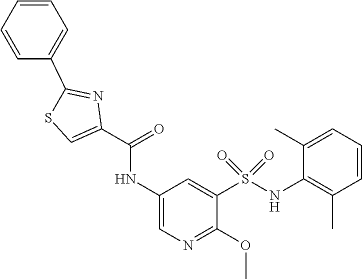 [SMILES: COc1ncc(NC(=O)c2csc(-c3ccccc3)n2)cc1S(=O)(=O)Nc1c(C)cccc1C] |
| 1193 | 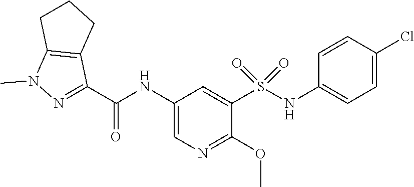 [SMILES: COc1ncc(NC(=O)c2nn(C)c3c2CCC3)cc1S(=O)(=O)Nc1ccc(Cl)cc1] |
| 1194 | 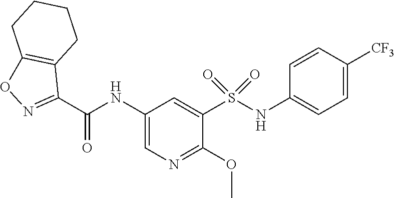 [SMILES: COc1ncc(NC(=O)c2noc3c2CCCC3)cc1S(=O)(=O)Nc1ccc(C(F)(F)F)cc1] |
| 1195 | 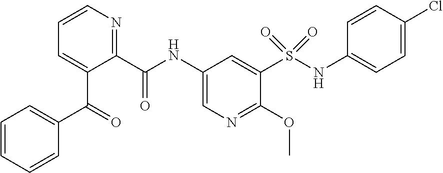 [SMILES: COc1ncc(NC(=O)c2ncccc2C(=O)c2ccccc2)cc1S(=O)(=O)Nc1ccc(Cl)cc1] |
| 1197 | 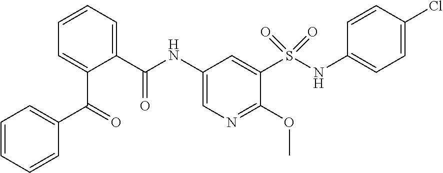 [SMILES: COc1ncc(NC(=O)c2ccccc2C(=O)c2ccccc2)cc1S(=O)(=O)Nc1ccc(Cl)cc1] |
| 1198 | 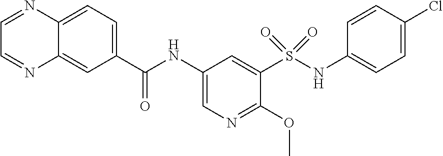 [SMILES: COc1ncc(NC(=O)c2ccc3nccnc3c2)cc1S(=O)(=O)Nc1ccc(Cl)cc1] |
| 1199 | 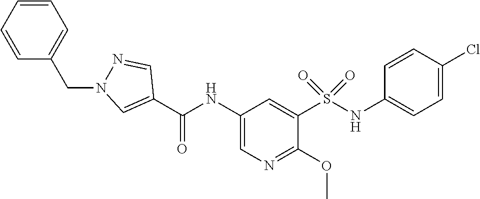 [SMILES: COc1ncc(NC(=O)c2cnn(Cc3ccccc3)c2)cc1S(=O)(=O)Nc1ccc(Cl)cc1] |
| 1200 | 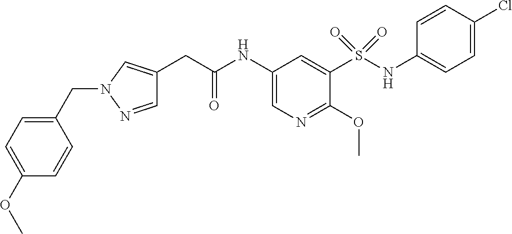 [SMILES: COc1ccc(Cn2cc(CC(=O)Nc3cnc(OC)c(S(=O)(=O)Nc4ccc(Cl)cc4)c3)cn2)cc1] |
| 1201 | 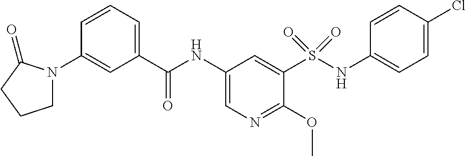 [SMILES: COc1ncc(NC(=O)c2cccc(N3CCCC3=O)c2)cc1S(=O)(=O)Nc1ccc(Cl)cc1] |
| 1202 | 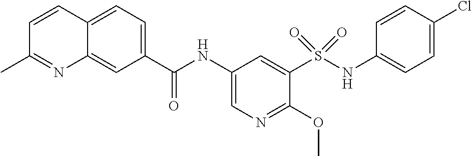 [SMILES: COc1ncc(NC(=O)c2ccc3ccc(C)nc3c2)cc1S(=O)(=O)Nc1ccc(Cl)cc1] |
| 1203 | 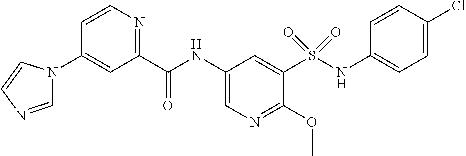 [SMILES: COc1ncc(NC(=O)c2cc(-n3ccnc3)ccn2)cc1S(=O)(=O)Nc1ccc(Cl)cc1] |
| 1204 | 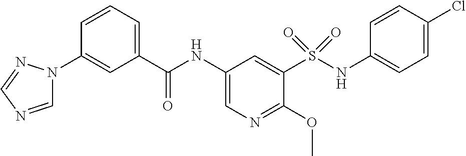 [SMILES: COc1ncc(NC(=O)c2cccc(-n3cncn3)c2)cc1S(=O)(=O)Nc1ccc(Cl)cc1] |
| 1206 | 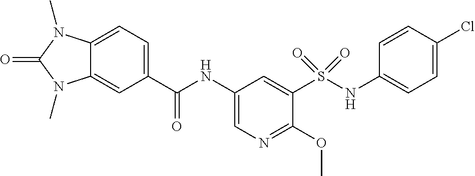 [SMILES: COc1ncc(NC(=O)c2ccc3c(c2)n(C)c(=O)n3C)cc1S(=O)(=O)Nc1ccc(Cl)cc1] |
| 1213 | 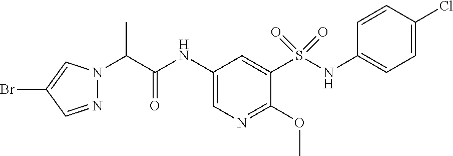 [SMILES: COc1ncc(NC(=O)C(C)n2cc(Br)cn2)cc1S(=O)(=O)Nc1ccc(Cl)cc1] |
| 1294 | 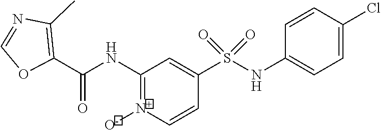 [SMILES: Cc1ncoc1C(=O)Nc1cc(S(=O)(=O)Nc2ccc(Cl)cc2)cc[n+]1[O-]] |
| 1303 | 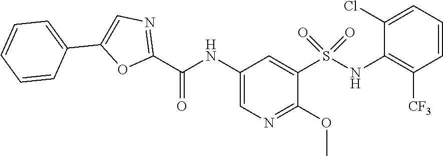 [SMILES: COc1ncc(NC(=O)c2ncc(-c3ccccc3)o2)cc1S(=O)(=O)Nc1c(Cl)cccc1C(F)(F)F] |
| 1312 | 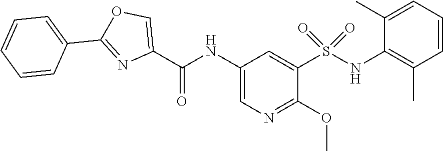 [SMILES: COc1ncc(NC(=O)c2coc(-c3ccccc3)n2)cc1S(=O)(=O)Nc1c(C)cccc1C] |
| 1321 | 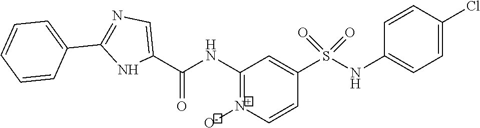 [SMILES: O=C(Nc1cc(S(=O)(=O)Nc2ccc(Cl)cc2)cc[n+]1[O-])c1cnc(-c2ccccc2)[nH]1] |
| 1350 | 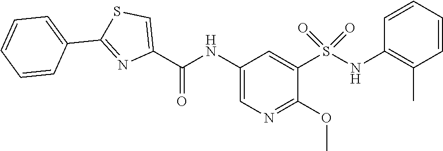 [SMILES: COc1ncc(NC(=O)c2csc(-c3ccccc3)n2)cc1S(=O)(=O)Nc1ccccc1C] |
| 1351 | 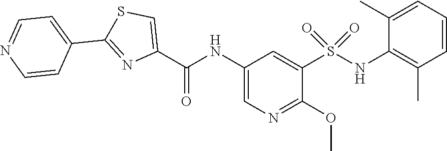 [SMILES: COc1ncc(NC(=O)c2csc(-c3ccncc3)n2)cc1S(=O)(=O)Nc1c(C)cccc1C] |
| 1358 | 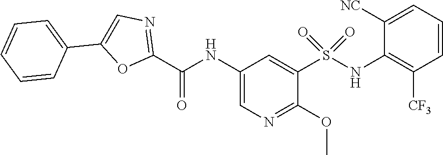 [SMILES: COc1ncc(NC(=O)c2ncc(-c3ccccc3)o2)cc1S(=O)(=O)Nc1c(C#N)cccc1C(F)(F)F] |
| 1359 | 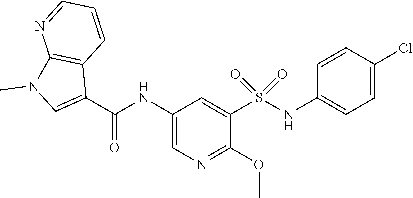 [SMILES: COc1ncc(NC(=O)c2cn(C)c3ncccc23)cc1S(=O)(=O)Nc1ccc(Cl)cc1] |
| 1361 | 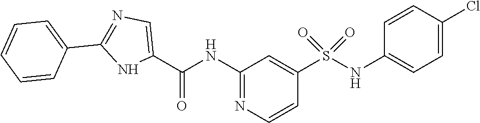 [SMILES: O=C(Nc1cc(S(=O)(=O)Nc2ccc(Cl)cc2)ccn1)c1cnc(-c2ccccc2)[nH]1] |
| 1364 | 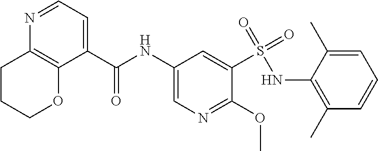 [SMILES: COc1ncc(NC(=O)c2ccnc3c2OCCC3)cc1S(=O)(=O)Nc1c(C)cccc1C] |
| 1365 | 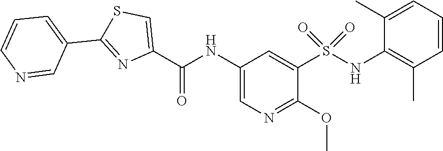 [SMILES: COc1ncc(NC(=O)c2csc(-c3cccnc3)n2)cc1S(=O)(=O)Nc1c(C)cccc1C] |
| 1366 | 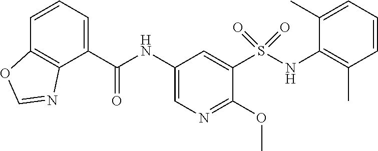 [SMILES: COc1ncc(NC(=O)c2cccc3ocnc23)cc1S(=O)(=O)Nc1c(C)cccc1C] |
| 1367 | 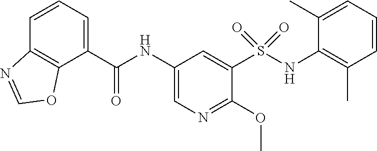 [SMILES: COc1ncc(NC(=O)c2cccc3ncoc23)cc1S(=O)(=O)Nc1c(C)cccc1C] |
| 1377 | 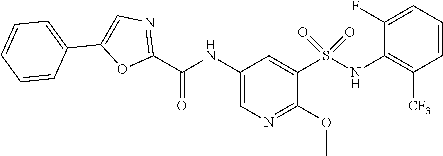 [SMILES: COc1ncc(NC(=O)c2ncc(-c3ccccc3)o2)cc1S(=O)(=O)Nc1c(F)cccc1C(F)(F)F] |
| 1379 | 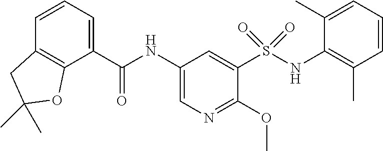 [SMILES: COc1ncc(NC(=O)c2cccc3c2OC(C)(C)C3)cc1S(=O)(=O)Nc1c(C)cccc1C] |
| 1380 | 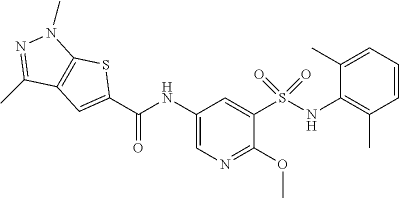 [SMILES: COc1ncc(NC(=O)c2cc3c(C)nn(C)c3s2)cc1S(=O)(=O)Nc1c(C)cccc1C] |
| 1382 | 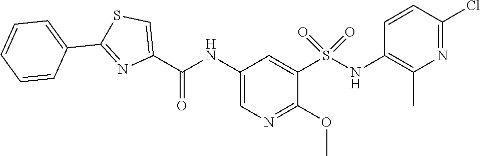 [SMILES: COc1ncc(NC(=O)c2csc(-c3ccccc3)n2)cc1S(=O)(=O)Nc1ccc(Cl)nc1C] |
| 1383 | 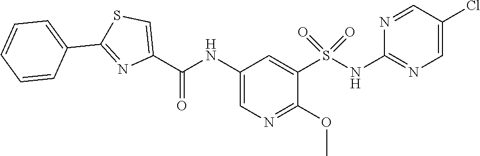 [SMILES: COc1ncc(NC(=O)c2csc(-c3ccccc3)n2)cc1S(=O)(=O)Nc1ncc(Cl)cn1] |
| 1385 | 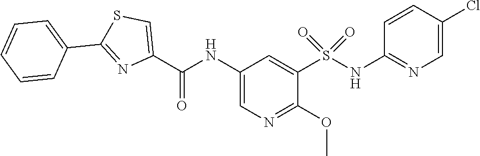 [SMILES: COc1ncc(NC(=O)c2csc(-c3ccccc3)n2)cc1S(=O)(=O)Nc1ccc(Cl)cn1] |
| 1386 | 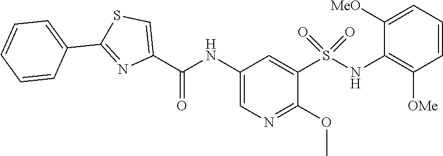 [SMILES: COc1cccc(OC)c1NS(=O)(=O)c1cc(NC(=O)c2csc(-c3ccccc3)n2)cnc1OC] |
| 1388 | 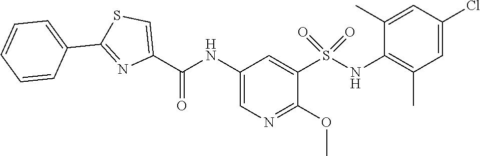 [SMILES: COc1ncc(NC(=O)c2csc(-c3ccccc3)n2)cc1S(=O)(=O)Nc1c(C)cc(Cl)cc1C] |
| 1389 | 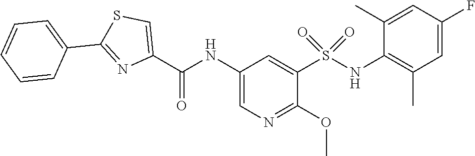 [SMILES: COc1ncc(NC(=O)c2csc(-c3ccccc3)n2)cc1S(=O)(=O)Nc1c(C)cc(F)cc1C] |
| 1390 | 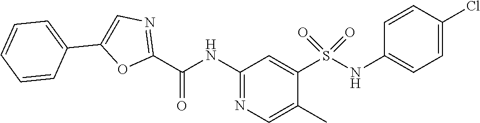 [SMILES: Cc1cnc(NC(=O)c2ncc(-c3ccccc3)o2)cc1S(=O)(=O)Nc1ccc(Cl)cc1] |
| 1393 | 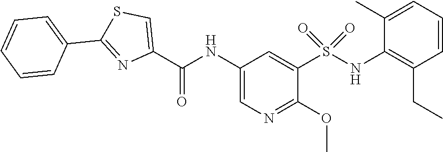 [SMILES: CCc1cccc(C)c1NS(=O)(=O)c1cc(NC(=O)c2csc(-c3ccccc3)n2)cnc1OC] |
| 1394 | 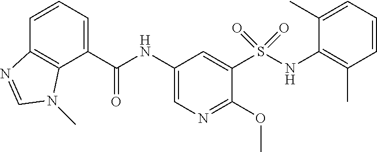 [SMILES: COc1ncc(NC(=O)c2cccc3ncn(C)c23)cc1S(=O)(=O)Nc1c(C)cccc1C] |
| 1395 | 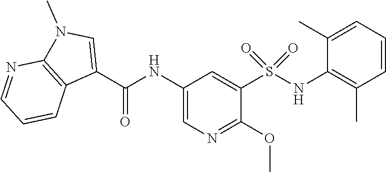 [SMILES: COc1ncc(NC(=O)c2cn(C)c3ncccc23)cc1S(=O)(=O)Nc1c(C)cccc1C] |
| 1396 | 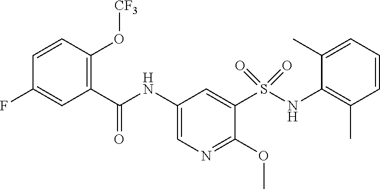 [SMILES: COc1ncc(NC(=O)c2cc(F)ccc2OC(F)(F)F)cc1S(=O)(=O)Nc1c(C)cccc1C] |
| 1398 | 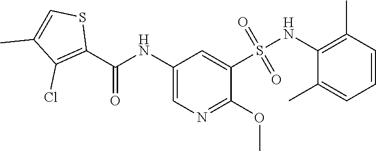 [SMILES: COc1ncc(NC(=O)c2scc(C)c2Cl)cc1S(=O)(=O)Nc1c(C)cccc1C] |
| 1400 | 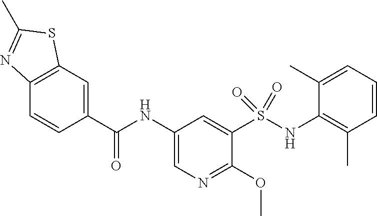 [SMILES: COc1ncc(NC(=O)c2ccc3nc(C)sc3c2)cc1S(=O)(=O)Nc1c(C)cccc1C] |
| 1401 | 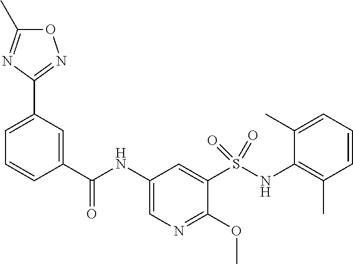 [SMILES: COc1ncc(NC(=O)c2cccc(-c3noc(C)n3)c2)cc1S(=O)(=O)Nc1c(C)cccc1C] |
| 1402 | 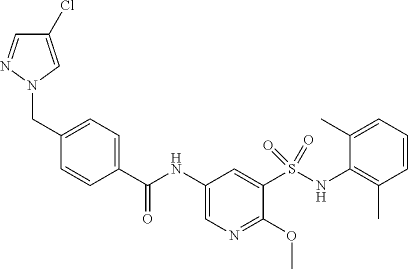 [SMILES: COc1ncc(NC(=O)c2ccc(Cn3cc(Cl)cn3)cc2)cc1S(=O)(=O)Nc1c(C)cccc1C] |
| 1403 | 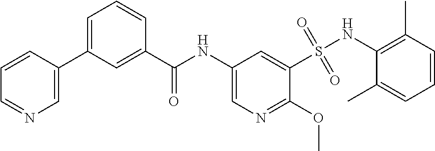 [SMILES: COc1ncc(NC(=O)c2cccc(-c3cccnc3)c2)cc1S(=O)(=O)Nc1c(C)cccc1C] |
| 1404 | 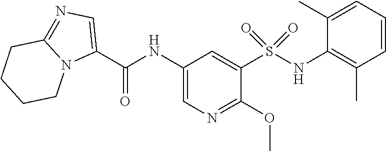 [SMILES: COc1ncc(NC(=O)c2cnc3n2CCCC3)cc1S(=O)(=O)Nc1c(C)cccc1C] |
| 1406 | 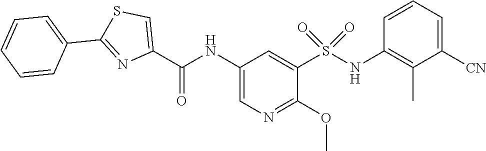 [SMILES: COc1ncc(NC(=O)c2csc(-c3ccccc3)n2)cc1S(=O)(=O)Nc1cccc(C#N)c1C] |
| 1413 | 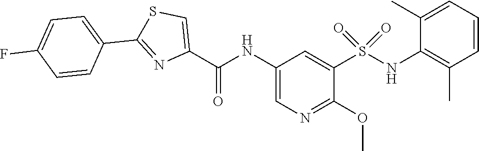 [SMILES: COc1ncc(NC(=O)c2csc(-c3ccc(F)cc3)n2)cc1S(=O)(=O)Nc1c(C)cccc1C] |
| 1417 | 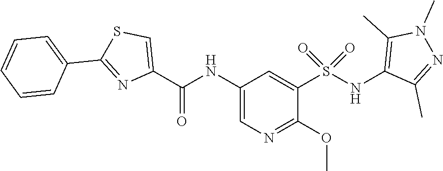 [SMILES: COc1ncc(NC(=O)c2csc(-c3ccccc3)n2)cc1S(=O)(=O)Nc1c(C)nn(C)c1C] |
| 1418 | 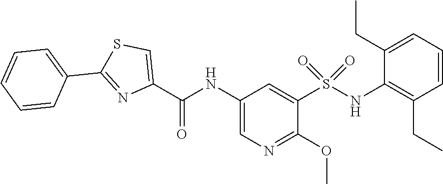 [SMILES: CCc1cccc(CC)c1NS(=O)(=O)c1cc(NC(=O)c2csc(-c3ccccc3)n2)cnc1OC] |
| 1420 | 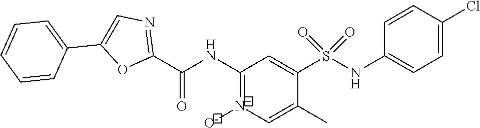 [SMILES: Cc1c[n+]([O-])c(NC(=O)c2ncc(-c3ccccc3)o2)cc1S(=O)(=O)Nc1ccc(Cl)cc1] |
| 1421 | 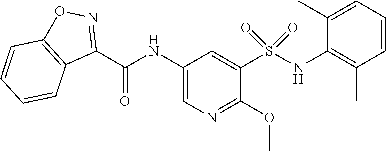 [SMILES: COc1ncc(NC(=O)c2noc3ccccc23)cc1S(=O)(=O)Nc1c(C)cccc1C] |
| 1422 | 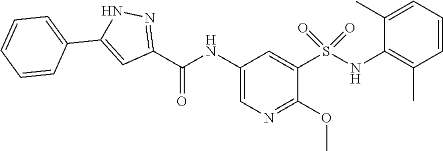 [SMILES: COc1ncc(NC(=O)c2cc(-c3ccccc3)[nH]n2)cc1S(=O)(=O)Nc1c(C)cccc1C] |
| 1423 | 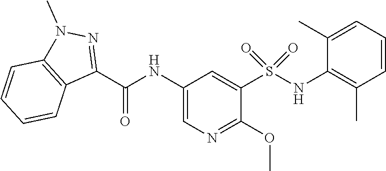 [SMILES: COc1ncc(NC(=O)c2nn(C)c3ccccc23)cc1S(=O)(=O)Nc1c(C)cccc1C] |
| 1424 | 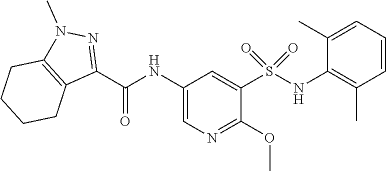 [SMILES: COc1ncc(NC(=O)c2nn(C)c3c2CCCC3)cc1S(=O)(=O)Nc1c(C)cccc1C] |
| 1425 | 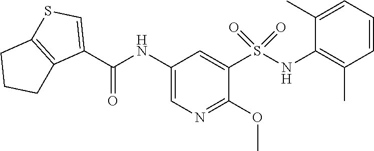 [SMILES: COc1ncc(NC(=O)c2csc3c2CCC3)cc1S(=O)(=O)Nc1c(C)cccc1C] |
| 1481 | 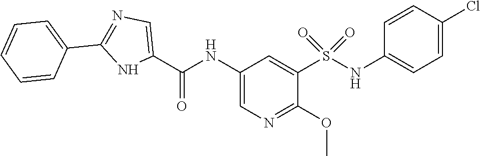 [SMILES: COc1ncc(NC(=O)c2cnc(-c3ccccc3)[nH]2)cc1S(=O)(=O)Nc1ccc(Cl)cc1] |
| 1483 | 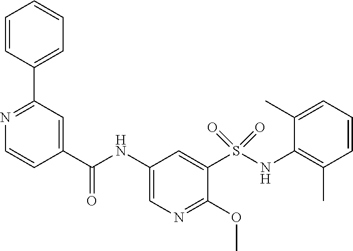 [SMILES: COc1ncc(NC(=O)c2ccnc(-c3ccccc3)c2)cc1S(=O)(=O)Nc1c(C)cccc1C] |
| 1484 | 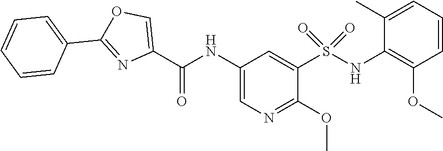 [SMILES: COc1cccc(C)c1NS(=O)(=O)c1cc(NC(=O)c2coc(-c3ccccc3)n2)cnc1OC] |
| 1485 | 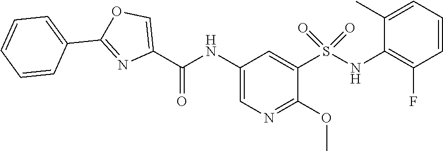 [SMILES: COc1ncc(NC(=O)c2coc(-c3ccccc3)n2)cc1S(=O)(=O)Nc1c(C)cccc1F] |
| 1486 | 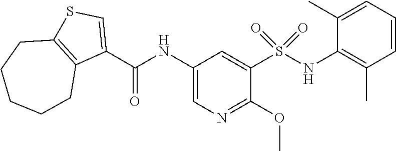 [SMILES: COc1ncc(NC(=O)c2csc3c2CCCCC3)cc1S(=O)(=O)Nc1c(C)cccc1C] |
| 1503 | 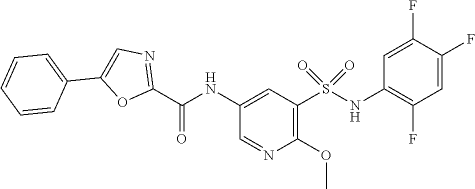 [SMILES: COc1ncc(NC(=O)c2ncc(-c3ccccc3)o2)cc1S(=O)(=O)Nc1cc(F)c(F)cc1F] |
| 1511 | 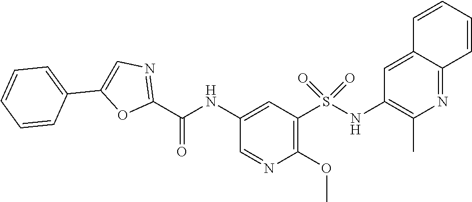 [SMILES: COc1ncc(NC(=O)c2ncc(-c3ccccc3)o2)cc1S(=O)(=O)Nc1cc2ccccc2nc1C] |
| 1513 | 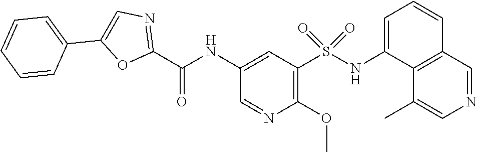 [SMILES: COc1ncc(NC(=O)c2ncc(-c3ccccc3)o2)cc1S(=O)(=O)Nc1cccc2cncc(C)c12] |
| 1514 | 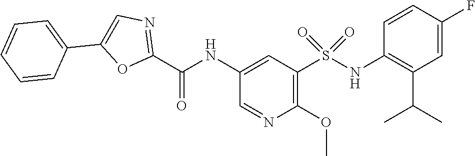 [SMILES: COc1ncc(NC(=O)c2ncc(-c3ccccc3)o2)cc1S(=O)(=O)Nc1ccc(F)cc1C(C)C] |
| 1519 | 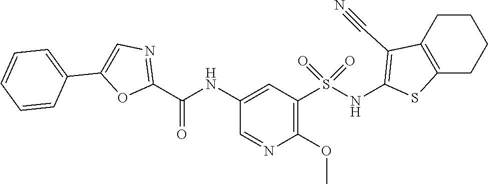 [SMILES: COc1ncc(NC(=O)c2ncc(-c3ccccc3)o2)cc1S(=O)(=O)Nc1sc2c(c1C#N)CCCC2] |
| 1525 | 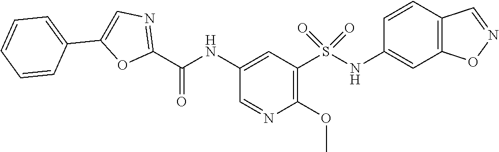 [SMILES: COc1ncc(NC(=O)c2ncc(-c3ccccc3)o2)cc1S(=O)(=O)Nc1ccc2cnoc2c1] |
| 1538 | 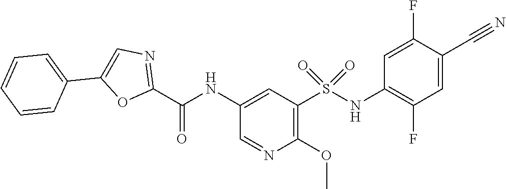 [SMILES: COc1ncc(NC(=O)c2ncc(-c3ccccc3)o2)cc1S(=O)(=O)Nc1cc(F)c(C#N)cc1F] |
| 1552 | 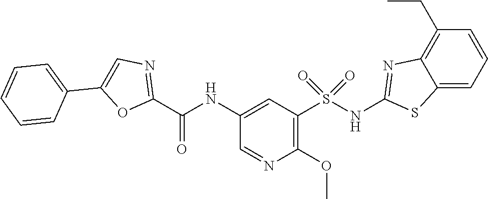 [SMILES: CCc1cccc2sc(NS(=O)(=O)c3cc(NC(=O)c4ncc(-c5ccccc5)o4)cnc3OC)nc12] |
| 1567 | 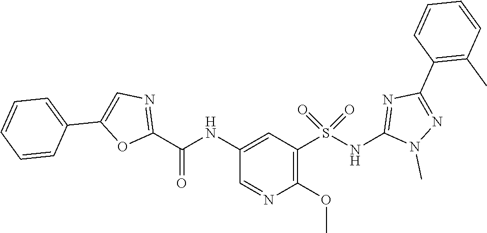 [SMILES: COc1ncc(NC(=O)c2ncc(-c3ccccc3)o2)cc1S(=O)(=O)Nc1nc(-c2ccccc2C)nn1C] |
| 1568 | 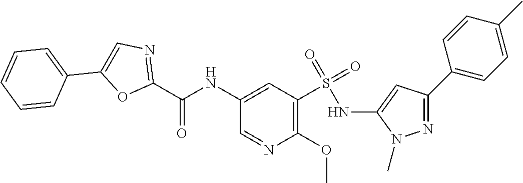 [SMILES: COc1ncc(NC(=O)c2ncc(-c3ccccc3)o2)cc1S(=O)(=O)Nc1cc(-c2ccc(C)cc2)nn1C] |
| 1571 | 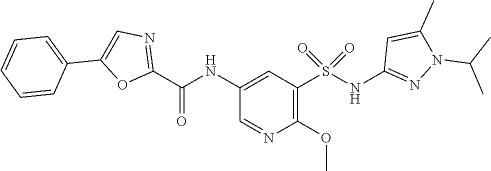 [SMILES: COc1ncc(NC(=O)c2ncc(-c3ccccc3)o2)cc1S(=O)(=O)Nc1cc(C)n(C(C)C)n1] |
| 1624 | 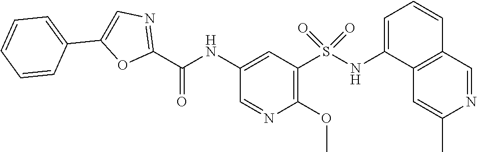 [SMILES: COc1ncc(NC(=O)c2ncc(-c3ccccc3)o2)cc1S(=O)(=O)Nc1cccc2cnc(C)cc12] |
| 1640 | 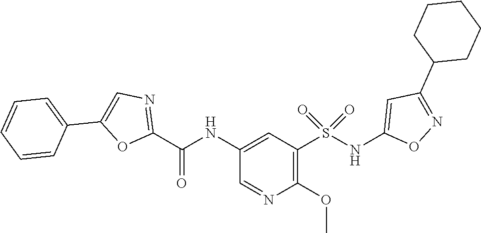 [SMILES: COc1ncc(NC(=O)c2ncc(-c3ccccc3)o2)cc1S(=O)(=O)Nc1cc(C2CCCCC2)no1] |
| 1648 | 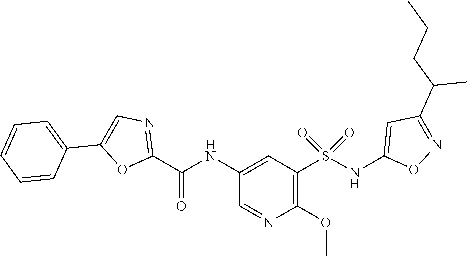 [SMILES: CCCC(C)c1cc(NS(=O)(=O)c2cc(NC(=O)c3ncc(-c4ccccc4)o3)cnc2OC)on1] |
| 1661 | 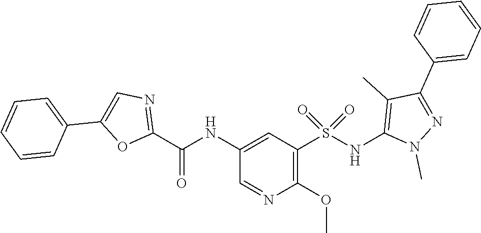 [SMILES: COc1ncc(NC(=O)c2ncc(-c3ccccc3)o2)cc1S(=O)(=O)Nc1c(C)c(-c2ccccc2)nn1C] |
| 1706 | 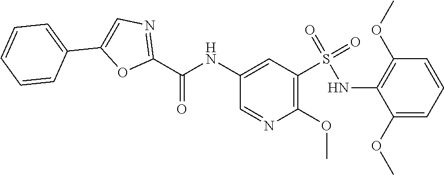 [SMILES: COc1cccc(OC)c1NS(=O)(=O)c1cc(NC(=O)c2ncc(-c3ccccc3)o2)cnc1OC] |
| 1729 | 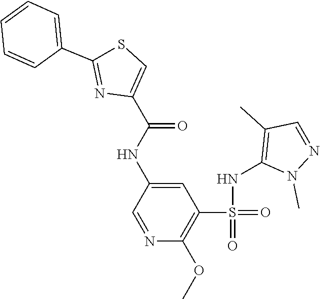 [SMILES: COc1ncc(NC(=O)c2csc(-c3ccccc3)n2)cc1S(=O)(=O)Nc1c(C)cnn1C] |
| 1746 | 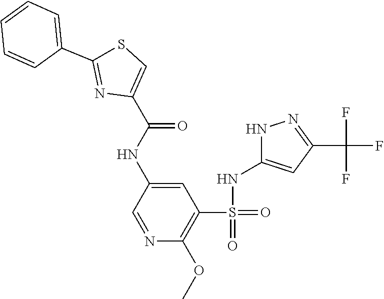 [SMILES: COc1ncc(NC(=O)c2csc(-c3ccccc3)n2)cc1S(=O)(=O)Nc1cc(C(F)(F)F)n[nH]1] |
| 1754 | 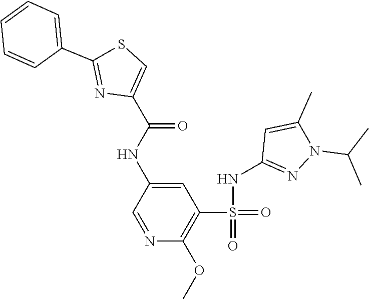 [SMILES: COc1ncc(NC(=O)c2csc(-c3ccccc3)n2)cc1S(=O)(=O)Nc1cc(C)n(C(C)C)n1] |
| 1760 | 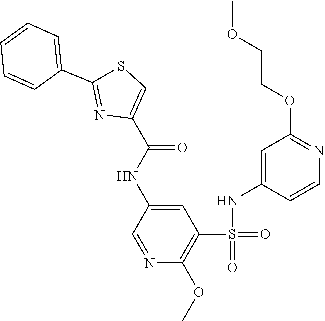 [SMILES: COCCOc1cc(NS(=O)(=O)c2cc(NC(=O)c3csc(-c4ccccc4)n3)cnc2OC)ccn1] |
| 1768 | 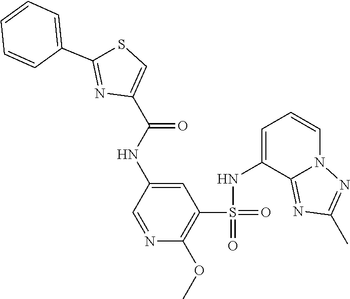 [SMILES: COc1ncc(NC(=O)c2csc(-c3ccccc3)n2)cc1S(=O)(=O)Nc1cccn2nc(C)nc12] |
| 1773 | 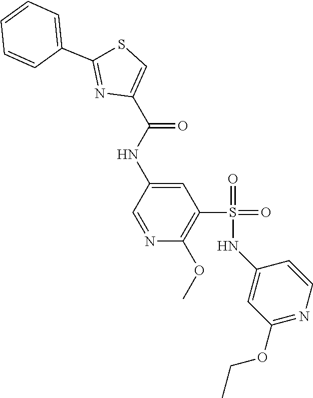 [SMILES: CCOc1cc(NS(=O)(=O)c2cc(NC(=O)c3csc(-c4ccccc4)n3)cnc2OC)ccn1] |
| 1779 | 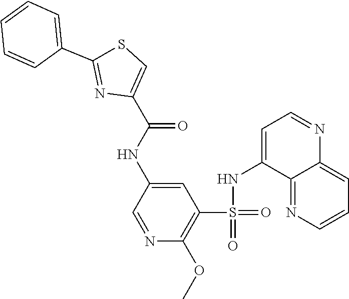 [SMILES: COc1ncc(NC(=O)c2csc(-c3ccccc3)n2)cc1S(=O)(=O)Nc1ccnc2cccnc12] |
| 1780 | 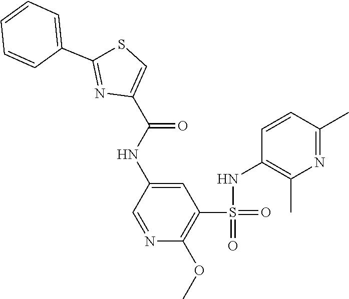 [SMILES: COc1ncc(NC(=O)c2csc(-c3ccccc3)n2)cc1S(=O)(=O)Nc1ccc(C)nc1C] |
| 1784 | 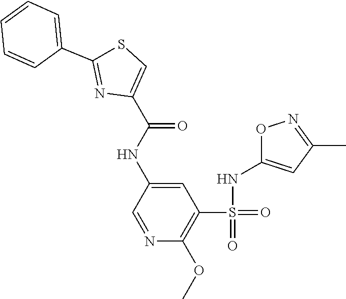 [SMILES: COc1ncc(NC(=O)c2csc(-c3ccccc3)n2)cc1S(=O)(=O)Nc1cc(C)no1] |
| 1785 | 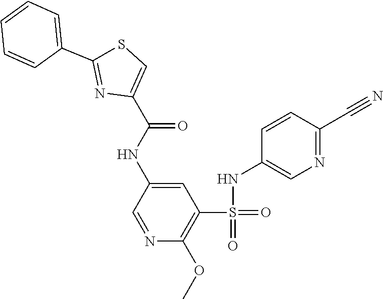 [SMILES: COc1ncc(NC(=O)c2csc(-c3ccccc3)n2)cc1S(=O)(=O)Nc1ccc(C#N)nc1] |
| 1792 | 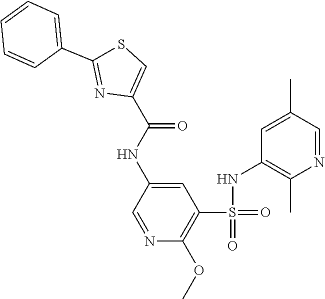 [SMILES: COc1ncc(NC(=O)c2csc(-c3ccccc3)n2)cc1S(=O)(=O)Nc1cc(C)cnc1C] |
| 1808 | 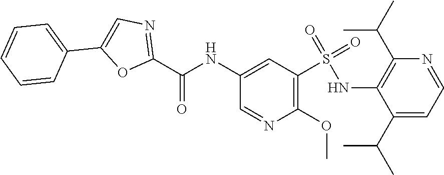 [SMILES: COc1ncc(NC(=O)c2ncc(-c3ccccc3)o2)cc1S(=O)(=O)Nc1c(C(C)C)ccnc1C(C)C] |
| 1824 | 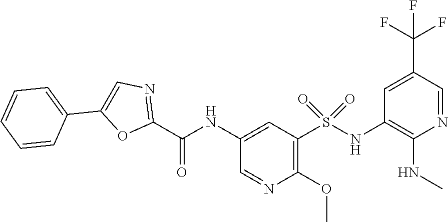 [SMILES: CNc1ncc(C(F)(F)F)cc1NS(=O)(=O)c1cc(NC(=O)c2ncc(-c3ccccc3)o2)cnc1OC] |
| 1839 | 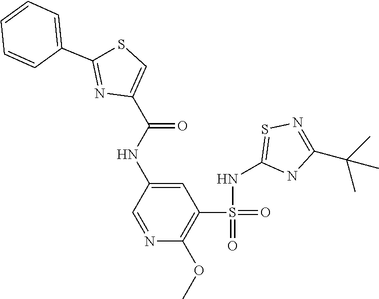 |
| 1878 | 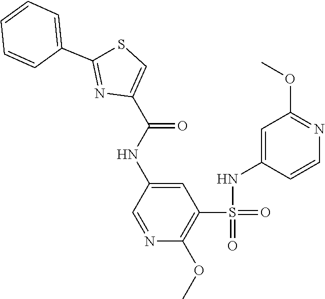 [SMILES: COc1cc(NS(=O)(=O)c2cc(NC(=O)c3csc(-c4ccccc4)n3)cnc2OC)ccn1] |
| 1895 | 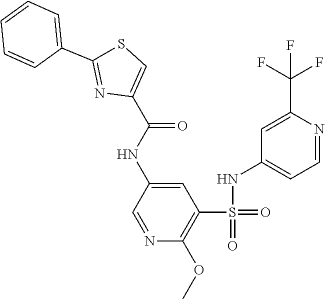 [SMILES: COc1ncc(NC(=O)c2csc(-c3ccccc3)n2)cc1S(=O)(=O)Nc1ccnc(C(F)(F)F)c1] |
| 1898 | 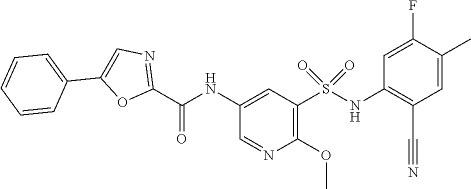 [SMILES: COc1ncc(NC(=O)c2ncc(-c3ccccc3)o2)cc1S(=O)(=O)Nc1cc(F)c(C)cc1C#N] |
| 1908 | 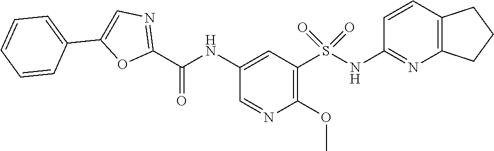 [SMILES: COc1ncc(NC(=O)c2ncc(-c3ccccc3)o2)cc1S(=O)(=O)Nc1ccc2c(n1)CCC2] |
| 1911 | 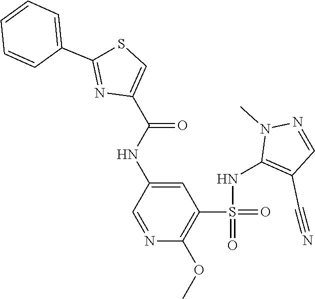 [SMILES: COc1ncc(NC(=O)c2csc(-c3ccccc3)n2)cc1S(=O)(=O)Nc1c(C#N)cnn1C] |
| 1912 | 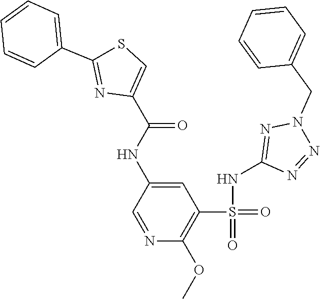 [SMILES: COc1ncc(NC(=O)c2csc(-c3ccccc3)n2)cc1S(=O)(=O)Nc1nnn(Cc2ccccc2)n1] |
| 1926 | 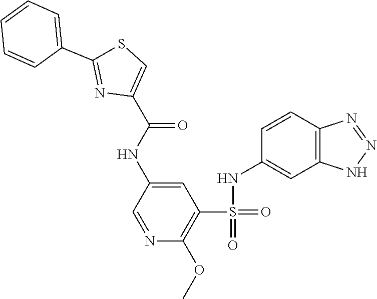 [SMILES: COc1ncc(NC(=O)c2csc(-c3ccccc3)n2)cc1S(=O)(=O)Nc1ccc2nn[nH]c2c1] |
| 1935 | 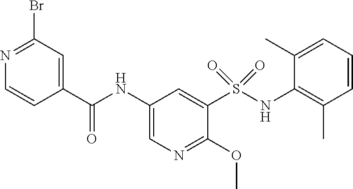 [SMILES: COc1ncc(NC(=O)c2ccnc(Br)c2)cc1S(=O)(=O)Nc1c(C)cccc1C] |
| 1937 | 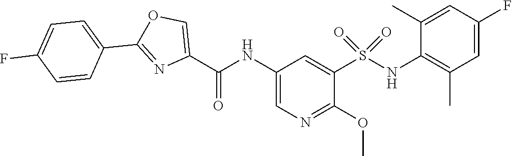 [SMILES: COc1ncc(NC(=O)c2coc(-c3ccc(F)cc3)n2)cc1S(=O)(=O)Nc1c(C)cc(F)cc1C] |
| 1940 | 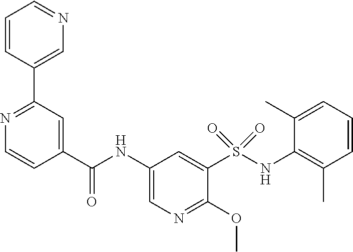 [SMILES: COc1ncc(NC(=O)c2ccnc(-c3cccnc3)c2)cc1S(=O)(=O)Nc1c(C)cccc1C] |
| 1941 | 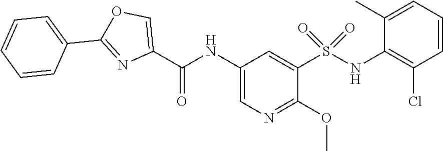 [SMILES: COc1ncc(NC(=O)c2coc(-c3ccccc3)n2)cc1S(=O)(=O)Nc1c(C)cccc1Cl] |
| 1942 | 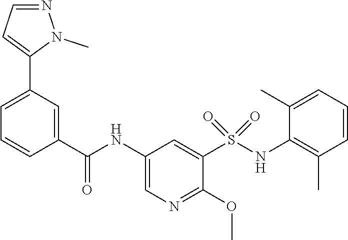 [SMILES: COc1ncc(NC(=O)c2cccc(-c3ccnn3C)c2)cc1S(=O)(=O)Nc1c(C)cccc1C] |
| 1943 | 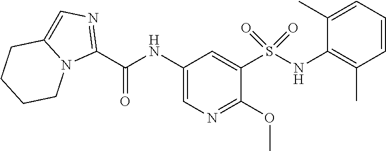 [SMILES: COc1ncc(NC(=O)c2ncc3n2CCCC3)cc1S(=O)(=O)Nc1c(C)cccc1C] |
| 1944 | 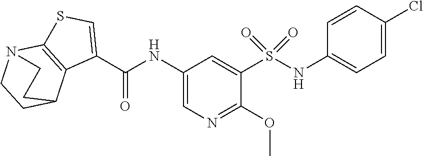 [SMILES: COc1ncc(NC(=O)c2csc3c2C2CCN3CC2)cc1S(=O)(=O)Nc1ccc(Cl)cc1] |
| 1946 | 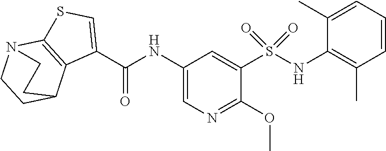 [SMILES: COc1ncc(NC(=O)c2csc3c2C2CCN3CC2)cc1S(=O)(=O)Nc1c(C)cccc1C] |
| 1949 | 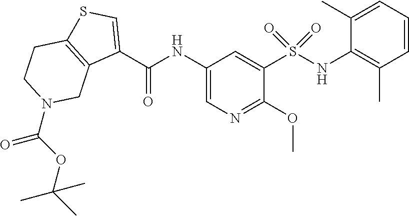 [SMILES: COc1ncc(NC(=O)c2csc3c2CN(C(=O)OC(C)(C)C)CC3)cc1S(=O)(=O)Nc1c(C)cccc1C] |
| 1950 | 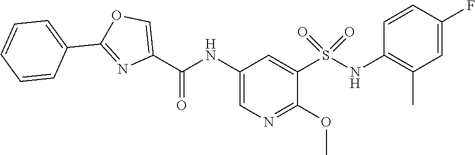 [SMILES: COc1ncc(NC(=O)c2coc(-c3ccccc3)n2)cc1S(=O)(=O)Nc1ccc(F)cc1C] |
| 1953 | 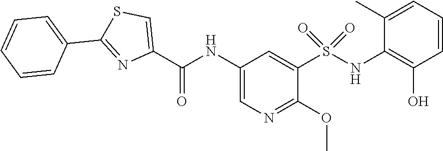 [SMILES: COc1ncc(NC(=O)c2csc(-c3ccccc3)n2)cc1S(=O)(=O)Nc1c(C)cccc1O] |
| 1954 | 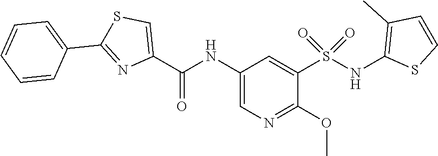 [SMILES: COc1ncc(NC(=O)c2csc(-c3ccccc3)n2)cc1S(=O)(=O)Nc1sccc1C] |
| 1956 | 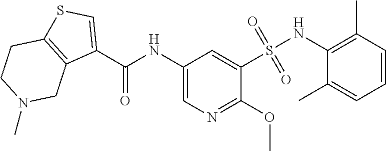 [SMILES: COc1ncc(NC(=O)c2csc3c2CN(C)CC3)cc1S(=O)(=O)Nc1c(C)cccc1C] |
| 1959 | 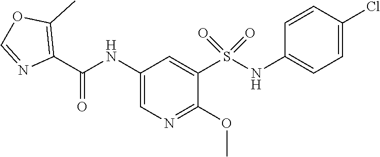 [SMILES: COc1ncc(NC(=O)c2ncoc2C)cc1S(=O)(=O)Nc1ccc(Cl)cc1] |
| 1962 | 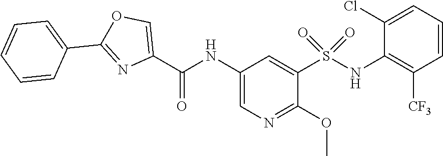 [SMILES: COc1ncc(NC(=O)c2coc(-c3ccccc3)n2)cc1S(=O)(=O)Nc1c(Cl)cccc1C(F)(F)F] |
| 1964 | 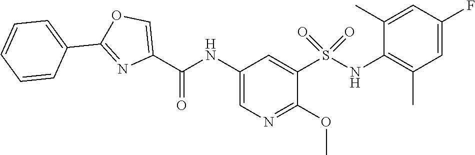 [SMILES: COc1ncc(NC(=O)c2coc(-c3ccccc3)n2)cc1S(=O)(=O)Nc1c(C)cc(F)cc1C] |
| 1965 | 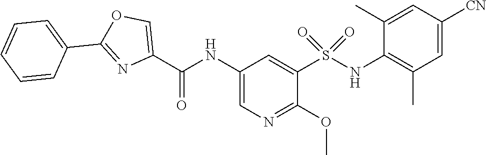 [SMILES: COc1ncc(NC(=O)c2coc(-c3ccccc3)n2)cc1S(=O)(=O)Nc1c(C)cc(C#N)cc1C] |
| 1966 | 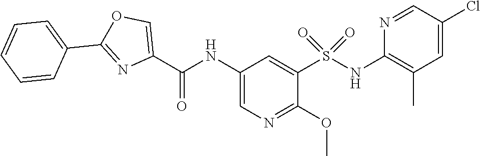 [SMILES: COc1ncc(NC(=O)c2coc(-c3ccccc3)n2)cc1S(=O)(=O)Nc1ncc(Cl)cc1C] |
| 1970 | 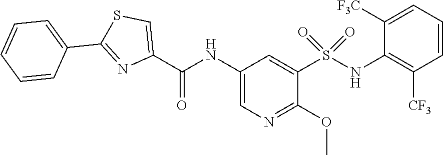 [SMILES: COc1ncc(NC(=O)c2csc(-c3ccccc3)n2)cc1S(=O)(=O)Nc1c(C(F)(F)F)cccc1C(F)(F)F] |
| 1972 | 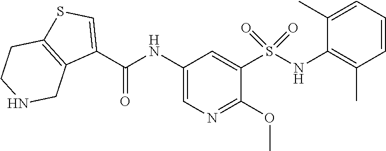 [SMILES: COc1ncc(NC(=O)c2csc3c2CNCC3)cc1S(=O)(=O)Nc1c(C)cccc1C] |
| 1977 | 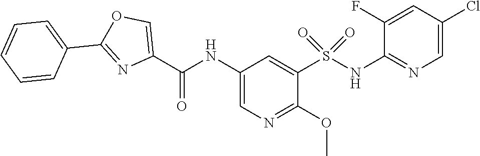 [SMILES: COc1ncc(NC(=O)c2coc(-c3ccccc3)n2)cc1S(=O)(=O)Nc1ncc(Cl)cc1F] |
| 1985 | 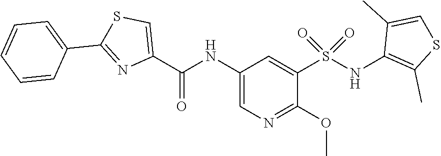 [SMILES: COc1ncc(NC(=O)c2csc(-c3ccccc3)n2)cc1S(=O)(=O)Nc1c(C)csc1C] |
| 1986 | 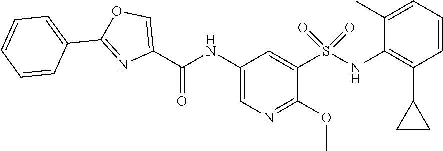 [SMILES: COc1ncc(NC(=O)c2coc(-c3ccccc3)n2)cc1S(=O)(=O)Nc1c(C)cccc1C1CC1] |
| 1990 | 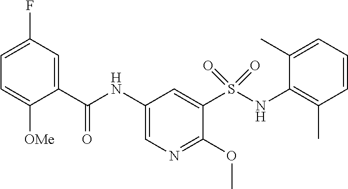 [SMILES: COc1ccc(F)cc1C(=O)Nc1cnc(OC)c(S(=O)(=O)Nc2c(C)cccc2C)c1] |
| 1991 | 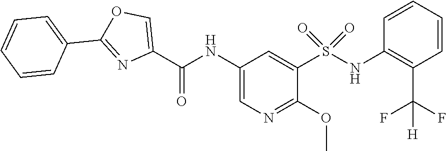 [SMILES: COc1ncc(NC(=O)c2coc(-c3ccccc3)n2)cc1S(=O)(=O)Nc1ccccc1C(F)F] |
| 2116 | 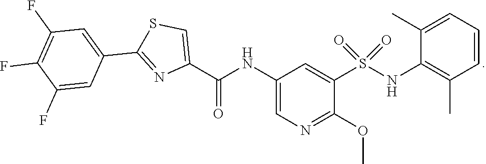 [SMILES: COc1ncc(NC(=O)c2csc(-c3cc(F)c(F)c(F)c3)n2)cc1S(=O)(=O)Nc1c(C)cccc1C] |

63. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

| No. | Formula |
|---|---|
| 1178 | 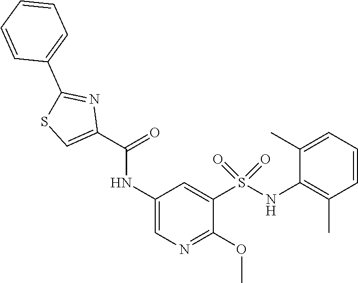 [SMILES: COc1ncc(NC(=O)c2csc(-c3ccccc3)n2)cc1S(=O)(=O)Nc1c(C)cccc1C] |
| 1206 | 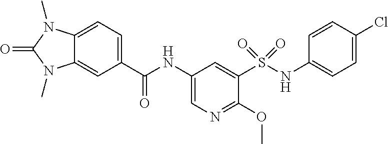 [SMILES: COc1ncc(NC(=O)c2ccc3c(c2)n(C)c(=O)n3C)cc1S(=O)(=O)Nc1ccc(Cl)cc1] |
| 1385 | 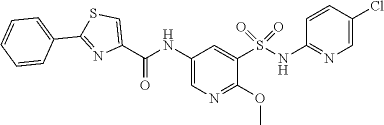 [SMILES: COc1ncc(NC(=O)c2csc(-c3ccccc3)n2)cc1S(=O)(=O)Nc1ccc(Cl)cn1] |
| 1481 | 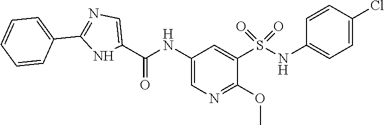 [SMILES: COc1ncc(NC(=O)c2cnc(-c3ccccc3)[nH]2)cc1S(=O)(=O)Nc1ccc(Cl)cc1] |
| 2116 | 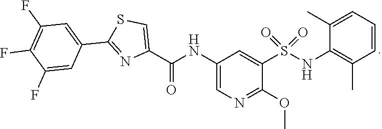 [SMILES: COc1ncc(NC(=O)c2csc(-c3cc(F)c(F)c(F)c3)n2)cc1S(=O)(=O)Nc1c(C)cccc1C] |

64. The compound of claim 1, or a pharmaceutically acceptable salt thereof, wherein the compound is of the formula:

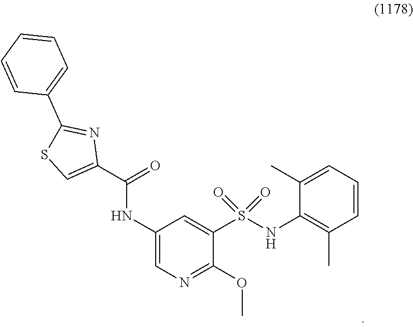 [SMILES: COc1ncc(NC(=O)c2csc(-c3ccccc3)n2)cc1S(=O)(=O)Nc1c(C)cccc1C]

65. The compound of claim 1, or a pharmaceutically acceptable salt thereof, wherein the compound is of the formula:

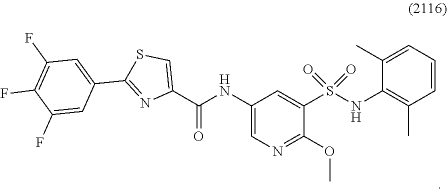 [SMILES: COc1ncc(NC(=O)c2csc(-c3cc(F)c(F)c(F)c3)n2)cc1S(=O)(=O)Nc1c(C)cccc1C]

66. The compound of claim 1, or a pharmaceutically acceptable salt thereof, wherein the compound is of the formula:

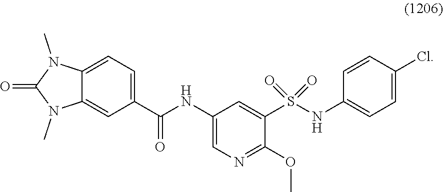 [SMILES: COc1ncc(NC(=O)c2ccc3c(c2)n(C)c(=O)n3C)cc1S(=O)(=O)Nc1ccc(Cl)cc1]

67. The compound of claim 1, or a pharmaceutically acceptable salt, solvate, hydrate, polymorph, co-crystal, tautomer, stereoisomer, isotopically labeled compound, or prodrug thereof, wherein the compound is of the formula:

| No. | Formula |
|---|---|
| 2000 | 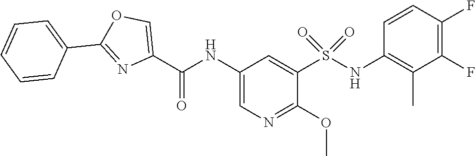 [SMILES: COc1ncc(NC(=O)c2coc(-c3ccccc3)n2)cc1S(=O)(=O)Nc1ccc(F)c(F)c1C] |
| 2002 | 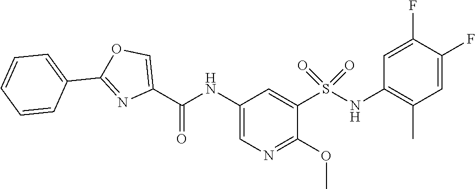 [SMILES: COc1ncc(NC(=O)c2coc(-c3ccccc3)n2)cc1S(=O)(=O)Nc1cc(F)c(F)cc1C] |
| 2004 | 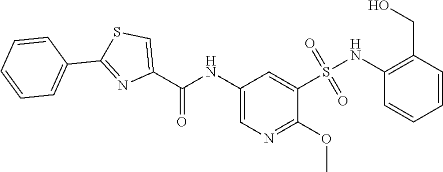 [SMILES: COc1ncc(NC(=O)c2csc(-c3ccccc3)n2)cc1S(=O)(=O)Nc1ccccc1CO] |
| 2005 | 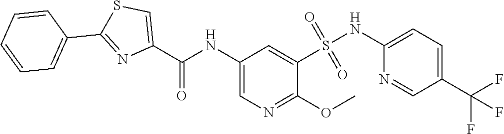 [SMILES: COc1ncc(NC(=O)c2csc(-c3ccccc3)n2)cc1S(=O)(=O)Nc1ccc(C(F)(F)F)cn1] |
| 2068 | 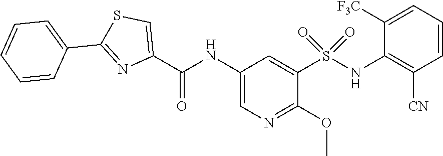 [SMILES: COc1ncc(NC(=O)c2csc(-c3ccccc3)n2)cc1S(=O)(=O)Nc1c(C#N)cccc1C(F)(F)F] |
| 2069 | 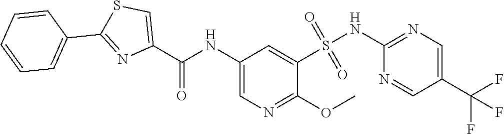 [SMILES: COc1ncc(NC(=O)c2csc(-c3ccccc3)n2)cc1S(=O)(=O)Nc1ncc(C(F)(F)F)cn1] |
| 2072 | 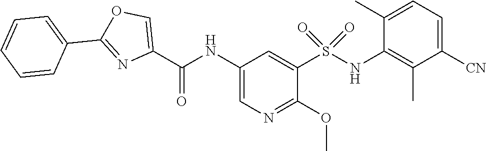 [SMILES: COc1ncc(NC(=O)c2coc(-c3ccccc3)n2)cc1S(=O)(=O)Nc1c(C)ccc(C#N)c1C] |
| 2088 | 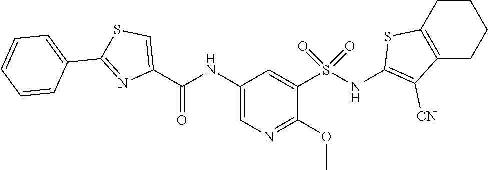 [SMILES: COc1ncc(NC(=O)c2csc(-c3ccccc3)n2)cc1S(=O)(=O)Nc1sc2c(c1C#N)CCCC2] |
| 2092 | 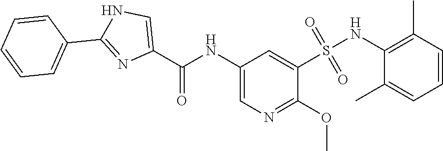 [SMILES: COc1ncc(NC(=O)c2c[nH]c(-c3ccccc3)n2)cc1S(=O)(=O)Nc1c(C)cccc1C] |
| 2119 | 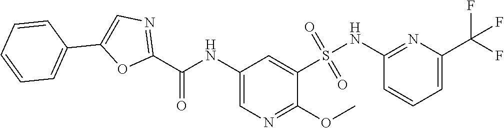 [SMILES: COc1ncc(NC(=O)c2ncc(-c3ccccc3)o2)cc1S(=O)(=O)Nc1cccc(C(F)(F)F)n1] |
| 2142 | 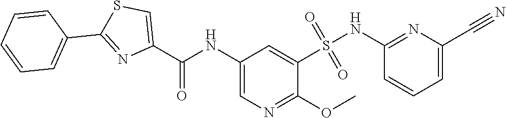 [SMILES: COc1ncc(NC(=O)c2csc(-c3ccccc3)n2)cc1S(=O)(=O)Nc1cccc(C#N)n1] |
| 2148 | 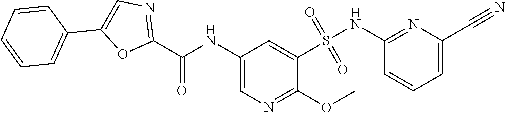 [SMILES: COc1ncc(NC(=O)c2ncc(-c3ccccc3)o2)cc1S(=O)(=O)Nc1cccc(C#N)n1] |
| 2151 | 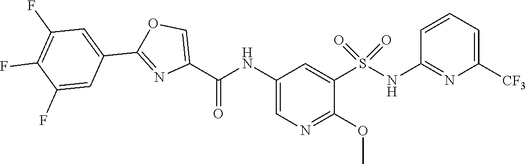 [SMILES: COc1ncc(NC(=O)c2coc(-c3cc(F)c(F)c(F)c3)n2)cc1S(=O)(=O)Nc1cccc(C(F)(F)F)n1] |
| 2155 | 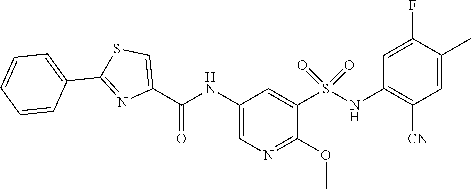 [SMILES: COc1ncc(NC(=O)c2csc(-c3ccccc3)n2)cc1S(=O)(=O)Nc1cc(F)c(C)cc1C#N] |
| 2156 | 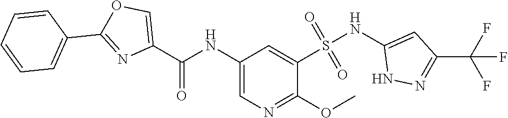 [SMILES: COc1ncc(NC(=O)c2coc(-c3ccccc3)n2)cc1S(=O)(=O)Nc1cc(C(F)(F)F)n[nH]1] |
| 2157 | 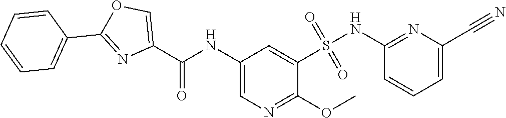 [SMILES: COc1ncc(NC(=O)c2coc(-c3ccccc3)n2)cc1S(=O)(=O)Nc1cccc(C#N)n1] |
| 2361 | 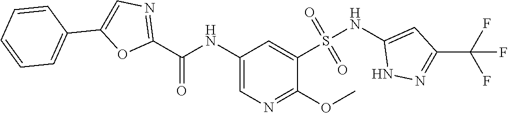 [SMILES: COc1ncc(NC(=O)c2ncc(-c3ccccc3)o2)cc1S(=O)(=O)Nc1cc(C(F)(F)F)n[nH]1] |
| 2365 | 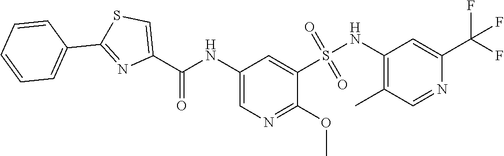 [SMILES: COc1ncc(NC(=O)c2csc(-c3ccccc3)n2)cc1S(=O)(=O)Nc1cc(C(F)(F)F)ncc1C] |
| 2366 | 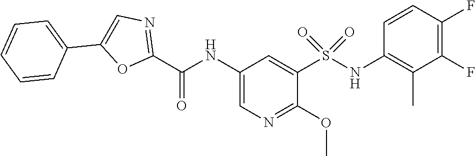 [SMILES: COc1ncc(NC(=O)c2ncc(-c3ccccc3)o2)cc1S(=O)(=O)Nc1ccc(F)c(F)c1C] |
| 2381 | 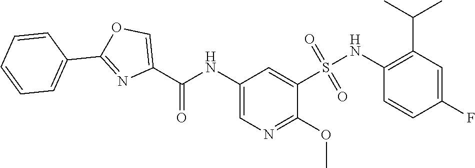 [SMILES: COc1ncc(NC(=O)c2coc(-c3ccccc3)n2)cc1S(=O)(=O)Nc1ccc(F)cc1C(C)C] |
| 2387 | 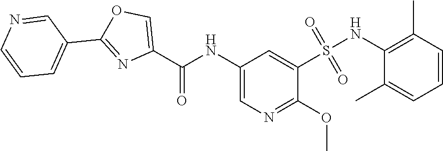 [SMILES: COc1ncc(NC(=O)c2coc(-c3cccnc3)n2)cc1S(=O)(=O)Nc1c(C)cccc1C] |
| 2390 | 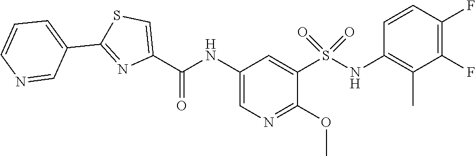 [SMILES: COc1ncc(NC(=O)c2csc(-c3cccnc3)n2)cc1S(=O)(=O)Nc1ccc(F)c(F)c1C] |
| 2396 | 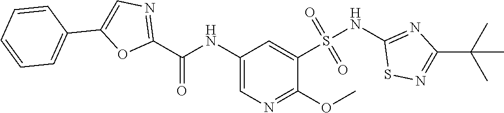 [SMILES: COc1ncc(NC(=O)c2ncc(-c3ccccc3)o2)cc1S(=O)(=O)Nc1nc(C(C)(C)C)ns1] |
| 2397 | 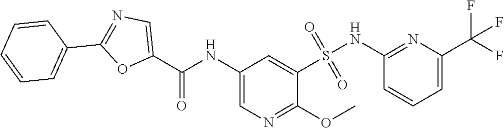 [SMILES: COc1ncc(NC(=O)c2cnc(-c3ccccc3)o2)cc1S(=O)(=O)Nc1cccc(C(F)(F)F)n1] |
| 2403 | 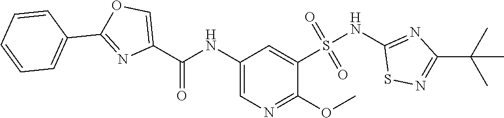 [SMILES: COc1ncc(NC(=O)c2coc(-c3ccccc3)n2)cc1S(=O)(=O)Nc1nc(C(C)(C)C)ns1] |
| 2407 | 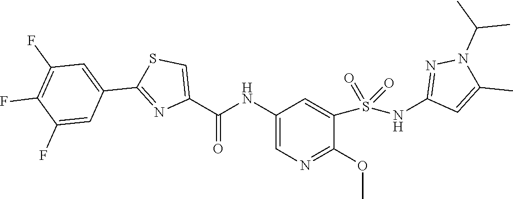 [SMILES: COc1ncc(NC(=O)c2csc(-c3cc(F)c(F)c(F)c3)n2)cc1S(=O)(=O)Nc1cc(C)n(C(C)C)n1] |
| 2418 | 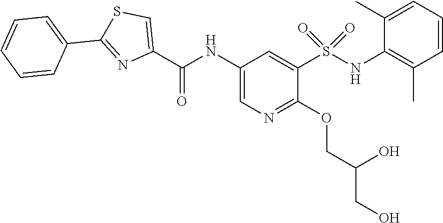 [SMILES: Cc1cccc(C)c1NS(=O)(=O)c1cc(NC(=O)c2csc(-c3ccccc3)n2)cnc1OCC(O)CO] |
| 2427 | 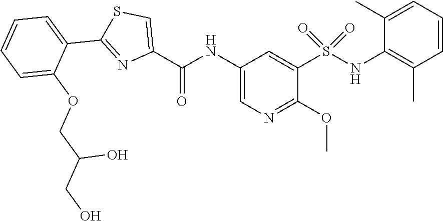 [SMILES: COc1ncc(NC(=O)c2csc(-c3ccccc3OCC(O)CO)n2)cc1S(=O)(=O)Nc1c(C)cccc1C] |
| 2431 | 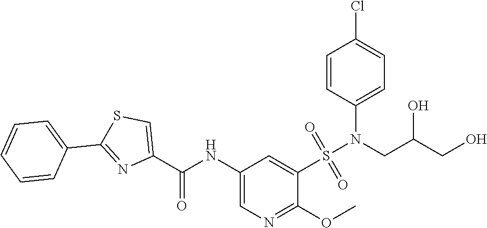 [SMILES: COc1ncc(NC(=O)c2csc(-c3ccccc3)n2)cc1S(=O)(=O)N(CC(O)CO)c1ccc(Cl)cc1] |
| 2445 | 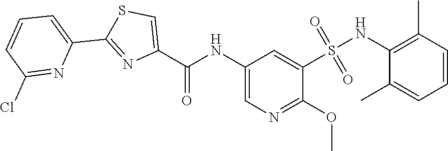 [SMILES: COc1ncc(NC(=O)c2csc(-c3cccc(Cl)n3)n2)cc1S(=O)(=O)Nc1c(C)cccc1C] |
| 2447 | 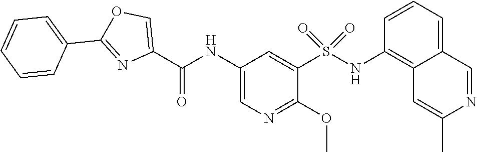 [SMILES: COc1ncc(NC(=O)c2coc(-c3ccccc3)n2)cc1S(=O)(=O)Nc1cccc2cnc(C)cc12] |
| 2454 | 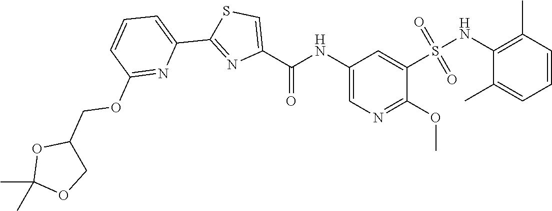 [SMILES: COc1ncc(NC(=O)c2csc(-c3cccc(OCC4COC(C)(C)O4)n3)n2)cc1S(=O)(=O)Nc1c(C)cccc1C] |
| 2463 | 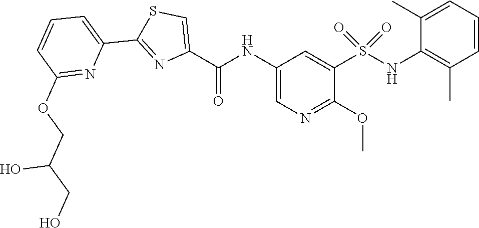 [SMILES: COc1ncc(NC(=O)c2csc(-c3cccc(OCC(O)CO)n3)n2)cc1S(=O)(=O)Nc1c(C)cccc1C] |
| 2466 | 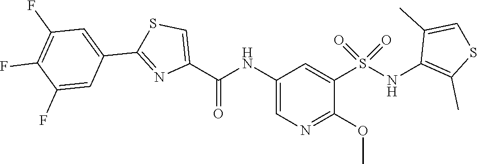 [SMILES: COc1ncc(NC(=O)c2csc(-c3cc(F)c(F)c(F)c3)n2)cc1S(=O)(=O)Nc1c(C)csc1C] |
| 2470 | 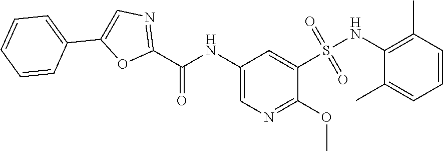 [SMILES: COc1ncc(NC(=O)c2ncc(-c3ccccc3)o2)cc1S(=O)(=O)Nc1c(C)cccc1C] |
| 2474 | 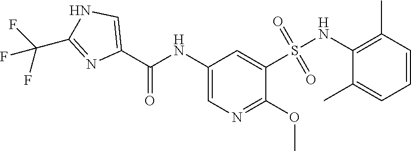 [SMILES: COc1ncc(NC(=O)c2c[nH]c(C(F)(F)F)n2)cc1S(=O)(=O)Nc1c(C)cccc1C] |
| 2487 | 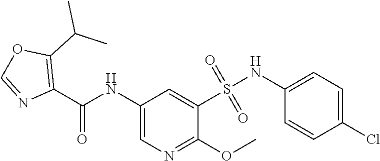 [SMILES: COc1ncc(NC(=O)c2ncoc2C(C)C)cc1S(=O)(=O)Nc1ccc(Cl)cc1] |
| 2492 | 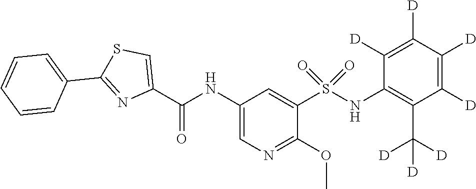 [SMILES: [2H]c1c([2H])c([2H])c(C([2H])([2H])[2H])c(NS(=O)(=O)c2cc(NC(=O)c3csc(-c4ccccc4)n3)cnc2OC)c1[2H]] |
| 2493 | 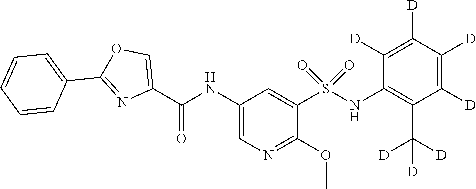 [SMILES: [2H]c1c([2H])c([2H])c(C([2H])([2H])[2H])c(NS(=O)(=O)c2cc(NC(=O)c3coc(-c4ccccc4)n3)cnc2OC)c1[2H]] |
| 2496 | 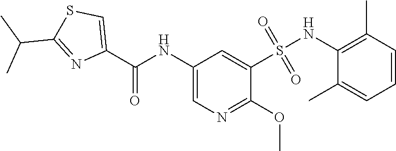 [SMILES: COc1ncc(NC(=O)c2csc(C(C)C)n2)cc1S(=O)(=O)Nc1c(C)cccc1C] |
| 2497 | 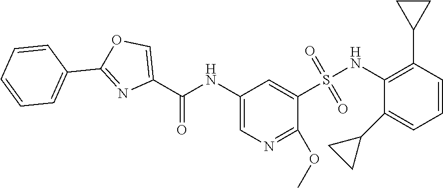 [SMILES: COc1ncc(NC(=O)c2coc(-c3ccccc3)n2)cc1S(=O)(=O)Nc1c(C2CC2)cccc1C1CC1] |
| 2498 | 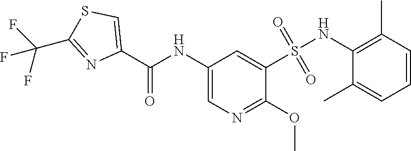 [SMILES: COc1ncc(NC(=O)c2csc(C(F)(F)F)n2)cc1S(=O)(=O)Nc1c(C)cccc1C] |
| 2499 | 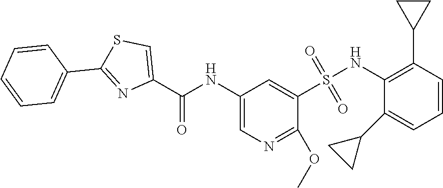 [SMILES: COc1ncc(NC(=O)c2csc(-c3ccccc3)n2)cc1S(=O)(=O)Nc1c(C2CC2)cccc1C1CC1] |
| 2500 | 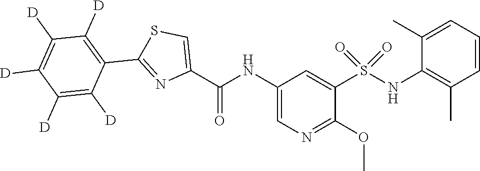 [SMILES: [2H]c1c([2H])c([2H])c(-c2nc(C(=O)Nc3cnc(OC)c(S(=O)(=O)Nc4c(C)cccc4C)c3)cs2)c([2H])c1[2H]] |
| 2501 | 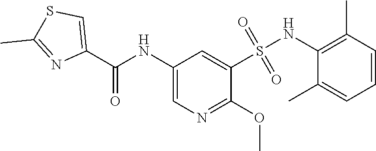 [SMILES: COc1ncc(NC(=O)c2csc(C)n2)cc1S(=O)(=O)Nc1c(C)cccc1C] |
| 2502 | 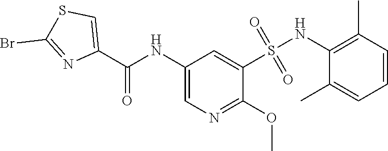 [SMILES: COc1ncc(NC(=O)c2csc(Br)n2)cc1S(=O)(=O)Nc1c(C)cccc1C] |
| 2506 | 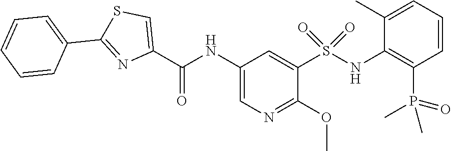 [SMILES: COc1ncc(NC(=O)c2csc(-c3ccccc3)n2)cc1S(=O)(=O)Nc1c(C)cccc1P(C)(C)=O] |
| 2512 | 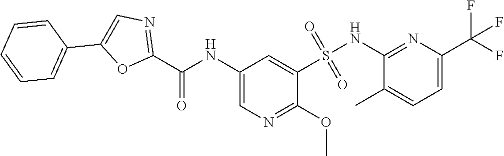 [SMILES: COc1ncc(NC(=O)c2ncc(-c3ccccc3)o2)cc1S(=O)(=O)Nc1nc(C(F)(F)F)ccc1C] |
| 2523 | 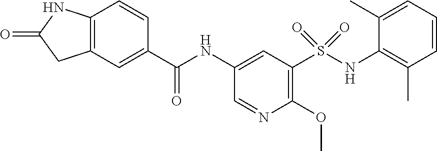 [SMILES: COc1ncc(NC(=O)c2ccc3c(c2)CC(=O)N3)cc1S(=O)(=O)Nc1c(C)cccc1C] |
| 2524 | 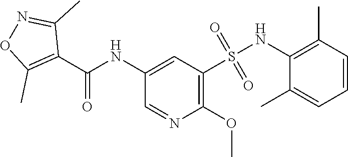 [SMILES: COc1ncc(NC(=O)c2c(C)noc2C)cc1S(=O)(=O)Nc1c(C)cccc1C] |
| 2525 | 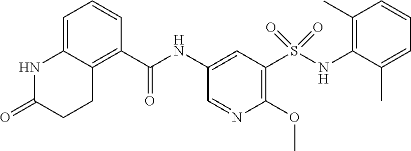 [SMILES: COc1ncc(NC(=O)c2cccc3c2CCC(=O)N3)cc1S(=O)(=O)Nc1c(C)cccc1C] |
| 2526 | 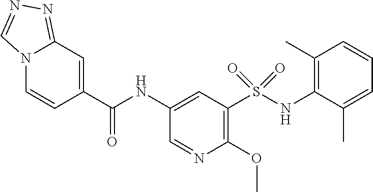 [SMILES: COc1ncc(NC(=O)c2ccn3cnnc3c2)cc1S(=O)(=O)Nc1c(C)cccc1C] |
| 2528 | 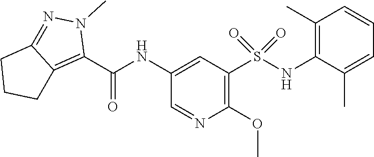 [SMILES: COc1ncc(NC(=O)c2c3c(nn2C)CCC3)cc1S(=O)(=O)Nc1c(C)cccc1C] |
| 2529 | 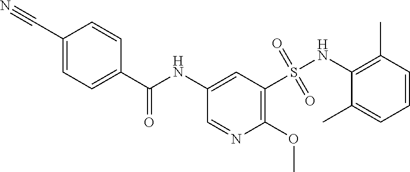 [SMILES: COc1ncc(NC(=O)c2ccc(C#N)cc2)cc1S(=O)(=O)Nc1c(C)cccc1C] |
| 2531 | 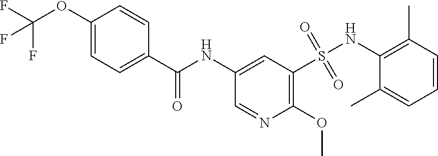 [SMILES: COc1ncc(NC(=O)c2ccc(OC(F)(F)F)cc2)cc1S(=O)(=O)Nc1c(C)cccc1C] |
| 2532 | 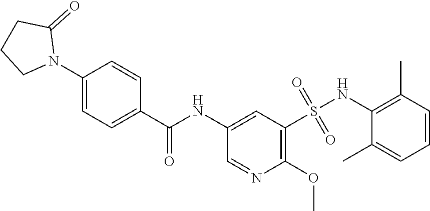 [SMILES: COc1ncc(NC(=O)c2ccc(N3CCCC3=O)cc2)cc1S(=O)(=O)Nc1c(C)cccc1C] |
| 2535 | 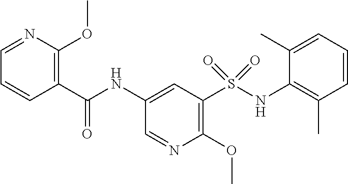 [SMILES: COc1ncccc1C(=O)Nc1cnc(OC)c(S(=O)(=O)Nc2c(C)cccc2C)c1] |
| 2536 | 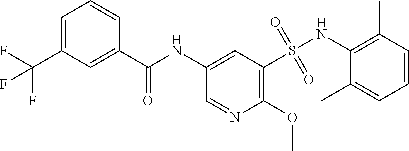 [SMILES: COc1ncc(NC(=O)c2cccc(C(F)(F)F)c2)cc1S(=O)(=O)Nc1c(C)cccc1C] |
| 2537 | 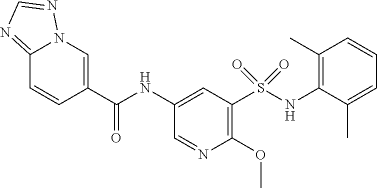 [SMILES: COc1ncc(NC(=O)c2ccc3ncnn3c2)cc1S(=O)(=O)Nc1c(C)cccc1C] |
| 2538 | 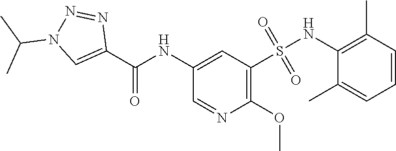 [SMILES: COc1ncc(NC(=O)c2cn(C(C)C)nn2)cc1S(=O)(=O)Nc1c(C)cccc1C] |
| 2539 | 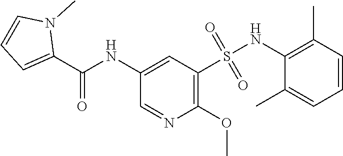 [SMILES: COc1ncc(NC(=O)c2cccn2C)cc1S(=O)(=O)Nc1c(C)cccc1C] |
| 2541 | 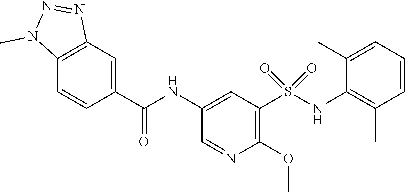 [SMILES: COc1ncc(NC(=O)c2ccc3c(c2)nnn3C)cc1S(=O)(=O)Nc1c(C)cccc1C] |
| 2542 | 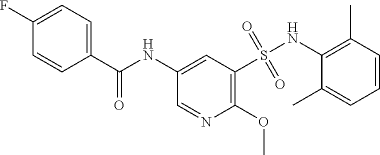 [SMILES: COc1ncc(NC(=O)c2ccc(F)cc2)cc1S(=O)(=O)Nc1c(C)cccc1C] |
| 2543 | 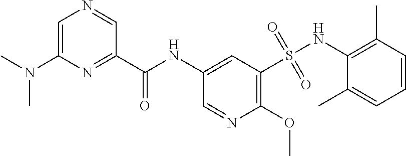 [SMILES: COc1ncc(NC(=O)c2cncc(N(C)C)n2)cc1S(=O)(=O)Nc1c(C)cccc1C] |
| 2544 | 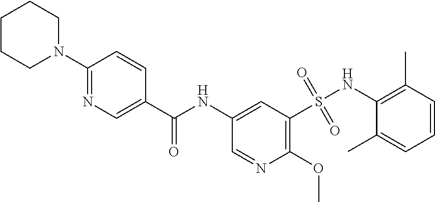 [SMILES: COc1ncc(NC(=O)c2ccc(N3CCCCC3)nc2)cc1S(=O)(=O)Nc1c(C)cccc1C] |
| 2545 | 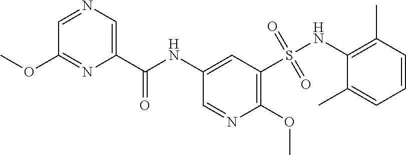 [SMILES: COc1cncc(C(=O)Nc2cnc(OC)c(S(=O)(=O)Nc3c(C)cccc3C)c2)n1] |
| 2546 | 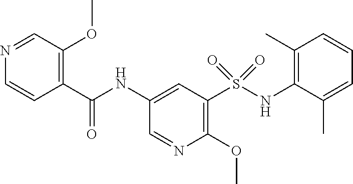 [SMILES: COc1cnccc1C(=O)Nc1cnc(OC)c(S(=O)(=O)Nc2c(C)cccc2C)c1] |
| 2547 | 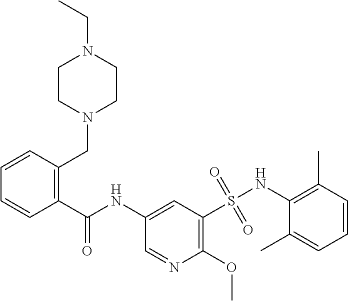 [SMILES: CCN1CCN(Cc2ccccc2C(=O)Nc2cnc(OC)c(S(=O)(=O)Nc3c(C)cccc3C)c2)CC1] |
| 2548 | 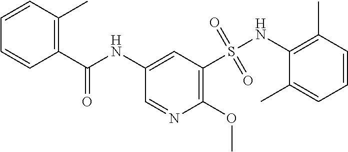 [SMILES: COc1ncc(NC(=O)c2ccccc2C)cc1S(=O)(=O)Nc1c(C)cccc1C] |
| 2549 | 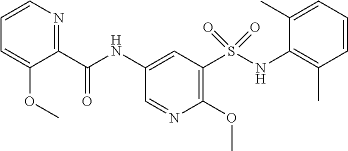 [SMILES: COc1cccnc1C(=O)Nc1cnc(OC)c(S(=O)(=O)Nc2c(C)cccc2C)c1] |
| 2550 | 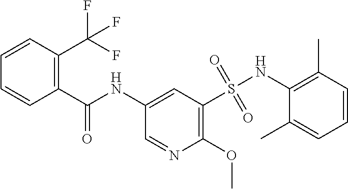 [SMILES: COc1ncc(NC(=O)c2ccccc2C(F)(F)F)cc1S(=O)(=O)Nc1c(C)cccc1C] |
| 2551 | 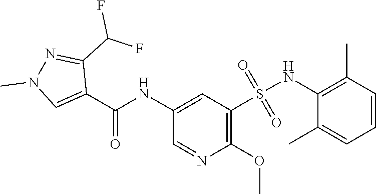 [SMILES: COc1ncc(NC(=O)c2cn(C)nc2C(F)F)cc1S(=O)(=O)Nc1c(C)cccc1C] |
| 2552 | 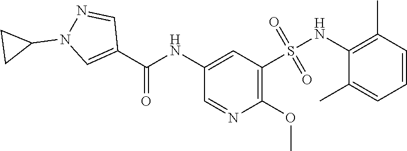 [SMILES: COc1ncc(NC(=O)c2cnn(C3CC3)c2)cc1S(=O)(=O)Nc1c(C)cccc1C] |
| 2553 | 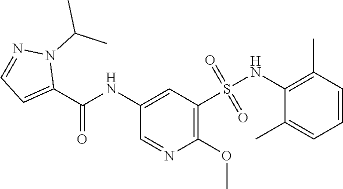 [SMILES: COc1ncc(NC(=O)c2ccnn2C(C)C)cc1S(=O)(=O)Nc1c(C)cccc1C] |
| 2554 | 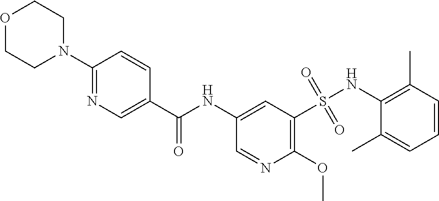 [SMILES: COc1ncc(NC(=O)c2ccc(N3CCOCC3)nc2)cc1S(=O)(=O)Nc1c(C)cccc1C] |
| 2555 | 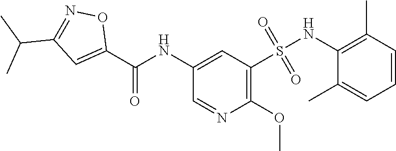 [SMILES: COc1ncc(NC(=O)c2cc(C(C)C)no2)cc1S(=O)(=O)Nc1c(C)cccc1C] |
| 2556 | 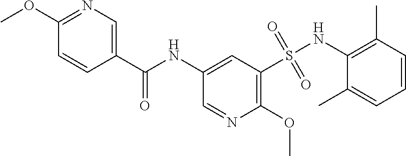 [SMILES: COc1ccc(C(=O)Nc2cnc(OC)c(S(=O)(=O)Nc3c(C)cccc3C)c2)cn1] |
| 2557 | 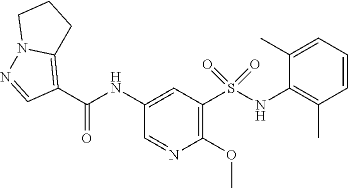 [SMILES: COc1ncc(NC(=O)c2cnn3c2CCC3)cc1S(=O)(=O)Nc1c(C)cccc1C] |
| 2558 | 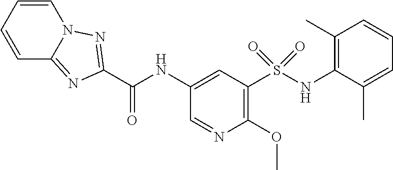 [SMILES: COc1ncc(NC(=O)c2nc3ccccn3n2)cc1S(=O)(=O)Nc1c(C)cccc1C] |
| 2559 | 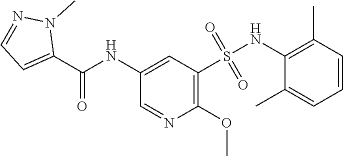 [SMILES: COc1ncc(NC(=O)c2ccnn2C)cc1S(=O)(=O)Nc1c(C)cccc1C] |
| 2561 | 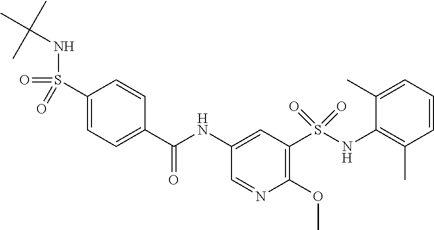 [SMILES: COc1ncc(NC(=O)c2ccc(S(=O)(=O)NC(C)(C)C)cc2)cc1S(=O)(=O)Nc1c(C)cccc1C] |
| 2562 | 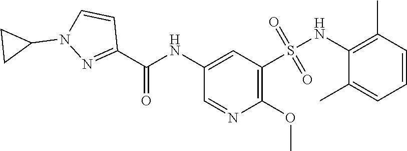 [SMILES: COc1ncc(NC(=O)c2ccn(C3CC3)n2)cc1S(=O)(=O)Nc1c(C)cccc1C] |
| 2564 | 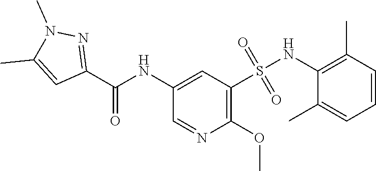 [SMILES: COc1ncc(NC(=O)c2cc(C)n(C)n2)cc1S(=O)(=O)Nc1c(C)cccc1C] |
| 2566 | 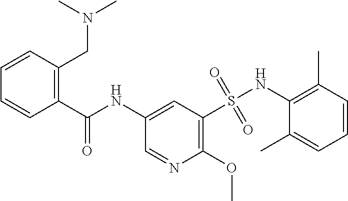 [SMILES: COc1ncc(NC(=O)c2ccccc2CN(C)C)cc1S(=O)(=O)Nc1c(C)cccc1C] |
| 2567 | 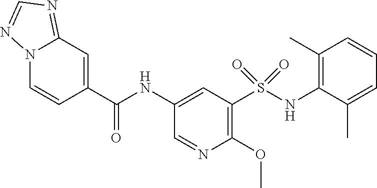 [SMILES: COc1ncc(NC(=O)c2ccn3ncnc3c2)cc1S(=O)(=O)Nc1c(C)cccc1C] |
| 2568 | 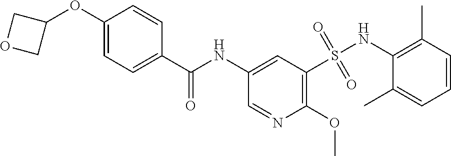 [SMILES: COc1ncc(NC(=O)c2ccc(OC3COC3)cc2)cc1S(=O)(=O)Nc1c(C)cccc1C] |
| 2569 | 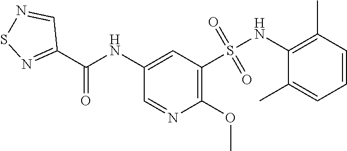 [SMILES: COc1ncc(NC(=O)c2cnsn2)cc1S(=O)(=O)Nc1c(C)cccc1C] |
| 2570 | 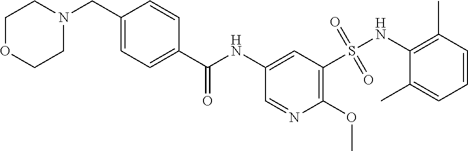 [SMILES: COc1ncc(NC(=O)c2ccc(CN3CCOCC3)cc2)cc1S(=O)(=O)Nc1c(C)cccc1C] |
| 2571 | 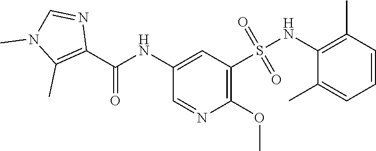 [SMILES: COc1ncc(NC(=O)c2ncn(C)c2C)cc1S(=O)(=O)Nc1c(C)cccc1C] |
| 2572 | 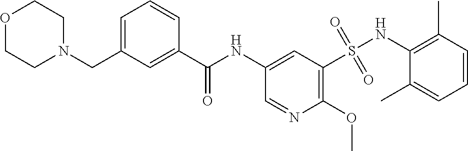 [SMILES: COc1ncc(NC(=O)c2cccc(CN3CCOCC3)c2)cc1S(=O)(=O)Nc1c(C)cccc1C] |
| 2573 | 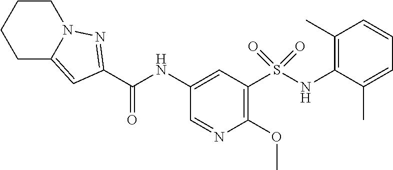 [SMILES: COc1ncc(NC(=O)c2cc3n(n2)CCCC3)cc1S(=O)(=O)Nc1c(C)cccc1C] |
| 2574 | 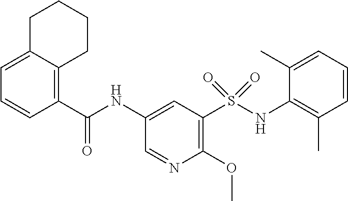 [SMILES: COc1ncc(NC(=O)c2cccc3c2CCCC3)cc1S(=O)(=O)Nc1c(C)cccc1C] |
| 2575 | 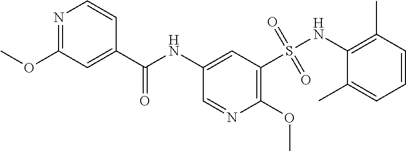 [SMILES: COc1cc(C(=O)Nc2cnc(OC)c(S(=O)(=O)Nc3c(C)cccc3C)c2)ccn1] |
| 2576 | 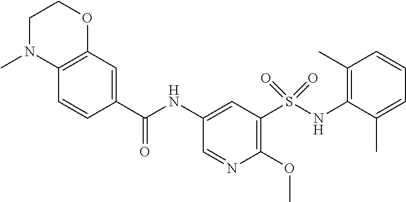 [SMILES: COc1ncc(NC(=O)c2ccc3c(c2)OCCN3C)cc1S(=O)(=O)Nc1c(C)cccc1C] |
| 2577 | 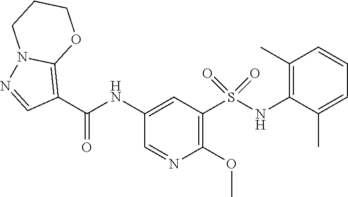 [SMILES: COc1ncc(NC(=O)c2cnn3c2OCCC3)cc1S(=O)(=O)Nc1c(C)cccc1C] |
| 2578 | 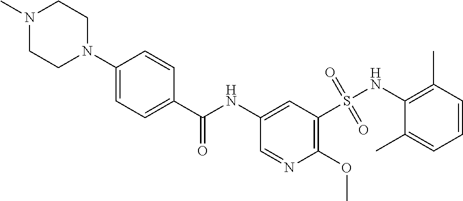 [SMILES: COc1ncc(NC(=O)c2ccc(N3CCN(C)CC3)cc2)cc1S(=O)(=O)Nc1c(C)cccc1C] |
| 2580 | 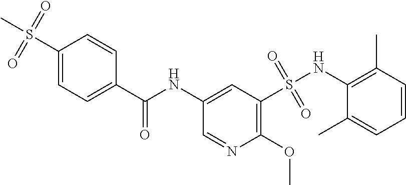 [SMILES: COc1ncc(NC(=O)c2ccc(S(C)(=O)=O)cc2)cc1S(=O)(=O)Nc1c(C)cccc1C] |
| 2581 | 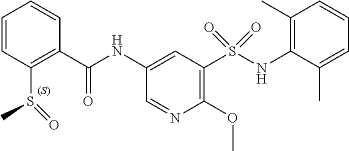 |
| 2582 | 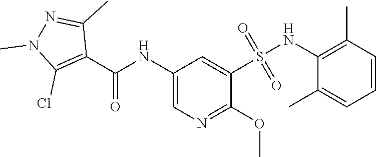 [SMILES: COc1ncc(NC(=O)c2c(C)nn(C)c2Cl)cc1S(=O)(=O)Nc1c(C)cccc1C] |
| 2583 | 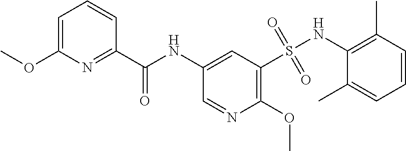 [SMILES: COc1cccc(C(=O)Nc2cnc(OC)c(S(=O)(=O)Nc3c(C)cccc3C)c2)n1] |
| 2584 | 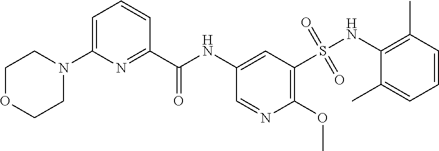 [SMILES: COc1ncc(NC(=O)c2cccc(N3CCOCC3)n2)cc1S(=O)(=O)Nc1c(C)cccc1C] |
| 2586 | 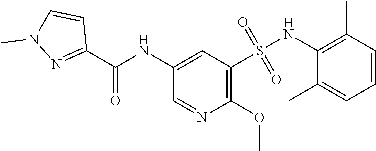 [SMILES: COc1ncc(NC(=O)c2ccn(C)n2)cc1S(=O)(=O)Nc1c(C)cccc1C] |
| 2587 | 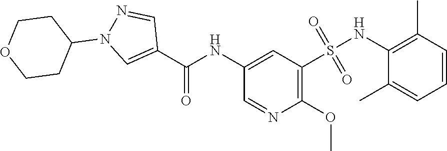 [SMILES: COc1ncc(NC(=O)c2cnn(C3CCOCC3)c2)cc1S(=O)(=O)Nc1c(C)cccc1C] |
| 2590 | 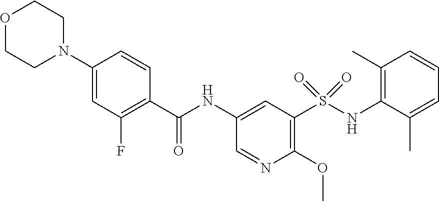 [SMILES: COc1ncc(NC(=O)c2ccc(N3CCOCC3)cc2F)cc1S(=O)(=O)Nc1c(C)cccc1C] |
| 2591 | 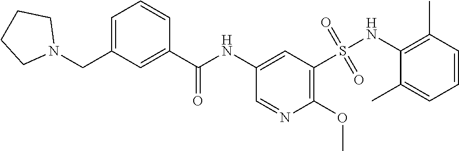 [SMILES: COc1ncc(NC(=O)c2cccc(CN3CCCC3)c2)cc1S(=O)(=O)Nc1c(C)cccc1C] |
| 2593 | 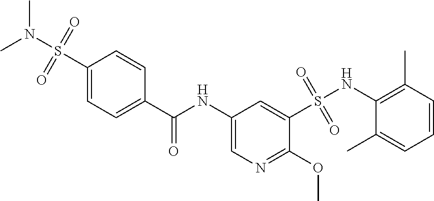 [SMILES: COc1ncc(NC(=O)c2ccc(S(=O)(=O)N(C)C)cc2)cc1S(=O)(=O)Nc1c(C)cccc1C] |
| 2594 | 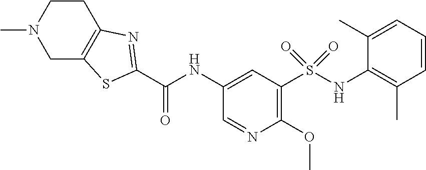 [SMILES: COc1ncc(NC(=O)c2nc3c(s2)CN(C)CC3)cc1S(=O)(=O)Nc1c(C)cccc1C] |
| 2595 | 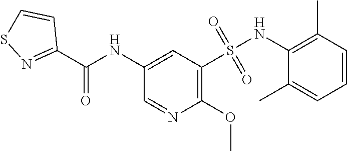 [SMILES: COc1ncc(NC(=O)c2ccsn2)cc1S(=O)(=O)Nc1c(C)cccc1C] |
| 2597 | 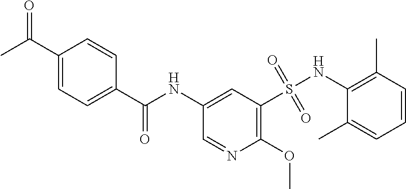 [SMILES: COc1ncc(NC(=O)c2ccc(C(C)=O)cc2)cc1S(=O)(=O)Nc1c(C)cccc1C] |
| 2599 | 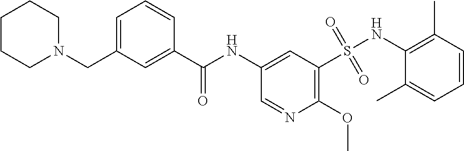 [SMILES: COc1ncc(NC(=O)c2cccc(CN3CCCCC3)c2)cc1S(=O)(=O)Nc1c(C)cccc1C] |
| 2600 | 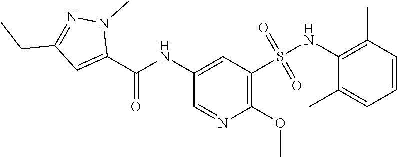 [SMILES: CCc1cc(C(=O)Nc2cnc(OC)c(S(=O)(=O)Nc3c(C)cccc3C)c2)n(C)n1] |
| 2601 | 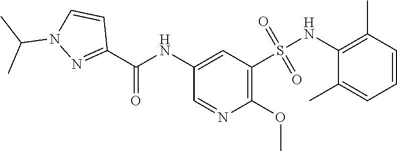 [SMILES: COc1ncc(NC(=O)c2ccn(C(C)C)n2)cc1S(=O)(=O)Nc1c(C)cccc1C] |
| 2602 | 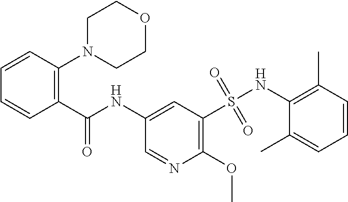 [SMILES: COc1ncc(NC(=O)c2ccccc2N2CCOCC2)cc1S(=O)(=O)Nc1c(C)cccc1C] |
| 2607 | 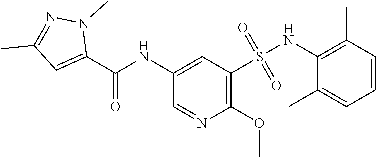 [SMILES: COc1ncc(NC(=O)c2cc(C)nn2C)cc1S(=O)(=O)Nc1c(C)cccc1C] |
| 2608 | 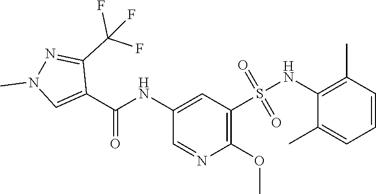 [SMILES: COc1ncc(NC(=O)c2cn(C)nc2C(F)(F)F)cc1S(=O)(=O)Nc1c(C)cccc1C] |
| 2609 | 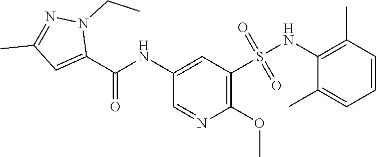 [SMILES: CCn1nc(C)cc1C(=O)Nc1cnc(OC)c(S(=O)(=O)Nc2c(C)cccc2C)c1] |
| 2611 | 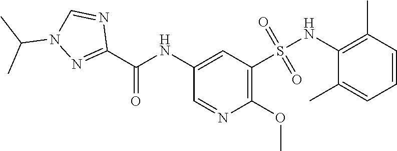 [SMILES: COc1ncc(NC(=O)c2ncn(C(C)C)n2)cc1S(=O)(=O)Nc1c(C)cccc1C] |
| 2612 | 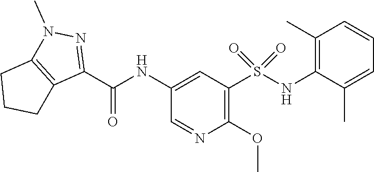 [SMILES: COc1ncc(NC(=O)c2nn(C)c3c2CCC3)cc1S(=O)(=O)Nc1c(C)cccc1C] |
| 2613 | 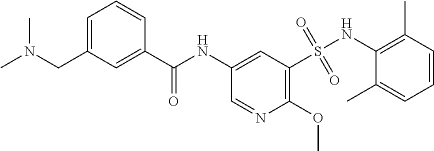 [SMILES: COc1ncc(NC(=O)c2cccc(CN(C)C)c2)cc1S(=O)(=O)Nc1c(C)cccc1C] |
| 2614 | 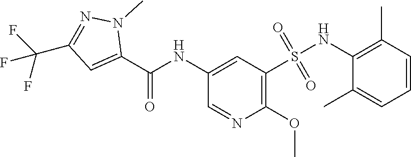 [SMILES: COc1ncc(NC(=O)c2cc(C(F)(F)F)nn2C)cc1S(=O)(=O)Nc1c(C)cccc1C] |
| 2615 | 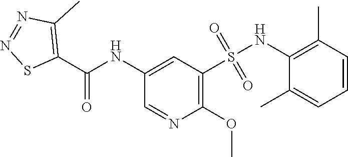 [SMILES: COc1ncc(NC(=O)c2snnc2C)cc1S(=O)(=O)Nc1c(C)cccc1C] |
| 2617 | 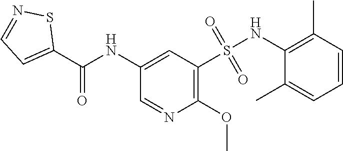 [SMILES: COc1ncc(NC(=O)c2ccns2)cc1S(=O)(=O)Nc1c(C)cccc1C] |
| 2618 | 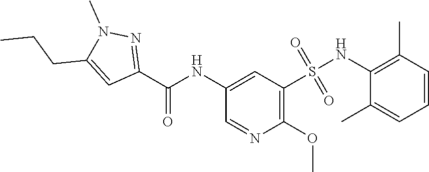 [SMILES: CCCc1cc(C(=O)Nc2cnc(OC)c(S(=O)(=O)Nc3c(C)cccc3C)c2)nn1C] |
| 2619 | 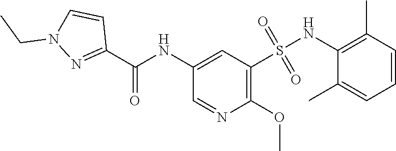 [SMILES: CCn1ccc(C(=O)Nc2cnc(OC)c(S(=O)(=O)Nc3c(C)cccc3C)c2)n1] |
| 2621 | 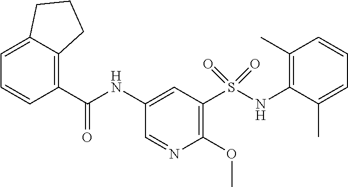 [SMILES: COc1ncc(NC(=O)c2cccc3c2CCC3)cc1S(=O)(=O)Nc1c(C)cccc1C] |
| 2622 | 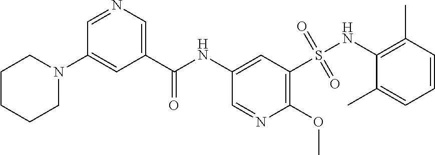 [SMILES: COc1ncc(NC(=O)c2cncc(N3CCCCC3)c2)cc1S(=O)(=O)Nc1c(C)cccc1C] |
| 2624 | 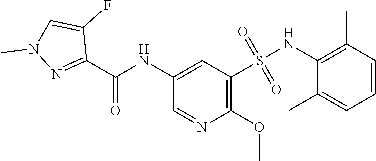 [SMILES: COc1ncc(NC(=O)c2nn(C)cc2F)cc1S(=O)(=O)Nc1c(C)cccc1C] |
| 2625 | 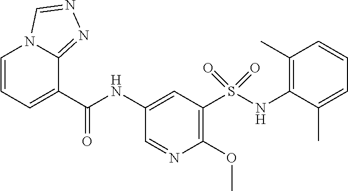 [SMILES: COc1ncc(NC(=O)c2cccn3cnnc23)cc1S(=O)(=O)Nc1c(C)cccc1C] |
| 2626 | 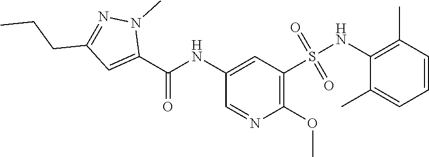 [SMILES: CCCc1cc(C(=O)Nc2cnc(OC)c(S(=O)(=O)Nc3c(C)cccc3C)c2)n(C)n1] |
| 2627 | 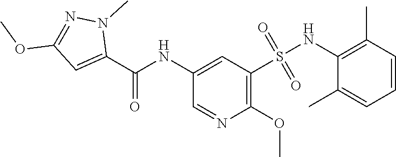 [SMILES: COc1cc(C(=O)Nc2cnc(OC)c(S(=O)(=O)Nc3c(C)cccc3C)c2)n(C)n1] |
| 2628 | 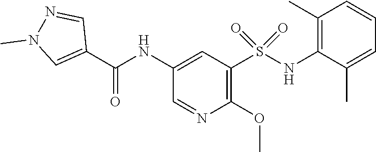 [SMILES: COc1ncc(NC(=O)c2cnn(C)c2)cc1S(=O)(=O)Nc1c(C)cccc1C] |
| 2629 | 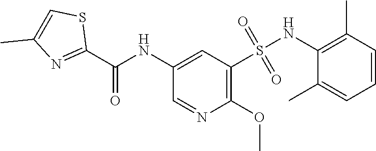 [SMILES: COc1ncc(NC(=O)c2nc(C)cs2)cc1S(=O)(=O)Nc1c(C)cccc1C] |
| 2630 | 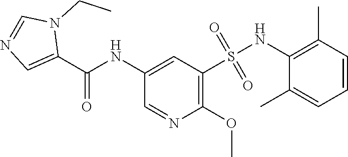 [SMILES: CCn1cncc1C(=O)Nc1cnc(OC)c(S(=O)(=O)Nc2c(C)cccc2C)c1] |
| 2631 | 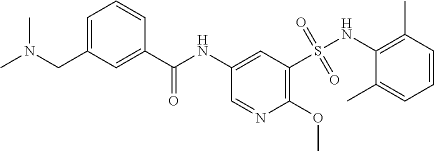 [SMILES: COc1ncc(NC(=O)c2cccc(CN(C)C)c2)cc1S(=O)(=O)Nc1c(C)cccc1C] |
| 2632 | 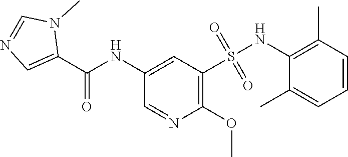 [SMILES: COc1ncc(NC(=O)c2cncn2C)cc1S(=O)(=O)Nc1c(C)cccc1C] |
| 2633 | 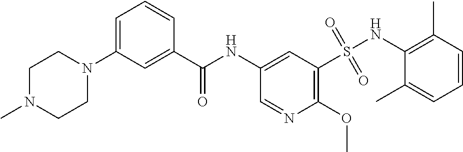 [SMILES: COc1ncc(NC(=O)c2cccc(N3CCN(C)CC3)c2)cc1S(=O)(=O)Nc1c(C)cccc1C] |
| 2634 | 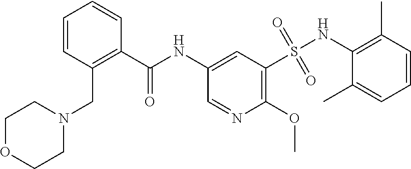 [SMILES: COc1ncc(NC(=O)c2ccccc2CN2CCOCC2)cc1S(=O)(=O)Nc1c(C)cccc1C] |
| 2635 | 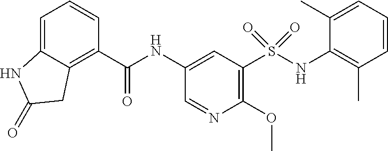 [SMILES: COc1ncc(NC(=O)c2cccc3c2CC(=O)N3)cc1S(=O)(=O)Nc1c(C)cccc1C] |
| 2636 | 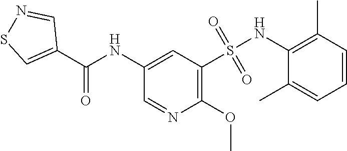 [SMILES: COc1ncc(NC(=O)c2cnsc2)cc1S(=O)(=O)Nc1c(C)cccc1C] |
| 2637 | 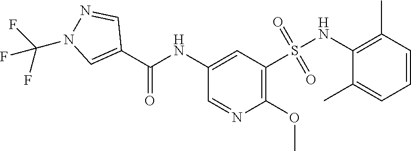 [SMILES: COc1ncc(NC(=O)c2cnn(C(F)(F)F)c2)cc1S(=O)(=O)Nc1c(C)cccc1C] |
| 2638 | 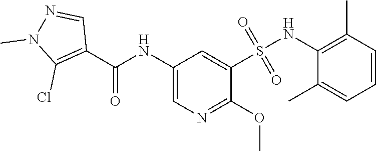 [SMILES: COc1ncc(NC(=O)c2cnn(C)c2Cl)cc1S(=O)(=O)Nc1c(C)cccc1C] |
| 2639 | 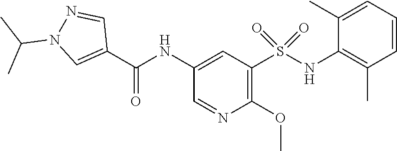 [SMILES: COc1ncc(NC(=O)c2cnn(C(C)C)c2)cc1S(=O)(=O)Nc1c(C)cccc1C] |
| 2640 | 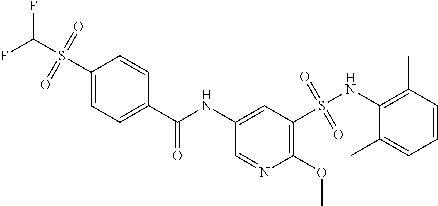 [SMILES: COc1ncc(NC(=O)c2ccc(S(=O)(=O)C(F)F)cc2)cc1S(=O)(=O)Nc1c(C)cccc1C] |
| 2641 | 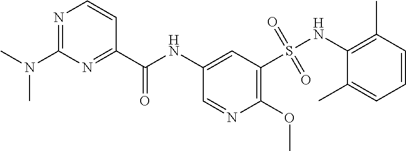 [SMILES: COc1ncc(NC(=O)c2ccnc(N(C)C)n2)cc1S(=O)(=O)Nc1c(C)cccc1C] |
| 2642 | 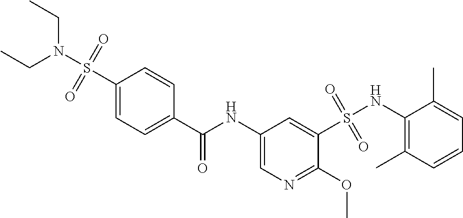 [SMILES: CCN(CC)S(=O)(=O)c1ccc(C(=O)Nc2cnc(OC)c(S(=O)(=O)Nc3c(C)cccc3C)c2)cc1] |
| 2643 | 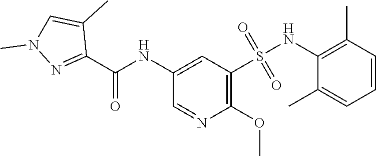 [SMILES: COc1ncc(NC(=O)c2nn(C)cc2C)cc1S(=O)(=O)Nc1c(C)cccc1C] |
| 2644 | 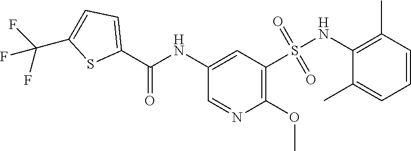 [SMILES: COc1ncc(NC(=O)c2ccc(C(F)(F)F)s2)cc1S(=O)(=O)Nc1c(C)cccc1C] |
| 2646 | 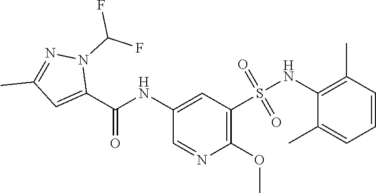 [SMILES: COc1ncc(NC(=O)c2cc(C)nn2C(F)F)cc1S(=O)(=O)Nc1c(C)cccc1C] |
| 2649 | 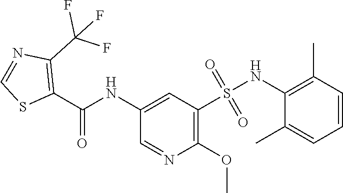 [SMILES: COc1ncc(NC(=O)c2scnc2C(F)(F)F)cc1S(=O)(=O)Nc1c(C)cccc1C] |
| 2650 | 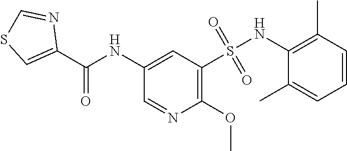 [SMILES: COc1ncc(NC(=O)c2cscn2)cc1S(=O)(=O)Nc1c(C)cccc1C] |
| 2651 | 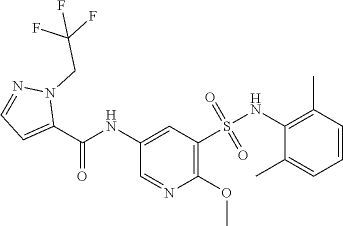 [SMILES: COc1ncc(NC(=O)c2ccnn2CC(F)(F)F)cc1S(=O)(=O)Nc1c(C)cccc1C] |
| 2653 | 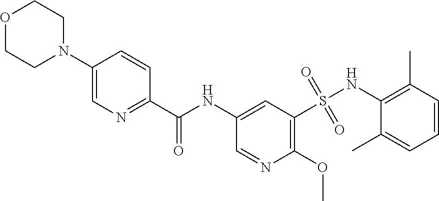 [SMILES: COc1ncc(NC(=O)c2ccc(N3CCOCC3)cn2)cc1S(=O)(=O)Nc1c(C)cccc1C] |
| 2654 | 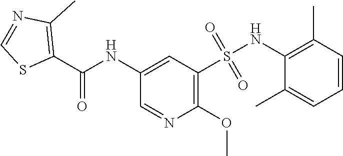 [SMILES: COc1ncc(NC(=O)c2scnc2C)cc1S(=O)(=O)Nc1c(C)cccc1C] |
| 2655 | 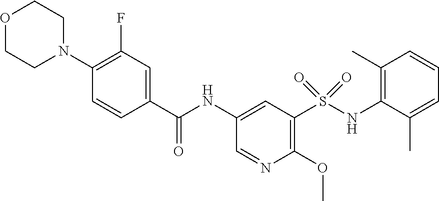 [SMILES: COc1ncc(NC(=O)c2ccc(N3CCOCC3)c(F)c2)cc1S(=O)(=O)Nc1c(C)cccc1C] |
| 2656 | 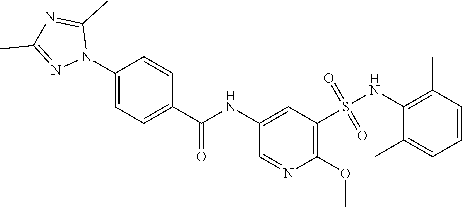 [SMILES: COc1ncc(NC(=O)c2ccc(-n3nc(C)nc3C)cc2)cc1S(=O)(=O)Nc1c(C)cccc1C] |
| 2657 | 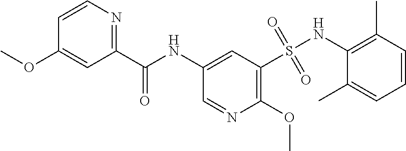 [SMILES: COc1ccnc(C(=O)Nc2cnc(OC)c(S(=O)(=O)Nc3c(C)cccc3C)c2)c1] |
| 2658 | 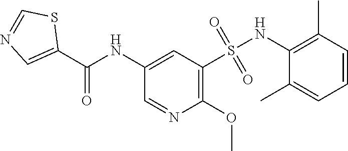 [SMILES: COc1ncc(NC(=O)c2cncs2)cc1S(=O)(=O)Nc1c(C)cccc1C] |
| 2659 | 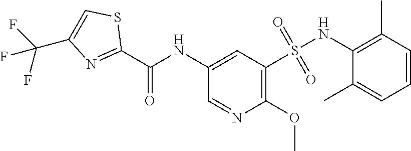 [SMILES: COc1ncc(NC(=O)c2nc(C(F)(F)F)cs2)cc1S(=O)(=O)Nc1c(C)cccc1C] |
| 2660 | 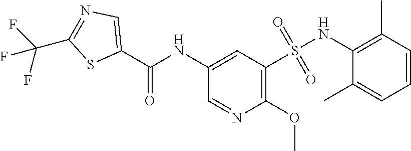 [SMILES: COc1ncc(NC(=O)c2cnc(C(F)(F)F)s2)cc1S(=O)(=O)Nc1c(C)cccc1C] |
| 2661 | 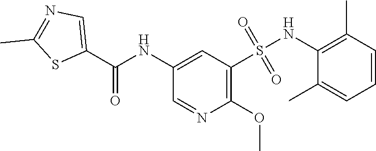 [SMILES: COc1ncc(NC(=O)c2cnc(C)s2)cc1S(=O)(=O)Nc1c(C)cccc1C] |
| 2662 | 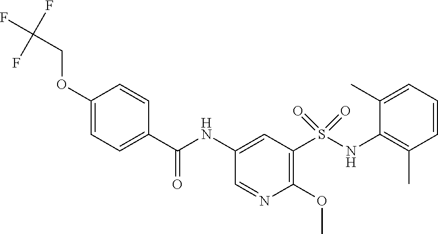 [SMILES: COc1ncc(NC(=O)c2ccc(OCC(F)(F)F)cc2)cc1S(=O)(=O)Nc1c(C)cccc1C] |
| 2663 | 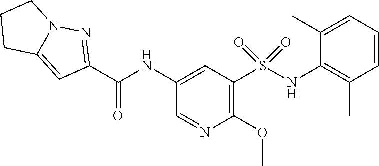 [SMILES: COc1ncc(NC(=O)c2cc3n(n2)CCC3)cc1S(=O)(=O)Nc1c(C)cccc1C] |
| 2665 | 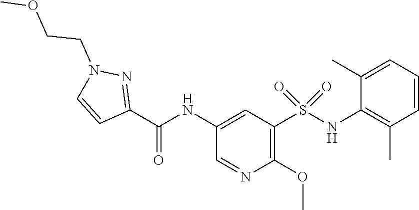 [SMILES: COCCn1ccc(C(=O)Nc2cnc(OC)c(S(=O)(=O)Nc3c(C)cccc3C)c2)n1] |
| 2666 | 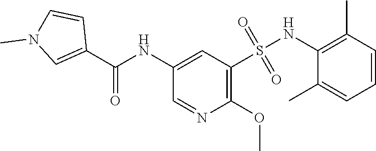 [SMILES: COc1ncc(NC(=O)c2ccn(C)c2)cc1S(=O)(=O)Nc1c(C)cccc1C] |
| 2667 | 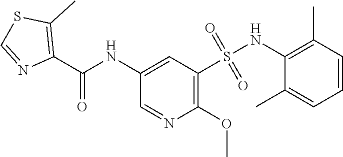 [SMILES: COc1ncc(NC(=O)c2ncsc2C)cc1S(=O)(=O)Nc1c(C)cccc1C] |
| 2669 | 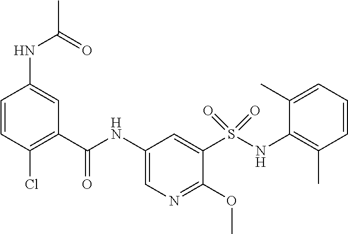 [SMILES: COc1ncc(NC(=O)c2cc(NC(C)=O)ccc2Cl)cc1S(=O)(=O)Nc1c(C)cccc1C] |
| 2670 | 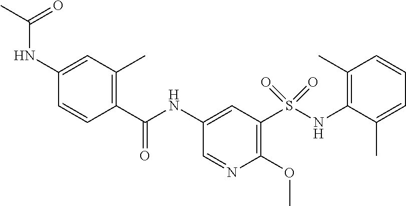 [SMILES: COc1ncc(NC(=O)c2ccc(NC(C)=O)cc2C)cc1S(=O)(=O)Nc1c(C)cccc1C] |
| 2671 | 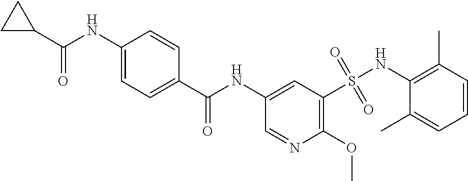 [SMILES: COc1ncc(NC(=O)c2ccc(NC(=O)C3CC3)cc2)cc1S(=O)(=O)Nc1c(C)cccc1C] |
| 2672 | 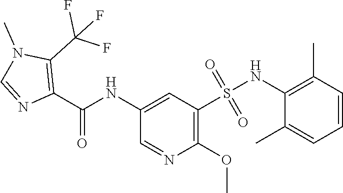 [SMILES: COc1ncc(NC(=O)c2ncn(C)c2C(F)(F)F)cc1S(=O)(=O)Nc1c(C)cccc1C] |
| 2673 | 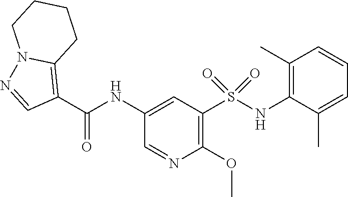 [SMILES: COc1ncc(NC(=O)c2cnn3c2CCCC3)cc1S(=O)(=O)Nc1c(C)cccc1C] |
| 2674 | 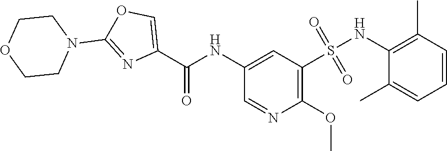 [SMILES: COc1ncc(NC(=O)c2coc(N3CCOCC3)n2)cc1S(=O)(=O)Nc1c(C)cccc1C] |
| 2675 | 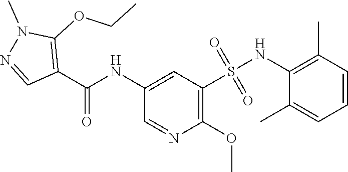 [SMILES: CCOc1c(C(=O)Nc2cnc(OC)c(S(=O)(=O)Nc3c(C)cccc3C)c2)cnn1C] |
| 2677 | 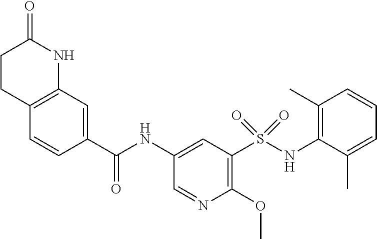 [SMILES: COc1ncc(NC(=O)c2ccc3c(c2)NC(=O)CC3)cc1S(=O)(=O)Nc1c(C)cccc1C] |
| 2678 | 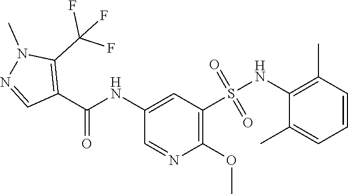 [SMILES: COc1ncc(NC(=O)c2cnn(C)c2C(F)(F)F)cc1S(=O)(=O)Nc1c(C)cccc1C] |
| 2679 | 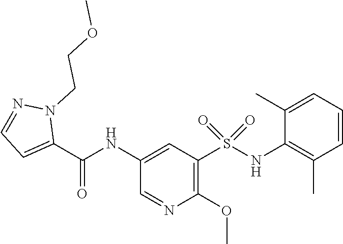 [SMILES: COCCn1nccc1C(=O)Nc1cnc(OC)c(S(=O)(=O)Nc2c(C)cccc2C)c1] |
| 2682 | 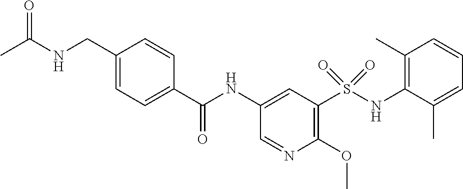 [SMILES: COc1ncc(NC(=O)c2ccc(CNC(C)=O)cc2)cc1S(=O)(=O)Nc1c(C)cccc1C] |
| 2683 | 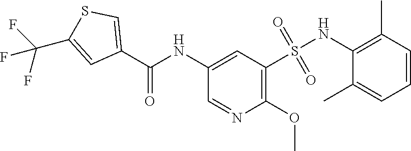 [SMILES: COc1ncc(NC(=O)c2csc(C(F)(F)F)c2)cc1S(=O)(=O)Nc1c(C)cccc1C] |
| 2684 | 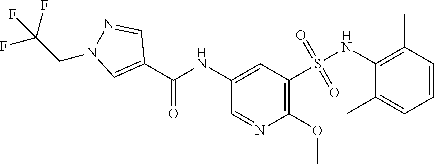 [SMILES: COc1ncc(NC(=O)c2cnn(CC(F)(F)F)c2)cc1S(=O)(=O)Nc1c(C)cccc1C] |
| 2687 | 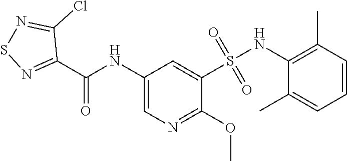 [SMILES: COc1ncc(NC(=O)c2nsnc2Cl)cc1S(=O)(=O)Nc1c(C)cccc1C] |
| 2688 | 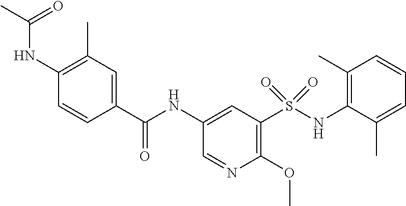 [SMILES: COc1ncc(NC(=O)c2ccc(NC(C)=O)c(C)c2)cc1S(=O)(=O)Nc1c(C)cccc1C] |
| 2689 | 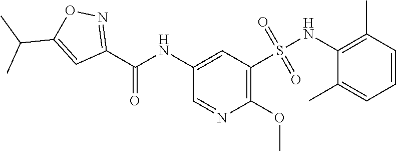 [SMILES: COc1ncc(NC(=O)c2cc(C(C)C)on2)cc1S(=O)(=O)Nc1c(C)cccc1C] |
| 2690 | 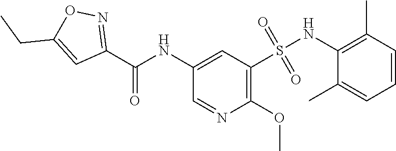 [SMILES: CCc1cc(C(=O)Nc2cnc(OC)c(S(=O)(=O)Nc3c(C)cccc3C)c2)no1] |
| 2691 | 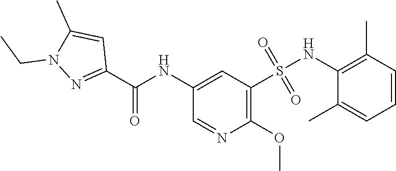 [SMILES: CCn1nc(C(=O)Nc2cnc(OC)c(S(=O)(=O)Nc3c(C)cccc3C)c2)cc1C] |
| 2692 | 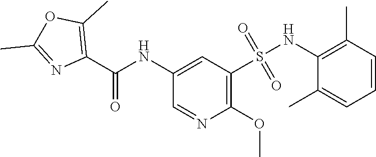 [SMILES: COc1ncc(NC(=O)c2nc(C)oc2C)cc1S(=O)(=O)Nc1c(C)cccc1C] |
| 2693 | 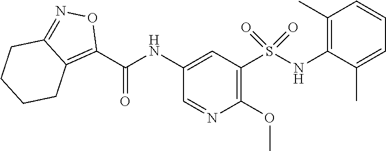 [SMILES: COc1ncc(NC(=O)c2onc3c2CCCC3)cc1S(=O)(=O)Nc1c(C)cccc1C] |
| 2694 | 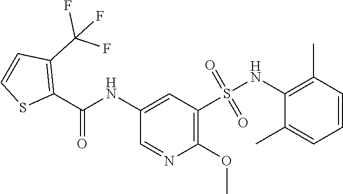 [SMILES: COc1ncc(NC(=O)c2sccc2C(F)(F)F)cc1S(=O)(=O)Nc1c(C)cccc1C] |
| 2695 | 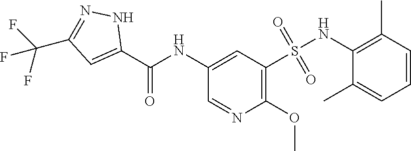 [SMILES: COc1ncc(NC(=O)c2cc(C(F)(F)F)n[nH]2)cc1S(=O)(=O)Nc1c(C)cccc1C] |
| 2696 | 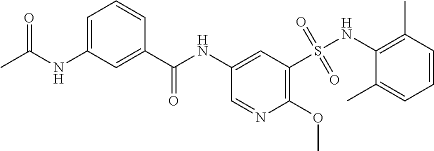 [SMILES: COc1ncc(NC(=O)c2cccc(NC(C)=O)c2)cc1S(=O)(=O)Nc1c(C)cccc1C] |
| 2697 | 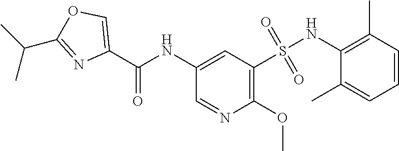 [SMILES: COc1ncc(NC(=O)c2coc(C(C)C)n2)cc1S(=O)(=O)Nc1c(C)cccc1C] |
| 2699 | 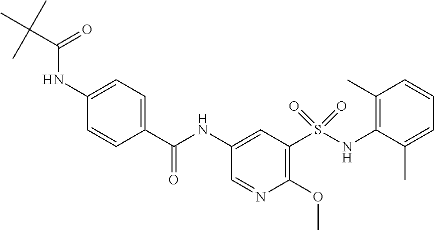 [SMILES: COc1ncc(NC(=O)c2ccc(NC(=O)C(C)(C)C)cc2)cc1S(=O)(=O)Nc1c(C)cccc1C] |
| 2700 | 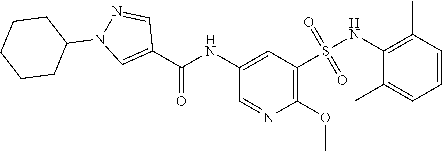 [SMILES: COc1ncc(NC(=O)c2cnn(C3CCCCC3)c2)cc1S(=O)(=O)Nc1c(C)cccc1C] |
| 2701 | 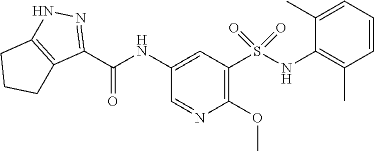 [SMILES: COc1ncc(NC(=O)c2n[nH]c3c2CCC3)cc1S(=O)(=O)Nc1c(C)cccc1C] |
| 2703 | 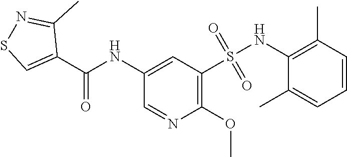 [SMILES: COc1ncc(NC(=O)c2csnc2C)cc1S(=O)(=O)Nc1c(C)cccc1C] |
| 2706 | 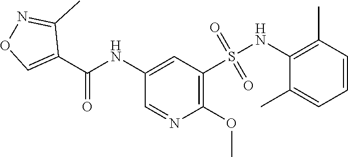 [SMILES: COc1ncc(NC(=O)c2conc2C)cc1S(=O)(=O)Nc1c(C)cccc1C] |
| 2708 | 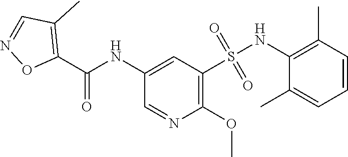 [SMILES: COc1ncc(NC(=O)c2oncc2C)cc1S(=O)(=O)Nc1c(C)cccc1C] |
| 2709 | 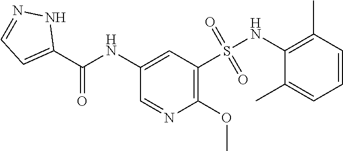 [SMILES: COc1ncc(NC(=O)c2ccn[nH]2)cc1S(=O)(=O)Nc1c(C)cccc1C] |
| 2710 | 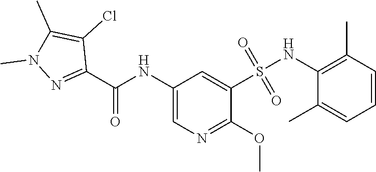 [SMILES: COc1ncc(NC(=O)c2nn(C)c(C)c2Cl)cc1S(=O)(=O)Nc1c(C)cccc1C] |
| 2711 | 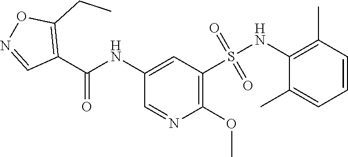 [SMILES: CCc1oncc1C(=O)Nc1cnc(OC)c(S(=O)(=O)Nc2c(C)cccc2C)c1] |
| 2712 | 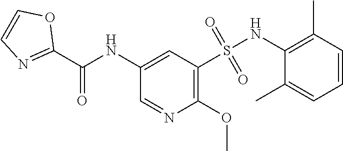 [SMILES: COc1ncc(NC(=O)c2ncco2)cc1S(=O)(=O)Nc1c(C)cccc1C] |
| 2713 | 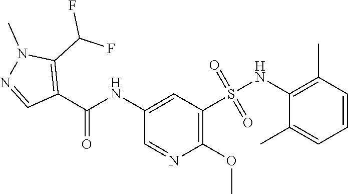 [SMILES: COc1ncc(NC(=O)c2cnn(C)c2C(F)F)cc1S(=O)(=O)Nc1c(C)cccc1C] |
| 2714 | 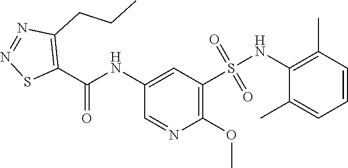 [SMILES: CCCc1nnsc1C(=O)Nc1cnc(OC)c(S(=O)(=O)Nc2c(C)cccc2C)c1] |
| 2715 | 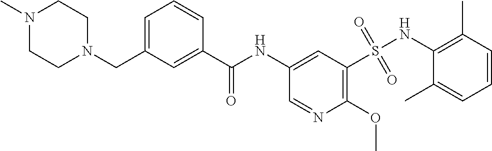 [SMILES: COc1ncc(NC(=O)c2cccc(CN3CCN(C)CC3)c2)cc1S(=O)(=O)Nc1c(C)cccc1C] |
| 2716 | 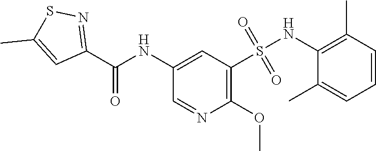 [SMILES: COc1ncc(NC(=O)c2cc(C)sn2)cc1S(=O)(=O)Nc1c(C)cccc1C] |
| 2718 | 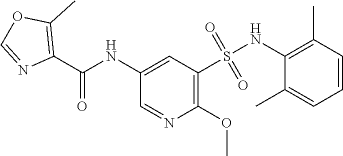 [SMILES: COc1ncc(NC(=O)c2ncoc2C)cc1S(=O)(=O)Nc1c(C)cccc1C] |
| 2719 | 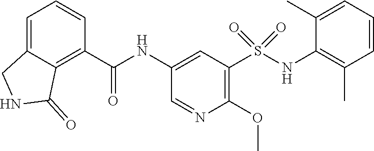 [SMILES: COc1ncc(NC(=O)c2cccc3c2C(=O)NC3)cc1S(=O)(=O)Nc1c(C)cccc1C] |
| 2720 | 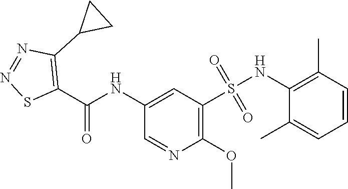 [SMILES: COc1ncc(NC(=O)c2snnc2C2CC2)cc1S(=O)(=O)Nc1c(C)cccc1C] |
| 2724 | 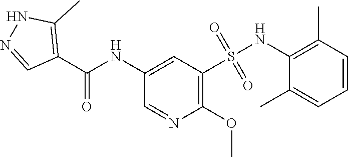 [SMILES: COc1ncc(NC(=O)c2cn[nH]c2C)cc1S(=O)(=O)Nc1c(C)cccc1C] |
| 2725 | 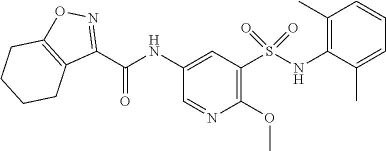 [SMILES: COc1ncc(NC(=O)c2noc3c2CCCC3)cc1S(=O)(=O)Nc1c(C)cccc1C] |
| 2726 | 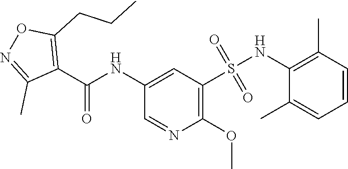 [SMILES: CCCc1onc(C)c1C(=O)Nc1cnc(OC)c(S(=O)(=O)Nc2c(C)cccc2C)c1] |
| 2727 | 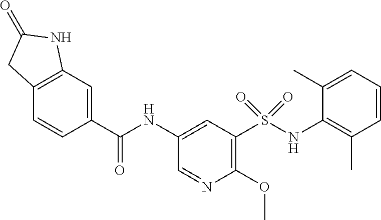 [SMILES: COc1ncc(NC(=O)c2ccc3c(c2)NC(=O)C3)cc1S(=O)(=O)Nc1c(C)cccc1C] |
| 2729 | 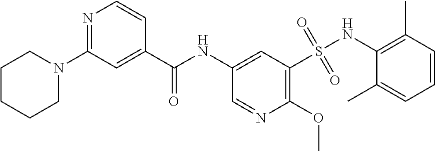 [SMILES: COc1ncc(NC(=O)c2ccnc(N3CCCCC3)c2)cc1S(=O)(=O)Nc1c(C)cccc1C] |
| 2730 | 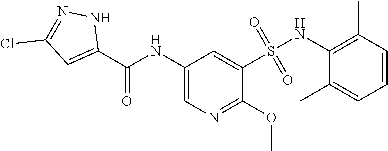 [SMILES: COc1ncc(NC(=O)c2cc(Cl)n[nH]2)cc1S(=O)(=O)Nc1c(C)cccc1C] |
| 2731 | 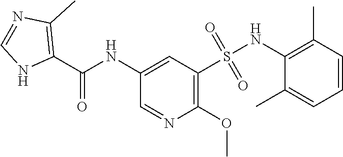 [SMILES: COc1ncc(NC(=O)c2[nH]cnc2C)cc1S(=O)(=O)Nc1c(C)cccc1C] |
| 2732 | 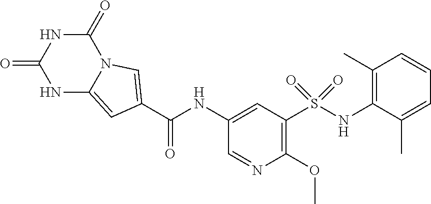 [SMILES: COc1ncc(NC(=O)c2cc3[nH]c(=O)[nH]c(=O)n3c2)cc1S(=O)(=O)Nc1c(C)cccc1C] |
| 2733 | 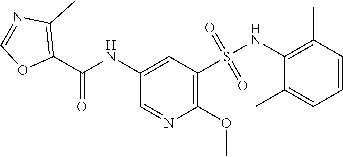 [SMILES: COc1ncc(NC(=O)c2ocnc2C)cc1S(=O)(=O)Nc1c(C)cccc1C] |
| 2734 | 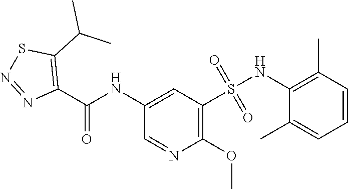 [SMILES: COc1ncc(NC(=O)c2nnsc2C(C)C)cc1S(=O)(=O)Nc1c(C)cccc1C] |
| 2735 | 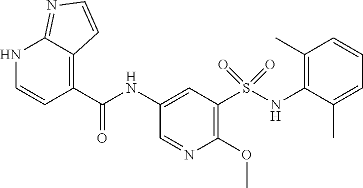 [SMILES: COc1ncc(NC(=O)c2cc[nH]c3nccc2-3)cc1S(=O)(=O)Nc1c(C)cccc1C] |
| 2736 | 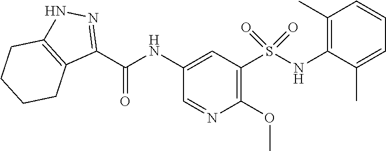 [SMILES: COc1ncc(NC(=O)c2n[nH]c3c2CCCC3)cc1S(=O)(=O)Nc1c(C)cccc1C] |
| 2737 | 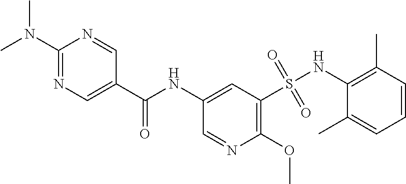 [SMILES: COc1ncc(NC(=O)c2cnc(N(C)C)nc2)cc1S(=O)(=O)Nc1c(C)cccc1C] |
| 2738 | 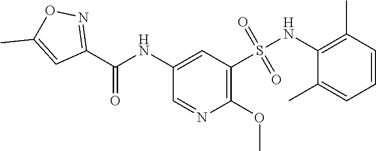 [SMILES: COc1ncc(NC(=O)c2cc(C)on2)cc1S(=O)(=O)Nc1c(C)cccc1C] |
| 2740 | 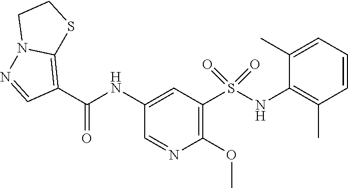 [SMILES: COc1ncc(NC(=O)c2cnn3c2SCC3)cc1S(=O)(=O)Nc1c(C)cccc1C] |
| 2741 | 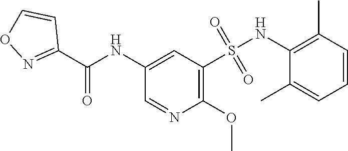 [SMILES: COc1ncc(NC(=O)c2ccon2)cc1S(=O)(=O)Nc1c(C)cccc1C] |
| 2742 | 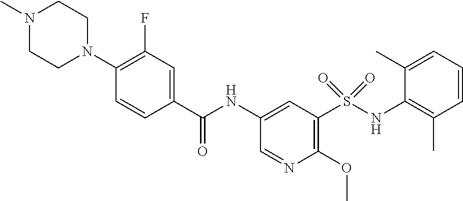 [SMILES: COc1ncc(NC(=O)c2ccc(N3CCN(C)CC3)c(F)c2)cc1S(=O)(=O)Nc1c(C)cccc1C] |
| 2743 | 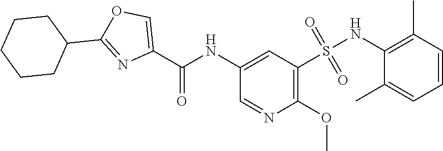 [SMILES: COc1ncc(NC(=O)c2coc(C3CCCCC3)n2)cc1S(=O)(=O)Nc1c(C)cccc1C] |
| 2744 | 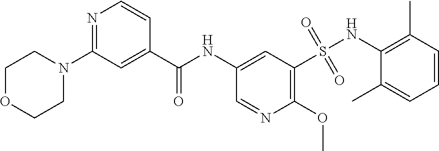 [SMILES: COc1ncc(NC(=O)c2ccnc(N3CCOCC3)c2)cc1S(=O)(=O)Nc1c(C)cccc1C] |
| 2745 | 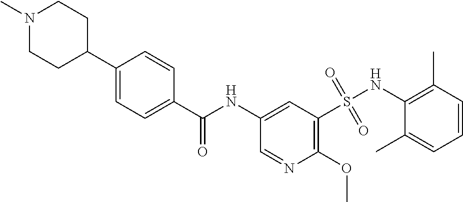 [SMILES: COc1ncc(NC(=O)c2ccc(C3CCN(C)CC3)cc2)cc1S(=O)(=O)Nc1c(C)cccc1C] |
| 2746 | 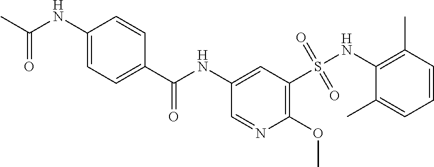 [SMILES: COc1ncc(NC(=O)c2ccc(NC(C)=O)cc2)cc1S(=O)(=O)Nc1c(C)cccc1C] |
| 2747 | 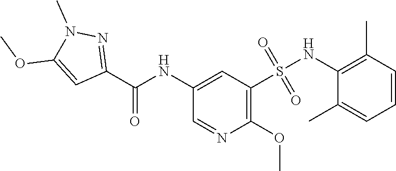 [SMILES: COc1ncc(NC(=O)c2cc(OC)n(C)n2)cc1S(=O)(=O)Nc1c(C)cccc1C] |
| 2749 | 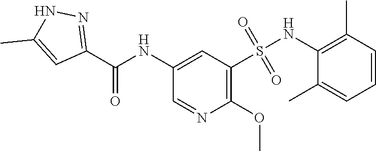 [SMILES: COc1ncc(NC(=O)c2cc(C)[nH]n2)cc1S(=O)(=O)Nc1c(C)cccc1C] |
| 2750 | 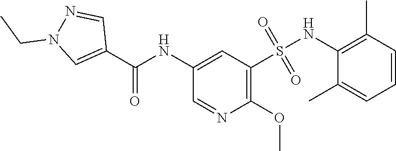 [SMILES: CCn1cc(C(=O)Nc2cnc(OC)c(S(=O)(=O)Nc3c(C)cccc3C)c2)cn1] |
| 2751 | 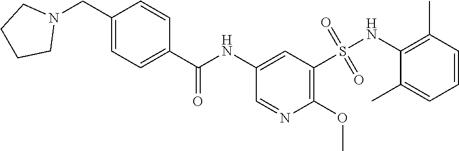 [SMILES: COc1ncc(NC(=O)c2ccc(CN3CCCC3)cc2)cc1S(=O)(=O)Nc1c(C)cccc1C] |
| 2752 | 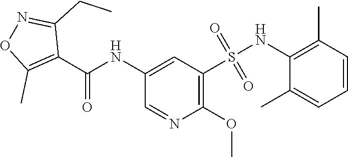 [SMILES: CCc1noc(C)c1C(=O)Nc1cnc(OC)c(S(=O)(=O)Nc2c(C)cccc2C)c1] |
| 2753 | 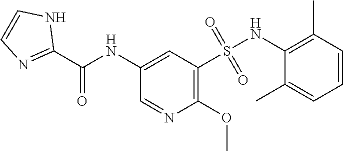 [SMILES: COc1ncc(NC(=O)c2ncc[nH]2)cc1S(=O)(=O)Nc1c(C)cccc1C] |
| 2754 | 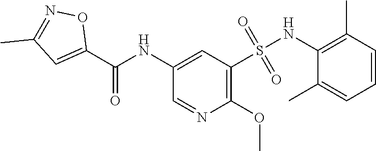 [SMILES: COc1ncc(NC(=O)c2cc(C)no2)cc1S(=O)(=O)Nc1c(C)cccc1C] |
| 2755 | 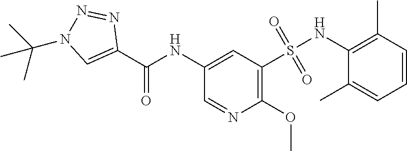 [SMILES: COc1ncc(NC(=O)c2cn(C(C)(C)C)nn2)cc1S(=O)(=O)Nc1c(C)cccc1C] |
| 2756 | 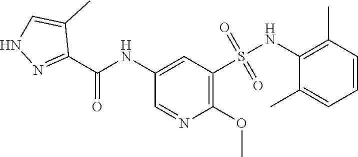 [SMILES: COc1ncc(NC(=O)c2n[nH]cc2C)cc1S(=O)(=O)Nc1c(C)cccc1C] |
| 2758 | 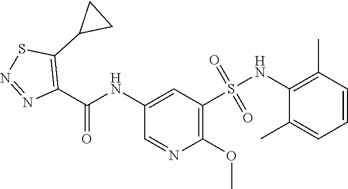 [SMILES: COc1ncc(NC(=O)c2nnsc2C2CC2)cc1S(=O)(=O)Nc1c(C)cccc1C] |
| 2759 | 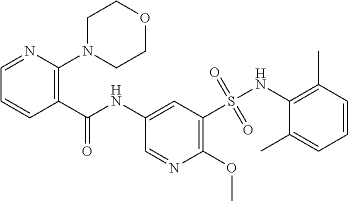 [SMILES: COc1ncc(NC(=O)c2cccnc2N2CCOCC2)cc1S(=O)(=O)Nc1c(C)cccc1C] |
| 2761 | 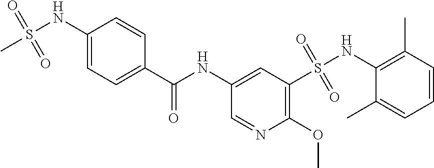 [SMILES: COc1ncc(NC(=O)c2ccc(NS(C)(=O)=O)cc2)cc1S(=O)(=O)Nc1c(C)cccc1C] |
| 2762 | 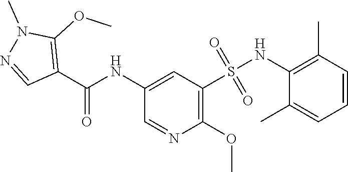 [SMILES: COc1ncc(NC(=O)c2cnn(C)c2OC)cc1S(=O)(=O)Nc1c(C)cccc1C] |
| 2764 | 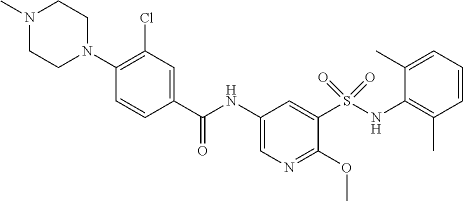 [SMILES: COc1ncc(NC(=O)c2ccc(N3CCN(C)CC3)c(Cl)c2)cc1S(=O)(=O)Nc1c(C)cccc1C] |
| 2766 | 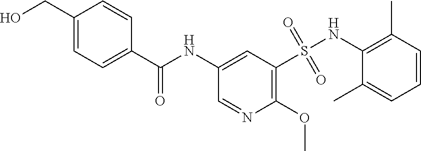 [SMILES: COc1ncc(NC(=O)c2ccc(CO)cc2)cc1S(=O)(=O)Nc1c(C)cccc1C] |
| 2767 | 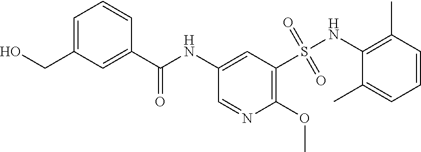 [SMILES: COc1ncc(NC(=O)c2cccc(CO)c2)cc1S(=O)(=O)Nc1c(C)cccc1C] |
| 2768 | 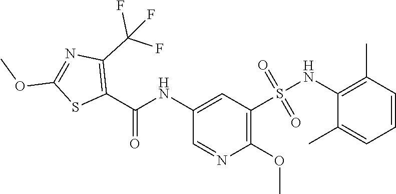 [SMILES: COc1nc(C(F)(F)F)c(C(=O)Nc2cnc(OC)c(S(=O)(=O)Nc3c(C)cccc3C)c2)s1] |
| 2769 | 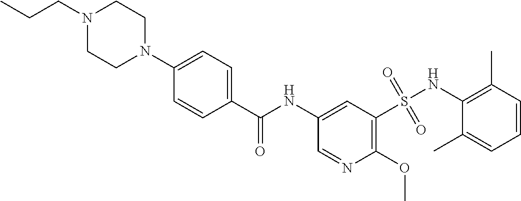 [SMILES: CCCN1CCN(c2ccc(C(=O)Nc3cnc(OC)c(S(=O)(=O)Nc4c(C)cccc4C)c3)cc2)CC1] |
| 2770 | 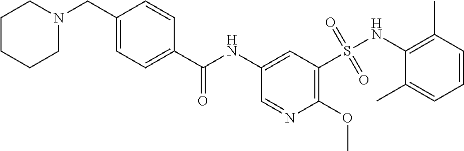 [SMILES: COc1ncc(NC(=O)c2ccc(CN3CCCCC3)cc2)cc1S(=O)(=O)Nc1c(C)cccc1C] |
| 2771 | 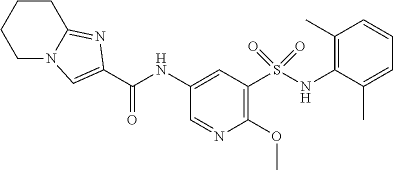 [SMILES: COc1ncc(NC(=O)c2cn3c(n2)CCCC3)cc1S(=O)(=O)Nc1c(C)cccc1C] |
| 2772 | 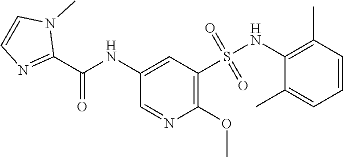 [SMILES: COc1ncc(NC(=O)c2nccn2C)cc1S(=O)(=O)Nc1c(C)cccc1C] |
| 2774 | 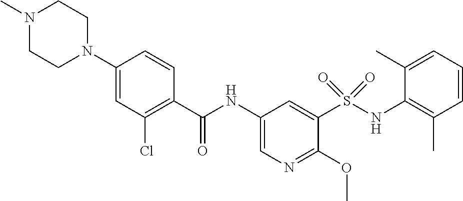 [SMILES: COc1ncc(NC(=O)c2ccc(N3CCN(C)CC3)cc2Cl)cc1S(=O)(=O)Nc1c(C)cccc1C] |
| 2775 | 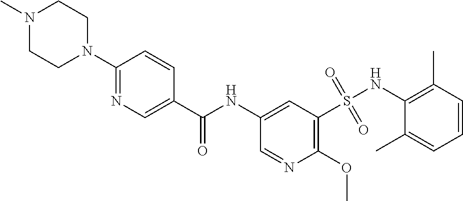 [SMILES: COc1ncc(NC(=O)c2ccc(N3CCN(C)CC3)nc2)cc1S(=O)(=O)Nc1c(C)cccc1C] |
| 2776 | 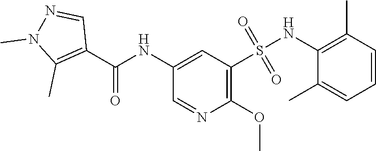 [SMILES: COc1ncc(NC(=O)c2cnn(C)c2C)cc1S(=O)(=O)Nc1c(C)cccc1C] |
| 2777 | 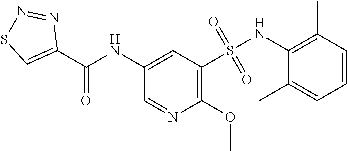 [SMILES: COc1ncc(NC(=O)c2csnn2)cc1S(=O)(=O)Nc1c(C)cccc1C] |
| 2779 | 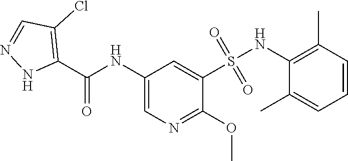 [SMILES: COc1ncc(NC(=O)c2[nH]ncc2Cl)cc1S(=O)(=O)Nc1c(C)cccc1C] |
| 2780 | 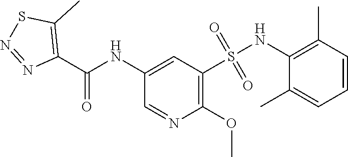 [SMILES: COc1ncc(NC(=O)c2nnsc2C)cc1S(=O)(=O)Nc1c(C)cccc1C] |
| 2781 | 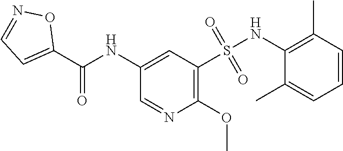 [SMILES: COc1ncc(NC(=O)c2ccno2)cc1S(=O)(=O)Nc1c(C)cccc1C] |
| 2782 | 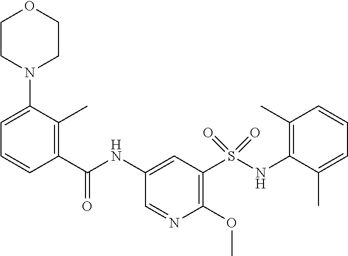 [SMILES: COc1ncc(NC(=O)c2cccc(N3CCOCC3)c2C)cc1S(=O)(=O)Nc1c(C)cccc1C] |
| 2784 | 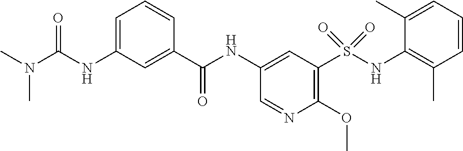 [SMILES: COc1ncc(NC(=O)c2cccc(NC(=O)N(C)C)c2)cc1S(=O)(=O)Nc1c(C)cccc1C] |
| 2785 | 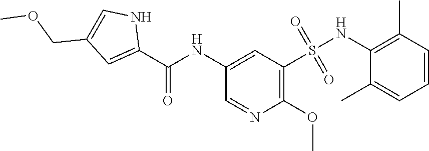 [SMILES: COCc1c[nH]c(C(=O)Nc2cnc(OC)c(S(=O)(=O)Nc3c(C)cccc3C)c2)c1] |
| 2786 | 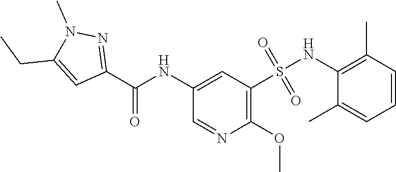 [SMILES: CCc1cc(C(=O)Nc2cnc(OC)c(S(=O)(=O)Nc3c(C)cccc3C)c2)nn1C] |
| 2787 | 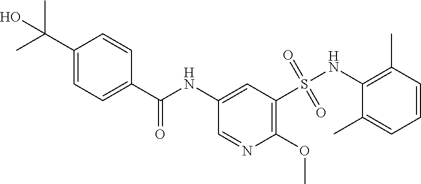 [SMILES: COc1ncc(NC(=O)c2ccc(C(C)(C)O)cc2)cc1S(=O)(=O)Nc1c(C)cccc1C] |
| 2788 | 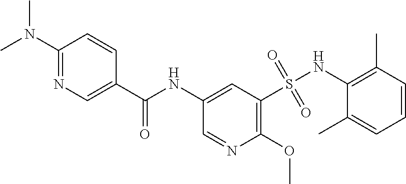 [SMILES: COc1ncc(NC(=O)c2ccc(N(C)C)nc2)cc1S(=O)(=O)Nc1c(C)cccc1C] |
| 2789 | 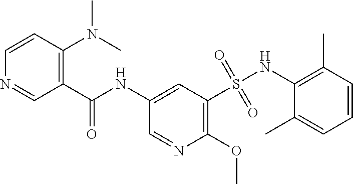 [SMILES: COc1ncc(NC(=O)c2cnccc2N(C)C)cc1S(=O)(=O)Nc1c(C)cccc1C] |
| 2790 | 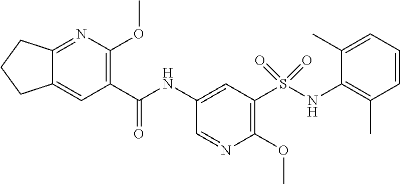 [SMILES: COc1nc2c(cc1C(=O)Nc1cnc(OC)c(S(=O)(=O)Nc3c(C)cccc3C)c1)CCC2] |
| 2791 | 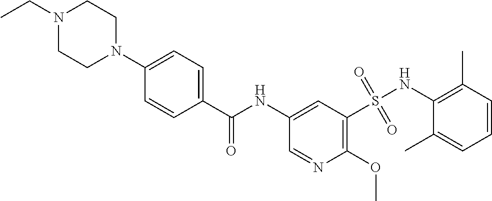 [SMILES: CCN1CCN(c2ccc(C(=O)Nc3cnc(OC)c(S(=O)(=O)Nc4c(C)cccc4C)c3)cc2)CC1] |
| 2792 | 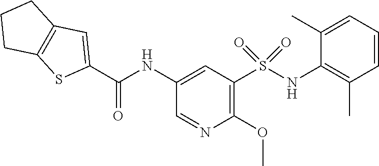 [SMILES: COc1ncc(NC(=O)c2cc3c(s2)CCC3)cc1S(=O)(=O)Nc1c(C)cccc1C] |
| 2793 | 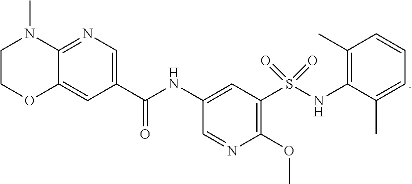 [SMILES: COc1ncc(NC(=O)c2cnc3c(c2)OCCN3C)cc1S(=O)(=O)Nc1c(C)cccc1C] |

68. The compound of any one of claims 1-67, or a pharmaceutically acceptable salt thereof.

69. A pharmaceutical composition comprising:
a compound of any one of claims 1-67, or a pharmaceutically acceptable salt, solvate, hydrate, polymorph, co-crystal, tautomer, stereoisomer, isotopically labeled compound, or prodrug thereof; and
optionally a pharmaceutically acceptable excipient.

70. The pharmaceutical composition of claim 69 further comprising an additional pharmaceutical agent.

71. The pharmaceutical composition of claim 70, wherein the additional pharmaceutical agent is metformin.

72. A kit comprising:
a compound of any one of claims 1-67, or a pharmaceutically acceptable salt, solvate, hydrate, polymorph, co-crystal, tautomer, stereoisomer, isotopically labeled compound, or prodrug thereof, or a pharmaceutical composition of any one of claims 69-71; and
instructions for using the compound, or a pharmaceutically acceptable salt, solvate, hydrate, polymorph, co-crystal, tautomer, stereoisomer, isotopically labeled compound, or prodrug thereof, or the pharmaceutical composition.

73. A method of treating a disease in a subject in need thereof, the method comprising administering to the subject in need thereof an effective amount of a compound of any one of claims 1-67, or a pharmaceutically acceptable salt, solvate, hydrate, polymorph, co-crystal, tautomer, stereoisomer, isotopically labeled compound, or prodrug thereof, or a pharmaceutical composition of any one of claims 69-71.

74. A method of preventing a disease in a subject in need thereof, the method comprising administering to the subject in need thereof an effective amount of a compound of any one of claims 1-67, or a pharmaceutically acceptable salt, solvate, hydrate, polymorph, co-crystal, tautomer, stereoisomer, isotopically labeled compound, or prodrug thereof, or a pharmaceutical composition of any one of claims 69-71.

75. The method of any one of claims 73-74, wherein the disease is associated with decreased expression and/or activity of tumor necrosis factor (TNF) receptor associated protein 1 (TRAP1).

76. The method of claim 75, wherein the disease is associated with decreased activity of TRAP1.

77. The method of any one of claims 73-76, wherein the disease is associated with a mutation in the gene encoding tumor necrosis factor (TNF) receptor associated protein 1 (TRAP1).

78. The method of any one of claims 73-77, wherein the effective amount is effective in increasing the expression and/or activity of tumor necrosis factor (TNF) receptor associated protein 1 (TRAP1).

79. The method of claim 78, wherein the effective amount is effective in increasing the activity of TRAP1.

80. The method of any one of claims 73-79, wherein the disease is associated with decreased expression and/or activity of PTEN induced putative kinase 1 (PINK1).

81. The method of any one of claims 73-80, wherein the disease is associated with a mutation in the gene encoding PTEN induced putative kinase 1 (PINK1).

82. The method of any one of claims 73-81, wherein the disease is associated with increased protein misfolding and/or protein aggregation.

83. The method of any one of claims 73-82, wherein the disease is a proteopathy.

84. The method of claim 83, wherein the disease is a synucleinopathy.

85. The method of claim 82, wherein the protein is α-synuclein.

86. The method of claim 84, wherein the disease is Lewy body dementia or multiple system atrophy.

87. The method of any one of claims 73-86, wherein the disease is associated with decreased health, quality, function, quantity, and/or activity of mitochondria.

88. The method of any one of claims 73-87, wherein the disease is a mitochondrial disease.

89. The method of any one of claims 73-88, wherein the disease is associated with increased production of reactive oxygen species.

90. The method of any one of claims 73-89, wherein the disease is a neurodegenerative disease.

91. The method of claim 90, wherein the disease is Parkinson's disease.

92. The method of claim 90, wherein the disease is Parkinson's disease associated with a mutation in the gene encoding PINK1.

93. The method of claim 90, wherein the disease is Huntington's disease, Alzheimer's disease, dementia, amyotrophic lateral sclerosis, or Friedreich's ataxia.

94. The method of claim 90, wherein the disease is frontotemporal dementia.

95. The method of any one of claims 73-85 and 87-89, wherein the disease is a kidney disease.

96. The method of claim 95, wherein the disease is polycystic kidney disease.

97. The method of claim 95, wherein the disease is autosomal dominant polycystic kidney disease.

98. The method of any one of claims 73-85 and 87-89, wherein the disease is a lysosomal storage disease.

99. The method of claim 98, wherein the disease is Tay-Sachs disease, Sandhoff disease, Niemann-Pick disease, Fabry disease, or Gaucher's disease.

100. The method of any one of claims 73-85 and 87-89, wherein the disease is chronic pain, fatigue, gastrointestinal dysmotility, congenital abnormality of the kidney and urinary tract, VACTERL association, or cardiac hypertrophy.

101. A method of increasing the expression and/or activity of tumor necrosis factor (TNF) receptor associated protein 1 (TRAP1) in a subject in need thereof, the method comprising administering to the subject in need thereof an effective amount of a compound of any one of claims 1-67, or a pharmaceutically acceptable salt, solvate, hydrate, polymorph, co-crystal, tautomer, stereoisomer, isotopically labeled compound, or prodrug thereof, or a pharmaceutical composition of any one of claims 69-71.

102. A method of increasing the health, quality, function, quantity, and/or activity of mitochondria in a subject in need thereof, the method comprising administering to the subject in need thereof an effective amount of a compound of any one of claims 1-67, or a pharmaceutically acceptable salt, solvate, hydrate, polymorph, co-crystal, tautomer, stereoisomer, isotopically labeled compound, or prodrug thereof, or a pharmaceutical composition of any one of claims 69-71.

103. The method of any one of claims 73-102, wherein the subject is a human.

104. A method of increasing the expression and/or activity of tumor necrosis factor (TNF) receptor associated protein 1 (TRAP1) in a cell, tissue, or biological sample, the method comprising contacting the cell, tissue, or biological sample with an effective amount of a compound of any one of claims 1-67, or a pharmaceutically acceptable salt, solvate, hydrate, polymorph, co-crystal, tautomer, stereoisomer, isotopically labeled compound, or prodrug thereof, or a pharmaceutical composition of any one of claims 69-71.

105. A method of increasing the health, quality, function, quantity, and/or activity of mitochondria in a cell, tissue, or biological sample, the method comprising contacting the cell, tissue, or biological sample with an effective amount of a compound of any one of claims 1-67, or a pharmaceutically acceptable salt, solvate, hydrate, polymorph, co-crystal, tautomer, stereoisomer, isotopically labeled compound, or prodrug thereof, or a pharmaceutical composition of any one of claims 69-71.

106. The method of any one of claims 104-105, wherein the cell, tissue, or biological sample is in vitro.
